(19) United States
(12) Patent Application Publication
(10) Pub. No.: US 2009/0131395 A1
(43) Pub. Date: May 21, 2009

(54) BIPHENYLAZETIDINONE CHOLESTEROL ABSORPTION INHIBITORS

(75) Inventors: Stephen Antonelli, Lynn, MA (US); Regina Lundrigan, Charlestown, MA (US); Eduardo J. Martinez, St. Louis, MO (US); Wayne C. Schairer, Westboro, MA (US); John J. Talley, Somerville, MA (US); Timothy C. Barden, Salem, MA (US); JingJing Yang, Boxborough, MA (US); Daniel P. Zimmer, Somerville, MA (US)

Correspondence Address: HESLIN ROTHENBERG FARLEY & MESITI PC, 5 COLUMBIA CIRCLE, ALBANY, NY, 12203, US

(73) Assignee: MICROBIA, INC., Cambridge, MA (US)

(21) Appl. No.: 11/913,461

(22) PCT Filed: May 5, 2006
(86) PCT No.: PCT/US06/17412

§ 371 Date: May 30, 2008

Related U.S. Application Data
(60) Provisional application No. 60/677,976, filed on May 5, 2005.

(51) Int. Cl. A61K 31/397; C07D 205/08; A61P 9/10

(52) U.S. Cl. 514/210.02; 540/360

(57) ABSTRACT

The invention relates to a chemical genus of 4-biphenyl-1-phenylazetidin-2-ones useful in the treatment of hypercholesterolemia and other disorders. The compounds have the general formula I:

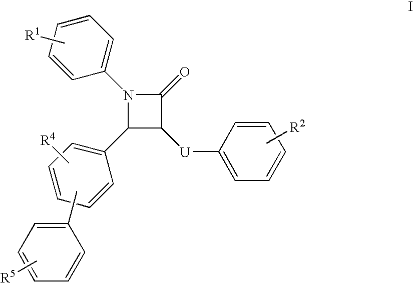

Pharmaceutical compositions and methods for treating cholesterol- and lipid-associated diseases are also disclosed.

### FIELD OF THE INVENTION

The present invention relates to a chemical genus of 4-biphenyl-1-phenylazetidin-2-ones, useful in the treatment of hypercholesterolemia and other disorders.

### BACKGROUND OF THE INVENTION

1,4-Diphenylazetidin-2-ones and their utility for treating disorders of lipid metabolism are described in U.S. Pat. No. 6,498,156, USRE37721, in U.S. published patent applications 2004/0067913, 2004/0082561 and 2004/0198700, in PCT applications WO02/50027, WO2004/005247, WO2004/087655 and in European applications EP 1 362 855 and 1 522 541, the disclosures of which are incorporated herein by reference.

4-Biaryl-1-phenylazetidin-2-ones and their utility for treating disorders of lipid metabolism are described in copending commonly owned U.S. patent application Ser. No. 10/986,570 filed Nov. 10, 2004, the disclosure of which is incorporated herein by reference.

### SUMMARY OF THE INVENTION

In a first aspect, the invention relates to compounds of formula I

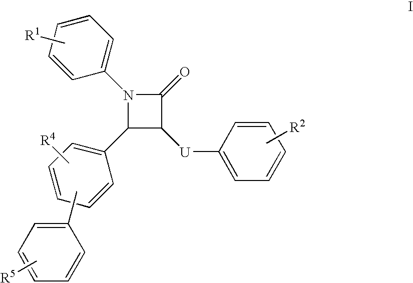

wherein
R¹ and R² are selected independently from the group consisting of hydrogen, halogen, —OH, and lower alkoxy;
R⁴ is selected from the group consisting of hydrogen, —OH, and a glucuronide;
R⁵ is selected from the group consisting of —OH, —PO₃H₂, —SO₃H, a sugar, a polyol, a glucuronide and a sugar carbamate; and
U is (C₂-C₆)-alkylene in which one or more —CH₂— is replaced by a radical chosen from —S—, —S(═O)—, —SO₂—, —O—, —C(═O)—, —CH(glucuronide)-, and —CH(OH)—.

The invention also includes pharmaceutically acceptable salts thereof, in any stereoisomeric form, or a mixture of any such compounds in any ratio.

The compounds described herein can be included in pharmaceutical formulations comprising a pharmaceutically acceptable carrier and one or more of: (1) a dyslipidemic agent, (2) an anti-diabetic agent, (3) an anti-hypertensive agent, (4) an anti-obesity agent, (5) an agent used to treat autoimmune disorders, (6) an agent used to treat demylenation and its associated disorders, (7) an agent used to treat Alzheimer's disease, (8) a blood modifier, (9) a hormone replacement agent/composition, (10) a chemotherapeutic agent, (11) a peptide which mitigates one or more symptoms of atherosclerosis, (12) an anti-cancer agent, (13) an agent used to treat bone loss and associated disorders, and (14) other agents.

The compounds and pharmaceutical formulations described herein can be used in methods for treating a condition for which a cholesterol absorption inhibitor is indicated; preventing or treating a cholesterol related disease; inhibiting the absorption of or reducing plasma or tissue concentration of one or more sterols or stanols; preventing or treating sistoserolemia; preventing or treating vascular diseases/disorders and conditions, dyslipidemia, mixed dyslipidemia, hypo α-lipoproteinemia, LDL pattern B, LDL pattern A, primary dysbetalipoproteinemia (Frederickson Type III), hyperlipidemia (including but not limited to hypercholesterolemia, hypertriglyceridemia, sitosterolemia), hypertension, angina pectoris, cardiac arrhythmias, congestive heart failure, and stroke; reducing the incidence of cardiovascular disease-related events; preventing or treating vascular conditions and associated thrombotic events; preventing or treating vascular inflammation; reducing blood plasma or serum concentrations of C-reactive protein; preventing, treating, or ameliorating symptoms of Alzheimer's Disease (AD); regulating production or levels of at least one amyloid β (Aβ) peptide; regulating the amount of ApoE isoform 4 in the bloodstream and/or brain; preventing or treating cognitive related disorders (including dementia); preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cholesterol associated tumors; inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; modulating lipid raft structure; and preventing or treating osteopenia disorders (bone loss disorders). The methods comprise administering a therapeutically effective amount of a compound or pharmaceutical formulation described herein.

The invention relates to an article of manufacture comprising a container, instructions, and a pharmaceutical formulation as described above. The instructions are for the administration of the pharmaceutical formulation for a purpose chosen from: treating a condition for which a cholesterol absorption inhibitor is indicated; preventing or treating a cholesterol related disease; inhibiting the absorption of or reducing plasma or tissue concentration of one or more sterols or stanols; preventing or treating sistoserolemia; preventing or treating vascular diseases/disorders and conditions, dyslipidemia, mixed dyslipidemia, hypo α-lipoproteinemia, LDL pattern B, LDL pattern A, primary dysbetalipoproteinemia (Frederickson Type III), hyperlipidemia (including but not limited to hypercholesterolemia, hypertriglyceridemia, sitosterolemia), hypertension, angina pectoris, cardiac arrhythmias, congestive heart failure, and stroke; reducing the incidence of cardiovascular disease-related events; preventing or treating vascular conditions and associated thrombotic events; preventing or treating vascular inflammation; reducing blood plasma or serum concentrations of C-reactive protein; preventing, treating, or ameliorating symptoms of Alzheimer's Disease (AD); regulating production or levels of at least one amyloid β (Aβ) peptide; regulating the amount of ApoE isoform 4 in the bloodstream and/or brain; preventing or treating cognitive related disorders (including dementia); preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cholesterol associated tumors; inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; modulating lipid raft structure; treating or preventing a diseasee associated with lipid raft structure; and preventing or treating osteopenia disorders (bone loss disorders).

These and other embodiments of the present invention will become apparent in conjunction with the description and claims that follow.

### DETAILED DESCRIPTION OF THE INVENTION

The substituents and variables defined herein retain the same definition throughout.

In the present compounds of formula I, a biphenyl is located at the 4-position of the azetidinone ring. Thus, one subgenus includes compounds of formula Ia

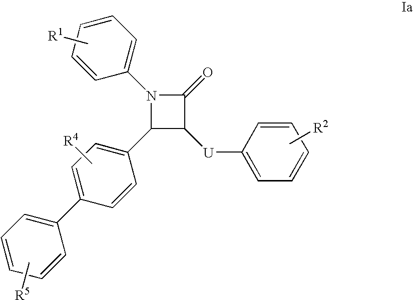

However, the invention is not limited to this particular embodiment depicted by formula Ia.

The phenyl group having substituent R² is linked to the 3-position of the azetidinone ring via a (C₂-C₆)-alkylene in which one or more —CH₂— is replaced by a radical chosen from —S—, —S(═O)—, —SO₂—, —O—, —C(═O)—, —CH(glucuronide)-, and —CH(OH)—. “U” represents this alkylene in formulae I and Ia. Often U is (C₂-C₄)-alkylene in which one or more —CH₂— is replaced.

In some embodiments, one or more —CH₂— in the alkylene chain is replaced by —CH(OH)—. In other embodiments, one or more —CH₂— is replaced by —CH(glucuronide)-.

Examples of U linkages include, but are not limited to the following: —SCH₂CH(OH)—; S(═O)CH₂CH(OH)—; —SO₂CH₂CH(OH)—; —SCH₂C(═O)—; S(═O)CH₂C(═O)—; —SO₂CH₂C(═O)—; —OCH₂CH(OH)—; —OCH₂C(═O)—; —CH(OH)CH₂O—; —C(═O)CH₂O—; —CH(OH)CH₂S—; —C(═O)CH₂S—; —CH₂CH(OH)—; —CH₂CH₂CH₂CH(OH)—; —OCH₂CH(═O)—; —SCH₂CH₂CH(OH)—; —SCH₂CH₂C(═O)—; —S(═O)CH₂CH₂CH(OH)—; —OCH₂CH₂CH(OH)—; —SO₂CH₂CH₂CH(OH)—; —SO₂CH₂CH₂C(═O)—; —CH(OH)CH₂CH(OH)—; —OCH₂CH₂CH(OH)—; —SCH₂CH(glucuronide)-; —S(═O)CH₂CH(glucuronide)-; —SO₂CH₂CH(glucuronide)-; —OCH₂CH(glucuronide)-; —CH(glucuronide)CH₂O—; —CH(glucuronide)CH₂S—; —CH₂CH(glucuronide)-; —CH₂CH₂CH₂CH(glucuronide)-; —SCH₂CH₂CH(glucuronide)-; —S(═O)CH₂CH₂CH(glucuronide)-; —OCH₂CH₂CH(glucuronide)-; —SO₂CH₂CH₂CH(glucuronide)-; —CH(glucuronide)CH₂CH(glucuronide)-; and —OCH₂CH₂CH(glucuronide)-. Other variations for U would be obvious to those of ordinary skill in the chemical arts. In addition, embodiments of C₅- and C₆-alkylenes, in which one or more —CH₂— is replaced would be obvious to one of ordinary skill based on the examples provided herein.

As previously mentioned, the invention covers all stereoisomeric forms of compounds having formulae I and Ia. Thus, in certain embodiments, the 3-position of the azetidinone ring is of the (S)-configuration, and the 4-position of the azetidinone ring is of the (R)-configuration. In certain other embodiments, the 3-position of the azetidinone ring is of the (R)-configuration, and the 4-position of the azetidinone ring is of the (S)-configuration.

In addition, when the U chain includes one or more (OH) or (glucuronide) substituents, i.e. —CH₂— is replaced by —CH(OH)— or —(CH(glucuronide), all stereoisomeric forms of the substituents are encompassed in the present invention. Thus, in some embodiments, each (OH) or (glucuronide) substituent in the U chain is of the (S)-configuration. In others, each (OH) or (glucuronide) is of the (R)-configuration.

In compounds of formula I, as well as in those of formula Ia, R¹ and R² are each independently hydrogen, halogen, —OH, or lower alkoxy. In some embodiments, R¹ is hydrogen, and in some embodiments of the compounds, R² is fluorine. In certain embodiments, R¹ is in the para position on its corresponding phenyl ring. Likewise, in certain embodiments, R² is a para substituent.

In addition, R⁴ is hydrogen, —OH, or a glucuronide. In certain embodiments, R⁴ is —OH. However, in certain other embodiments, R⁴ is a glucuronide, as defined herein. Often, R⁴ is located ortho to the azetidinone ring.

In the present compounds, R⁵ is —OH, —PO₃H₂, —SO₃H, a sugar, a polyol, a glucuronide or a sugar carbamate, as defined herein. In certain embodiments R⁵ is located at the para position, and in certain other embodiments, R⁵ is a meta substituent on its corresponding phenyl ring.

Some of the compounds of the invention may contain basic or acidic residues, allowing them to be presented as salts. The term “pharmaceutically acceptable salt” refers to salts whose counter ion derives from pharmaceutically acceptable non-toxic acids and bases. When the compounds contain a quat or a basic residue, suitable pharmaceutically acceptable base addition salts for the compounds of the present invention include inorganic acids, organic acids and, in the case of quats, water (which formally furnishes the hydroxide anion). Examples include hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate, and the like. When the compounds contain an acidic residue, suitable pharmaceutically acceptable base addition salts for the compounds of the present invention include ammonium, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Other base addition salts includes those made from: arecoline, arginine, barium, benethamine, benzathine, betaine, bismuth, clemizole, copper, deanol, diethylamine, diethylaminoethanol, epolamine, ethylenediamine, ferric, ferrous, glucamine, glucosamine, histidine, hydrabamine, imidazole, isopropylamine, manganic, manganous, methylglucamine, morpholine, morpholineethanol, n-ethylmorpholine, n-ethylpiperidine, piperazine, piperidine, polyamine resins, purines, theobromine, triethylamine, trimethylamine, tripropylamine, trolamine, and tromethamine.

### Therapeutic Indications

The present invention further provides methods for treating a condition for which a cholesterol absorption inhibitor is indicated; preventing or treating a cholesterol related disease; inhibiting the absorption of or reducing plasma or tissue concentration of one or more sterols or stanols; preventing or treating sistoserolemia; preventing or treating vascular diseases/disorders and conditions, dyslipidemia, mixed dyslipidemia, hypo α-lipoproteinemia, LDL pattern B, LDL pattern A, primary dysbetalipoproteinemia (Frederickson Type III), hyperlipidemia (including but not limited to hypercholesterolemia, hypertriglyceridemia, sitosterolemia), hypertension, angina pectoris, cardiac arrhythmias, congestive heart failure, and stroke; reducing the incidence of cardiovascular disease-related events; preventing or treating vascular conditions and associated thrombotic events; preventing or treating vascular inflammation; reducing blood plasma or serum concentrations of C-reactive protein; preventing, treating, or ameliorating symptoms of Alzheimer's Disease (AD); regulating production or levels of at least one amyloid β (Aβ) peptide; regulating the amount of ApoE isoform 4 in the bloodstream and/or brain; preventing or treating cognitive related disorders (including dementia); preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cholesterol associated tumors; inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; modulating lipid raft structure; and preventing or treating osteopenia disorders (bone loss disorders). The methods comprise administering a therapeutically effective amount of a compound described herein.

The compounds described herein may inhibit cholesterol absorption and thus reduce cholesterol levels in vivo. The compositions and therapeutic methods described herein are useful for treating any condition for which a cholesterol absorption inhibitor is indicated. When administered to a patient, the compositions and pharmaceutical formulations described herein can lead to one or more of: reduced blood plasma or serum concentrations of low-density lipoprotein cholesterol (LDL-C); reduced blood plasma or serum concentrations of very low-density lipoprotein cholesterol (VLDL-C); reduced blood plasma or serum concentrations of intermediate-density lipoprotein cholesterol (IDL-C); reduced concentrations of cholesterol and cholesterol ester in the blood plasma or serum; reduced blood plasma or serum concentrations of apolipoprotein B; reduced blood plasma or serum concentrations of triglycerides; increased clearance of triglycerides; increased blood plasma or serum concentrations of high density lipoprotein cholesterol (HDL-C); reduced blood plasma or serum concentrations of non high-density lipoprotein cholesterol (non HDL-C); reduced levels of lipoprotein(a) (Lp(a)); increased ratio of HDL-C to LDL-C; inhibition of saponified and/or non-saponified fatty acid synthesis; reduced blood plasma or serum concentrations apolipoprotein C-II; reduced blood plasma or serum concentrations apolipoprotein C-III; increased blood plasma or serum concentrations of HDL associated proteins (including but not limited to apo A-I, apo A-II, apo A-IV, and apo E), and increased fecal excretion of cholesterol.

The compounds and pharmaceutical formulations described herein can be used alone or in combination therapy with one or more additional agents (e.g., one or more dyslipidemic agents, peptides which mitigates one or more symptoms of atherosclerosis, other agents, including combinations thereof) to prevent or treat a cholesterol related disease. Cholesterol related diseases include but are not limited to diseases involving elevated levels of LDL cholesterol, diseases involving regulation of LDL receptors, diseases involving reduced levels of HDL cholesterol, dyslipidemia, diseases involving elevated levels of non-esterified fatty acids, diseases involving reduced or deficient lipoprotein lipase levels or activity (including reductions or deficiencies resulting from lipoprotein lipase mutations), diseases involving elevated levels of ketone bodies (e.g., β-OH butyric acid), hyperlipidemia, elevated LDL Pattern B, elevated LDL Pattern A, primary dysbetalipoproteinemia (Frederickson Type III), hypercholesterolemia, hypo α-lipoproteinemia (low HDL cholesterol syndrome), hyperlipoproteinemia, elevated Lp(a) levels, hypertriglyceridemia (including Frederickson types IV and V), other aberrations of apolipoprotein B metabolism, homozygous familial hypercholesterolemia, heterozygous familial hypercholesterolemia, presumed familial combined and non-familial (non-FH) forms of primary hypercholesterolemia (including Frederickson Types IIa and IIb), cholesterol ester storage disease, and cholesterol ester transfer protein disease.

The compounds and pharmaceutical formulations described herein can be used alone or in combination therapy with one or more additional agents (e.g., one or more dyslipidemic agents, peptides which mitigates one or more symptoms of atherosclerosis, other agents, including combinations thereof) to inhibit the absorption of or reduce plasma or tissue concentration of one or more sterols (referring to, for example: (from any source and in any form: α, β and γ) saturated or hydrogenated sterols including all natural or synthesized forms and derivatives thereof, and isomers including but not limited to cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol) or stanols (referring to, for example: (from any source and in any form: α, β and γ) saturated or hydrogenated stanols including all natural or synthesized forms and derivatives thereof, and isomers, including but not limited to sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, and pollinastastanol and 5α-stanols (e.g., cholestanol, 5α-campestanol, 5α-sitostanol) or mixtures thereof in a subject in need of such treatment, for example, a sitosterolemic subject. Sterols and stanols also include free sterols and stanols, esterified sterols and stanols with aliphatic or aromatic acids (thereby forming aliphatic or aromatic esters, respectively), phenolic acid esters, cinnamate esters, ferulate esters, phytosterol and phytostanol glycosides and acylated glycosides or acylglycosides. Thus, terms the sterols and stanols encompasses all analogues, which may further have a double bond at the 5-position in the cyclic unit as in most natural sterols, or one or more double bonds at other positions in the rings (for example, 6, 7, 8(9), 8(14), 14 5/7) or no double bonds in the cyclic unit as in stanols.

The compounds and pharmaceutical formulations described herein can be used alone or in combination therapy with one or more additional agents (e.g., one or more dyslipidemic agents, peptides which mitigates one or more symptoms of atherosclerosis, other agents, including combinations thereof) to prevent or treat sistoserolemia in patients who are either at risk of developing sistoserolemia or already exhibit sistoserolemia, for example, as described in US20020169134. Sitosterolemia is a genetic lipid storage disorder characterized by increased levels of sitosterol and other plant sterols in the plasma and other tissues due to increased non-selective intestinal absorption of sterols and decreased hepatic removal. Individuals having sitosterolemia can exhibit one or more of the following conditions: tendon and tuberous xanthomas, arthritis, hemolytic episodes, accelerated atherosclerosis and myocardial infarctions, and can die at an early age due to extensive coronary atherosclerosis (see Nguyen et al. Journal of Lipid Research, 32: 1941-1948).

The compounds and pharmaceutical formulations described herein can be used alone or in combination therapy with one or more additional agents (e.g., one or more anti-hypertensive agents, dyslipidemic agents, peptides which mitigates one or more symptoms of atherosclerosis, other agents, including combinations thereof) to prevent or treat vascular diseases/disorders and conditions (including but not limited to arteriosclerosis, atherosclerosis, acute vascular syndromes, peripheral arterial disease, cardiovascular disease, cerebrovascular disease (e.g., cerebral infarction or stroke (caused by vessel blockage or hemorrhage), or transient ischemia attack (TIA), syncope, atherosclerosis of the intracranial and/or extracranial arteries, and the like), renovascular disease, mesenteric vascular disease, pulmonary vascular disease, ocular vascular disease, microvascular disease (such as nephropathy, neuropathy, retinopathy), and peripheral vascular disease), hyperlipidemia (including but not limited to hypercholesterolemia, hypertriglyceridemia, sitosterolemia), hypertension, angina pectoris (including stable, chronic stable, vasospastic, and unstable angina), cardiac arrhythmias, congestive heart failure, and stroke in patients who are at risk for such a disease/condition or in need of such treatment for example, as described, in US2002147184 and US20030069221. Vascular disease is a term that broadly encompasses all disorders of blood vessels including small and large arteries and veins and blood flow. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders e.g., coronary artery disease, aortic aneurysm, arterial disease of the lower extremities and cerebrovascular disease. Other vascular conditions frequently coexist with cholesterol levels associated with atherosclerosis. These may include hypertension, angina and/or arrhythmia. Vascular conditions may be caused or aggravated by hypertension which is defined as persistently high blood pressure. Generally, adults are classified as being hypertensive when systolic blood pressure is persistently above 140 mmHg or when diastolic blood pressure is above 90 mmHG. Elevated blood pressure is a risk factor for atherosclerosis, cardiovascular and cerebrovascular disease.

The compounds and pharmaceutical formulations described herein can be used alone or in combination therapy with one or more additional agents (e.g., one or more anti-hypertensive agents, dyslipidemic agents, peptides which mitigates one or more symptoms of atherosclerosis, other agents, including combinations thereof) to reduce the incidence of cardiovascular disease-related events, for example, as described in US20050080071. Thus the compounds and pharmaceutical formulations described herein can be used to prevent or reduce the risk of an occurrence of a fatal or non-fatal cardiovascular event in patients having no history of clinically evident coronary heart disease, as well as patients having a history of clinically evident coronary heart disease (CHD). A total cholesterol level in excess of 225-250 mg/dl is associated with significant elevation of risk of CHD. The newly revised NCEP ATP III low density lipoprotein (LDL-C) goal for patients with CHD or CHD risk equivalent is <100 mg/dL (2.59 mmol/L), for individuals with two or more risk factors is <130 mg/dL (3.37 mmol/L) and for individuals with fewer than two risk factors is <160 mg/dL (4.14 mmol/L). The phrase “cardiovascular event” includes but is not limited to fatal and non-fatal acute major coronary events, coronary revascularization procedures, myocardial revascularization procedures, peripheral vascular disease, stable angina and cerebrovascular insufficiency e.g., stroke. The phrase “acute major coronary event” includes fatal myocardial infarction, witnessed and unwitnessed cardiac death and sudden death occurring from 1 hour up to 24 hours after collapse, non-fatal myocardial infarction including definite acute Q-wave myocardial infarction, non-Q-wave myocardial infarction, and silent subclinical (remote) myocardial infarction, and unstable angina pectoris. Myocardial infarction includes recurrent myocardial infarction, Q-wave myocardial infarction, non-Q-wave myocardial infarction and silent subclinical (remote) myocardial infarction. The compounds and formulations described herein can be used alone or in combination therapy with one or more additional agents (e.g., one or more blood modifiers, anti-hypertensive agents, dyslipidemic agents, peptides which mitigates one or more symptoms of atherosclerosis, other agents, including combinations thereof) to reduce the risk of mortality following a myocardial infarction or other cardiovascular or acute major coronary event. In certain embodiments, the compound or formulation (e.g., compound of the invention with a statin (e.g., atorvastatin, rosuvastatin, simvastatin, lovastatin)) is administered within 6, 12, 18, 24, 36, or 48 hours after hospital admission for a myocardial infarction or other cardiovascular or acute major coronary event (Fonarow G C 2005 Chest 128:3641-51). In some embodiments the compound or formulation is administered within 24 hours after hospital admission. In some embodiments the compound or formulation is administered at any time before hospital discharge. In some embodiments the formulation consists, consists essentially of, or comprises ezetimibe. In some embodiments the formulation consists, consists essentially of, or comprises Vytorin (ezetimibe and simivastatin). In some embodiments the formulation consists, consists essentially of, or comprises ezetimibe and one or more blood modifiers, anti-hypertensive agents, dyslipidemic agents (e.g., a statin such as atorvastatin, rosuvastatin), peptides which mitigates one or more symptoms of atherosclerosis, other agents, including combinations thereof.

The compounds and formulations described herein can used alone or in combination therapy with one or more additional agents (e.g., one or more blood modifiers, anti-hypertensive agents, dyslipidemic agents, peptides which mitigates one or more symptoms of atherosclerosis, other agents, including combinations thereof) to reverse or partially reverse the build-up of plaque in coronary arteries and thus may be associated with reduced plaque volume. In some embodiments, administration of the compounds or formulations described herein stops the progression of heart disease, leads to regression of heart disease. In some embodiments the formulation consists, consists essentially of, or comprises ezetimibe. In some embodiments the formulation consists, consists essentially of, or comprises Vytorin (ezetimibe and simivastatin). In some embodiments the formulation consists, consists essentially of, or comprises ezetimibe and one or more blood modifiers, anti-hypertensive agents, dyslipidemic agents (e.g., a statin such as atorvastatin, rosuvastatin), peptides which mitigates one or more symptoms of atherosclerosis, other agents, including combinations thereof.

The compounds and pharmaceutical formulations described herein can be used alone or in combination therapy with one or more additional agents (e.g., one or more blood modifiers, anti-hypertensive agents, dyslipidemic agents, peptides which mitigates one or more symptoms of atherosclerosis, other agents, including combinations thereof) to prevent or treat vascular conditions and associated thrombotic events as described, for example, in US20020147184. Vascular diseases and conditions are often associated with thrombotic events sometimes resulting in myocardial infarction, stroke and ischemic attack. A thrombotic event is one associated with the formation or presence of a thrombus (e.g., blood clot). Thrombotic events include but are not limited to arterial thrombosis, coronary thrombosis, heart valve thrombosis, coronary stenosis, stent thrombosis and graft thrombosis. Blood clots associated with thrombic events result from an aggregation of blood factors, primarily platelets and fibrin with entrapment of cellular elements and frequently cause vascular obstruction at the point of their formation. Blood coagulation is a process consisting of a complex interaction of various blood components, or factors, which eventually gives rise to a fibrin clot. It is often desirable to selectively block or inhibit the coagulation cascade in subjects at risk for or exhibiting a vascular disease or condition with blood modifiers e.g., heparin, coumarin, derivatives of coumarin, indandione derivatives, thrombin inhibitors, factor Xa inhibitors, or other agents. For example, in the case of atherosclerosis, proliferation of smooth muscle cells (SMCs) in the vessel wall is an important event in the formation of vascular lesions after vascular reconstruction or in response to other vascular injury. SMC proliferation typically occurs within the first few weeks and up to six months after injury. Thrombosis and or SMC proliferation are also involved in restenosis, which is the re-occlusion of the blood vessel or valve after surgical treatment e.g., angioplasty or bypass grafts. Thus, the compounds and pharmaceutical formulations described herein can be used to prevent or treat restenosis. The compounds and pharmaceutical formulations described herein can also be used to improve coagulation homeostasis (including reducing plasminogen activating inhibitor (PAI)-1 activity, reducing fibrinogen, managing high levels of fibrinogen, promoting fibrinolysis, and/or reducing platelet aggregation, and/or improving endothelial function). The compounds and pharmaceutical formulations described herein can used as coatings on surgical devices (e.g., catheters) and implants (e.g., stents) to reduce the risk of restenosis and thrombosis associated with invasive procedures used in the treatment of cardiovascular diseases.

The compounds and pharmaceutical formulations described herein can be used alone or in combination therapy with one or more additional agents (e.g., one or more anti-hypertensive agents, dyslipidemic agents, peptides which mitigates one or more symptoms of atherosclerosis, other agents, including combinations thereof) to prevent or treat vascular (e.g., cardiovascular, cerebrovascular, peripheral vascular, renovascular disease, mesenteric vascular, pulmonary vascular disease, ocular vascular) inflammation in a subject in need of such treatment, for example, as described in US20030119757 and to reduce blood plasma or serum concentrations of C-reactive protein (CRP) in a subject in need of such treatment, for example, as described in US20030119757. Vascular inflammation can lead to atherosclerosis or coronary heart disease. Atherosclerosis is often indicated by a thickening and build-up of plaque in the arteries and typically occurs when the innermost layer of an artery, the endothelium, becomes damaged by cholesterol, toxins, oxidants, infectious agents and the like. The damaged endothelial cells in the artery walls produce adhesion molecules that allow white blood cells to accumulate in the vessel wall. Fats and cholesterol also build-up with the white blood cells causing inflammation of the artery. Such build-up can thicken to a point where the artery becomes vulnerable to blockage from a clot resulting in heart attack or stroke. The compounds and pharmaceutical formulations described herein can be used alone or in combination therapy to slow the progression or cause regression of atherosclerotic plaques or lesions in, for example, coronary arteries, carotid arteries, the peripheral arterial system. Vascular inflammation often precedes the development and the continual process of atherosclerotic coronary heart disease. Vascular inflammation, beginning with an injury or change in the endothelial wall of the artery, may cause an alteration in the intimal layer that increases platelet adhesion to the endothelium. Vascular stimuli to mammals, e.g., cellular injury or inflammation, may lead to the production of various proteins, commonly called acute response proteins, in the body. CRP (C-reactive protein) is an acute response protein. Manufactured in the liver and deposited in damaged tissue, CRP is found in high levels in inflammatory fluids and in both the intimal layer of the atherosclerotic artery and within the lesions of atherosclerotic plaque. Studies have shown a positive association between CRP and coronary artery disease. For example, in a survey of 388 British men aged 50-69, the prevalence of coronary artery disease increased 1.5 fold for each doubling of CRP level (Mendall et al. (1996) BMJ. 312:1061-1065). Multiple prospective studies have also demonstrated that baseline CRP is a good marker of future cardiovascular events (Riker et al. 1998. J Investig Med. 46:391-395). Patients with CRP levels greater than about 0.4 mg/dL have been reported as having increased vascular inflammation and increased risk for vascular disease as compared to patients with levels less than 0.4 mg/dL. (L. Gruberb, “Inflammatory Markers in Acute Coronary Syndromes: C-reactive Protein (CRP) and Chlamydia”, American Heart Assoc. Scientific Sessions 2000). Patients with levels greater 3.4 mg/dL of c-reactive protein were reported to be in the highest quartile of risk. Patients in the second quartile (0.4 to 1.0 mg/dL of c-reactive protein) and third quartile (1.0 to 3.4 mg/dL of c-reactive protein) also have increased risk of vascular disease as compared to patients in the lowest quartile (<0.4 mg/dL c-reactive protein). CRP assays and methodologies for the same are available from Dade Behring Inc., Deerfield, Ill. Methods for analyzing CRPs are described, for example, in U.S. Pat. No. 5,358,852, U.S. Pat. No. 6,040,147, U.S. Pat. No. 6,277,584, and US20030119757.

The compounds and pharmaceutical formulations described herein can be used alone or in combination therapy with one or more additional agents (e.g., one or more agents used to treat Alzheimer's disease, other agents, including combinations thereof) to prevent, treat, or ameliorate symptoms of Alzheimer's Disease (AD), regulate production or levels of at least one amyloid β (Aβ) peptide and/or regulate the amount of ApoE isoform 4 in the bloodstream and/or brain of a subject, for example, as described in US2003013699 and U.S. Pat. No. 6,080,778. The compositions can be administered to a subject that exhibits no symptoms of AD, has AD, has a family history of AD or dementia illness, is a human, is a human and has trisomy 21 (Down's syndrome), is a human and carries one or more mutations in the genes that encode β amyloid precursor protein (presenilin-1 or presinilin-2), is a human and carries the Apolipoprotein E isoform 4 gene, is a human and is greater than about 40 years of age, is a human and is greater than about 60 years of age. The subject can have an elevated blood cholesterol level, a total serum cholesterol level that is at least about 200 mg/dl, a total low density lipoprotein (LDL) level that is greater than about 100 mg/dl. In some circumstances, the subject has an elevated level of at least one Aβ peptide in the bloodstream and/or brain. In various circumstances, the subject has an elevated level of Aβ-42 in the bloodstream and/or brain, has a level of Aβ-42 peptide greater than about 30 pM in the bloodstream, has a level of Aβ-42 peptide greater than about 40 pM in the bloodstream, has a level of Aβ-42 peptide ranging from about 30 pM to about 80 pM in the bloodstream, has a level Aβ-42 peptide of greater than about 50 pmol/gram of wet brain tissue. In various circumstances, the subject has a level of Aβ-40 peptide greater than about 200 pM in the bloodstream, has a level of Aβ-40 peptide greater than about 400 pM in the bloodstream, has a level of Aβ-40 peptide ranging from about 200 pM to about 800 pM in the bloodstream, has a level of Aβ-40 peptide greater than about 10 pmol/gram of wet brain tissue. In certain circumstances, the subject's level of Aβ peptide in the bloodstream is reduced from about 10 to about 100 percent from a level of Aβ peptides prior to administration of a composition of the present invention.

The compounds and pharmaceutical formulations described herein can be used alone or in combination therapy with one or more additional agents (e.g., one or more agents used to treat Alzheimer's disease, other agents, including combinations thereof) to enhance memory or to prevent, treat, or ameliorate symptoms of one or more of dementia, vascular dementia, Huntington's Disease, hydrocephalus, amnesia, AIDs-related dementia, Pick's Disease, Creutzfeldt-Jakob Syndrome, electroconvulsive therapy, Huntington's disease, amyotropic lateral sclerosis, Down syndrome, mental retardation, Parkinson's Disease, mild cognitive impairment, and memory loss.

The compounds and pharmaceutical formulations described herein can be used alone or in combination therapy with one or more additional agents (e.g., one or more anti-obesity agents, other agents, including combinations thereof) to prevent or treat obesity in a subject in need of such treatment, for examples as described in US20030119428. Obesity is a common medical problem in developed countries and is a risk factor for other illnesses, e.g., hypertension, diabetes, degenerative arthritis and myocardial infarction. Weight loss medications may be appropriate for use in selected patients who are obese or who are overweight with co-morbid conditions. One measure for defining obesity is known as a body mass index (BMI), which is weight in kilograms divided by height in meters squared. A BMI of 18.5 to 24.9 is generally classified as normal, a BMI of 25.0 to 29.9 is generally classified as overweight and a BMI of 30 or greater is generally classified as obese. Alternatively, obesity may be defined as the top percentile, e.g., 15 percent, of a population's weight for a given height. Such definitions of obesity, however, are not a measure of body composition and different people may have higher or lower levels of body fat or muscle mass for their height. Nevertheless, these definitions of obesity are useful characterizations for general populations of people.

The compounds and pharmaceutical formulations described herein can be used alone or in combination therapy with one or more additional agents to prevent or decrease the incidence of xanthomas in a subject in need of such treatment, for example, as described in US20030119809. Xanthomas are benign fatty tumors associated with the accumulation of fatty materials under the surface of the skin and are often associated with those who have high triglyceride and cholesterol levels. Xanthoma itself may be indicative of an underlying disease e.g., diabetes, primary biliary cirrhosis, some types of cancer, or hypercholesterolemia.

The compounds and pharmaceutical formulations described herein can be used alone or in combination therapy with one or more additional agents (e.g., one or more anti-hypertensive agents, dyslipidemic agents, peptides which mitigates one or more symptoms of atherosclerosis, other agents, including combinations thereof) to prevent or minimize muscular degeneration and related side effects associated with certain HMG-CoA reductase inhibitors (statins), for example, as described in US20030119808. Muscle degeneration encompasses all side effects relating to muscle degradation, aches, and/or weakness that may be associated with the administration of certain statins, including rhabdomyolysis and/or myopathy. Rhabdomyolysis is the destruction or degeneration of skeletal muscle tissue that is accompanied by the release of muscle cell contents (as myoglobin and potassium) into the bloodstream resulting in hypovolemia, hyperkalemia, and sometimes acute renal failure. Certain statins, allegedly have caused severe muscle degeneration in patients; cerivastatin allegedly has been associated with deaths due to rhabdomyolysis. Myopathies which refer to disorders of muscle tissue or muscles include muscle aches and muscle weakness in conjunction with increases in creatine phosphokinase (CPK) values over ten times the upper limit of normal. Risk of myopathy may be increased during use of high dose statins and/or when statins are administered with other drugs e.g., fibrates, niacin, azole, antifungals, erythromycin, and cyclosporin. The subjects to which the compound or pharmaceutical formulation is administered include those that have or are at risk for a vascular condition, a cardiovascular condition, hypercholesterolemia, atherosclerosis, arteriosclerosis. Suitable subjects include those having no history of clinically evident heart disease as well as those having a history of clinically evident heart disease.

The compounds and pharmaceutical formulations described herein can be used alone or in combination therapy with one or more additional agents (e.g., one more anti-diabetic agents, anti-hypertensive agents, dyslipidemic agents, peptides which mitigates one or more symptoms of atherosclerosis, other agents, including combinations thereof) to prevent or treat diabetes and associated conditions in a subject in need of such treatment, for example, as described in US20040214811. Diabetes mellitus, commonly called diabetes, refers to a disease process derived from multiple causative factors and characterized by elevated levels of plasma glucose, referred to as hyperglycemia. There are two major forms of diabetes: Type 1 diabetes (also referred to as insulin-dependent diabetes or IDDM) and Type 2 diabetes (also referred to as noninsulin dependent diabetes or NIDDM). Type 1 diabetes is the result of an absolute deficiency of insulin, the hormone that regulates glucose utilization. Type 1 diabetes has two forms: Immune-Mediated Diabetes Mellitus, which results from a cellular mediated autoimmune destruction of the β cells of the pancreas; and Idiopathic Diabetes Mellitus, which refers to forms of the disease that have no known etiologies. Type 2 diabetes is a disease characterized by insulin resistance accompanied by relative, rather than absolute, insulin deficiency. Premature development of atherosclerosis and increased rate of cardiovascular and peripheral vascular diseases are characteristic features of patients with diabetes. Diabetes and associated conditions include but are not limited to Type 1 diabetes, Type 2 diabetes, gestational diabetes mellitus (GDM), maturity onset of diabetes of the young (MODY), pancreatitis, polycystic ovarian disease, impaired glucose tolerance, insulin resistance, hyperglycemia, hyperinsulinemia, elevated blood levels of fatty acids or glycerol, obesity, Syndrome X, dysmetabolic syndrome and related diseases, diabetic complications (including retinopathy, neuropathy, nerphropathy) and sexual dysfunction. The conditions, diseases, and maladies collectively referenced to as “Syndrome X” or Dysmetabolic Syndrome (as detailed in Johanson, J. Clin. Endocrinol. Metab., 1997, 82, 727-734, and other publications) include hyperglycemia and/or prediabetic insulin resistance syndrome, and is characterized by an initial insulin resistant state generating hyperinsulinemia, dyslipidemia, and impaired glucose tolerance, which can progress to Type II diabetes, characterized by hyperglycemia, which can progress to diabetic complications.

The compounds and pharmaceutical formulations described herein can be used alone or in combination therapy with one or more additional agents (e.g., one more agents used to treat autoimmune disorders, other agents, including combinations thereof) to prevent or treat at least one autoimmune disorder in a subject in need of such treatment, for example, as described (including the rationale for the therapy) in US20040092499. Autoimmune disorders include, but are not limited to: Alopecia Areata, Ankylosing Spondylitis, Antiphospholipid Syndrome, aplastic anemia, myelodysplastic syndromes, paroxysmal nocturnal hemoglobulinemia, pure red cell aplasia, chronic neutropenias, amegakaryocytic thrombocytopenia, antiphospholipid syndromes, autoimmune thrombocytopenia, autoimmune hemolytic syndromes, antiphospholipid syndromes, autoimmune gastritis, achlorhydria, Autoimmune Addison's Disease, Autoimmune Diabetes, Autoimmune Hemolytic Anemia, Autoimmune Hepatitis, Autoimmune chronic Hepatitis, Autoimmune hypophysitis, Autoimmune orchiditis, autoimmune ovarian failure, Behcet's Disease, Bullous Pemphigoid, Cardiomyopathy, Celiac Sprue-Dermatitis, Cicatrical pemphigoid, Chronic Fatigue Immune Dysfunction Syndrome (CFIDS), Chronic Inflammatory Demyelinating Polyneuropathy, Interstitial cystitis, Churg-Strauss Syndrome, Cicatricial Pemphigoid, CREST Syndrome, Cold Agglutinin Disease, Crohn's Disease, Dermatitis herpetiformis, Discoid Lupus, Drug-induced autoimmune disorders, Endometriosis, Epidermolysis bullosa acquisita, Essential Mixed Cryoglobulinemia, Fibromyalgia-Fibromyositis, Glomerulonephritis, Good Pasture Syndrome, Graft Versus Host Disease, Graves' Disease, Guillain-Barr, Hashimoto's Thyroiditis, Idiopathic Inflammatory Myopathies, Idiopathic Pulmonary Fibrosis, Idiopathic Thrombocytopenia Purpura (ITP), IgA Nephropathy, Insulin Dependent Diabetes, Juvenile Arthritis, Lichen Planus, Systemic Lupus Erythmatosus, Mnire's Disease, Metal-induced autoimmunity disorders, Mixed Connective Tissue Disease, Multiple Sclerosis, Myasthenia Gravis, Myocarditis, Myositis, Optic neuritis, Painless/postpartum thyroiditis, Peripheral nerve vasculitis, Pemphigus Foliaceus, Pemphigus Vulgaris, Pernicious Anemia, Polyarteritis Nodosa, Polychondritis, Polyglandular Syndromes, Polymyalgia Rheumatica, Polymyositis and Dermatomyositis, Postinfectious autoimmune disorders, Primary Agammaglobulinemia, Primary Biliary Cirrhosis, Psoriasis, Psoriatic Arthritis, Reactive Arthritis, Raynaud's Phenomenon, Reiter's Syndrome, Rheumatic Fever, Rheumatoid Arthritis, Sarcoidosis, Scleritis, Scleroderma, Sjogren's Syndrome, Stiff-Man Syndrome, Takayasu Arteritis, Temporal Arteritis/Giant-cell Arteritis, Ulcerative Colitis, Uveitis, Vasculitis, Vitiligo, Kawasaki Disease, and Wegener's Granulomatosis.

The compounds and pharmaceutical formulations described herein can be used alone or in combination therapy with one or more additional agents (e.g., one more agents used to treat demylenation and its associated disorders, other agents, including combinations thereof) to prevent or treat demyelination and associated disorders in a subject in need of such treatment, for example, as described (including the rationale for the therapy) in US20040092500. Nerve fibers are wrapped with multiple layers of insulation known as myelin sheath. Demyelination can occur through disease and results in the destruction or removal of the myelin sheath. Primary demyelinating disorders include but are not limited to multiple sclerosis, acute disseminated encephalomyelitis, adrenoleukodystrophy, adrenomyeloneuropathy, Leber's hereditary optic atrophy and HTLV-associated myelopathy. Other disorders associated with demyelination include but are not limited to Tay-Sachs disease, Niemann-Pick disease, Gaucher's disease and Hurler's syndrome; or stroke, inflammation, immune diseases, metabolic disorders, poison or drugs.

The compounds and pharmaceutical formulations described herein can be used alone or in combination therapy with one or more additional agents (e.g., one or more chemotherapeutic agents, anti-cancer agents, other agents, including combinations thereof) to prevent or treat cholesterol associated tumors in patients who are either at risk of developing a cholesterol-associated tumor or already exhibit a cholesterol associated tumor, for example, as described in US20040116358. The compounds of the invention may reduce both cholesterol levels in vivo and epoxycholesterol formation and thereby inhibit initiation and progression of benign and malignant cholesterol-associated tumors or cholesterol-associated cell growth or cell-masses. The tumors may be benign cholesterol-associated tumors or cholesterol-associated cell growth or cell-masses including but not limited to benign tumors associated with prostate, colon, endometrial, or breast tissues or prostate, colon, breast, or endometrial cancer. Thus the compounds and pharmaceutical formulations described herein, for example, are useful to prevent or treat benign prostatic hypertrophy. The tumors may be malignant cholesterol-associated tumors or cholesterol-associated cell growth or cell-masses including but not limited to malignant tumors associated with prostate, colon, endometrial, or breast tissues or prostate, colon, breast, or endometrial cancer.

The compounds and pharmaceutical formulations described herein can be used alone or in combination therapy with one or more additional agents (e.g., one or more chemotherapeutic agents, anti-cancer agents, other agents, including combinations thereof) for one or more of: inhibiting the expression of at least one multiple (“multi”)-drug resistance gene or protein in an animal cell, enhancing the effectiveness of a chemotherapeutic agent in an animal having cancer, and reversing a multi-drug resistance phenotype exhibited by an animal cell all of which are, for example, described in WO05/030225. Co-administration (though not necessarily concurrent or proximal consecutive) of cholesterol absorption inhibitors and chemotherapeutic agents can inhibit the expression of multi-drug resistance genes. Thus the compounds and pharmaceutical formulations described herein can be used alone or in combination therapy in one or more of: a) treating or alleviating a cancer; b) preventing, treating or alleviating tumour growth; c) inhibiting or reducing the expression of one or more multiple drug resistance genes; d) inhibiting or reducing the production of one or more proteins expressed by multiple drug resistance genes; e) enhancing the effectiveness of a chemotherapeutic agent in treating a cancer; and f) sensitizing a cell to one or more chemotherapeutic agents. Multiple drug resistance genes include but are not limited to ABCB1 (MDR-1), ABCA2 (ABC2), ABCB2 (TAP), ABCB3 (TAP), ABCC1 (MRP-1), and ABCC3 (MRP-3).

The compounds and pharmaceutical formulations described herein can be used alone or in combination therapy with one or more additional agents (e.g., dyslipidemic agents, anti-diabetic agents, anti-hypertensive agents, anti-obesity agents, agents used to treat autoimmune disorders, agents used to treat demylenation and its associated disorders, agents used to treat Alzheimer's disease, blood modifiers, hormone replacement agent/compositions, chemotherapeutic agents, peptides which mitigate one or more symptoms of atherosclerosis, anti-cancer agents, agents used to treat bone loss and associated disorders, other agents, including combinations thereof) to modulate lipid raft structure (for example by reducing the level of cholesterol in the lipid raft), for example, as described (including the related rationale) in WO05023305. Lipid rafts are discrete microdomains in the plasma membrane which are rich in sphingolipids and contain ordered cholesterol (Field et al., J. Biol. Chem., 1997, 272, 4276-4280). In a number of cells, it has become clear that certain membrane associated proteins preferentially partition into these lipid rafts (Foster, de Hoog and Mann, PNAS, 2003, 100, 5813-8). These include various seven transmembrane domain receptors and their associated G proteins and various proteins that are attached to the inner membrane leaflet through lipid moieties such as prenylation, including small molecular weight G proteins, such as Ras, Rac, cdc42 and Rho. Disruption of lipid rafts results in an uncoupling of efficient signal transduction through receptors such as G protein coupled receptors, the T cell receptor and the high affinity IgE receptor. Compounds which modulate lipid raft structure may be useful in the treatment or prophylaxis of a wide variety of diseases and conditions. The compounds and pharmaceutical formulations described herein can be used alone or in combination therapy to prevent or treat a disease or condition associated with lipid raft structure such as respiratory tract/obstructive airways diseases and disorders (including: acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), antitussive activity, asthma (such as bronchial, allergic, intrinsic, extrinsic and dust asthma particularly chronic or inveterate asthma (e.g., late asthma and airways hyper-responsiveness)), bronchitis (including chronic and eosinophilic bronchitis), emphysema, chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), chronic obstructive pulmonary disease (COPD) (such as irreversible COPD), chronic sinusitis, conjunctivitis (e.g., allergic conjunctivitis), cystic fibrosis, fanner's lung and related diseases, fibroid lung, hypersensitivity lung diseases, hypersensitivity pneumonitis, idiopathic interstitial pneumonia, nasal congestion, nasal polyposis, otitis media, and chronic cough associated with inflammation or iatrogenic induced); systemic anaphylaxis or hypersensitivity responses (such as drug allergies (e.g., to penicillin, cephalosporins), insect sting allergies, pet allergies, house dust mite allergies, pollen allergies, and food related allergies which may have effects remote from the gut (such as migraine, rhinitis and eczema)); gastro-intestinal diseases and disorders (such as irritable bowel syndrome (IBS), inflammatory bowel disease (IBD), Crohn's disease, ulcerative colitis, gastric and duodenal ulceration), Fabry's disease; Kimura's disease; multiple sclerosis; wound healing; liver hepatitis and cirrhosis; rheumatoid arthritis; juvenile rheumatoid arthritis; systemic lupus erythematosus; degenerative joint disease; connective tissue diseases; ankylosing spondylitis; soft tissue rheumatism (e.g., tendonitis, bursitis); Sjogren's syndrome; psoriasis; psoriatic arthritis; neuralgia; synovitis; glomerulonephritis; vasculitis; sacoidosis; inflammations that occur as sequellae to influenza; the common cold and other viral infections; gout; pseudogout; contact dermatitis; low back and neck pain; dysmenorrhea; headache; dementias; toothache; sprains; strains; myositis; burns; injuries; pain and inflammation that follows surgical and dental procedures in a patient; Parkinsons disease; muscular dystrophy; neoplasia; hyperparathyroidism; sepsis and septic shock; infections by intracellular pathogens (including, for example, bacteria (such as Salmonella, Chlamydiae, Listeria, Mycobacteria tuberculosis), viruses (such as HIV, Measles virus, Papilloma viruses, Epstein-Barr virus, Respiratory Syncytial Virus (RSV), Hepatitis, Herpes viruses, Influenza virus, Ebola and Marburg viruses), parasites (such as Plasmodium (malaria), Leishmania, Trypanosoma (sleeping sickness), Toxoplasma gondii)); and bacterial infections including Shigella, Escherichia Coli (including 0157), Campylobacter, Vibrio cholerae, Clostridium difficile and Clostridium tetani.

The compounds and pharmaceutical formulations described herein can be used alone or in combination therapy with one or more additional agents (e.g., one or more dyslipidemic agents, peptides which mitigates one or more symptoms of atherosclerosis, hormone replacement agents/compositions, anti-cancer agents, chemotherapeutic agents, agents used to treat bone loss and associated disorders, other agents, including combinations thereof) to prevent or treat osteopenia disorders (bone loss disorders) in subjects in need of such treatment. There is a well documented co-morbid development of bone loss disorders (e.g., osteoporosis) and cardiovascular disease. For example, Gas-6, osteocalcin, matrix gammacarboxy glutamate protein and protein S function in both bone formation and arterial calcification. Bone loss disorders and associated conditions include but are not limited to: osteoporosis, Paget's disease (osteitis deformans), bone loss, bone fractures, bone segmental defects, abnormally increased bone turnover, conditions associated with bone fracture or deficiency, rheumatoid arthritis (including bone loss attendant rheumatoid arthritis), osteoarthritis, osteolysis (including familial expansile osteolysis and periprostetic osteolysis), osteolytic metastases, osteolytic bone disease, metastatic bone disease, osteosarcoma, osteonecrosis, osteogenesis imperfecta, osteomyelitis (e.g., an infectious lesion in bone leading to bone loss), cleiodocranial dysplasia (CCD), prosthetic loosening, periodontal disease (e.g., periodontitis) and defects, and other tooth repair processes, tooth loss, primary or secondary hyparathyroidism, hypercalcemia (including hypercalcemia of malignancy, and multiple myeloma), cartilage defects or disorders (including cartilage degeneration), conditions associated with connective tissue repair (e.g., healing or regeneration of cartilage defects or injury), metabolic bone diseases, and transplant and drug-induced bone loss. Osteoporosis includes primary osteoporosis, secondary osteoporosis, medication-induced osteoporosis (e.g., corticosteroid-induced osteoporosis, transplant-bone disease), age-related osteoporosis in females or males, post-menopausal osteoporosis, glucocorticoid-induced osteoporosis, idiopathic osteoporosis, disease-induced arthritis (e.g., rheumatoid arthritis induced), disuse osteoporosis and arthritis, diabetes-related osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Rile-Day syndrome) and osteoporosis due to immobilization of extremities. The compounds and pharmaceutical formulations described herein can be used alone or in combination therapy in one more of the following: enhancing/promoting bone formation; preventing bone loss; repair of bone defects and deficiencies, such as those occurring in closed, open and nonunion fractures; prophylactic use in closed and open fracture reduction; promotion of bone healing in plastic surgery; stimulation of bone ingrowth into non-cemented prosthetic joints and dental implants; elevation of peak bone mass in perimenopausal women; prevention or treatment of growth deficiencies; prevention or treatment of increased bone formation during distraction osteogenesis; prevention or treatment of any condition that benefits from stimulation of bone formation; repair of congenital, trauma-induced or surgical resection of bone (for instance, for cancer treatment), and in cosmetic surgery; treatment of wound healing or tissue repair; treatment of subjects undergoing facial reconstruction surgery; treatment of subjects undergoing orthopedic or oral surgery; alleviation of bone pain; prevention or treatment of localized bone loss associated with periprosthetic osteolysis and bone fractures, etc.; rapid inhibition of bone resorption in a subject while obtaining a rapid reduction of bone turnover and biomarkers; rapid increase of bone mineral density; and rapid reduction of fractures. The compounds and pharmaceutical formulations described herein can be used alone or in combination therapy to stimulate bone regeneration. The bone regeneration may be following reconstruction of bone defects in cranio-maxillofacial surgery, or following an implant into bone, for example a dental implant, bone supporting implant, or prosthesis. The bone regeneration may also be following a bone fracture.

The compounds and pharmaceutical formulations described herein can be used alone or in combination therapy with one or more additional agents for preventing and treating malignant lesions (such as ductal carcinoma in situ of the breast and lobular carcinoma in situ of the breast), premalignant lesions (such as fibroadenoma of the breast and prostatic intraepithelial neoplasia (PIN), gastrointestinal malignancies, liposarcomas and various other epithelial tumors (including breast, prostate, colon, ovarian, gastric and lung), cancer-induced asthenia (fatigue), irritable bowel syndrome, Crohn's disease, gastric ulceritis, and gallstones, and HIV infection, other infectious diseases, drug-induced lipodystrophy, and proliferative diseases such as psoriasis, for example, as described in US20050085497.

As described, for example, in US20050101565, the compounds and pharmaceutical formulations described herein can be used alone or in combination therapy with one or more additional agents to prevent or treat: cancers (including but not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease (In certain embodiments, the cancers that are treated or prevented by administering the compounds described herein are insulin resistance or Syndrome X related cancers, including but not limited to breast, prostate and colon cancer); PPAR-associated disorders (including but not limited to rheumatoid arthritis; multiple sclerosis; psoriasis; inflammatory bowel diseases; breast; colon or prostate cancer; low levels of blood HDL (HDL may be elevated in lymph and/or cerebral fluid); low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity; renal diseases including but not limited to glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal disease, medullary sponge kidney, medullary cystic disease, nephrogenic diabetes, and renal tubular acidosis), tubulointerstitial diseases (including but not limited to pyelonephritis, drug and toxin induced tubulointerstitial nephritis, hypercalcemic nephropathy, and hypokalemic nephropathy), acute and rapidly progressive renal failure, chronic renal failure, nephrolithiasis, or tumors (including but not limited to renal cell carcinoma and nephroblastoma) (In certain embodiments, renal diseases that are treated by the compounds described herein are vascular diseases, including but not limited to hypertension, nephrosclerosis, microangiopathic hemolytic anemia, atheroembolic renal disease, diffuse cortical necrosis, and renal infarcts); and septicemia and impotence (for example, which may result from cardiovascular disease).

In addition to the other subjects discussed herein, the compositions and pharmaceutical formulations described herein can be administered alone or in combination therapy with one or more additional agents (e.g., one or more hormone replacement agents/compositions, dyslipidemic agents, peptides which mitigates one or more symptoms of atherosclerosis, anti-hypertensive agents, anti-diabetic agents, anti-obesity agents, other agents, including combinations thereof) to post-menopausal women, for example, as described in US20030119796. In various embodiments, the compositions and pharmaceutical formulations described herein can be administered alone or in combination therapy with one or more additional agents to, for example, (1) a subject in need of or who has undergone an organ transplant (e.g., a kidney transplant), (2) a subject who is either at risk of developing systemic lupus erythematosus or already exhibits systemic lupus erythematosus, (3) a subject who has undergone or is undergoing hemodialysis, (4) a subject who is either at risk of developing hyperhomocysteine levels or already exhibits hyperhomocysteine levels, (5) a subject who is either at risk of developing hypothyroidism or already exhibits hypothyroidism, (6) a subject who is either at risk of developing obstructive liver disease or already exhibits obstructive liver disease, (7) a subject who is either at risk of developing kidney disease or already exhibits kidney disease, (8) a subject who has undergone cardiac bypass surgery, and (9) a subject who has undergone percutaneous transluminal coronary angioplasty.

The compounds and pharmaceutical formulations described herein can be administered alone or in combination therapy with one or more additional agents to a non-human animal for a veterinary use for treating, preventing, or managing a disease or disorder disclosed herein. Non limiting examples of non-human examples include cows, horses, sheep, pigs, cats, dog, mice, rats, rabbits, guinea pigs, and fowl species (e.g., chicken, turkey, duck, goose, quail). In addition to veterinary uses, the compounds and pharmaceutical formulations described herein can be used to reduce the fat content of livestock to produce leaner meats and to reduce the cholesterol content of eggs by administering the compounds to a chicken, quail, or duck hen. For non-human animal uses, the compounds and pharmaceutical formulations described herein can be administered via the animals' feed or orally as a drench composition.

Certain compounds of the invention may have the additional advantage that they suppress serum cholesterol and/or LDL levels while themselves not being appreciably absorbed into the mammalian circulation upon oral administration. As a result of the low-to-insignificant serum levels, fewer side-effects, such as drug-drug interactions, are observed.

### DEFINITIONS

Throughout this specification the terms and substituents retain their definitions.

Alkyl is intended to include linear, branched, or cyclic hydrocarbon structures and combinations thereof. When not otherwise restricted, the term refers to alkyl of 20 or fewer carbons. Lower alkyl refers to alkyl groups of 1, 2, 3, 4, 5 and 6 carbon atoms. Examples of lower alkyl groups include, but are not limited to, methyl, ethyl, propyl, isopropyl, butyl, s- and t-butyl and the like. In certain embodiments the lower alkyl group is methyl. Alkylene refers to divalent alkyl groups. Preferred alkyl and alkylene groups are those of C₂₀ or below (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀). Cycloalkyl is a subset of alkyl and includes cyclic hydrocarbon groups of 3, 4, 5, 6, 7, and 8 carbon atoms. Examples of cycloalkyl groups include c-propyl, c-butyl, c-pentyl, norbornyl, adamantyl and the like.

The term lower alkylene-OH refers to a lower alkylene group in which one C—H bond has been replaced by C—OH.

C₁ to C₂₀ Hydrocarbon (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀) includes alkyl, cycloalkyl, alkenyl, alkynyl, aryl and combinations thereof. Examples include benzyl, phenethyl, cyclohexylmethyl, camphoryl and naphthylethyl. The term “phenylene” refers to ortho, meta or para residues of the formulae:

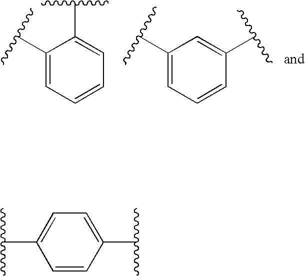

Alkoxy or alkoxyl refers to groups of 1, 2, 3, 4, 5, 6, 7 or 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower alkoxy refers to groups containing one to four carbons. In certain embodiments the lower alkoxy group is methoxy.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶96, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Polyol refers to a compound or residue having a plurality of —OH groups, as defined in Hawley's Condensed Chemical Dictionary, 12^{th} Edition, Richard J. Lewis, Sr.; Van Nostrand Reinhold Co. New York (1993). Polyols may be thought of as alkyls in which a plurality of C—H bonds have been replaced by C—OH bonds. More particularly, a polyol, also referred to as an alditol, is an alcohol of general formula CH₂OH(CH₂OH)ₙCH₂OH, wherein n is 1 to 5. Common polyol compounds include for example glycerol, erythritol, sorbitol, xylitol, mannitol and inositol. Linear polyol residues will generally be of the empirical formula —C_{y}H_{2y+1}O_{y}, and cyclic polyol residues will generally be of the formula —C_{y}H_{2y-1}O_{y}. Those in which y is 3, 4, 5 and 6 are preferred. Cyclic polyols also include reduced sugars, such as glucitol.

Acyl refers to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, propionyl, isobutyryl, t-butoxycarbonyl, benzoyl, benzyloxycarbonyl and the like. Lower-acyl refers to groups containing one to four carbons.

Aryl and heteroaryl refer to aromatic or heteroaromatic rings, respectively, as substituents. Heteroaryl contains one, two or three heteroatoms selected from O, N, or S. Both refer to monocyclic 5- or 6-membered aromatic or heteroaromatic rings, bicyclic 9- or 10-membered aromatic or heteroaromatic rings and tricyclic 13- or 14-membered aromatic or heteroaromatic rings. Aromatic 6, 7, 8, 9, 10, 11, 12, 13 and 14-membered carbocyclic rings include, e.g., benzene, naphthalene, indane, tetralin, and fluorene and the 5, 6, 7, 8, 9 and 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, lower alkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine.

The term “sugar” is used in its normal sense, as defined in Hawley's Condensed Chemical Dictionary, 12^{th} Edition, Richard J. Lewis, Sr.; Van Nostrand Reinhold Co. New York (1993). It encompasses any carbohydrate comprised of one or two saccharose groups. The monosaccharide sugars (often called simple sugars) are composed of chains of 2-7 carbon atoms. One of the carbons carries aldehydic or ketonic oxygen, which may be combined in acetal or ketal forms. The remaining carbons usually have hydrogen atoms and hydroxyl groups (or protecting groups for hydroxyl, such as acetate). Monosaccharides, which would be considered within the term “sugars” as intended in this application, include, but are not limited to, allose, altrose, gulose, idose, talose, arabinose, ribose, xylose, lyxose, ribulose, xylulose, deoxyribose, galactose, glucose, mannose, fructose, sorbose, tagatose, fucose, quinovose, rhamnose, manno-heptulose and sedoheptulose. Disaccharides include, but are not limited to, sucrose, lactose, maltose, and cellobiose. Unless specifically modified, the general term “sugar” refers to both D-sugars and L-sugars. The sugar may also be protected. Sugars may also be indirectly attached to any aglycone that has a free phenol by the method of Kværnø, Werder, Hauser and Carreira “Carbohydrate Sulfonyl Chlorides for Simple, Convenient Access to Glycoconjugates” [Org. Lett. 7(6): 1145-48 (2005).], the disclosure of which is incorporated herein by reference. This method provides sugars linked via a sulfonate.

Reduced C-attached sugars or C-glycosyl compounds are also encompassed by the invention. The reduced sugars (e.g. glucitol) could be classified either as polyols or as sugars. In certain embodiments, the reduced sugar is D-glucitol having the following structure

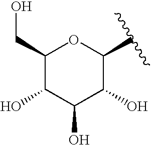

wherein the wavy line indicates the point of attachment to the phenyl ring. However, the invention is not limited to D-glucitol.

The term “glucuronide” is also used in its normal sense to refer to a glycoside of glucuronic acid. In certain embodiments, the glucuronide residue is:

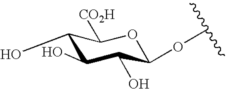

wherein the wavy line indicates the point of attachment. However, the invention is not limited to this particular glucuronide.

The term “sugar carbamate” refers to mono-, di- and oligosaccharides in which one or more hydroxyls have been derivatized as carbamates, particularly as phenyl carbamates and substituted phenyl carbamates. [See Detmers et al. Biochim Biophys. Acta 1486, 243-252 (2000), which is incorporated herein by reference.] In certain embodiments the sugar carbamate is:

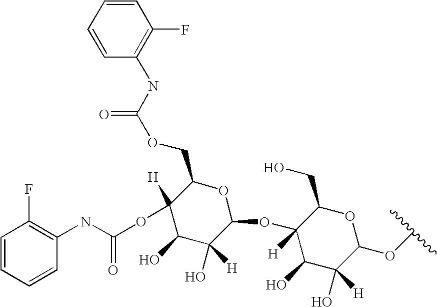

wherein the wavy line indicates the point of attachment. However, the invention is not limited to this sugar carbamate.

The term “prodrug” refers to a compound that is made more active in vivo. Commonly the conversion of prodrug to drug occurs by enzymatic processes in the liver or blood of the mammal. Many of the compounds of the invention may be chemically modified without absorption into the systemic circulation, and in those cases, activation in vivo may come about by chemical action (as in the acid-catalyzed cleavage in the stomach) or through the intermediacy of enzymes and microflora in the gastrointestinal GI tract.

It will be recognized that the compounds of this invention can exist in forms in which one isotope of a particular atom may be replaced with a different isotope of that same atom. For example, “hydrogen” may be ¹H, ²H or ³H; “carbon” may be ¹²C, ¹³C, or ¹⁴C; “nitrogen” may be ¹⁴N or ¹⁵N; “oxygen” may be ¹⁶O, ¹⁷O or ¹⁸O; and the like. It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, iodine and chlorine include ³H, ¹⁴C ³⁵S, ¹⁸F, ³²P, ³³P, ¹²⁵I, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of the invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

The compounds of the invention can also exist in other labeled forms. US20020009714 discloses methods of labeling and uses of labeled cholesterol absorption inhibitors. The labels can be primary labels (where the label comprises an element which is detected directly) or secondary labels (where the detected label binds to a primary label, e.g., as is common in immunological labeling). An introduction to labels, labeling procedures and detection of labels is found in Introduction to Immunocytochemistry, (2d ed.) Polak and Van Noorden, Springer Verlag, N.Y. (1997) and in Handbook of Fluorescent Probes and Research Chemicals, Haugland (1996), a combined handbook and catalogue published by Molecular Probes, Inc., Eugene, Oreg. Primary and secondary labels can include undetected elements as well as detected elements. Useful primary and secondary labels in the present invention can include spectral labels, which include fluorescent labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™ and the like), radiolabels (including those described above), enzymes (e.g., horseradish peroxidase, alkaline phosphatase etc.) spectral colorimetric labels such as colloidal gold or colored glass or plastic (e.g., polystyrene, polypropylene, latex, etc.) beads. The label may be coupled directly or indirectly to the compound of the invention according to methods well known in the art. As indicated above, a wide variety of labels may be used, with the choice of label depending on sensitivity required, ease of conjugation with the compound, stability requirements, available instrumentation, and disposal provisions. In general, a detector which monitors a protein/inhibitory agent interaction is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill.

Nonlimiting examples of labels include those which utilize 1) chemiluminescence (using horseradish peroxidase or alkaline phosphatase with substrates that produce photons as breakdown products) with kits being available, e.g., from Molecular Probes, Amersham, Boehringer-Mannheim, and Life Technologies/Gibco BRL; 2) color production (using both horseradish peroxidase or alkaline phosphatase with substrates that produce a colored precipitate) (kits available from Life Technologies/Gibco BRL, and Boehringer-Mannheim); 3) fluorescence (e.g., using Cy-5 (Amersham), fluorescein, and other fluorescent tags); 5) radioactivity. Other methods for labeling and detection will be readily apparent to one skilled in the art.

In one embodiment, the label is a fluorescent label. Fluorescent labels have the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, digoxigenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent tags are commercially available from the SIGMA chemical company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka ChemicaBiochemika Analytika (Fluka Chemie AG, Buchs, Switzerland), and Applied Biosystems (Foster City, Calif.), as well as many other commercial sources known to one of skill.

The labels may be covalently bound to the compounds of the invention by a tether group. The tether group can be any moiety capable of covalently linking to the inhibitors and to the labels. Preferred groups are substituted or unsubstituted alkylene, alkenylene or alkynylene of 1 to 10 carbon atoms, more preferably 1 to 4 carbon atoms. Particularly preferred groups are unsubstituted alkynylenes.

The terms “methods of treating or preventing” mean amelioration, prevention or relief from the symptoms and/or effects associated with lipid disorders. The term “preventing” as used herein refers to administering a medicament beforehand to forestall or obtund an acute episode or, in the case of a chronic condition to diminish the likelihood or seriousness of the condition. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis.

Throughout this application, various references are referred to. The disclosures of these publications in their entireties are hereby incorporated by reference as if written herein.

The term “mammal” is used in its dictionary sense. The term “mammal” includes, for example, mice, hamsters, rats, cows, sheep, pigs, goats, and horses, monkeys, dogs (e.g., Canis familiaris), cats, rabbits, guinea pigs, and primates, including humans.

The compounds described herein contain two or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

The term “enantiomeric excess” is well known in the art and is defined for a resolution of ab into a+b as

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

Terminology related to “protecting”, “deprotecting” and “protected” functionalities occurs throughout this application. Such terminology is well understood by persons of skill in the art and is used in the context of processes, which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference. Particular attention is drawn to the chapters entitled “Protection for the Hydroxyl Group, Including 1,2- and 1,3-Diols” (pages 10-86).

The abbreviations Me, Et, Ph, Bn, Tf, Ts and Ms represent methyl, ethyl, phenyl, benzyl, trifluoromethanesulfonyl, toluenesulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

While it may be possible for the compounds of the invention to be administered as the raw chemical, it is preferable to present them as a pharmaceutical composition. According to a further aspect, the present invention provides a pharmaceutical composition comprising a compound of formula I or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

Examples of excipients for use as the pharmaceutically acceptable carriers and the pharmaceutically acceptable inert carriers and the aforementioned additional ingredients include, but are not limited to:

BINDERS: polyethylene oxide, corn starch, citric acid monohydrate, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose, carboxymethyl cellulose calcium, sodium carboxymethyl cellulose, polyvinyl pyrrolidone, polyvinyl alcohol, methyl cellulose, pre-gelatinized starch (e.g., STARCH 1500® and STARCH 1500 LM®, sold by Colorcon, Ltd.), hydroxypropyl methyl cellulose, methyl cellulose, hydroxymethyl cellulose, hydroxypropyl cellulose, microcrystalline cellulose (e.g., AVICEL™, such as, AVICEL-PH-101™, -103™ and -105™, sold by FMC Corporation, Marcus Hook, Pa., USA), or mixtures thereof;

FILLERS: talc, sodium choloride, aluminum oxide, iron oxides (e.g., yellow, black, red), red ferric oxide, yellow ferric oxide, magnesium carbonate, magnesium hydroxide, magnesium aluminate, aluminum magnesium hydroxide, calcium carbonate (e.g., granules or powder), calcium dihydroxide, dibasic calcium phosphate, dibasic calcium phosphate anhydrous, triacetin, lactose, hydrous lactose, tribasic calcium phosphate, calcium sulfate (e.g., granules or powder), microcrystalline cellulose, silicified microcrystalline cellulose, soybean lecithin, xanthar gum, silicic anhyride, powdered cellulose, dextrates, kaolin, mannitol, silicic acid, sorbitol, starch, pre-gelatinized starch, or mixtures thereof;

DISINTEGRANTS: agar-agar, alginic acid, calcium carbonate, simethicone emulsion, lactose monohydrate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, or mixtures thereof;

SURFACTANTS: Tween 80 or polyoxyethylene-polyoxypropylene copolymer, polyoxyethylene sorbitan, or mixtures thereof;

LUBRICANTS: calcium stearate, magnesium stearate, mineral oil, light mineral oil, glycerin, sorbitol, mannitol, palmitic acid, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Degussa Co., Plano, Tex. USA), a pyrogenic silicon dioxide (CAB-O-SIL, Cabot Co., Boston, Mass. USA), or mixtures thereof;

ANTI-CAKING AGENTS: calcium silicate, magnesium silicate, silicon dioxide, colloidal silicon dioxide, talc, or mixtures thereof;

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzo ate, sodium dehydroacetate, sodium propionate, polysorbate, sorbic acid, thimersol, thymo, or mixtures thereof;

COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol (e.g., polyethylene glycol 8000, polyethylene glycol 3000), polyvinyl acetate phthalate, shellac, sucrose, titanium dioxide, carnuba wax, candelilla wax, microcrystalline wax, or mixtures thereof;

COLORANTS: FD&C blue no. 1, D&C yellow #10 aluminum lake, FD&C yellow #6/sunset yellow FCF aluminum lake, FD&C carmine aluminum lake and FD&C blue #1, or mixtures thereof; and

ANTIOXIDANTS: butylated hydroxyanisole, sodium ascorbate, sodium metabisulfate, malic acid, citric acid, ascorbic acid, butylated hydroxytoluene, vitamin C, propyl gallate, or mixtures thereof.

Solid oral dosage forms may optionally be treated with coating systems (e.g., Opadry® fx film coating system, for example Opadry® blue (OY-LS-20921), Opadry® white (YS-2-7063), Opadry® white (YS-1-7040), and black ink (S-1-8106).

The dose range for adult humans is generally from 0.005 mg to 10 g/day orally. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

### Combination Therapy

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc. Combination therapy can also include the administration of two or more agents via different routes or locations. For example, (a) one agents is administered orally and another agents is administered intravenously or (b) one agent is administered orally and another is administered locally into the site of injury (e.g., an artery). In each case, the agents can either simultaneously or sequentially. Approximated dosages for some of the combination therapy agents described herein are found in the “BNF Recommended Dose” column of tables on pages 11-17 of WO01/76632 (the data in the tables being attributed to the March 2000 British National Formulary) and can also be found in other standard formularies and other drug prescribing directories. For some drugs, the customary prescribed dose for an indication will vary somewhat from country to country.

### Dyslipidemic Agents

The compounds described herein can be used in therapeutic combination with one or more dyslipidemic agents. Suitable dyslipidemic agents for use in therapeutic combination with a compound described herein includebile acid sequestrants such as cholestyramine (a styrene-divinylbenzene copolymer containing quaternary ammonium cationic groups capable of binding bile acids, such as Questran® or Questran Light® cholestyramine which are available from Bristol-Myers Squibb), colesevelam hydrochloride (such as WelChol® Tablets (polyallylamine hydrochloride) cross-linked with epichlorohydrin and alkylated with 1-bromodecane and (6-bromohexyl)-trimethylammonium bromide) which are available from Sankyo), colestipol (a copolymer of diethylenetriamine and 1-chloro-2,3-epoxypropane, such as Colestid® tablets which are available from Pharmacia), dialkylaminoalkyl derivatives of a cross-linked dextran, LoCholest®, DEAE-Sephadex (Secholex®, Polidexide®), water soluble derivatives such as 3,3-ioene, N-(cycloalkyl)alkylamines and poliglusam, insoluble quaternized polystyrenes, saponins and mixtures thereof and those bile acid sequestrants disclosed in WO97/11345, WO98/57652, U.S. Pat. No. 3,692,895, and U.S. Pat. No. 5,703,188. Suitable inorganic cholesterol sequestrants include bismuth salicylate plus montmorillonite clay, aluminum hydroxide and calcium carbonate antacids.

HMG-CoA reductase inhibitors are dyslipidemic agents that can be used in therapeutic combinations with compounds described herein. Suitable HMG-CoA reductase inhibitors for use in therapeutic combination with a compounds described herein include: atorvastatin (Lipitor®; disclosed in U.S. Pat. No. 4,681,893, U.S. Pat. No. 5,385,929 and U.S. Pat. No. 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, cerivastatin (Baycol®; disclosed in U.S. Pat. No. 5,006,530, U.S. Pat. No. 5,502,199, and U.S. Pat. No. 5,177,080), crilvastatin, dalvastatin/RG 12561 (disclosed in EP738510A2), fluvastatin (Lescol®; disclosed in U.S. Pat. No. 4,739,073 and U.S. Pat. No. 534772), glenvastatin, fluindostatin/XU 62-320 (disclosed in EP363934A1), velostatin (visinolin; disclosed in U.S. Pat. No. 4,448,784 and U.S. Pat. No. 4,450,171), lovastatin (mevinolin; MK-803; Mevacor® (Merck and Co.) and related compounds disclosed in U.S. Pat. No. 4,231,938), Advicor/Nicostatin (a lovastatin-niacin mixture), mevastatin (and related compound disclosed in U.S. Pat. No. 3,983,140), compactin/ML 236B/CS 500/6-demethylmevinolin (and related compounds disclosed in U.S. Pat. No. 4,804,770), pitavastatin (also known as NK-104, itavastatin, nisvastatin, nisbastatin disclosed in U.S. Pat. No. 5,102,888), pravastatin (Eptastatin; CS-514; Pravachol® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin/rosuvastatin calcium (Crestor®; also known as ZD-4522 or atavastatin or visastatin; disclosed in U.S. Pat. No. 5,260,440), simvastatin (MMK-733; Zocor® (Merck and Co.) and related compounds as disclosed in U.S. Pat. No. 4,448,784 and U.S. Pat. No. 4,450,171), sirrivastatin, CI-981, PD134967-15, PD 135022, BMY-22089, PD 135023-15, BMY21950, PD 134965, dihydroxy open statins as disclosed in US2005010561, compounds disclosed in WO03/033481, US20050085497, U.S. Pat. No. 4,231,938, U.S. Pat. No. 4,444,784, U.S. Pat. No. 4,647,576, U.S. Pat. No. 4,686,237, U.S. Pat. No. 4,499,289, U.S. Pat. No. 4,346,227, U.S. Pat. No. 5,753,675, U.S. Pat. No. 4,613,610, EP0221025, and EP491226, and optical or geometric isomers thereof, and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzim-idazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Prodrugs of HMG CoA reductase inhibitors are also dyslipidemic agents. In certain embodiments, the prodrug is a lipophilic ester comprising an ester prodrug linkage to the HMG-like moiety of the statin drug and a lipophilic group described, for example, in WO05023305. Lipophilic alcohols available which may be used to form such statin prodrugs, include, but are not limited to, methanol, ethanol, propan-1-ol, propan-2-ol, butan-1-ol, butan-2-ol, pentan-1-ol, hexan-1-ol, heptan-1-ol, octan-1-ol, nonan-1-ol, decan-1-ol, 2-ethyl-hexan-1-ol, 3,3,5-trimethyl-cyclohexanol, 2-ethoxyethanol, and menthol. Examples of such lipophilic ester statin prodrugs include but are not limited to (3R,5R)-3,5-Dihydroxy-7-(2-isopropyl-4,5-diphenyl-3-phenylcarbamoyl-pyrrol-1-yl)-heptanoic acid, (E)-(3R,5S)-3,5-Dihydroxy-7-(1-isopropyl-3-phenyl-1H-indol-2-yl)-hept-6-enoic acid, (E)-(3R,5S)-3,5-Dihydroxy-7-[4-isopropyl-2-(methanesulfonyl-methyl-amino)-6-phenyl-pyrimidin-5-yl]-hept-6-enoic acid, (E)-(3R,5S)-7-(2-Cyclopropyl-4-phenyl-quinolin-3-yl)-3,5-dihydroxy-hept-6-enoic acid, (E)-(3R,5S)-7-(2,6-Diisopropyl-5-methoxymethyl-4-phenylpyridin-3-yl)-3,5-dihydroxy-hept-6-enoic acid, including free acid and pharmaceutically acceptable salt forms thereof. Hydroxylated statin forms and ester prodrugs thereof as described, for example, in WO05023305 are also dyslipidemic agents. Hydroxylated statins include but are not limited to (3R,5R)-3-, 5-Dihydroxy-7-[2-(4-hydroxy-phenyl)-5-isopropyl-3-phenyl-4-phenylcarbamoyl-pyrrol-1-yl]-heptanoic acid, (E)-(3R,5S)-3,5-Dihydroxy-7-[3-(4-hydroxy-phenyl)-1-isopropyl-1H-indol-2-yl]-hept-6-enoic acid, (E)-(3R,5S)-3,5-Dihydroxy-7-[4-(4-hydroxy-phenyl)-6-isopropyl-2-(methanesulfonyl-methyl-amino)-pyrimidin-5-yl]-hept-6-enoic acid, (E)-(3R,5S)-7-[2-Cyclopropyl-4-(4-hydroxy-phenyl)-quinolin-3-yl]-3,5-dihydroxy-hept-6-enoic acid, (E)-(3R,5S)-3,5-Dihydroxy-7-[4-(4-hydroxy-phenyl)-2,6-diisopropyl-5-methoxymethyl-pyridin-3-yl]-hept-6-enoic acid, including free acid and pharmaceutically acceptable salt forms thereof. Ester prodrugs of hydroxylated statins are of the general formula X—Y, where is X is a hydroxylated statin (e.g., (3R,5R)-3-, 5-Dihydroxy-7-[2-(4-hydroxy-phenyl)-5-isopropyl-3-phenyl-4-phenylcarbamoyl-pyrrol-1-yl]-heptanoic acid, (E)-(3R,5S)-3,5-Dihydroxy-7-[3-(4-hydroxy-phenyl)-1-isopropyl-1H-indol-2-yl]-hept-6-enoic acid, (E)-(3R,5S)-3,5-Dihydroxy-7-[4-(4-hydroxy-phenyl)-6-isopropyl-2-(methanesulfonyl-methyl-amino)-pyrimidin-5-yl]-hept-6-enoic acid, (E)-(3R,5S)-7-[2-Cyclopropyl-4-(4-hydroxy-phenyl)-quinolin-3-yl]-3,5-dihydroxy-hept-6-enoic acid and (E)-(3R,5S)-3,5-Dihydroxy-7-[4-(4-hydroxy-phenyl)-2,6-diisopropyl-5-methoxymethyl-pyridin-3-yl]-hept-6-enoic acid) and Y is chosen from formic acid, acetic acid, propan-1-oic acid, propan-2-oic acid, butan-1-oic acid, butan-2-oic acid, pentan-1-oic acid, hexan-1-oic acid, heptan-1-oic acid, octan-1-oic acid, nonan-1-oic acid, decan-1-oic acid, benzoic acid, cinnamic acid and 1-hydroxy-benzoic acid. Not limiting examples of ester prodrugs of hydroxylated statins are shown in WO05023305, FIG. 10.

Lipid modulating agents are dyslipidemic agents which function as high density lipoprotein (HDL), including synthetic HDL which contains lipid such as phosphotidyl choline, phosphatidyl serine, phosphatidyl ethanolamine, and other phospholipids in combination with HDL associated proteins such as ApoA-I or variants thereof including ApoAI-Milano (R173C) and biologically active peptides derived therefrom, the ApoA-I Paris variant (R151C), the reverse lipid transport (RLT) peptides, enzymes associated with HDL such as paraoxonase, and apo E, alone or formulated in combination with liposomes or emulsions (an example of a liposomal formulation is found in WO95/23592), see, for example, US20030109442 and US20050096307. HDL associated proteins include sequences present in HDL associated proteins that associate with HDL and synthetic peptides having equivalent binding or functional characteristics. HDL-associated proteins further include apolipoproteins such as Apo E, proApoA-I, ApoA-IParis, ApoA-II, proApoA-II, ApoA-IV, ApoC-I, ApoC-II, ApoC-III, including variants thereof which have been modified to include one or more sulfhydral groups, as described by Bielicki and Oda, Biochemistry 41:2089-2096 (2002). HDL-associated proteins further include paraoxonase, cholesteryl ester transfer protein, Lecithin Cholesterol Acyltransferase (LCAT), phospholipid transfer protein, including combinations thereof complexed with and without lipid. HDL-associated proteins can be used alone, in combination, complexed to one or more lipids alone or in combination complexed to one or more lipids. Non limiting examples include complexes comprising ApoA-I and lipid, complexes comprising paraoxanase and lipid, and complexes comprising ApoA-I, paraoxonase and lipid. HDL-associated proteins and lipids can be mixed in an aqueous solution in appropriate ratios complexed by methods known in the art and including freeze-drying, detergent solubilization followed by dialysis, microfluidization, sonication, and homogenization. Complex efficiency can be optimized, for example, by varying pressure, ultrasonic frequency, or detergent concentration. An example of a detergent commonly used to prepared HDL-associated protein-lipid complexes is sodium cholate. In some cases it is desirable to mix the lipid and the HDL-associated protein prior to administration. Lipids may be in solution or in the form of liposomes or emulsions formed using standard techniques such as sonication or extrusion. Sonication is generally performed with a tip sonifier, such as a Branson tip sonifier, in an ice bath. Typically, the suspension is subjected to several sonication cycles. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. or through a polycarbonate filter or other types of polymerized materials (i.e. plastics) commonly known. In some cases, the dyslipidemic agent comprises an HDL-associated protein with little or no lipid. Non limiting examples of lipids include phospholipids (such as soy phosphatidylcholine, egg phosphatidylcholine, soy phosphatidylglycerol, egg phosphatidylglycerol, palmitoyl-oleoyl-phosphatidylcholine distearoylphosphatidylcholine, distearoylphosphatidylglycerol, phosphatidylcholine, phosphatidylglycerol, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, distearoylphosphatidylethanolamine, dimyrstoyl-phosphatidylserine, and dioleyl-phosphatidylcholine) and non-phosphorus containing lipids (such as stearylamine, docecylamine, acetyl palmitate, and fatty acid amides). Additional lipids suitable for use are well known to persons of skill in the art and are cited in a variety of well known sources, e.g., McCutcheon's Detergents and Emulsifiers and McCutcheon's Functional Materials, Allured Publishing Co., Ridgewood, N.J. Generally, it is desirable that the lipids are liquid-crystalline at 37° C., 35° C., or 32° C. The concentration of the lipid in the formulation may vary. Persons of skill may vary these concentrations to optimize treatment with different lipid components or of particular patients. ApoA1 is combined with lipid in a ratio by weight of between 1:0.5 to 1:3. In certain embodiments, more lipid being preferred for clearance of cholesterol. The ratio may be around 1:1 is to produce the most homogenous population and for purposes of producing stable and reproducible batches. In certain embodiments, the lipid modulating agent is ETC-216, which is a synthetic HDL complex composed of 14 mg/mL of recombinant apolipoprotein A-I Milano and 13 mg/mL of 1-palmitoyl-2-oleoyl phosphatidyl choline (POPC) complex in sucrose-mannitol-phosphate buffer solution (sterile 6.4% sucrose, 0.8% mannitol in 6 mmol/L phosphate buffer, pH 7.4) (Esperion Therapeutics, Inc.), as a ready to inject solution or saline.

Other dyslipidemic agents which can be used a therapeutic combination with a compound described herein include:

peptides and peptide analogues that mimic the structural and pharmacological properties of human ApoA-I including those disclosed, for example in U.S. Pat. No. 6,004,925;
apolipoprotein E (apoE) and isoforms thereof including that produced by the methods disclosed in WO04/108922 and U.S. Pat. No. 5,834,596;
apolipoprotein A (apoA) and isoforms thereof including that produced by the methods disclosed in WO04/108922;
ApoA-I agonists including the peptides described in U.S. Pat. No. 6,004,925 and U.S. Pat. No. 6,037,323;
   HMG-CoA synthase inhibitors such as L-659,699 ((E,E)-11-[3′R-(hydroxy-methyl)-4′-oxo-2′R-oxetanyl]-3,5,7R-trimethyl-2,4-undecadienoic acid) and those disclosed in U.S. Pat. No. 5,120,729, U.S. Pat. No. 5,064,856, and U.S. Pat. No. 4,847,271;
   cholesterol absorption inhibitors such as plant sterols, plant stanols and/or fatty acid esters of plant stanols such as sitostanol ester used in Benecol® margarine, stanol esters, beta-sitosterol, sterol glycosides such as tiqueside, (3R,4S)-1-(4-fluorophenyl)-3-[(3S)-3-(4-fluorophenyl)-3-hydroxypropyl]-4-(4-hydroxyphenyl)azetidin-2-one (Zetia®), and the compounds disclosed in USRE37721, WO9302048, WO05044256, WO05033100, WO05021495, WO05021497, WO05009955, WO05000353, WO04087655, WO04000804, WO04000803, WO02050068, WO02050060, WO02050027, WO04000805, WO02066464, WO03026672, WO05042692, WO05042692, and WO04005247;
   phytoestrogen compounds such as disclosed in WO00/30665 including isolated soy bean protein, soy protein concentrate or soy flour as well as an isoflavone such as genistein, daidzein, glycitein or equol, or phytosterols, phytostanol or tocotrienol as disclosed in WO00/015201;
   an α-glucosidase inhibitor, an aldose reductase inhibitor and/or an LDL catabolism promoter such as disclosed in EP1022272;
   a matrix metalloproteinase inhibitor including but not limited to (±)-4-(4′-Chloro-biphenyl-4-yl)-4-hydroxy-butyric acid, (L)-2-(Dibenzofuran-2-sulfonylamino)-3-mercapto-propionic acid, (L)-2-(Dibenzofuran-2-sulfonylamino)-3-methyl-butyric acid, (L)-2-(Dibenzofuran-2-sulfonylamino)-3-phenyl-propionic acid, (L)-2-(Dibenzofuran-2-sulfonylamino)-3-tritylsulfanyl-propionic acid, (L)-2-(Dibenzofuran-2-sulfonylamino)-4-methyl-pentanoic acid, (S)-2-(4′-Amino-biphenyl-4-sulfonylamino)-3-methyl-butyric acid, (S)-2-(4′-Bromo-biphenyl-4-sulfonylamino)-3-methyl-butyric acid, (S)-2-(4′-Bromo-biphenyl-4-sulfonylamino)-3-phenyl-propionic acid, (S)-2-(Dibenzofuran-2-sulfonylamino)-4-phenyl-butyric acid, (S)-2-(dibenzofuran-3-sulfonylamino)-3-methyl-butyric acid, (S)-2-(dibenzofuran-3-sulfonylamino)-succinic acid, (S)-2-[4-(4-Benzyl-piperidin-1-yl)-benzenesulfonyl-amino]-3-phenyl-propionic acid, (S)-2-{4-[-4-(4-Methoxy-phenyl)-piperazin-1-yl]-benzenesulfonylamino}-phenylpropionic acid, (S)-2-Acetylamino-4-dibenzofuran-2-yl-4-oxo-butyric acid, (S)-2-Amino-4-dibenzofuran-2-yl-4-oxo-butyric acid, (S)-3-Methyl-2-(4′-nitro-biphenyl-4-sulfonylamino)-butyric acid, (S)-3-Phenyl-2-[4-(4-phenyl-piperidin-1-yl)-benzenesulfonyl-amino]-propionic acid, (S)-4-Dibenzofuran-2-yl-4-oxo-2-(2,2,2-trifluoroacetylamino)-butyric acid, (S)-4-Dibenzofuran-2-yl-4-oxo-2-(3-phenyl-propionylamino)-butyric acid, (S)-4-Dibenzofuran-2-yl-4-oxo-2-phenylacetylamino-butyric acid, [4-(4-Phenyl-piperidin-1-yl)-benzenesulfonylamino]-acetic acid, 2-(4′-bromobiphenyl-4-sulfonylamino)-3-methylbutyric acid, 2-(4′-Bromo-biphenyl-4-sulfonylamino)-3-methyl-butyric acid, 4-(4′-Bromo-biphenyl-4-yl)-4-hydroxyimino-butyric acid, 4-(4′-Chloro-biphenyl-4-yl)-4-(dimethylhydrazono)-butyric acid, 4-(4′-Chloro-biphenyl-4-yl)-4-hydroxyimino-butyric acid, 4-Oxo-4-[4-(4-phenyl-piperazin-1-yl)-phenyl]-butyric acid, 4-Oxo-4-[4-(4-phenyl-piperidin-1-yl)-phenyl]-butyric acid, batimastat, CDP-845 (Celltech), CGS27023A (Ciba-Giegy), CI-1026, fenbufen and related compounds disclosed in U.S. Pat. No. 3,784,701 and by Child et al., J Pharm Sci 1977; 66:466-476, galardin, marimastat, N-Hydroxy-2-[4-(4-phenyl-piperidin-1-yl)-benzenesulfonyl-amino]-acetamide, N-Hydroxy-4-oxo-4-[4-(4-phenyl-piperidin-1-yl)-phenyl]butyramide, PD 166793, RO-31-9790 (Roche), U24522 (Merck), and the compounds and/or peptides disclosed in EP0236872, EP0274453, EP0489577, EP0489579, EP0497192, EP0574758, EP2321081, U.S. Pat. No. 4,599,361, U.S. Pat. No. 5,183,900, U.S. Pat. No. 5,256,657, U.S. Pat. No. 5,270,326, U.S. Pat. No. 5,300,501, U.S. Pat. No. 5,304,604, U.S. Pat. No. 5,455,258, U.S. Pat. No. 5,524,190, U.S. Pat. No. 5,525,629, U.S. Pat. No. 5,530,128, U.S. Pat. No. 5,530,161, WO90/05716, WO90/05719, WO91/02716, WO92/09563, WO92/13831, WO92/17460, WO92/22523, WO93/09090, WO93/09097, WO93/20047, WO93/244, WO93/24449, WO94/02446, WO94/02447, WO95/13289, WO96/11209, WO97/27174, and US20050020607 (including the compounds specifically disclosed by chemical formula and name);
   a sodium-proton exchange inhibitor such as disclosed in DE19622222;
   an LDL-receptor inducer or a steroidal glycoside such as disclosed in U.S. Pat. No. 5,698,527 and GB2304106;
   LUV (large unilamellar vesicles) products including ETC-588 (Pfizer);
   acyl coenzyme A-cholesterol acyl transferase (ACAT) inhibitors such as avasimibe (Current Opinion in Investigational Drugs. 3(9):291-297 (2003)), eflucimibe, HL-004, lecimibe, (DuP-1), KY505, SMP 797, TS-962 (Taisho Pharmaceutical Co. Ltd), F-1394, CS-505 (pactimibe), F-12511, K-10085 and YIC-C8-434, CL-277,082 (Clin Pharmacol Ther. 48(2):189-94 (1990)) and those disclosed in Drugs of the Future 24, 9-15 (1999); Nicolosi et al, Atherosclerosis (1998), 137(1), 77-85; Ghiselli and Giancarlo, Cardiovasc. Drug Rev. (1998), 16(1), 16-30; Smith, et al, Bioorg. Med. Chem. Lett. (1996), 6(1), 47-50; Krause et al, Inflammation: Mediators Pathways (1995), 173-98, Publisher: CRC, Boca Raton, Fla.; Sliskovic et al, Curr. Med. Chem. (1994), 1(3), 204-25; Stout et al, Chemtracts: Org. Chem. (1995), 8(6), 359-62 and U.S. Pat. No. 5,510,379, WO96/26948 and WO96/10559;
   CETP inhibitors such as JTT 705 identified in Nature 406, (6792):203-7 (2000), torcetrapib (CP-529,414 described in US20030186952 and WO00/017164), CP 532,632, BAY63-2149, CeTi-1, SC 591, SC 795 (Pharmacia), SC 744 (Pharmacia) and the like including those described in Current Opinion in Investigational Drugs. 4(3):291-297 (2003) and those disclosed in J. Antibiot., 49(8): 815-816 (1996), and Bioorg. Med. Chem. Lett., 6:1951-1954 (1996) and patent publications U.S. Pat. No. 5,512,548, U.S. Pat. No. 6,147,090, WO99/20302, WO99/14204, WO99/41237, WO95/04755, WO96/15141, WO96/05227, WO038721, WO/0038722, EP796846, EP818197, EP818448, EP992496, DE19704244, DE19741051, DE19741399, DE197042437, DE19709125, DE19627430, DE19832159, DE19741400, JP 11049743, and JP 09059155;
   squalene synthetase inhibitors such as squalestatin-1, and those disclosed in U.S. Pat. No. 4,871,721, U.S. Pat. No. 4,924,024, U.S. Pat. No. 5,712,396 (α-phosphono-sulfonates), Biller et al (1988) J. Med. Chem., 31:1869 (e.g., isoprenoid (phosphinyl-methyl)phosphonates), Biller et al (1996) Current Pharmaceutical Design, 2:1, P. Ortiz de Montellano et al (1977) J. Med. Chem. 20:243 (terpenoid pyrophosphates), Corey and Volante (1976) J. Am. Chem. Soc., 98:1291 (farnesyl diphosphate analog A and presqualene pyrophosphate (PSQ-PP) analogs), McClard et al (1987) J.A.C.S., 109:5544 (phosphinylphosphonates), Capson, T. L., PhD dissertation, June, 1987, Dept. Med. Chem. U of Utah, Abstract, Table of Contents, pp 16, 17, 40-43, 48-51, Summary, (cyclopropanes), Curr. Op. Ther. Patents (1993) 861, and patent publications EP0567026A1, EP0645378A1, EP0645377A1, EP0611749A1, EP0705607A2, EP0701725A1, US20040072830, and WO96/09827;
   antioxidants such as probucol (and related compounds disclosed in U.S. Pat. No. 3,674,836), probucol derivatives such as AGI-1067 (and other derivatives disclosed in U.S. Pat. No. 6,121,319 and U.S. Pat. No. 6,147,250), tocopherol, ascorbic acid, retinol (as disclosed in WO94/15592), β-carotene, selenium, vitamin C and pharmaceutically acceptable salts and esters thereof, an antihomocysteine agent such as folic acid, folate, vitamin E, vitamin B₆, vitamin B₁₂ and pharmaceutically acceptable salts and esters thereof;
   PPARα agonists such as those disclosed in U.S. Pat. No. 6,028,109 (fluorophenyl compounds), WO00/75103 (substituted phenylpropionic compounds), WO98/43081 and fibric acid derivatives (fibrates) such as beclofibrate, benzafibrate, bezafibrate (CAS RN 41859-67-0, see U.S. Pat. No. 3,781,328), binifibrate (CAS RN 69047-39-8, see BE884722), ciprofibrate (CAS RN 52214-84-3, see U.S. Pat. No. 3,948,973), clinofibrate (CAS RN 30299-08-2, see U.S. Pat. No. 3,716,583), clofibrate (such as ethyl 2-(p-chlorophenoxy)-2-methyl-propionate, e.g., Atromid-S® capsules (Wyeth-Ayerst), clofibric acid, etofibrate, pirifibrate, ronifibrate, simfibrate, theofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g., Lopid® tablets (Parke Davis)), lifibrol, GW 7647, BM 170744, LY518674 and those fibrate and fibrate acid derivatives disclosed in WO03/033456, WO03/033481, WO03/043997, WO03/048116, WO03/053974, WO03/059864, and WO03/05875; FXR receptor modulators such as GW 4064 (described in WO00/37077, Glaxo Group Limited), SR 103912, 3-deoxychenodeoxycholic acid, QRX-401 (QuatRx), (E)- and (Z)-guggulsterone, chenodeoxycholic acid (CDCA), 6alpha-ethyl-chenodeoxycholic acid (6-ECDCA), compounds disclosed in WO05056554 (including the compounds specifically named in claims 3, 7, 9, 11, 13, 17, 19, 21 23, 27, 29, 31, 33, 35, 37, 39, and 41), compounds disclosed in U.S. Pat. No. 6,906,057 (such as (Z)-5-[2-(5,5,8,8-tetramethyl-5,6,7,8-tetrahydronapthalen-2-yl)-2-(trimethylsilyl)vinyl]thiophene-2-carboxylic acid, (Z)-5-[2-(5,5,8,8-tetramethyl-5,6,7,8-tetrahydronapthalen-2-yl)-2-(trimethylsilyl)vinyl]thiophene-2-carboxylic acid and the compounds described in claims 26-28), farnesol (3,7,11-trimethyl-2,6,10-dodecatrienol), farnesal, farnesyl acetate, farnesoic acid, geranylgeraniol, and juvenile hormone III (7-methyl-9-(3,3-dimethyloxiranyl)-3-methyl-2,6-nonadienoic acid methyl ester), methyl farnesyl ether, ethyl farnesyl ether, methyl turesoztce, ethyl Carnesoate, 7-methyl-9-(3,3-dimethyloxiranyl)-3-methyl-2,6-nonadienoic acid methyl ester, and 7-methyl-9-(3,3-dimethyloxiranyl)-3-methyl-2,6-nonadienoic acid ethyl ester, compounds disclosed in WO04/048349 (such as 3-{[(4-{[3-(2,6-Dichlorophenyl)-5-isopropylisoxazol-4-yl]methoxy}-2-methylphenyl)-(methyl)amino]methyl}benzoic acid; Methyl 4-0[(4-{[3-(2,6-dichlorophenyl)-5-isopropyl-4-isoxazolyl]methoxy}-2-dimethylanilino) methyl]benzoate; 3-{[(2-Chloro-4-{[3-(2,6-dichlorophenyl)-5-isopropylisoxazol-4-yl]methoxy}-phenyl)amino]methyl}benzoic acid; 5-[(4-{[3-(2,6-Dichlorophenyl)-5-isopropyl-4-isoxazolyl]methoxy}-2-dimethyl-anilin) methyl]-2-furoic acid; 4-[(4-1-[3-(2,6-Dichlorophenyl)-5-isopropyl-4-isoxazolyllmethoxy}-2-dimethyl-anilino) methyl]benzoic acid; Methyl 2-[(4-{[3-(2,6-dichlorophenyl)-5-isopropyl-4-isoxazolyl]methoxy}-2-dimethylanilino) methyl]-3-furoate; N-(2,1,3-Benzoxadiazol-5-ylmethyl)-4-{[3-(2,6-dichlorophenyl)-5-isopropyl-4-isoxazolyl]methoxy}-N,2-dimethylaniline; N-(4-{[3-(2,6-Dichlorophenyl)-5-isopropyl-4-isoxazolyl]methoxy}-2-methylphenyl)-N-methyl-N-[4-(1,2,3-thiadiazol-4-yl) benzyl]amine; 4-[(4-1 [3-(2,6-Dichlorophenyl)-5-isopropyl-4-isoxazolyllmethoxy}-2-dimethyl-anilino) methyl]benzonitrile; 2-[(4-{[3-(2,6-Dichlorophenyl)-5-isopropyl-4-isoxazolyl]methoxy}-2-dimethyl-anilin) methyl]-3-furoic acid; {3-[(4-{[3-(2,6-Dichlorophenyl)-5-isopropyl-4-isoxazolyl]methoxy}-2-dimethylanilino) methyl]phenyl}methanol; {4-[(4-{[3-(2,6-Dichlorophenyl)-5-isopropyl-4-isoxazolyl]methoxy}-2-dimethylanilino)methyl]phenyl}methanol; 3-[(4-{[3-(2,6-Dichlorobenzyl)-5-ethyl-4-isoxazolyl]methoxy}-2-dimethylanilino) methyl]benzoic acid; 3-{[(4-{[5-Isopropyl-3-(2,4,6-trichlorophenyl)isoxazol-4-yl]methoxy}-2-methylphenyl)-(methyl)amino]methyl}benzoic acid; 3-[(4-1 [3-(2,6-Dichlorobenzyl)-5-isopropyl-4-isoxazolyi]methoxy}-2-dimethylanilino) methyl]benzoic acid; 3-{[(4-{[3-(2-Chlorobenzyl)-5-isopropylisoxazol-4-yl]methoxy}-2-methylphenyl)-(methyl)-amino]methyl}benzoic acid; 3-[(4-{[5-Cyclopropyl-3-(2,6-dichlorobenzyl)-4-isoxazolyl]methoxy}-2-dimethylanilino) methyl]benzoic acid; 5-[5-({5-Isopropyl-3-[2-(trifluoromethoxy)phenyl]-4-isoxazolyl}methoxy)-2-dimethylanilino]methyl}-2-furoic acid; 4-{[4-({5-isopropyl-3-[2-(trifluoromethoxy)phenyl]-4-isoxazolyl}methoxy)-2-dimethylanitino]methyl}benzoic acid; 3-{[4-({5-Isopropyl-3-[2-(trifluoromethoxy)phenyl]-4-isoxazolyl}methoxy)-2-dimethylanilino]methyl}benzoic acid; Methyl 5-{[4-(5-isopropyl-3-[2-(trifluoromethoxy)phenyl]-4-isoxazolyl}methoxy)-2-dimethylanilino]methyl}-2-furoate; Methyl 4-{[4-({5-isopropyl-3-[2-(trifluoromethoxy)phenyl]-4-isoxazolyl}methoxy)-2-dimethylanilino]methyl}benzoate; 4-{[(2-Chloro-4-1 [3-(2,6-dichlorophenyl)-5-isopropylisoxazol-4-yl]methoxy}phenyl)-amino]carbonyl}benzoic acid; Methyl 3-[(2-chloro-4-{[3-(2,6-dichlorophenyl)-5-isopropyl-4-isoxazolyl]methoxy}-anilino) carbonyl]benzoate; Methyl 4-[(2-chloro-4-{[3-(2,6-dichlorophenyl)-5-isopropyl-4-isoxazolyl]methoxy}-anilino) carbonyl]benzoate; 3-[(4-{[3-(2,6-Dichlorophenyl)-5-isopropyl-4-isoxazolyl]methoxy}-2-methylanilino)-carbonyl]benzoic acid; 4-[(4-{[3-(2,6-Dichlorophenyl)-5-isopropyl-4-isoxazolyl]methoxy}-2-methylanilino)carbonyl]benzoic acid; 3-[(2-Chloro-4-{[3-(2,6-dichlorophenyl)-5-isopropyl-4-isoxazolyl]methoxy}-anilin) carbonyl]benzoic acid; 3-[(4-{[3-(2,6-Dichlorophenyl)-5-isopropyl-4-isoxazolyl]methoxy}-2-dimethylanilino) carbonyl]benzoic acid; 3-[(2-Chloro-4-{[3-(2,6-dichlorophenyl)-5-isopropyl-4-isoxazolyl]methoxy}-methylanilino) carbonyl]benzoic acid; 4-[(2-Chloro-4-{[3-(2,6-dichlorophenyl)-5-isopropyl-4-isoxazolyl]methoxy}-methylanilino) carbonyl]benzoic acid; 3-{[4-({5-isopropyl-3-[2-(trifluoromethoxy)phenyl]-4-isoxazolyl}methoxy)-2-dimethylanilino]carbonyl}benzoic acid; 3-{[4-({5-Isopropyl-3-[2-(trifluoromethoxy)phenyl]-4-isoxazolyl}methoxy)-2-methylanilino]carbonyl}benzoic acid; 3-{[2-Chloro-4-({5-isopropyl-3[2-(trifluoromethoxy)phenyl]-4-isoxazolyl}-methoxy)methylanilino]carbonyl}benzoic acid; Methyl 3-{[(4-{[3-(2,6-dichlorophenyl)-5-isopropylisoxazol-4-yl]methoxy}-2-methylphenyl) (methyl)amino]sulfonyl}benzoate; Methyl 3-{[(2-chloro-4-{[3-(2,6-dichlorophenyl)-5-isopropylisoxazol-4-yl]methoxy}-phenyl) amino]sulfonyl}benzoate; Methyl 3-{[(4-{[3-(2,6-dichlorophenyl)-5-isopropylisoxazol-4-yl]methoxy}-2-methylphenyl)amino]sulfonyl}benzoate; Methyl 3-{[(4-{[3-(2,6-dichlorophenyl)-5-isopropylisoxazol-4 yl]methoxy}phenyl)-amino]-sulfonyl}benzoate; 3-{[(4-{[3-(2,6-Dichlorophenyl)-5-isopropylisoxazol-4-yl]methoxy}-2-methylphenyl)-amino]sulfonyl}benzoic acid; 3-{[(4-{[3-(2,6-Dichlorophenyl)-5-isopropylisoxazol-4-yl]methoxy}phenyl)amino-sulfonyl}benzoic acid; Methyl 3-([(2-chloro-4-{[3-(2,6-dichlorophenyl)-5-isopropylisoxazol-4-yl]methoxy}-phenyl) (methyl)amino]sulfonyl}benzoate; Methyl 3-{[(4-{[3-(2,6-dichlorophenyl)-5-isopropylisoxazol-4-yl]methoxy}phenyl)-(methyl)amino]sulfonyl}benzoate; Methyl 3-{[(2-chloro-4-{[3-(2,6-dichlorophenyl)-5-isopropylisoxazol-4-yl]methoxy}-phenyl) (ethyl)amino]sulfonyl}benzoate; Methyl 3-{[(4-{[3-(2,6-dichlorophenyl)-5-isopropylisoxazol-4-yl]methoxy}-2-methyl-phenyl) (ethyl)amino]sulfonyl}benzoate; Methyl 3-[(4-{[3-(2,6-dichlorophenyl)-5-isopropyl isoxazol-4-yl]methoxy}phenyl)-(ethyl)-amino]sulfonyl}benzoate; 3-{[(2-Chloro-4-{[3-(2,6-dichlorophenyl)-5-isopropylisoxazol-4-yl]methoxy}phenyl)amino]sulfonyl}benzoic acid; 3-{[(2-Chloro-4-{[3-(2,6-dichlorophenyl)-5-isopropylisoxazol-4-yl]methoxy}phenyl)-(methyl)amino]sulfonyl}benzoic acid; 3-{[(4-{[3-(2,6-Dichlorophenyl)-5-isopropylisoxazol-4-yl]methoxy}-2-methylphenyl)-(methyl)amino]sulfonyl}benzoic acid; 3-{[(4-{[3-(2,6-Dichlorophenyl)-5-isopropylisoxazol-4-yl]methoxy}phenyl)(methyl)-amino]sulfonyl}benzoic acid; 3-{[(2-Chloro-4-{[3-(2,6-dichlorophenyl)-5-isopropylisoxazol-4-yl]methoxy}phenyl)-(ethyl)amino]benzoic acid; 3-{[(4-{[3-(2,6-Dichlorophenyl)-5-isopropylisoxazol-4-yl]methoxy}-2-methylphenyl)-(ethyl)amino]sulfonyl}benzoic acid; 3-{[(4-{[3-(2,6-Dichlorophenyl)-5-isopropylisoxazol-4-yl]methoxy}phenyl)(ethyl)-amino]benzoic acid; Methyl 4-{[(2-chloro-4-{[3-(2,6-dichlorophenyl)-5-isopropylisoxazol-4-yl]methoxy}-phenyl)amino]sulfonyl}benzoate; Methyl 4-{[(4-{[3-(2,6-dichlorophenyl)-5-isopropylisoxazol-4-yl]methoxy}-2-methyl-phenyl)amino]sulfonyl}benzoate; Methyl 4-{[(4-{[3-(2,6-dichlorophenyl)-5-isopropylisoxazol-4-yl]methoxy}phenyl)-amino]benzoate; 4-{[(2-Chloro-4-{[3-(2,6-dichlorophenyl)-5-isopropylisoxazol-4-yl]methoxy}phenyl)-amino]sulfonyl}benzoic acid; 4-{[(4-{[3-(2,6-Dichlorophenyl)-5-isopropyl isoxazol-4-yl]methoxy}-2-methylphenyl)-amino]sulfonyl}benzoic acid; 4-{[(4-{[3-(2,6-Dichlorophenyl)-5-isopropylisoxazol-4-yl]methoxy}phenyl)amino]-sulfonyl}benzoic acid; Methyl 4-{[(4-{[3-(2,6-dichlorophenyl)-5-isopropylisoxazol-4-yl]methoxy}-2-methyl-phenyl) (methyl)amino]benzoate; Methyl 4-{[(4-{[3-(2,6-dichlorophenyl)-5-isopropylisoxazol-4-yl]methoxy}phenyl)-(methyl)amino]sulfonyl}benzoate; Methyl 4-{[(4-{[3-(2,6-dichlorophenyl)-5-isopropylisoxazol-4-yl]methoxy}phenyl)(ethyl)amino]sulfonyl}benzoate; 4-{[(2-Chloro-4-{[3-(2,6-dichlorophenyl)-5-isopropylisoxazol-4-yl]methoxy}phenyl)-(methyl)aminolsulfonyllbenzoic acid; 4-[(4-1-[3-(2,6-Dichlorophenyl)-5-isopropylisoxazol-4-yl]methoxy}-2-methylphenyl)(methyl)amino]sulfonyl}benzoic acid; 4-{[(4-{[3-(2,6-Dichlorophenyl)-5-isopropylisoxazol-4-yl]methoxy}phenyl) (methyl)-amino]sulfonyl}benzoic acid; 4-1 [(2-Chloro-4-1 [3-(2,6-dichlorophenyl)-5-isopropylisoxazol-4-yl]methoxy}phenyl)(ethyl)amino]sulfonyl}benzoic acid; 4-{[(4-{[3-(2,6-Dichlorophenyl)-5-isopropylisoxazol-4-yl]methoxy}-2-methyl phenyl)-(ethyl)amino]sulfonyl}benzoic acid; 4-{[(4-{[3-(2,6-Dichlorophenyl)-5-isopropylisoxazol-4-yl]methoxy}phenyl)(ethyl)amino]-sulfonyl}benzoic acid; 3-({[2-Chloro-4-({5-isopropyl-3-[2-(trifluoromethoxy)phenyl]isoxazol-4-yl}methoxy)-phenyl]amino}sulfonyl)benzoic acid; 3-({[4-({5-isopropyl-3-[2-(trifluoromethoxy)phenyl]isoxazol-4-yl}methoxy)-2-methyl-phenyl]amino}sulfonyl)benzoic acid; Methyl 3-{[[2-chloro-4-({5-isopropyl-3-[2-(trifluoromethoxy)phenyl]-isoxazol-4-yl}methoxy)phenyl (methyl)amino]sulfonyl}benzoate; Methyl 3-{[[4-({5-isopropyl-3-[2-(trifluoromethoxy)phenyl] isoxazol-4-yl}methoxy)-2-methylphenyl](methyl)amino]sulfonyl}benzoate; 3-[loro-4-({5-isopropyl-3-[2-(trifluoromethoxy)phenyl] isoxazol-4-yl}methoxy)-phenyl] (methyl)amino]sulfonyl}benzoic acid; 3-{[[4-({5-Isopropyl-3-[2-(trifluoromethoxy)phenyl]isoxazol-4-yl}methoxy)-2-methyl-phenyl] (methyl)amino]sulfonyl}benzoic acid; 3-1[(4-{[3-(2,6-Dichlorobenzyl)-5-ethylisoxazole-4-yl]methoxy}-2-methylphenyl)(methyl)-amino]sulfonyl}benzoic acid; Methyl 4-[(2-chloro-4-{[3-(2,6-dichlorophenyl)-5-isopropylisoxazol-4-yl]methoxy}-benzyl) oxy]benzoate; Methyl 3-[(2-chloro-4-{[3-(2,6-dichlorophenyl)-5-isopropylisoxazol-4-yl]methoxy}-benzyl)oxy]benzoate; 3-[(2-Chloro-4-{[3-(2,6-dichlorophenyl)-5-isopropylisoxazol-4-yl]methoxy}-benzyl) oxy]-benzoic acid; 3-[loro-4-{[3-(2,6-dichlorophenyl)-5-isopropylisoxazol-4-yl]methoxy}-benzyl)thio]-benzoic acid; 3-[(4-{[3-(2,6-Dichlorophenyl)-5-isopropylisoxazol-4-yl]methoxy}-2-methylbenzyl)-oxy]-benzoic acid; 3-[(4-{[3-(2,6-Dichlorophenyl)-5-isopropylisoxazol-4-yl]methoxy}-2-methylbenzyl)-thio]-benzoic acid; 4-[(2-Chloro-4-1 [3-(2,6-dichlorophenyl)-5-isopropylisoxazol-4-yl]methoxy}benzyl)-oxy]benzoic acid; 4-[(2-Chloro-4-{[3-(2,6-dichlorophenyl)-5-isopropyl isoxazol-4-yl]methoxy}benzyl)-thio]-benzoic acid; Methyl 3-{[2-chloro-4-({5-isopropyl-3-[2-(trifluoromethoxy)-phenyl] isoxazol-4-yl methoxy)benzyl]oxy}benzoate; Methyl 3-{[4-({5-isopropyl-3-[2-(trifluoromethoxy)phenyl]isoxazol-4-yl}methoxy)-2-methyl-benzyl]oxy}benzoate; 3-{[2-Chloro-4-({5-isopropyl-3-[2-(trifluoromethoxy)phenyl] isoxazol-4-yl}methoxy)-benzyl]oxy}benzoic acid; 3-{[4-({5-Isopropyl-3-[2-(trifluoromethoxy)phenyl]isoxazol-4-yl}methoxy)-2-methyl-benzyl]oxy}benzoic acid; 3-[(2-Chloro-4-{[3-(2,6-dichlorobenzyl)-5-ethylisoxazole-4-yl]methoxy}benzyl)oxy]-benzoic acid; Methyl 3-{[2-chloro-4-({5-isopropyl-3-[2-(trifluoro-methoxy)phenyl] isoxazol-4-yl}methoxy)benzyl]thio}benzoate; Methyl 3-{[4-({5-isopropyl-3-[2-(trifluoromethoxy)phenyl] isoxazol-4-yl}methoxy)-2-methylbenzyl]thio}benzoate; 3-{[2-Chloro-4-({5-isopropyl-3-[2-(trifluoromethoxy)phenyl] isoxazol-4-yl}methoxy)-benzyl]thio}benzoic acid; 3-{[4-({5-Isopropyl-3-[2-(trifluoromethoxy)-phenyl] isoxazol-4-yl}methoxy)-2-methyl-benzyl]thio}benzoic acid; Methyl 3-[(2-chloro-4-{[3-(2,6-dichlorophenyl)-5-isopropylisoxazol-4-yl]methoxy}benzyl)(methyl)amino]benzoate; 3-[(2-Chloro-4-{[3-(2,6-dichlorophenyl)-5-isopropylisoxazol-4-yl]methoxy}benzyl)-(methyl)amino]benzoic acid; 3-[(2-Chloro-4-{[3-(2,6-dichlorophenyl)-5-isopropylisoxazol-4-yl]methoxy}benzyl)-amino]benzoic acid; Ethyl 3-{[2-chloro-4-({5-isopropyl-3-[2-trifluoromethoxy)phenyl]-4-isoxazolyl}-methoxy)benzyl]amino}benzoate; 3-{[2-Chloro-4-({5-isopropyl-3-[2-(trifluoromethoxy)phenyl]-4-isoxazolyl}methoxy)benzyl]amino}benzoic acid; 3-[[2-Chloro-4-({5-isopropyl-3-[2-(trifluoromethoxy)phenyl]-4-isoxazolyl}methoxy)-benzyl] (methyl)amino]benzoic acid; Methyl 4-[(4-{[3-(2,6-dichlorophenyl)-5-isopropylisoxazol-4-yl]methoxy}-2-methylphenoxy)methyl]benzoate; Methyl 3-[(2-chloro-4-{[3-(2,6-dichlorophenyl)-5-isopropyl isoxazol-4-yl]methoxy}-phenoxy)methyl]benzoate; Methyl 3-[(4-{[3-(2,6-dichlorophenyl)-5-isopropylisoxazol-4-yl]methoxy}-2-methyl-phenoxy)methyl]benzoate; 3-[(2-Chloro-4-{[3-(2,6-dichlorophenyl)-5-isopropylisoxazol-4-yl]methoxy}phenoxy)-methyl]benzoic acid; 3-[(4-{[3-(2,6-Dichlorophenyl)-5-isopropylisoxazol-4-yl]methoxy}-2-methylphenoxy)-methyl]benzoic acid; and Methyl 4-[(4-{[3-(2,6-dichlorophenyl)-5-isopropylisoxazol-4-yl]methoxy}-2-methyl-phenoxy)methyl]benzoate) and the like;
   LXR receptor modulators such as α-hydroxycholesterol, 25-hydroxycholesterol, 27-hydroxy-cholesterol, 4β-hydroxycholesterol, 24-hydroxycholesterol, 20(S)-hydroxycholesterol, 22(R)-hydroxycholesterol, 20,22-dihydroxycholesterol, GW 3965, T9013137, and XTC0179628, and those disclosed in US20030125357, WO03/045382, WO03/053352, WO03/059874, and the like;
   HM74 and HM74A (human HM74A is Genbank Accession No. AY148884 and rat HM74A is EMM_patAR098624) receptor agonists such as nicotinic acid (niacin) and derivatives thereof (e.g., compounds comprising a pyridine-3-carboxylate structure or a pyrazine-2-carboxylate structure, including acid forms, salts, esters, zwitterions and tautomers, where available) including but not limited to those disclosed in Wise et al (2003) J. Biol. Chem. 278: 9869 (e.g., 5-methylpyrazole-3-carboxylic acid and acifran (4,5-dihydro-5-methyl-4-oxo-5-phenyl-2-furan carboxylic acid pyradine-3-acetic acid)), as well as trans-metanicotine, epibatidine or one of its analogs, pyridol or derivatives thereof, piperidine alkaloids (such as lobeline and analogs thereof) and imidacloprid or one of its analogs, 5-methyl nicotinic acid, nicotinuric acid, aluminum nicotinate, nicoclonate, nicomol, niceritrol, oxiniacic acid, nicofuranose, acipimox (5-methylpyrazine-2-carboxylic acid 4-oxide), Niaspan® (niacin extended-release tablets; Kos) and those which can be easily identified by one skilled in the art which bind to and agonize the HM74A or HM74 receptor (for example using the assays disclosed in Wise et al (2003) J. Biol. Chem. 278:9869 (nicotine binding and [³⁵S]-GTPγS binding assays), Soga et al (2003) Biochem. Biophys. Res. Comm. 303:364 (radiolabel binding assay using the HM74 receptor which could be adapted to the HM74A receptor), Tunaru et al (2003) Nature Medicine 9:352 (calcium mobilization assay using the HM74 receptor which could be adapted to the HM74A receptor) and U.S. Pat. No. 6,420,183 (FLIPR assays are described generally in and may be adapted to the HM74A or HM74 receptor);
   renin angiotensin system inhibitors;
   bile acid reabsorption inhibitors (bile acid reuptake inhibitors), such as BARI1453, SC435, PHA384640, S8921, AZD7706, and the like;
   PPARδ agonists (including partial agonists) such as GW 501516, and GW 590735, and those disclosed in U.S. Pat. No. 5,859,051 (acetophenols), WO03/024395, WO97/28149, WO01/79197, WO02/14291, WO02/46154, WO02/46176, WO02/076957, WO03/016291, WO03/033493, WO99/20275 (quinoline phenyl compounds), WO99/38845 (aryl compounds), WO00/63161 (1,4-disubstituted phenyl compounds), WO01/00579 (aryl compounds), WO01/12612, WO05/028453, & WO01/12187 (benzoic acid compounds), and WO97/31907 (substituted 4-hydroxy-phenylalconic acid compound);
   sterol biosynthesis inhibitors such as DMP-565;
   a sterol regulating element binding protein-I (SREBP-1) as disclosed in WO00/050574, for example, a sphingolipid, such as ceramide, or neutral sphingomyelenase (N-SMase) or fragment thereof,
   triglyceride synthesis inhibitors;
   microsomal triglyceride transport (MTTP or MTP) inhibitors, such as inplitapide, LAB687, 9-[4-[4-[[2-(2,2,2-Trifluoroethoxy)benzoyl]amino]-1-piperidinyl]butyl]-N-(2,2,2-trifluoroethyl)-9H-fluorene-9-carboxamide, and CP346086, and those disclosed in U.S. Pat. No. 5,595,872; U.S. Pat. No. 5,739,135; U.S. Pat. No. 5,712,279; U.S. Pat. No. 5,760,246; U.S. Pat. No. 5,827,875; U.S. Pat. No. 5,885,983 and U.S. Pat. No. 5,962,440;
   HMG-CoA reductase gene expression inhibitors (e.g., compounds that decrease HMG-CoA reductase expression by affecting (e.g., blocking) transcription or translation of HMG-CoA reductase into protein or compounds that may be biotransformed into compounds that have the aforementioned attributes by one or more enzymes in the cholesterol biosynthetic cascade or may lead to the accumulation of an isoprene metabolite that has the aforementioned activities (such regulation is readily determined by those skilled in the art according to standard assays (Methods of Enzymology, 110:9-19 1985))) such as those disclosed in U.S. Pat. No. 5,041,432 (certain 15-substituted lanosterol derivatives) and E. I. Mercer (1993) Prog. Lip. Res. 32:357 (oxygenated sterols that suppress the biosynthesis of HMG-CoA reductase);
   squalene epoxidase inhibitors such as NB-598 ((E)-N-ethyl-N-(6,6-dimethyl-2-hepten-4-y-nyl)-3-[(3,3′-bithiophen-5-yl)methoxy]benzene-methanamine hydrochloride); low density lipoprotein (LDL) receptor inducers such as MD-700 (Taisho Pharmaceuticals, LY295427 (Eli Lilly), HOE-402 (an imidazolidinyl-pyrimidine derivative that directly stimulates LDL receptor activity, see Huettinger et al (1993) Arterioscler. Thromb. 13:1005);
   platelet aggregation inhibitors;
   5-LO or FLAP inhibitors;
   PPAR-α activators such as 2,4-dichlorophenoxyacetic acid, 2,4,5-trichlorophenoxyacetic acid, 2-methyl-4-chlorophenoxyacetic acid, 2-phenoxy-2-methylpropanoic acid ethyl ester, 2-(4-bromophenoxy)-2-methylpropanoic acid ethyl ester, 2-(4-iodophenoxy)-2-methylpropanoic acid ethyl ester, 2-(2-chlorophenoxy)-2-methylpropanoic acid ethyl ester, 2-(3-chlorophenoxy)-2-methylpropanoic acid ethyl ester, 2-(4-chlorophenoxy)-2-methylpropanoic acid ethyl ester, 2-(4-(4-chlorophenyl)phenoxy)-2-methylpropanoic acid ethyl ester, 2-(4-(4-chlorobenzoyl)phenoxy)-2-methylpropanoic acid isopropyl ester, 2-(4-(2,2-dichlorocyclopropyl)phenoxy)-2-methylpropanoic acid, 2-(4-(4-chlorobenzoylaminoethyl)phenoxy)-2-methylpropanoic acid, 2-(2,3-dimethyl-4-(1,2,3,4-tetrahydronaphth-1-yl)phenoxy)acetic acid, 2-(2-methyl-3-ethyl-4-(4-chlorobenzyl)phenoxy)acetic acid, (4-chloro-6-(2,3-xylidino)-2-pyrimidinyl)thioacetic acid, 2-((4-chloro-6-(2,3-xylidino)-2-pyrimidinyl)thioacetamido)ethanol, perfluoro-n-decanoic acid, di-(2-ethylhexyl)adipate, di-(2-ethylhexyl)phosphate, di-(2-ethylhexyl)sebacate, bis-(carboxymethylthio)-1,10-decane, ethyl 4-(4-chlorophenoxy)butanoate, 2-(2-nitro-5-(2-chloro-4-trifluoromethylphenoxy)benzoyloxy)propanoic acid ethyl ester, 2-(4-(4-chlorobenzoyl))phenoxy-2-(2-methylpropionamido)ethylsulfonic acid, tetradecyloxyacetic acid, tetradecyloxypropionic acid, perfluorobutanoic acid, perfluorooctanoic acid, tetradecylthioacetic acid, tetradecylthiopropionic acid, di-(2-ethylhexyl)phthalate, mono-(2-ethylhexyl)phthalate, 2-ethylhexanoic acid, 2-propylhexanoic acid, compounds described in WO04010992, WO04/010936 (such as 5-{3-[2-Chloro-4-(2,2,2-trifluoro-ethoxy)-phenoxy]-propoxy}-2-methyl-2,3-dihydro-benzofuran-2-carboxylic acid, 5-{3-[4-(2,2-Dimethyl-propyl)-2-propyl-phenoxy]-propoxy}-2-methyl-2,3-dihydro-benzofuran-2-carboxylic acid, 5-[3-(2-Chloro-4-trifluoromethoxy-phenoxy)-propoxy]-2-methyl-2,3-dihydro-benzofuran-2-carboxylic acid, 5-{3-[4-(2,2-Dimethyl-propyl)-2-propyl-phenoxy]-propoxy}-2-ethyl-2,3-dihydro-benzofuran-2-carboxylic acid, 2-Ethyl-5-[3-(2-propyl-4-trifluoromethylsulfanyl-phenoxy)-propoxy]-2,3-dihydro-benzofuran-2-carboxylic acid. 65-[3-(2-Chloro-4-trifluoromethylsulfanyl-phenoxy)-propoxy]-2-ethyl-2,3-dihydro-benzofuran-2-carboxylic acid, 5-[3-(4-tert-Butyl-2-chloro-phenoxy)propoxy]-2-ethyl-2,3-dihydro-benzofuran-2-carboxylic acid, 5-[3-(2-Chloro-4-trifluoromethyl-phenoxy)-propoxy]-2-ethyl-2,3-dihydro-benzofuran-2-carboxylic acid, 5-{3-[2-Chloro-4-(1,1-dimethyl-propyl)-phenoxy]-propoxy}-2-ethyl-2,3-dihydro-benzofuran-2-carboxylic acid, (2S)-5-[3-(2-Chloro-4-trifluoromethoxy-phenoxy)-propoxy]-2-ethyl-2,3-dihydro-benzofuran-2-carboxylic acid, (2S)-5-{3-[2-Chloro-4-(2,2-dimethyl-propyl)-phenoxy]-propoxy}-2-ethyl-2,3-dihydro-benzofuran-2-carboxylic acid, (2S)-5-{3-[2-Chloro-4-(2,2,2-trifluoro-ethoxy)-phenoxy]-propoxy}-2-ethyl-2,3-dihydro-benzofuran-2-carboxylic acid, (2S)-5-{3-[2-Chloro-4-(3,3,3-trifluoro-propyl)-phenoxy]-propoxy}-2-ethyl-2,3-dihydro-benzofuran-2-carboxylic acid, (2S)-5-{3-[2-Chloro-4-(2,2,2-trifluoro-ethyl)-phenoxy]-propoxy}-2-ethyl-2,3-dihydro-benzofuran-2-carboxylic acid, 6-[3-(2-Chloro-4-trifluoromethoxy-phenoxy)-propoxy]-2-ethyl-2,3-dihydro-benzofuran-2-carboxylic acid, (2S)-5-[4-(2-Chloro-4-trifluoromethoxy-phenyl)-butoxy]-2-ethyl-2,3-dihydro-benzofuran-2-carboxylic acid, (2R)-5-{3-[2-Chloro-4-(2,2-dimethyl-propyl)phenoxy]-propoxy}-2-isopropyl-2,3-dihydro-benzofuran-2-carboxylic acid, (2R)-5-[3-(2-Chloro-4-trifluoromethoxy-phenoxy)-propoxy]-2-isopropyl-2,3-dihydro-benzofuran-2-carboxylic acid, (2R)-5-3-[2-Chloro-4-(2,2,2-trifluoro-ethyl)-phenoxy]-propoxy}-2-isopropyl-2,3-dihydro-benzofuran-2-carboxylic acid, (2R)-5-[4-(2-Chloro-4-trifluoromethoxy-phenoxy)-butyl]-2-isopropyl-2,3-dihydro-benzofuran-2-carboxylic acid, (2R)-2-tert-Butyl-5-{3-[2-chloro-4-(2,2,2-trifluoro-ethyl)phenoxy]-propoxy}-2,3-dihydro-benzofuran-2-carboxylic acid, 5-{3-[2-Chloro-4-(2,2,2-trifluoro-ethyl)-phenoxy]-propoxy}-2-trifluoromethyl-2,3-dihydro-benzofuran-2-carboxylic acid, (2R)-5-[2-(2-Chloro-4-trifluoromethoxy-phenoxy)-ethoxy]-2-isopropyl-2,3-dihydro-benzofuran-2-carboxylic acid, and (2R)-2-tert-Butyl-5-[2-(2-chloro-4-trifluoromethoxy-phenoxy)-ethoxy]-2,3-dihydro-benzofuran-2-carboxylic acid).

PPAR modulators (including compounds that may have multiple functionality for activating various combinations of PPARα, PPARγ, and PPARδ) such as those disclosed in U.S. Pat. No. 6,008,237, U.S. Pat. No. 6,248,781, U.S. Pat. No. 6,166,049, WO00/12491, WO00/218355, WO00/23415, WO00/23416, WO00/23425, WO00/23442, WO00/23445, WO00/23451, WO00/236331, WO00/236332, WO00/238553, WO00/50392, WO00/53563, WO00/63153, WO00/63190, WO0/63196, WO0/63209, WO0/78312, WO0/78313, WO01/04351, WO01/14349, WO01/14350, WO01/16120, WO01/17994, WO01/21181, WO01/21578, WO01/25181, WO01/25225, WO01/25226, WO01/40192, WO01/79150, WO02/081428, WO02/100403, WO02/102780, WO02/79162, WO03/016265, WO03/033453, WO03/042194, WO03/043997, WO03/066581, WO97/25042, WO99/07357, WO99/11255, WO99/12534, WO99/15520, WO99/46232, and WO98/05331 (including GW2331 or (2-(4-[difluorophenyl]-1-heptylureido)ethyl]phenoxy)-2-methylbutyric));

lipoxygenase inhibitors including 15-lipoxygenase (15-LO) inhibitors such as those disclosed in WO97/12615 (benzimidazole derivatives), WO97/12613, WO96/38144 (isothiazolones), Sendobry et al. Brit. J. Pharmacology (1997) 120, 1199-1206, and Cornicelli et al, Current Pharmaceutical Design, 1999, 5, 11-20;
niacin-bound chromium, as disclosed in WO03/039535;
substituted acid derivatives disclosed in WO03/040114;
apolipoprotein B inhibitors such as those disclosed in WO02/090347, WO02/28835, WO03/045921, WO03/047575;
Factor Xa modulators such as those disclosed in WO03/047517, WO03/047520, WO03/048081;
ileal bile acid transport (“IBAT”) inhibitors (or apical sodium co-dependent bile acid transport (“ASBT”) inhibitors) such as benzothiepines (including 1,2-benzothiazepines; 1,4-benzothiazepines; 1,5-benzothiazepines; 1,2,5-benzothiadiazepines);
IBAT inhibitors include but are not limited to compounds (e.g., those in claim 1 and the named examples) described in WO93/16055, WO94/18183, WO94/18184, WO96/05188, WO96/08484, WO96/16051, WO97/33882, WO98/38182, WO99/35135, WO98/40375, WO99/64409, WO99/64410, WO00/01687, WO00/47568, WO00/61568, DE 19825804, WO00/38725, WO00/38726, WO00/38727 (including those compounds with a 2,3,4,5-tetrahydro-1-benzothiepine 1,1-dioxide structure), WO00/38728, WO00/38729, WO01/66533, WO02/50051, EP0864582 (e.g., (3R,5R)-3-butyl-3-ethyl-1,1-dioxido-5-phenyl-2,3,4,5-tetrahydro-1,4-benzothiazepin-8-yl(β-D-glucopyranosiduronic acid, WO94/24087, WO98/07749, WO98/56757, WO99/32478, WO99/35135, WO00/20392, WO00/20393, WO00/20410, WO00/20437, WO01/34570, WO00/35889, WO01/68637, WO01/68096, WO02/08211, WO03/020710, WO03/022825, WO03/022830, WO03/022286, JP10072371, U.S. Pat. No. 5,070,103, EP251315, EP417725, EP489423, EP549967, EP573848, EP624593, EP624594, EP624595, EP869121 and EP1070703; S-8921 (disclosed in EP597107); 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-1′-phenyl-1′-[N′-(2-sulphoethyl) carbamoyl]methyl}carbamoylmethoxy)-2,3,4,5-tetrahydro-1,5-benzothiazepine; 1,1-dioxo-3-butyl-3-ethyl-5-phenyl-7-methylthio-8-(N-{(R)-1′-phenyl-1′-[N′-(2-sulphoethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-(2-sulphoethyl) carbamoyl]-4-hydroxybenzyl-carbamtoylmethoxy)-2,3,4,5-tetrahydro-1,5-benzothiazepine; 1,1-dioxo-3-butyl-3-ethyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine; 1,1-dioxo-3-butyl-3-ethyl-5-phenyl-7-methylthio-8-(N—(R)-α-[N′-(2-carboxyethyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-(2-carboxyethyl) carbamoyl]-4-hydroxybenzyl}carbamoylmethoxy)-2,3,4,5-tetrahydro-1,5-benzothiazepine; 1,1-dioxo-3-butyl-3-ethyl-5-phenyl-7-methylthio-8-(N— (R)-α-[N′-(5-carboxypentyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-α-[N′-(2-sulphoethyl)carbamoyl]-2-fluorobenzyl}carbamoylmethoxy)-2,3,4,5-tetrahydro-1,5-benzothiazepine; 1,1-dioxo-3-butyl-3-ethyl-5-phenyl-7-methylthio-8-(N—(R)-α-[N′-(R)-(2-hydroxy-1-carboxyethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R))—[N′-(R)-(2-hydroxy-1-carboxyethyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-{N—[(R)-α-(N′—{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine; 1,1-dioxo-3-butyl-3-ethyl-5-phenyl-7-methylthio-8-(N-{a-[N′-(carboxymethyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{a-[N′-((ethoxy)(methyl)phosphoryl-methyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-{N—[(R)-α-(N′-{2-[(hydroxy)(methyl)phosphoryl]ethyl}carbamoyl)benzyl]carbamoylmethoxy}-2,3,4,5-tetrahydro-1,5-benzothiazepine; 1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-{N—[(R)-α-(N′-{2-[(methyl)(ethyl)phosphoryl]ethyl}carbamoyl)-4-hydroxybenzyl]carbamoylmethoxy}-2,3,4,5-tetrahydro-1,5-benzothiazepine; 1,1-dioxo-3,3-dibutyl-5-phenyl-7-methylthio-8-{N—[(R)-α-(N′-22-[(methyl)(hydroxy)phosphoryl]ethyl}carbamoyl)-4-hydroxybenzyl]carbamoylmethoxy}-2,3,4,5-tetrahydro-1,5-benzothiazepine; 1,1-dioxo-3,3-dibutyl-5-phenyl-7-methylthio-8-(N—{(R)-α-[(R)—N′-(2-methylsulphinyl-1-carboxyethyl) carbamoyl]benzyl}carbamoylmethoxy)-2,3,4,5-tetrahydro-1,5-benzothiazepine; and 1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine; compounds disclosed in claims 1-10 and examples 1-44 of WO03/020710; compounds disclosed in WO03/020710 (including 1,1-dioxo-3,3-dibutyl-5-phenyl-7-methylthio-8-(N— (R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{(R)-α-[N′-((S)-1-carbamoyl-2-hydroxyethyl)carbamoyl]benzyl}carbamyoylmethoxy)-2,3,4,5-tetrahydro-1,5-benzothiazepine; 1,1-dioxo-3-butyl-3-ethyl-5-phenyl-7-methylthio-8-(N-{(R)-α-[N′-(hydroxycarbamoyl-methyl) carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-[N((R)-α-{N′-[2-(N′-pyrimidin-2-ylureido)ethyl]carbamoyl}benzyl)carbamoylmethoxy]-2,3,4,5-tetrahydro-1,5-benzothiazepine; 1,1-dioxo-3-butyl-3-ethyl-5-phenyl-7-methylthio-8[N—((R)-α-{N′-[2-(N′-pyridin-2-ylureido)ethyl]carbamoyl}benzyl)carbamoylmethoxy]-2,3,4,5-tetrahydro-1,5-benzothiazepine; 1,1-dioxo-3-butyl-3-ethyl-5-phenyl-7-methylthio-8-(N-{(R)-α-[N′-(1-t-butoxycarbonylpiperidin-4-ylmethyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{(R))—[N′-(2,3-dihydroxypropyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-[N—((R)-α-{N′-[2-(3,4-dihydroxyphenyl)-2-methoxyethyl]carbamoyl}benzyl) carbamoylmethoxy]-2,3,4,5-tetrahydro-1,5-benzothiazepine 1,1-dioxo-3-butyl-3-ethyl-5-phenyl-7-methylthio-8-(N-{(R)-α-[N′-(2-aminoethyl)carbamoyl]benzyl}carbamoylmethoxy)-2,3,4,5-tetrahydro-1,5-benzothiazepine; 1,1-dioxo-3-butyl-3-ethyl-5-phenyl-7-methylthio-8-(N-{(R)-α-[N′-(piperidin-4-ylmethyl)carbamoyl]benzyl}carbamoylmethoxy)-2,3,4,5-tetrahydro-1,5-benzothiazepine; and 1,1-dioxo-3-butyl-3-ethyl-5-phenyl-7-methylthio-8-(N{(R)-α-[N′-(2-N,N-dimethylaminosulphamoylethyl) carbamoyl]benzyl}carbamoylmethoxy)-2,3,4,5-tetrahydro-1,5-benzothiazepine); compounds disclosed in claims 1-8 and examples 1-7 of WO03/022825; compounds disclosed in WO03/022825 (including 1-dioxo-3(R)-3-butyl-3-ethyl-5-(R)-5-phenyl-8-[N—((R)-α-carboxybenzyl) carbamoylmethoxy]-2,3,4,5-tetrahydro-1,4-benzothiazepine; 1,1-dioxo-3(S)-3-butyl-3-ethyl-5-(S)-5-phenyl-8-[N—((R)-α-carboxybenzyl)carbamoylmethoxy]-2,3,4,5-tetrahydro-1,4-benzothiazepine; 1,1-dioxo-3(R)-3-butyl-3-ethyl-5-(R)-5-phenyl-8-(N-{(R)-α-[N-(carboxymethyl)carbamoyl]benzyl}carbamoylmethoxy)-2,3,4,5-tetrahydro-1,4-benzothiazepine; 1,1-dioxo-3(S)-3-butyl-3-ethyl-5-(S)-5-phenyl-8-(N-{(R)-α-[N-(carboxymethyl)carbamoyl]benzyl}carbamoylmethoxy)-2,3,4,5-tetrahydro-1,4-benzothiazepine; 3,5-trans-1,1-dioxo-3-ethyl-3-butyl-5-phenyl-7-bromo-8-(N-{(R)-α-[N-(carboxymethyl)carbamoyl]benzyl}carbamoylmethoxy)-2,3,4,5-tetrahydro-1,4-benzothiazepine; 3,5-trans-1,1-dioxo-3-(S)-3-ethyl-3-butyl-4-hydroxy-5-(S)-5-phenyl-7-bromo-8-(N-{(R)-α-[N-(carboxymethyl)carbamoyl]benzyl}carbamoylmethoxy)-2,3,4,5-tetrahydro-1,4-benzothiazepine 3,5-trans-1,1-dioxo-3-(R)-3-ethyl-3-butyl-4-hydroxy-5-(R)-5-phenyl-7-bromo-8-(N-{(R)-α-[N-(carboxymethyl)carbamoyl]benzyl}carbamoylmethoxy)-2,3,4,5-tetrahydro-1,4-benzothiazepine; 3,5-trans-1,1-dioxo-3-ethyl-3-butyl-5-phenyl-7-methylthio-8-(N-{(R)-α-[N-(carboxymethyl)carbamoyl]benzyl}carbamoylmethoxy)-2,3,4,5-tetrahydro-1,4-benzothiazepine; 3,5-trans-1,1-dioxo-3-ethyl-3-butyl-5-phenyl-7-methylthio-8-(N-{(R)-α-[N-(2-sulphoethyl) carbamoyl]-4-hydroxybenzyl}carbamoylmethoxy)-2,3,4,5-tetrahydro-1,4-benzothiazepine ammonia salt; 1,1-dioxo-3-(S)-3-ethyl-3-butyl-5-(S)-5-phenyl-7-methylthio-8-(N-{(R)-α-[N-(carboxymethyl)carbamoyl]benzyl}carbamoylmethoxy)-2,3,4,5-tetrahydro-1,4-benzothiazepine diethylamine salt; and 1,1-dioxo-3-(R)-3-ethyl-3-butyl-5-(R)-5-phenyl-7-methylthio-8-(N-{(R)-α-[N-(carboxymethyl)carbamoyl]benzyl carbamoylmethoxy)-2,3,4,5-tetrahydro-1,4-benzothiazepine diethylamine salt); compounds disclosed in claims 1-8 and examples 1-4 of WO03/022830; compounds disclosed in WO03/022830 (including 1,1-dioxo-3-butyl-3-ethyl-4-hydroxy-5-phenyl-7-(N-{(R)-α-[N-(carboxymethyl)carbamoyl]benzyl}carbamoylmethylthio)-2,3,4,5-tetrahydrobenzothiepine 1,1-dioxo-3-butyl-3-ethyl-4-hydroxy-5-phenyl-7-(N-{(R)-α-[N-(2-sulphoethyl)carbamoyl]-4-hydroxybenzyl}carbamoylmethylthio)-2,3,4,5-tetrahydrobenzothiepine ammonia salt 1,1-dioxo-3-butyl-3-ethyl-4-hydroxy-5-phenyl-7-{N-[a-(carboxy)-2-fluorobenzyl]carbamoylmethylthio}-2,3,4,5-tetrahydrobenzothiepine; and 1,1-dioxo-3-butyl-3-ethyl-4-hydroxy-5-phenyl-7-{N-[1-(carboxy)-1-(thien-2-yl)methyl]carbamoylmethylthio}-2,3,4,5-tetrahydrobenzothiepine); compounds disclosed in claims 1-10 and examples 1-30 WO03/0222861; compounds disclosed in WO03/0222861 (including 1,1-dioxo-3,3-dibutyl-5-phenyl-7-methylthio-8-(N—(R)-α-[N((R)-1-carboxy-2-methylthioethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)hydroxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-—[N—((S)-1-carboxybutyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N— (R)-α-[N—((S)-1-carboxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N—{(R)-α-[N—((R)-1-carboxy-2-methylthioethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine; and 1,1-dioxo-3,3-dibutyl-5-phenyl-7-methylthio-8-[N—((R)-1-carboxy-4-hydroxybenzyl) carbamoylmethoxy]-2,3,4,5-tetrahydro-1,2,5-benzothiadiazepine); compounds having the structure of formula (EI) on page 58 of WO04/005247 (including 1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine; 1,1-Dioxo-3,3-dibutyl-5-phenyl-7-methylthio-8-(N—-(R)-α-[N-(2-(S)-3-(R)-4-(R)-5-(R)-2,3,4,5,6-pentahydroxyhexyl) carbamoyl]-4-hydroxybenzyl}carbamoylmethoxy)-2,3,4,5-tetrahydro-1,2,5-benzothiadiazepine; 1,1-Dioxo-3,3-dibutyl-5-phenyl-7-methylthio-8-[N—((R/S)-α-{N-[1-(R)-2-(S)-1-hydroxy-1-(3,4-dihydroxyphenyl)prop-2-yl]carbamoyl}benzyl)carbamoylmethoxy]-2,3,4,5-tetrahydro-1,2,5-benzothiadiazepine (both enantiomers); 1,1-Dioxo-3,3-dibutyl-5-phenyl-7-methylthio-8-{N—[(R)-α-(N-{2-(S)—[N-(carbamoylmethyl)carbamoyl]pyrrolidin-1-ylcarbonylmethyl}carbamoyl)benzyl]carbamoylmethoxy-2,3,4,5-tetrahydro-1,2,5-benzothiadiazepine; 1,1-Dioxo-3,3-bidutyl-5-phenyl-7-methylthio-8-[N—((R)-α-{N-[2-(3,4,5-trihydroxyphenyl)ethyl]carbamoyl}benzyl)carbamoylmethoxy]-2,3,4,5-tetrahydro-1,2,5-benzothiadiazepine; and 1,1-Dioxo-3,3-dibutyl-5-phenyl-7-methylthio-8-(N-{(R)-α-[N-(2 (R)-3-(S)-4-(S)-5-(R)-3,4,5,6-tetrahydroxytetrahydropyran-2-ylmethyl)carbamoyl]benzyl}carbamoylmethoxy)-2,3,4,5-tetrahydro-1,2,5-benzothiadiazepine); compounds having the structure of formula (FI) on page 62 of WO04/005247 including ((1,1-dioxo-3,3-dibutyl-5-phenyl-7-methylthio-8-(N-{(R)-α-[N′-((S)-1-carboxyethyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxypropyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxybutyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methylpropyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methylbutyl) carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-methylbutyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-hydroxypropyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-mesylethyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-methylsulphonylpropyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N—{(R)-α-[N′-((S)-1-carboxy-3-mesylpropyl)carbamoyl]benzyl}carbamoylmethoxy)-2,3,4,5-tetrahydro-1,5-benzothiazepine; 1,1-dioxo-3,3-dibutyl-5-phenyl-7-methylthio-8-(N—(R)-α-[N′-((S)-1-carboxyethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxybutyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxyl-2-methylpropyl)carbamoyl]-4-hydroxybenzyl carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methylbutyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-methylbutyl) carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-hydroxyethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R))-α-[N′-((S)-1-carboxy-2-hydroxypropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methylthioethyl) carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methylsulphinylethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R))—[N′-((S)-1-carboxy-2-mesylethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-2-methoxyethyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-methylthiopropyl) carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-methylsulphonylpropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxy-3-mesylpropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; and 1,1-dioxo-3,3-dibutyl-5-phenyl-7-methylthio-8-(N-{(R)-α-[N′-((S)-1-carboxyethyl) carbamoyl]benzyl}carbamoylmethoxy)-2,3,4,5-tetrahydro-1,5-benzothiazepine); compounds having the structure of formula (GI) on page 65 of WO04/005247 (including (+/−)-trans-1,1-dioxo-3-ethyl-3-butyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,4-benzothiazepine; (+/−)-trans-1,1-dioxo-3-ethyl-3-butyl-5-phenyl-7-methylthio-8-(N-{(R)-α-[N′-(2-(S)-3-(R)-4-(R)-5-(R)-2,3,4,5,6-pentahydroxyhexyl) carbamoyl]benzyl carbamoylmethoxy)-2,3,4,5-tetrahydro-1,4-benzothiazepine; 1,1-dioxo-3-ethyl-3-butyl-4-hydroxy-5-phenyl-7-(N-{α-[N′-(2-(S)-3-(R)-4-(R)-5-(R)-2,3,4,5,6-pentahydroxyhexyl)carbamoyl]-2-fluorobenzyl}carbamoylmethylthio)-2,3,4,5-tetrahydrobenzothiapine; and 1,1-dioxo-3-butyl-3-ethyl-4-hydroxy-5-phenyl-7-(N-{1-[N′-(2-(S)-3-(R)-4-(R)-5-(R)-2,3,4,5,6-pentahydroxyhexyl)carbamoyl]-1-(cyclohexyl)methyl icarbamoylmethylthio)-2,3,4,5-tetrahydrobenzothiepine); compounds disclosed as having IBAT activity disclosed in WO04/005247; compounds disclosed as having IBAT activity in Drugs of the Future, 24, 425-430 (1999); or a pharmaceutically acceptable salt, solvate, solvate of such salt or prodrug thereof, ATP citrate lyase inhibitors including those disclosed in U.S. Pat. No. 5,447,954; PPARδ activators such as disclosed in WO01/00603 (thiazole and oxazole derivates (e.g., CAS RN 317318-32-4), WO97/28149 (fluoro, chloro and thio phenoxy phenylacetic), U.S. Pat. No. 5,093,365 (non-1-oxidizable fatty acid analogues), and WO99/04815; and
other dyslipidemic agents such as benfluorex, β-Benzylbutyraimde, colmestrone, detaxtran, dextran sulphate sodium, eicosopentanoic acid, eritadenine, furazabol, meglutol, γ-Oryzanol, pantethine and derivatives thereof (as disclosed, for example, in US20050101565), pentaerythritol tetraacetate, α-phenylbutyramide, pirozadil, sultosilic acid, tiadenol, triparanol, and xenbucin, isoniazid (disclosed in WO97/35576), a lanosterol demethylase inhibitor (as disclosed in WO97/48701), cholestagel (Sankyo/Geltex), lipostabil (Rhone-Poulenc), Eisai E-5050 (an N-substituted ethanolamine derivative), imanixil (HOE-402), tetrahydrolipstatin (THL), istigmastanylphosphorylcholine (SPC, Roche), aminocyclodextrin (Tanabe Seiyoku), Ajinomoto AJ-814 (azulene derivative), melinamide (Sumitomo), nitric oxide synthase isoforms (e.g., endothelial (eNOS), neuronal (nNOS) and inducible (iNOS), for example purified, recombinant or virally/retrovirally expressed), the antisense oligonucleotides described in Kipshidze, et al., J. Am. Coll. Cardio. 39(10):1686-1691 (2002); the nuclear targeted lacZ- and TIMP-1-encoding adenoviruses coupled to peptide-motif (HWGF) described in Turunen, et al., Mol Ther 6(3):306 (2002), Sandoz 58-035, American Cyanamid CL-277,082 and CL-283,546 (disubstituted urea derivatives), compounds or combinations of compounds that result in the production or enhancement of nitric oxide (for example those disclosed in WO04091626, paragraphs 46-53), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride), pancreatic cholesteryl hydrolase (pCEH) inhibitors (such as WAY-121898), fish oil (which contains Omega 3 fatty acids (3-PUFA)), combinations of one or more anti-microbial agents (e.g., tetracyclin, ofloxacin, clinafloxacin, ciprofloxacin, clindamycin, doxycycline and minocycline, erythromycin or azalides such as trythromycin, roxithromycin, zithromycin, clarithromycin and azithromycin) with one or more metal chelators (e.g., desferrioxamine mesylate, haem derivatives, penicillamine, tiopronin, trientine, dihydrochloride, diethyldithiocarbamate, acetylsalicylic acid, disodium/trisodium, edetate, edetic acid, unithiol, copper chelators (penicillamine, tiopronin, trientine, dihydrochloride, diethyldithiocarbamate, acetylsalicylic acid)) as described in WO05034962, antisense inhibitors of apolipoprotein B (such as those disclosed in WO03097662, WO04044181, WO06020676 including ISIS 301012 (CAS RN 629167-92-6)), and ionenes such as disclosed in U.S. Pat. No. 4,027,009. Tests showing the efficacy of the therapy and the rationale for the combination therapy with a dyslipidemic agent are presented in US20030069221 (wherein the dyslipidemic agents are called ‘cardiovascular agents’).

### Anti-Diabetic Agents

The compounds described herein can be used in therapeutic combination with one or more anti-diabetic agents, including but not limited to:

PPARγ agonists such as glitazones (e.g., WAY-120,744, AD 5075, balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®), disclosed in U.S. Pat. No. 4,572,912), rivoglitazone (CS-011, Sankyo), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), N,N-2344 (Dr. Reddy/NN), YM-440 (Yamanouchi), LY-300512, LY-519818, R483 (Roche), T131 (Tularik), and the like and compounds disclosed in U.S. Pat. No. 4,687,777, U.S. Pat. No. 5,002,953, U.S. Pat. No. 5,741,803, U.S. Pat. No. 5,965,584, U.S. Pat. No. 6,150,383, U.S. Pat. No. 6,150,384, U.S. Pat. No. 6,166,042, U.S. Pat. No. 6,166,043, U.S. Pat. No. 6,172,090, U.S. Pat. No. 6,211,205, U.S. Pat. No. 6,271,243, U.S. Pat. No. 6,288,095, U.S. Pat. No. 6,303,640, U.S. Pat. No. 6,329,404, U.S. Pat. No. 5,994,554, WO97/10813, WO97/27857, WO97/28115, WO97/28137, WO97/27847, WO00/76488, WO03/000685, WO03/027112, WO03/035602, WO03/048130, WO03/055867, and pharmaceutically acceptable salts thereof;
biguanides such as metformin hydrochloride (N,N-dimethylimidodicarbonimidic diamide hydrochloride, such as Glucophage™, Bristol-Myers Squibb); metformin hydrochloride with glyburide, such as Glucovance™, Bristol-Myers Squibb); buformin (Imidodicarbonimidic diamide, N-butyl-); etoformine (1-Butyl-2-ethylbiguanide, Schering A. G.); other metformin salt forms (including where the salt is chosen from the group of, acetate, benzoate, citrate, fumarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octanoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate and phosphate), and phenformin;
protein tyrosine phosphatase-1B (PTP-1B) inhibitors, such as A-401,674, KR 61639, OC-060062, OC-83839, OC-297962, MC52445, MC52453, ISIS 113715, and those disclosed in WO99/585521, WO99/58518, WO99/58522, WO99/61435, WO03/032916, WO03/032982, WO03/041729, WO03/055883, WO02/26707, WO02/26743, JP2002114768, and pharmaceutically acceptable salts and esters thereof, sulfonylureas such as acetohexamide (e.g., Dymelor, Eli Lilly), carbutamide, chlorpropamide (e.g., Diabinese®, Pfizer), gliamilide (Pfizer), gliclazide (e.g., Diamcron, Servier Canada Inc), glimepiride (e.g., disclosed in U.S. Pat. No. 4,379,785, such as Amaryl™, Aventis), glipentide, glipizide (e.g., Glucotrol or Glucotrol XL Extended Release, Pfizer), gliquidone, glisolamide, glyburide/glibenclamide (e.g., Micronase or Glynase Prestab, Pharmacia & Upjohn and Diabeta, Aventis), tolazamide (e.g., Tolinase), and tolbutamide (e.g., Orinase), and pharmaceutically acceptable salts and esters thereof,
meglitinides such as repaglinide (e.g., Pranidin®, Novo Nordisk), KAD1229 (PF/Kissei), and nateglinide (e.g., Starlix®, Novartis), and pharmaceutically acceptable salts and esters thereof,
α glucoside hydrolase inhibitors (or glucoside inhibitors) such as acarbose (e.g., Precose™, Bayer disclosed in U.S. Pat. No. 4,904,769), miglitol (such as Glyset™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-alpha-D-glucopyranoside, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, U.S. Pat. No. 51091 and WO01/47528 (polyamines);
α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765;
SGLT2 inhibitors including those disclosed in U.S. Pat. No. 6,414,126 and U.S. Pat. No. 6,515,117;
an aP2 inhibitor such as disclosed in U.S. Pat. No. 6,548,529;
insulin secreatagogues such as linogliride, A-4166, forskilin, dibutyrl cAMP, isobutylmethylxanthine (IBMX), and pharmaceutically acceptable salts and esters thereof;
fatty acid oxidation inhibitors, such as clomoxir, and etomoxir, and pharmaceutically acceptable salts and esters thereof;
A2 antagonists, such as midaglizole, isaglidole, deriglidole, idazoxan, earoxan, and fluparoxan, and pharmaceutically acceptable salts and esters thereof, insulin and related compounds (e.g., insulin mimetics) such as biota, LP-100, novarapid, insulin detemir, insulin lispro, insulin glargine, insulin zinc suspension (lente and ultralente), Lys-Pro insulin, GLP-1 (1-36) amide, GLP-1 (73-7) (insulintropin, disclosed in U.S. Pat. No. 5,614,492), LY-315902 (Lilly), GLP-1 (7-36)-NH2), AL-401 (AutoImmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the physician's desk reference, 55.sup.th Ed. (2001) Medical Economics, Thomson Healthcare (disclosing other suitable human insulins);
non-thiazolidinediones such as JT-501 and farglitazar (GW-2570/GI-262579), and pharmaceutically acceptable salts and esters thereof;
PPARα/γ dual agonists such as AR-HO39242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methyl]benzamide), L-796449, LR-90, MK-0767 (Merck Kyorin/Banyu), SB 219994, muraglitazar (BMS), tesaglitzar (Astrazeneca), reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976, U.S. application Ser. No. 09/664,598, filed Sep. 18, 2000, Murakami et al. Diabetes 47, 1841-1847 (1998), and pharmaceutically acceptable salts and esters thereof;
other insulin sensitizing drugs;
VPAC2 receptor agonists;
GLK modulators, such as those disclosed in WO03/015774;
retinoid modulators such as those disclosed in WO03/000249;
GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like;
glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof;
ATP consumption promoters such as those disclosed in WO03/007990;
TRB3 inhibitors;
vanilloid receptor ligands such as those disclosed in WO03/049702;
hypoglycemic agents such as those disclosed in WO03/015781 and WO03/040114;
glycogen synthase kinase 3 inhibitors such as those disclosed in WO03/035663 agents such as those disclosed in WO99/51225, US20030134890, WO01/24786, and WO03/059870;
insulin-responsive DNA binding protein-1 (IRDBP-1) as disclosed in WO03/057827, and the like;
adenosine A2 antagonists such as those disclosed in WO03/035639, WO03/035640, and the like;
PPARδ agonists such as GW 501516, GW 590735, and compounds disclosed in JP10237049 and WO02/14291;
dipeptidyl peptidase IV (DP-IV) inhibitors, such as isoleucine thiazolidide, NVP-DPP728A (1-[[[2-[(5-cyanopyridin-2-yl)amino]ethyl]amino]acetyl]-2-cyano-(S)pyrrolidine, disclosed by Hughes et al, Biochemistry, 38(36), 11597-11603, 1999), P32/98, NVP-LAF-237, P3298, TSL225 (tryptophyl-1,2,3,4-tetrahydro-isoquinoline-3-carboxylic acid, disclosed by Yamada et al, Bioorg. & Med. Chem. Lett. 8 (1998) 1537-1540), valine pyrrolidide, TMC-2A/2B/2C, CD-26 inhibitors, FE999011, P9310/K364, VIP 0177, DPP4, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996), and the compounds disclosed in U.S. Pat. No. 6,395,767, U.S. Pat. No. 6,573,287, U.S. Pat. No. 6,395,767 (compounds disclosed include BMS-477118, BMS-471211 and BMS 538,305), WO99/38501, WO99/46272, WO99/67279, WO99/67278, WO99/61431WO03/004498, WO03/004496, EP1258476, WO02/083128, WO02/062764, WO03/000250, WO03/002530, WO03/002531, WO03/002553, WO03/002593, WO03/000180, and WO03/000181;
GLP-1 agonists such as exendin-3 and exendin-4 (including the 39 aa peptide synthetic exendin-4 called Exenatide®), and compounds disclosed in US2003087821 and NZ 504256, and pharmaceutically acceptable salts and esters thereof;
   peptides including amlintide and Symlin® (pramlintide acetate); and
   glycokinase activators such as those disclosed in US2002103199 (fused heteroaromatic compounds) and WO02/48106 (isoindolin-1-one-substituted propionamide compounds).

Showing the efficacy of the therapy and the rationale for the combination therapy with an anti-diabetic agent are presented in US20040214811.

### Anti-Hypertensive Agents

The compounds described herein can be used in therapeutic combination with one or more anti-hypertensive agents, including but not limited to:

diuretics, such as thiazides (e.g., chlorthalidone, cyclothiazide (CAS RN 2259-96-3), chlorothiazide (CAS RN 72956-09-3, which may be prepared as disclosed in U.S. Pat. No. 2,809,194), dichlorophenamide, hydroflumethiazide, indapamide, polythiazide, bendroflumethazide, methyclothazide, polythiazide, trichlormethazide, chlorthalidone, indapamide, metolazone, quinethazone, althiazide (CAS RN 5588-16-9, which may be prepared as disclosed in British Patent No. 902,658), benzthiazide (CAS RN 91-33-8, which may be prepared as disclosed in U.S. Pat. No. 3,108,097), buthiazide (which may be prepared as disclosed in British Patent Nos. 861,367), and hydrochlorothiazide), loop diuretics (e.g., bumetanide, ethacrynic acid, furosemide, and torasemide), potassium sparing agents (e.g., amiloride, and triamterene (CAS Number 396-01-0)), and aldosterone antagonists (e.g., spironolactone (CAS Number 52-01-7), epirenone, and the like);
β-adrenergic blockers such as Amiodarone (Cordarone, Pacerone), bunolol hydrochloride (CAS RN 31969-05-8, Parke-Davis), acebutolol (±N-[3-Acetyl-4-[2-hydroxy-3-[(1methylethyl)amino]propoxy]phenyl]-butanamide, or (±)-3′-Acetyl-4′-[2-hydroxy-3-(isopropylamino) propoxy]butyranilide), acebutolol hydrochloride (e.g., Sectral®, Wyeth-Ayerst), alprenolol hydrochloride (CAS RN 13707-88-5 see Netherlands Patent Application No. 6,605,692), atenolol (e.g., Tenormin®, AstraZeneca), carteolol hydrochloride (e.g., Cartrol® Filmtab®, Abbott), Celiprolol hydrochloride (CAS RN 57470-78-7, also see in U.S. Pat. No. 4,034,009), cetamolol hydrochloride (CAS RN 77590-95-5, see also U.S. Pat. No. 4,059,622), labetalol hydrochloride (e.g., Normodyne®, Schering), esmolol hydrochloride (e.g., Brevibloc®, Baxter), levobetaxolol hydrochloride (e.g., Betaxon™ Ophthalmic Suspension, Alcon), levobunolol hydrochloride (e.g., Betagan® Liquifilm® with C CAP® Compliance Cap, Allergan), nadolol (e.g., Nadolol, Corgard, Mylan), practolol (CAS RN 6673-35-4, see also U.S. Pat. No. 3,408,387), propranolol hydrochloride (CAS RN 318-98-9), sotalol hydrochloride (e.g., Betapace AF™, Berlex), timolol (2-Propanol, 1-[(1,1-dimethylethyl)amino]-3-[[4-4(4-morpholinyl)-1,2,5-thiadiazol-3-yl]oxy]-, hemihydrate, (S)-, CAS RN 91524-16-2), timolol maleate (S)-1-[(1,1-dimethylethyl)amino]-3-[[4-(4-morpholinyl)-1,2,5-thiadiazol-3-yl]oxy]-2-propanol (Z)-2-butenedioate (1:1) salt, CAS RN 26921-17-5), bisoprolol (2-Propanol, 1-[4-[[2-(1-methylethoxy)ethoxy]-methyl]phenoxyl]-3-[(1-meth-ylethyl)amino]-, (±), CAS RN 66722-44-9), bisoprolol fumarate (such as (±)-1-[4-[[2-(1-Methylethoxy)ethoxy]methyl]phenoxy]-3-[(1-methylethyl)amino]-2-propanol (E)-2-butenedioate (2:1) (salt), e.g., Zebeta™, Lederle Consumer), nebivalol (2H-1-Benzopyran-2-methanol, αα′-[iminobis(methylene)]bis[6-fluoro-3,4-dihydro-, CAS RN 99200-09-6 see also U.S. Pat. No. 4,654,362), cicloprolol hydrochloride, such 2-Propanol, 1-[4-[2-(cyclopropylmethoxy)ethoxy]phenoxy]-3-[1-methylethyl)amino]-, hydrochloride, A.A.S. RN 63686-79-3), dexpropranolol hydrochloride (2-Propanol, 1-[1-methylethy)-amino]-3-(1-naphthalenyloxy)hydrochloride (CAS RN 13071-11-9), diacetolol hydrochloride (Acetamide, N-[3-acetyl-4-[2-hydroxy-3-[(1-methyl-ethyl)amino]propoxy][phenyl]-, monohydrochloride CAS RN 69796-04-9), dilevalol hydrochloride (Benzamide, 2-hydroxy-5-[1-hydroxy-2-[1-methyl-3-phenylpropyl)amino]ethyl]-, monohydrochloride, CAS RN 75659-08-4), exaprolol hydrochloride (2-Propanol, 1-(2-cyclohexylphenoxy)-3-[(1-methylethyl)amino]-, hydrochloride CAS RN 59333-90-3), flestolol sulfate (Benzoic acid, 2-fluoro-3-[[2-[aminocarbonyl)amino]-1-dimethylethyl]amino]-2-hydroxypropyl ester, (±)-sulfate (1:1) (salt), CAS RN 88844-73-9; metalol hydrochloride (Methanesulfonamide, N-[4-[1-hydroxy-2-(methylamino)propyl]phenyl]-, monohydrochloride CAS RN 7701-65-7), metoprolol 2-Propanol, 1-[4-(2-methoxyethyl)phenoxy]-3-[1-methylethyl)amino]-; CAS RN 37350-58-6), metoprolol tartrate (such as 2-Propanol, 1-[4-(2-methoxyethyl)phenoxy]-3-[(1-methylethyl)amino]-, e.g., Lopressor®, Novartis), pamatolol sulfate (Carbamic acid, [2-[4-[2-hydroxy-3-[(1-methylethyl)amino]propoxyl]phenyl]-ethyl]-, methyl ester, (±) sulfate (salt) (2:1), CAS RN 59954-01-7), penbutolol sulfate (2-Propanol, 1-(2-cyclopentylphenoxy)-3-[1,1-dimethylethyl)amino]1, (S)-, sulfate (2:1) (salt), CAS RN 38363-32-5), practolol (Acetamide, N-[4-[2-hydroxy-3-[(1-methylethyl)amino]-propoxy]phenyl]-, CAS RN 6673-35-4) tiprenolol hydrochloride (Propanol, 1-[(1-methylethyl)amino]-3-[2-(methylthio)-phenoxy]-, hydrochloride, (±), CAS RN 39832-43-4), tolamolol (Benzamide, 4-[2-[[2-hydroxy-3-(2-methylphenoxy)propyl]amino]ethoxyl]-, CAS RN 38103-61-6), bopindolol, indenolol, pindolol (e.g., Visken), propanolol (e.g., Inderal, Inderal-LA), tertatolol, Coreg (carvedilol), and tilisolol, and the like;
calcium channel blockers such as besylate salt of amlodipine (such as 3-ethyl-5-methyl-2-(2-aminoethoxymethyl)-4-(2-chlorophenyl)-1,4-dihydro-6-methyl-3,5-pyridinedicarboxylate benzenesulphonate, e.g., Norvasc®, Pfizer), clentiazem maleate (1,5-Benzothiazepin-4(5H)-one, 3-(acetyloxy)-8-chloro-5-[2-(dimethylamino)ethyl]-2,3-dihydro-2-(4-methoxyphenyl)-(2S-cis)-, (Z)-2-butenedioate (1:1), see also U.S. Pat. No. 4,567,195), isradipine (3,5-Pyridinedicarboxylic acid, 4-(4-benzofurazanyl)-1,4-dihydro-2,6-dimethyl-, methyl 1-methylethyl ester, (±)-4(4-benzofurazanyl)-1,4-dihydro-2,6-dimethyl-3,5-pyridinedicarboxylate, see also U.S. Pat. No. 4,466,972); nimodipine (such as is isopropyl (2-methoxyethyl) 1,4-dihydro-2,6-dimethyl-4-(3-nitrophenyl)-3,5-pyridine-dicarboxylate, e.g., Nimotop®, Bayer), felodipine (such as ethyl methyl 4-(2,3-dichlorophenyl)-1,4-dihydro-2,6-dimethyl-3,5-pyridinedicarboxylate-, e.g., Plendil® Extended-Release, AstraZeneca LP), nilvadipine (3,5-Pyridinedicarboxylic acid, 2-cyano-1,4-dihydro-6-methyl-4-(3-nitrophenyl)-3-methyl 5-(1-methylethyl) ester, also see U.S. Pat. No. 3,799,934), nifedipine (such as 3,5-pyridinedicarboxylic acid, 1,4-dihydro-2,6-dimethyl-4-(2-nitrophenyl)-, dimethyl ester, e.g., Procardia XL® Extended Release Tablets, Pfizer), diltiazem hydrochloride (such as 1,5-Benzothiazepin-4(5H)-one, 3-(acetyloxy)-5 [2-(dimethylamino)ethyl]-2, -3-dihydro-2(4-methoxyphenyl)-, monohydrochloride, (+)-cis., e.g., Tiazac®, Forest), verapamil hydrochloride (such as benzeneacetronitrile, (alpha)-[[3-[[2-(3,4-dimethoxyphenyl)ethyl]methylamino]propyl]-3,4-dimethoxy-(alpha)-(1-methylethyl)hydrochloride, e.g., Isoptin® SR, Knoll Labs), teludipine hydrochloride (3,5-Pyridinedicarboxylic acid, 2-[(dimethylamino)methyl]-4-[2-[(1E)-3-(1,1-dimethylethoxy)-3-oxo-1-propenyl]phenyl]-1,4-dihydro-6-methyl-, diethyl ester, monohydrochloride) CAS RN 108700-03-4), belfosdil (Phosphonic acid, [2-(2-phenoxyethyl)-1,3-propane-diyl]bis-tetrabutyl ester CAS RN 103486-79-9), fostedil (Phosphonic acid, [[4-(2-benzothiazolyl)phenyl]methyl]-, diethyl ester CAS RN 75889-62-2), aranidipine, azelnidipine, barnidipine, benidipine, bepridil, cinaldipine, clevidipine, efonidipine, gallopamil, lacidipine, lemildipine, lercanidipine, monatepil maleate (1-Piperazinebutanamide, N-(6,11-Dihydrodibenzo(b,e)thiepin-11-yl)₄-(4-fluorophenyl)-, (±)-, (Z)-2-butenedioate (1:1) (±)-N-(6,11-Dihydrodibenzo(b,e)thiep-in-11-yl)-4-(p-fluorophenyl)-1-piperazinebutyramide maleate (1:1) CAS RN 132046-06-1), nicardipine, nisoldipine, nitrendipine, manidipine, pranidipine, and the like; T-channel calcium antagonists such as mibefradil;
angiotensin converting enzyme (ACE) inhibitors such as benazepril, benazepril hydrochloride (such as 3-[[1-(ethoxycarbonyl)-3-phenyl-(1S)-propyl]amino]-2,3,4,5-tetrahydro-2-oxo-1H-1-(3S)-benzazepine-1-acetic acid monohydrochloride, e.g., Lotrel®, Novartis), captopril (such as 1-[(2S)-3-mercapto-2-methylpropionyl]-L-proline, e.g., Captopril, Mylan, CAS RN 62571-86-2 and others disclosed in U.S. Pat. No. 4,046,889), ceranapril (and others disclosed in U.S. Pat. No. 4,452,790), cetapril (alacepril, Dainippon disclosed in Eur. Therap. Res. 39:671 (1986); 40:543 (1986)), cilazapril (Hoffman-LaRoche) disclosed in J. Cardiovasc. Pharmacol. 9:39 (1987), indalapril (delapril hydrochloride (2H-1,2,4-Benzothiadiazine-7-sulfonamide, 3-bicyclo[2.2.1]hept-5-en-2-yl-6-chloro-3,4-dihydro-, 1,1-dioxide CAS RN 2259-96-3); disclosed in U.S. Pat. No. 4,385,051), enalapril (and others disclosed in U.S. Pat. No. 4,374,829), enalopril, enaloprilat, fosinopril, ((such as trans-L-proline, 4-cyclohexyl-1-[[[2-methyl-1-(1-oxopropoxy)propoxy](4-phenylbutyl)phosphinyl]acetyl]-, sodium salt, e.g., Monopril, Bristol-Myers Squibb and others disclosed in U.S. Pat. No. 4,168,267), fosinopril sodium (L-Proline, 4-cyclohexyl-1-[[(R)-[(1S)-2-methyl-1-(1-ox-opropoxy)propox), imidapril, indolapril (Schering, disclosed in J. Cardiovasc. Pharmacol. 5:643, 655 (1983)), lisinopril (Merck), losinopril, moexipril, moexipril hydrochloride (3-Isoquinolinecarboxylic acid, 2-[(2S)-2-[[(1S)-1-(ethoxycarbonyl)-3-phenylpropyl]amino]-1-oxopropyl]-1,2,3,4-tetrahydro-6,7-dimethoxy-, monohydrochloride, (3S) CAS RN 82586-52-5), quinapril, quinaprilat, ramipril (Hoechsst) disclosed in EP 79022 and Curr. Ther. Res. 40:74 (1986), perindopril erbumine (such as 2S,3aS,7aS-1-[(S)—N—[(S)-1-Carboxybutyl]alanyl]hexahydro-2-indolinecarboxylic acid, 1-ethyl ester, compound with tert-butylamine (1:1), e.g., Aceon®, Solvay), perindopril (Servier, disclosed in Eur. J. clin. Pharmacol. 31:519 (1987)), quanipril (disclosed in U.S. Pat. No. 4,344,949), spirapril (Schering, disclosed in Acta. Pharmacol. Toxicol. 59 (Supp. 5):173 (1986)), tenocapril, trandolapril, zofenopril (and others disclosed in U.S. Pat. No. 4,316,906), rentiapril (fentiapril, disclosed in Clin. Exp. Pharmacol. Physiol. 10:131 (1983)), pivopril, YS980, teprotide (Bradykinin potentiator BPP9a CAS RN 35115-60-7), BRL 36,378 (Smith Kline Beecham, see EP80822 and EP60668), MC-838 (Chugai, see C. A. 102:72588v and Jap. J. Pharmacol. 40:373 (1986), CGS14824 (Ciba-Geigy, 3-([1-ethoxycarbonyl-3-phenyl-(1S)-propyl]amino)-2,3,4,5-tetrahydro-2-ox-o-1-(3S)-benzazepine-1 acetic acid HCl, see U.K. Patent No. 2103614), CGS16,617 (Ciba-Geigy, 3(S)-[[(1S)-5-amino-1-carboxypentyl]amino]-2,3,4, -5-tetrahydro-2-oxo-1H-1-benzazepine-1-ethanoic acid, see U.S. Pat. No. 4,473,575), Ru 44570 (Hoechst, see Arzneimittelforschung 34:1254 (1985)), R 31-2201 (Hoffman-LaRoche see FEBS Lett. 165:201 (1984)), CI925 (Pharmacologist 26:243, 266 (1984)), WY-44221 (Wyeth, see J. Med. Chem. 26:394 (1983)), and those disclosed in US2003006922 (paragraph 28), U.S. Pat. No. 4,337,201, U.S. Pat. No. 4,432,971 (phosphonamidates); neutral endopeptidase inhibitors such as omapatrilat (Vanlev®), CGS 30440, cadoxatril and ecadotril, fasidotril (also known as aladotril or alatriopril), sampatrilat, mixanpril, and gemopatrilat, AVE7688, ER4030, and those disclosed in U.S. Pat. No. 5,362,727, U.S. Pat. No. 5,366,973, U.S. Pat. No. 5,225,401, U.S. Pat. No. 4,722,810, U.S. Pat. No. 5,223,516, U.S. Pat. No. 4,749,688, U.S. Pat. No. 5,552,397, U.S. Pat. No. 5,504,080, U.S. Pat. No. 5,612,359, U.S. Pat. No. 5,525,723, EP0599444, EP0481522, EP0599444, EP0595610, EP0534363, EP534396, EP534492, EP0629627;
endothelin antagonists such as tezosentan, A308165, and YM62899, and the like; vasodilators such as hydralazine (apresoline), clonidine (clonidine hydrochloride (1H-Imidazol-2-amine, N-(2,6-dichlorophenyl)-4,5-dihydro-, monohydrochloride CAS RN 4205-91-8), catapres, minoxidil (loniten), nicotinyl alcohol (roniacol), diltiazem hydrochloride (such as 1,5-Benzothiazepin-4(5H)-one, 3-(acetyloxy)-5-[2-(dimethylamino) ethyl]-2, -3-dihydro-2(4-methoxyphenyl)-, monohydrochloride, (+)-cis, e.g., Tiazac®, Forest), isosorbide dinitrate (such as 1,4:3,6-dianhydro-D-glucitol 2,5-dinitrate e.g., Isordil® Titradose®, Wyeth-Ayerst), sosorbide mononitrate (such as 1,4:3,6-dianhydro-D-glucito-1,5-nitrate, an organic nitrate, e.g., Ismo®, Wyeth-Ayerst), nitroglycerin (such as 2,3 propanetriol trinitrate, e.g., Nitrostat® Parke-Davis), verapamil hydrochloride (such as benzeneacetonitrile, (±)-(alpha)[3-[[2-(3,4 dimethoxyphenyl)ethyl]methylamino]propyl]-3,4-dimethoxy-(alpha)-(1-methylethyl) hydrochloride, e.g., Covera HS® Extended-Release, Searle), chromonar (which may be prepared as disclosed in U.S. Pat. No. 3,282,938), clonitate (Annalen 1870 155), droprenilamine (which may be prepared as disclosed in DE2521113), lidoflazine (which may be prepared as disclosed in U.S. Pat. No. 3,267,104); prenylamine (which may be prepared as disclosed in U.S. Pat. No. 3,152,173), propatyl nitrate (which may be prepared as disclosed in French Patent No. 1,103,113), mioflazine hydrochloride (1-Piperazineacetamide, 3-(aminocarbonyl)₄-[4,4-bis(4-fluorophenyl)butyl]-N-(2,6-dichlorophenyl)-, dihydrochloride CAS RN 83898-67-3), mixidine (Benzeneethanamine, 3,4-dimethoxy-N-(1-methyl-2-pyrrolidinylidene)-Pyrrolidine, 2-[(3,4-dimethoxyphenethyl)imino]-1-methyl-1-Methyl-2-[(3,4-dimethoxyphenethyl) imino]pyrrolidine CAS RN 27737-38-8), molsidomine (1,2,3-Oxadiazolium, 5-[(ethoxycarbonyl)amino]-3-(4-morpholinyl)-, inner salt CAS RN 25717-80-0), isosorbide mononitrate (D-Glucitol, 1,4:3,6-dianhydro-, 5-nitrate CAS RN 16051-77-7), erythrityl tetranitrate (1,2,3,4-Butanetetrol, tetranitrate, (2R,3S)-rel-CAS RN 7297-25-8), clonitrate (1,2-Propanediol, 3-chloro-, dinitrate (7CI, 8CI, 9CI) CAS RN 2612-33-1), dipyridamole Ethanol, 2,2′,2″,2′″-[(4,8-di-1-piperidinylpyrimido[5,4-d]pyrimidine-2,6-diyl)dinitrilo]tetrakis-CAS RN 58-32-2), nicorandil (CAS RN 65141-46-03-), pyridinecarboxamide (N-[2-(nitrooxy)ethyl]-Nisoldipine-3,5-Pyridinedicarboxylic acid, 1,4-dihydro-2,6-dimethyl-4-(2-nitrophenyl)-, methyl 2-methylpropyl ester CAS RN 63675-72-9), nifedipine-3,5-Pyridinedicarboxylic acid, 1,4-dihydro-2,6-dimethyl-4-(2-nitrophenyl)-, dimethyl ester CAS RN 21829-25-4), perhexiline maleate (Piperidine, 2-(2,2-dicyclohexylethyl)-, (2Z)-2-butenedioate (1:1) CAS RN 6724-53-4), oxprenolol hydrochloride (2-Propanol, 1-[(1-methylethyl)amino]-3-[2-(2-propenyloxy)phenoxy]-, hydrochloride CAS RN 6452-73-9), pentrinitrol (1,3-Propanediol, 2,2-bis[(nitrooxy)methyl]-, mononitrate (ester) CAS RN 1607-17-6), verapamil (Benzeneacetonitrile, α-[3-[[2-(3,4-dimethoxyphenyl)ethyl]-methylamino]propyl]-3,4-dimethoxy-α-(1-methylethyl)-CAS RN 52-53-9) and the like;
angiotensin II receptor antagonists such as, aprosartan, zolasartan, olmesartan, pratosartan, FI6828K, RNH6270, candesartan (1H-Benzimidazole-7-carboxylic acid, 2-ethoxy-1-[[2′-(1H-tetrazol-5-yl)) [11′-biphenyl]-4-yl]methyl]-CAS RN 139481-59-7), candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole carboxylate, CAS RN 145040-37-5, U.S. Pat. No. 5,703,110 and U.S. Pat. No. 5,196,444), eprosartan (3-[1-4-carboxyphenylmethyl)-2-n-butyl-imidazol-5-yl]-(2-thienylmethyl) propenoic acid, U.S. Pat. No. 5,185,351 and U.S. Pat. No. 5,650,650), irbesartan (2-n-butyl-3-[[2′-(1h-tetrazol-5-yl)biphenyl-4-yl]methyl]1,3-diazazspiro[4,4]non-1-en-4-one, U.S. Pat. No. 5,270,317 and U.S. Pat. No. 5,352,788), losartan (2-N-butyl-4-chloro-5-hydroxymethyl-1-[(2′-(1H-tetrazol-5-yl)biphenyl-4-yl)-methyl]imidazole, potassium salt, U.S. Pat. No. 5,138,069, U.S. Pat. No. 5,153,197 and U.S. Pat. No. 5,128,355), tasosartan (5,8-dihydro-2,4-dimethyl-8-[(2′-(1H-tetrazol-5-yl)[1,1′-biphenyl]-4-yl)methyl]-pyrido[2,3-d]pyrimidin-7(6H)-one, US5149699), telmisartan (4′-[(1,4-dimethyl-2′-propyl-(2,6′-bi-1H-benzimidazol)-1′-yl)]-[1,1′-biphenyl]-2-carboxylic acid, CAS RN 144701-48-4, U.S. Pat. No. 5,591,762), milfasartan, abitesartan, valsartan (Diovan® (Novartis), (S)—N-valeryl-N-[[2′-(1H-tetrazol-5-yl)biphenyl-4-yl)methyl]valine, U.S. Pat. No. 5,399,578), EXP-3137 (2-N-butyl-4-chloro-1-[(2′-(1H-tetrazol-5-yl)biphenyl-4-yl)-methyl]imidazole-5-carboxylic acid, U.S. Pat. No. 5,138,069, U.S. Pat. No. 5,153,197 and U.S. Pat. No. 5,128,355), 3-(2′-(tetrazol-5-yl)-1,1′-biphen-4-yl)methyl-5,7-dimethyl-2-ethyl-3H-imidazo[4,5-b]pyridine, 4-[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′)-1H-tetrazol-5-yl)biphenyl-4-ylmethyl]quinazolin-4(3H)-one, 3-[2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]t-triazolo[1,5-c]pyrimidin-2(3H)-one, 2,7-diethyl-5-[[2′-(5-tetrazoly)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol[4,5-c]pyridine-5-ylmethyl]benzoic acid, ethyl ester, potassium salt, 3-methoxy-2,6-dimethyl-4-[[2′(H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′-1H-tetrazol-5-yl)biphenyl-4-yl]methyl]-3H-imidazo[4, 5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methylpyridine, 2-[[[2-butyl-1-([(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-e-[[2′-(H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidzole-5-carboxylic acid T-ethoxycarbonyloxyethyl ester, those disclosed in patent publications EP475206, EP497150, EP539086, EP539713, EP535463, EP535465, EP542059, EP497121, EP535420, EP407342, EP415886, EP424317, EP435827, EP433983, EP475898, EP490820, EP528762, EP324377, EP323841, EP420237, EP500297, EP426021, EP480204, EP429257, EP430709, EP434249, EP446062, EP505954, EP524217, EP514197, EP514198, EP514193, EP514192, EP450566, EP468372, EP485929, EP503162, EP533058, EP467207 EP399731, EP399732, EP412848, EP453210, EP456-442, EP470794, EP470795, EP495626, EP495627, EP499414, EP499416, EP499415, EP511791, EP516392, EP520723, EP520724, EP539066, EP438869, EP505893, EP530702, EP400835, EP400974, EP401030, EP407102, EP411766, EP409332, EP412594, EP419048, EP480659, EP481614, EP490587, EP467715, EP479479, EP502725, EP503838, EP505098, EP505111 EP513,979 EP507594, EP510812, EP511767, EP512675, EP512676, EP512870, EP517357, EP537937, EP534706, EP527534, EP540356, EP461040, EP540039, EP465368, EP498723, EP498722, EP498721, EP515265, EP503785, EP501892, EP519831, EP532410, EP498361, EP432737, EP504888, EP508393, EP508445, EP403159, EP403158, EP425211, EP427463, EP437103, EP481448, EP488532, EP501269, EP500409, EP540400, EP005528, EP028834, EP028833, EP411507, EP425921, EP430300, EP434038, EP442473, EP443568, EP445811, EP459136, EP483683, EP518033, EP520423, EP531876, EP531874, EP392317, EP468-470, EP470543, EP502314, EP529253, EP543263, EP540209, EP449699, EP465323, EP521768, EP415594, WO92/14468, WO93/08171, WO93/08169, WO91/00277, WO91/00281, WO91/14367, WO92/00067, WO92/00977, WO92/20342, WO93/04045, WO93/04046, WO91/15206, WO92/14714, WO92/09600, WO92/16552, WO93/05025, WO93/03018, WO91/07404, WO92/02508, WO92/13853, WO91/19697, WO91/11909, WO91/12001, WO91/11999, WO91/15209, WO91/15479, WO92/20687, WO92/20662, WO92/20661, WO93/01177, WO91/14679, WO91/13063, WO92/13564, WO91/17148, WO91/18888, WO91/19715, WO92/02257, WO92/04335, WO92/05161, WO92/07852, WO92/15577, WO93/03033, WO91/16313, WO92/00068, WO92/02510, WO92/09278, WO9210179, WO92/10180, WO92/10186, WO92/10181, WO92/10097, WO92/10183, WO92/10182, WO92/10187, WO92/10184, WO92/10188, WO92/10180, WO92/10185, WO92/20651, WO93/03722, WO93/06828, WO93/03040, WO92/19211, WO92/22533, WO92/06081, WO92/05784, WO93/00341, WO92/04343, WO92/04059, U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138,069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,048, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, U.S. Pat. No. 5,041,152, and U.S. Pat. No. 5,210,204, and pharmaceutically acceptable salts and esters thereof, α/β adrenergic blockers such as nipradilol, arotinolol, amosulalol, bretylium tosylate (CAS RN: 61-75-6), dihydroergtamine mesylate (such as ergotaman-3′,6′,18-trione, 9-10-dihydro-12′-hydroxy-2′-methyl-5′-(phenylmethyl)-(5′(α))-, monomethanesulfonate, e.g., DHE 45® Injection, Novartis), carvedilol (such as (±)-1-(Carbazol-4-yloxy)-3-[[2-(o-methoxyphenoxy)ethyl]amino]-2-propanol, e.g., Coreg®, SmithKline Beecham), labetalol (such as 5-[1-hydroxy-2-[(1-methyl-3-phenylpropyl)amino]ethyl]salicylamide monohydrochloride, e.g., Normodyne®, Schering), bretylium tosylate (Benzenemethanaminium, 2-bromo-N-ethyl-N,N-dimethyl, salt with 4-methylbenzenesulfonic acid (1:1) CAS RN 61-75-6), phentolamine mesylate (Phenol, 3-[[(4,5-dihydro-1H-imidazol-2-yl)methyl] (4-methylphenyl)amino]-, monomethanesulfonate (salt) CAS RN 65-28-1), solypertine tartrate (5H-1,3-Dioxolo[4,5-f]indole, 7-[2-[4-(2-methoxyphenyl)-1-piperazinyl]ethyl]-, (2R,3R)-2,3-dihydroxybutanedioate (1:1) CAS RN 5591-43-5), zolertine hydrochloride (Piperazine, 1-phenyl-4-[2-(1H-tetrazol-5-yl)ethyl]-, monohydrochloride (8Cl, 9Cl) CAS RN 7241-94-3) and the like;
α adrenergic receptor blockers, such as alfuzosin (CAS RN: 81403-68-1), terazosin, urapidil, prazosin (Minipress®), tamsulosin, bunazosin, trimazosin, doxazosin, naftopidil, indoramin, WHP 164, XEN010, fenspiride hydrochloride (which may be prepared as disclosed in U.S. Pat. No. 3,399,192), proroxan (CAS RN 33743-96-3), and labetalol hydrochloride and combinations thereof;
α 2 agonists such as methyldopa, methyldopa HCL, lofexidine, tiamenidine, moxonidine, rilmenidine, guanobenz, and the like;
aldosterone inhibitors, and the like;
angiopoietin-2-binding agents such as those disclosed in WO03/030833;
anti-angina agents such as ranolazine (hydrochloride-1-Piperazineacetamide, N-(2,6-dimethylphenyl)-4-[2-hydroxy-3-(2-methoxyphenoxy)propyl]-, dihydrochloride CAS RN 95635-56-6), betaxolol hydrochloride (2-Propanol, 1-[4-[2 (cyclopropylmethoxy)ethyl]phenoxy]-3-[(1-methylethyl)amino]-, hydrochloride CAS RN 63659-19-8), butoprozine hydrochloride (Methanone, [4-[3(dibutylamino)propoxy]phenyl] (2-ethyl-3-indolizinyl)-, monohydrochloride CAS RN 62134-34-3), cinepazet maleate-1-Piperazineacetic acid, 4-[1-oxo-3-(3,4,5-trimethoxyphenyl)-2-propenyl]-, ethyl ester, (2Z)-2-butenedioate (1:1) CAS RN 50679-07-7), tosifen (Benzenesulfonamide, 4-methyl-N-[[[(1S)-1-methyl-2-phenylethyl]amino]carbonyl]-CAS RN 32295-184), verapamilhydrochloride (Benzeneacetonitrile, α-[3-[[2-(3,4-dimethoxyphenyl)ethyl]methylamino]propyl]-3,4-dimethoxy-α-(1-methylethyl)-, monohydrochloride CAS RN 152-114), molsidomine (1,2,3-Oxadiazolium, 5-[(ethoxycarbonyl)amino]-3-(4-morpholinyl)-, inner salt CAS RN 25717-80-0), and ranolazine hydrochloride (1Piperazineacetamide, N-(2,6-dimethylphenyl)₄-[2-hydroxy-3-(2-meth-oxyphenoxy)propyl]-, dihydrochloride CAS RN 95635-56-6); tosifen (Benzenesulfonamide, 4-methyl-N-[[[(1S)-1-methyl-2-phenylethyl]amino]carbonyl]-CAS RN 32295-184);
adrenergic stimulants such as guanfacine hydrochloride (such as N-amidino-2-(2,6-dichlorophenyl)acetamide hydrochloride, e.g., Tenex® Tablets available from Robins); methyldopa-hydrochlorothiazide (such as levo-3-(3,4-dihydroxyphenyl)-2-methylalanine) combined with Hydrochlorothiazide (such as 6-chloro-3,4-dihydro-2H-1,2,4-benzothiadiazine-7-sulfonamide 1,1-dioxide, e.g., the combination as, e.g., Aldoril® Tablets available from Merck), methyldopa-chlorothiazide (such as 6-chloro-2H-1,2,4-benzothiadiazine-7-sulfonamide 1,1-dioxide and methyldopa as described above, e.g., Aldoclor®, Merck), clonidine hydrochloride (such as 2-(2,6-dichlorophenylamino)-2-imidazoline hydrochloride and chlorthalidone (such as 2-chloro-5-(1-hydroxy-3-oxo-1-isoindolinyl)benzenesulfonamide), e.g., Combipres®, Boehringer Ingelheim), clonidine hydrochloride (such as 2-(2,6-dichlorophenylamino)-2-imidazoline hydrochloride, e.g., Catapres®, Boehringer Ingelheim), clonidine (1H-Imidazol-2-amine, N-(2,6-dichlorophenyl)-4,5-dihydro-CAS RN 4205-90-7); and those agents disclosed in US20030069221. Tests showing the efficacy of the therapy and the rationale for the combination therapy with an anti-hypertensive agent are described, for example, in US20030069221.

### Anti-Obesity Agents

The compounds described herein can be used in therapeutic combination with one or more anti-obesity agents, including but not limited to:

11β HSD-1 (11-beta hydroxy steroid dehydrogenase type 1) inhibitors, such as BVT 3498, BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,13a-decahydro-1,2,4-triazolo[4,3-a][11]annulene, and those compounds disclosed in WO01/90091, WO01/90090, WO01/90092 and WO02/072084;
5HT antagonists such as those in WO03/037871, WO03/037887, and the like;
5HT1a modulators such as carbidopa, benserazide and those disclosed in U.S. Pat. No. 6,207,699, WO03/031439, and the like;
5HT2c (serotonin receptor 2c) agonists, such as BVT933, DPCA37215, IK264, PNU 22394, WAY161503, R-1065, SB 243213 (Glaxo Smith Kline) and YM 348 and those disclosed in U.S. Pat. No. 3,914,250, WO00/77010, WO02/36596, WO02/48124, WO02/10169, WO01/66548, WO02/44152, WO02/51844, WO02/40456, and WO02/40457;
5HT6 receptor modulators, such as those in WO03/030901, WO03/035061, WO03/039547, and the like;
acyl-estrogens, such as oleoyl-estrone, disclosed in del Mar-Grasa, M. et al., Obesity Research, 9:202-9 (2001) and Japanese Patent Application No. JP 2000256190;
anorectic bicyclic compounds such as 1426 (Aventis) and 1954 (Aventis), and the compounds disclosed in WO00/18749, WO01/32638, WO01/62746, WO01/62747, and WO03/015769;
CB 1 (cannabinoid-1 receptor) antagonist/inverse agonists such as rimonabant (Acomplia; Sanofi), SR-147778 (Sanofi), SR-141716 (Sanofi), BAY 65-2520 (Bayer), and SLV 319 (Solvay), and those disclosed in patent publications U.S. Pat. No. 4,973,587, U.S. Pat. No. 5,013,837, U.S. Pat. No. 5,081,122, U.S. Pat. No. 5,112,820, U.S. Pat. No. 5,292,736, U.S. Pat. No. 5,532,237, U.S. Pat. No. 5,624,941, U.S. Pat. No. 6,028,084, U.S. Pat. No. 6,509,367, U.S. Pat. No. 6,509,367, US20060069080 (specifically including those referenced or disclosed by formulae in paragraphs 28-168), WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/027114, WO03/037332, WO03/040107, WO03/086940, WO03/084943 and EP658546;
CCK-A (cholecystokinin-A) agonists, such as AR-R 15849, GI 181771 (GSK), JMV-180, A-71378, A-71623 and SR146131 (Sanofi), and those described in U.S. Pat. No. 5,739,106;
CNTF (Ciliary neurotrophic factors), such as GI-181771 (Glaxo-SmithKline), SR146131 (Sanofi Synthelabo), butabindide, PD170,292, and PD 149164 (Pfizer);
CNTF derivatives, such as Axokine® (Regeneron), and those disclosed in WO94/09134, WO98/22128, and WO99/43813;
dipeptidyl peptidase IV (DP-IV) inhibitors, such as isoleucine thiazolidide, valine pyrrolidide, NVP-DPP728, LAF237, P93/01, P 3298, TSL 225 (tryptophyl-1,2,3,4-tetrahydroisoquinoline-3-carboxylic acid; disclosed by Yamada et al, Bioorg. & Med. Chem. Lett. 8 (1998) 1537-1540), TMC-2A/2B/2C, CD26 inhibitors, FE 999011, P9310/K364, VIP 077, SDZ 274-444, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) and the compounds disclosed patent publications. WO99/38501, WO99/46272, WO99/67279 (Probiodrug), WO99/67278 (Probiodrug), WO99/61431 (Probiodrug), WO02/083128, WO02/062764, WO03/000180, WO03/000181, WO03/000250, WO03/002530, WO03/002531, WO03/002553, WO03/002593, WO03/004498, WO03/004496, WO03/017936, WO03/024942, WO03/024965, WO03/033524, WO03/037327 and EP1258476;
growth hormone secretagogue receptor agonists/antagonists, such as NN703, hexarelin, MK-0677 (Merck), SM-130686, CP-424391 (Pfizer), LY 444,711 (Eli Lilly), L-692,429 and L-163,255, and such as those disclosed in U.S. Ser. No. 09/662,448, U.S. provisional application 60/203,335, U.S. Pat. No. 6,358,951, US2002049196, US2002/022637, WO01/56592 and WO02/32888;
H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO02/15905, WO03/024928 and WO03/024929;
leptin derivatives, such as those disclosed in U.S. Pat. No. 5,552,524, U.S. Pat. No. 5,552,523, U.S. Pat. No. 5,552,522, U.S. Pat. No. 5,521,283, WO96/23513, WO96/23514, WO96/23515, WO96/23516, WO96/23517, WO96/23518, WO96/23519, and WO96/23520;
leptin, including recombinant human leptin (PEG-OB, Hoffman La Roche) and recombinant methionyl human leptin (Amgen);
lipase inhibitors, such as tetrahydrolipstatin (orlistat/Xenical®), Triton WR1339, RHC80267, lipstatin, teasaponin, diethylumbelliferyl phosphate, FL-386, WAY-121898, Bay-N-3176, valilactone, esteracin, ebelactone A, ebelactone B, and RHC 80267, and those disclosed in patent publications WO01/77094, U.S. Pat. No. 4,598,089, U.S. Pat. No. 4,452,813, U.S. Pat. No. 5,512,565, U.S. Pat. No. 5,391,571, U.S. Pat. No. 5,602,151, U.S. Pat. No. 4,405,644, U.S. Pat. No. 4,189,438, and U.S. Pat. No. 4,242,453;
lipid metabolism modulators such as maslinic acid, erythrodiol, ursolic acid uvaol, betulinic acid, betulin, and the like and compounds disclosed in WO03/011267;
Mc4r (melanocortin 4 receptor) agonists, such as CHIR86036 (Chiron), ME-10142, ME-10145, and HS-131 (Melacure), and those disclosed in PCT publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12166, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410;
Mc5r (melanocortin 5 receptor) modulators, such as those disclosed in WO97/19952, WO00/15826, WO00/15790, US20030092041;
melanin-concentrating hormone 1 receptor (MCHR) antagonists, such as T-226296 (Takeda), SB 568849, SNP-7941 (Synaptic), and those disclosed in patent publications WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480, JP13226269, and JP1437059;
mGluR5 modulators such as those disclosed in WO03/029210, WO03/047581, WO03/048137, WO03/051315, WO03/051833, WO03/053922, WO03/059904, and the like;
serotoninergic agents, such as fenfluramine (such as Pondimin® (Benzeneethanamine, N-ethyl-alpha-methyl-3-(trifluoromethyl)-, hydrochloride), Robbins), dexfenfluramine (such as Redux® (Benzeneethanamine, N-ethyl-alpha-methyl-3-(trifluoromethyl)-, hydrochloride), Interneuron) and sibutramine ((Meridia®, Knoll/Reductil®) including racemic mixtures, as optically pure isomers (+) and (−), and pharmaceutically acceptable salts, solvents, hydrates, clathrates and prodrugs thereof including sibutramine hydrochloride monohydrate salts thereof, and those compounds disclosed in U.S. Pat. No. 4,746,680, U.S. Pat. No. 4,806,570, and U.S. Pat. No. 5,436,272, US20020006964, WO01/27068, and WO01/62341;
NE (norepinephrine) transport inhibitors, such as GW 320659, despiramine, talsupram, and nomifensine;
NPY 1 antagonists, such as BIBP3226, J-115814, BIBO 3304, LY-357897, CP-671906, GI-264879A, and those disclosed in U.S. Pat. No. 6,001,836, WO96/14307, WO01/23387, WO99/51600, WO01/85690, WO01/85098, WO01/85173, and WO01/89528; NPY5 (neuropeptide Y Y5) antagonists, such as 152,804, GW-569180A, GW-594884A, GW-587081X, GW-548118X, FR235208, FR226928, FR240662, FR252384, 1229U91, GI-264879A, CGP71683A, LY-377897, LY-366377, PD-160170, SR-120562A, SR-120819A, JCF-104, and H409/22 and those compounds disclosed in patent publications U.S. Pat. No. 6,140,354, U.S. Pat. No. 6,191,160, U.S. Pat. No. 6,218,408, U.S. Pat. No. 6,258,837, U.S. Pat. No. 6,313,298, U.S. Pat. No. 6,326,375, U.S. Pat. No. 6,329,395, U.S. Pat. No. 6,335,345, U.S. Pat. No. 6,337,332, U.S. Pat. No. 6,329,395, U.S. Pat. No. 6,340,683, EP01010691, EP-01044970, WO97/19682, WO97/20820, WO97/20821, WO97/20822, WO97/20823, WO98/27063, WO00/107409, WO00/185714, WO00/185730, WO00/64880, WO00/68197, WO00/69849, WO01/0113917, WO01/09120, WO01/14376, WO01/85714, WO01/85730, WO01/07409, WO01/02379, WO01/23388, WO01/23389, WO01/44201, WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al., J. Med. Chem. 43:4288-4312 (2000); opioid antagonists, such as nalmefene (Revex®), 3-methoxynaltrexone, naloxone, and naltrexone and those disclosed in WO00/21509;
orexin antagonists, such as SB-334867-A and those disclosed in patent publications WO01/96302, WO01/68609, WO02/44172, WO02/51232, WO02/51838, WO02/089800, WO02/090355, WO03/023561, WO03/032991, and WO03/037847;
PDE inhibitors (e.g., compounds which slow the degradation of cyclic AMP (cAMP) and/or cyclic GMP (cGMP) by inhibition of the phosphodiesterases, which can lead to a relative increase in the intracellular concentration of cAMP and cGMP; possible PDE inhibitors are primarily those substances which are to be numbered among the class consisting of the PDE3 inhibitors, the class consisting of the PDE4 inhibitors and/or the class consisting of the PDE5 inhibitors, in particular those substances which can be designated as mixed types of PDE3/4 inhibitors or as mixed types of PDE3/4/5 inhibitors) such as those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9506623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE3116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702 and sildenafil (Viagra®)), PDE4 inhibitors (such as RO-20-1724, MEM 1414 (R1533/R1500; Pharmacia Roche), denbuflline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, amrinone, pimobendan, cilostazol, quazinone and N-(3,5-dichloropyrid-4-yl)-3-cyclopropylmethoxy-4-difluoromethoxybenzamide, PDE3 inhibitors (such as sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as cilomilast, fenoximone, pentoxifylline, roflumilast, tadalafil (Cialis®), theophylline, and vardenafil (Levitra®); Neuropeptide Y2 (NPY2) agonists include but are not limited to: peptide YY and fragments and variants thereof (e.g., YY3-36 (PYY3-36) (N. Engl. J. Med. 349:941, 2003; CAS RN. 870491-48-8) and PYY agonists such as those disclosed in WO03/026591, WO03/057235, and WO03/027637;
serotonin reuptake inhibitors, such as, paroxetine, fluoxetine (Prozac®), fluvoxamine, sertraline, citalopram, and imipramine, and those disclosed in U.S. Pat. No. 6,162,805, U.S. Pat. No. 6,365,633, WO03/00663, WO01/27060, and WO01/162341;
thyroid hormone β agonists, such as QRX-431 (QuatRx), GC-24 (described in US 20040110154), KB-2611 (KaroBioBMS), and those disclosed in WO02/15845, WO97/21993, WO99/00353, GB98/284425, U.S. Provisional Application No. 60/183,223, and Japanese Patent Application No. JP 2000256190; UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in WO99/00123;
β3 (beta adrenergic receptor 3) agonists, such as AJ9677/TAK677 (Dainippon/Takeda), L750355 (Merck), CP331648 (Pfizer), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), SR 59119A, and those disclosed in U.S. Pat. No. 5,541,204, U.S. Pat. No. 5,770,615, U.S. Pat. No. 5,491,134, U.S. Pat. No. 5,776,983, US488064, U.S. Pat. No. 5,705,515, U.S. Pat. No. 5,451,677, WO94/18161, WO95/29159, WO97/46556, WO98/04526 and WO98/32753, WO01/74782, WO02/32897, WO03/014113, WO03/016276, WO03/016307, WO03/024948, WO03/024953 and WO03/037881; noradrenergic agents including, but not limited to, diethylpropion (such as Tenuate® (1-propanone, 2-(diethylamino)-1-phenyl-, hydrochloride), Merrell), dextroamphetamine (also known as dextroamphetamine sulfate, dexamphetamine, dexedrine, Dexampex, Ferndex, Oxydess II, Robese, Spancap #1), mazindol ((or 5-(p-chlorophenyl)-2,5-dihydro-3H-imidazo[2,1-a]isoindol-5-ol) such as Sanorex®, Novartis or Mazanor®, Wyeth Ayerst), phenylpropanolamine (or Benzenemethanol, alpha-(1-aminoethyl)-, hydrochloride), phentermine ((or Phenol, 3-[[4,5-dihydro-1H-imidazol-2-yl)ethyl](4-methylphenyl)amino], monohydrochloride) such as Adipex-P®, Lemmon, FASTIN®, Smith-Kline Beecham and Ionamin®, Medeva), phendimetrazine ((or (2S,3S)-3,4-Dimethyl-2-phenylmorpholine L-(+)-tartrate (1:1)) such as Metra® (Forest), Plegine® (Wyeth-Ayerst), Prelu-2P® (Boehringer Ingelheim), and Statobex® (Lemmon), phendamine tartrate (such as Thephorin® (2,3,4,9-Tetrahydro-2-methyl-9-phenyl-1H-indenol[2,1-c]pyridine L-(+)-tartrate (1:1)), Hoffmann-LaRoche), methamphetamine (such as Desoxyn®, Abbot ((S)—N, (alpha)-dimethylbenzeneethanamine hydrochloride)), and phendimetrazine tartrate (such as Bontril® Slow-Release Capsules, Amarin-(3,4-Dimethyl-2-phenylmorpholine Tartrate);
fatty acid oxidation upregulator/inducers such as Famoxin® (Genset);
monamine oxidase inhibitors including but not limited to befloxatone, moclobemide, brofaromine, phenoxathine, esuprone, befol, toloxatone, pirlindol, amiflamine, sercloremine, bazinaprine, lazabemide, milacemide, caroxazone and other certain compounds as disclosed by WO01/12176; and
other anti-obesity agents such as 5HT-2 agonists, ACC (acetyl-CoA carboxylase) inhibitors such as those described in WO03/072197, alpha-lipoic acid (alpha-LA), AOD9604, appetite suppressants such as those in WO03/40107, ATL-962 (Alizyme PLC), benzocaine, benzphetamine hydrochloride (Didrex), bladderwrack (focus vesiculosus), BRS3 (bombesin receptor subtype 3) agonists, bupropion, caffeine, CCK agonists, chitosan, chromium, conjugated linoleic acid, corticotropin-releasing hormone agonists, dehydroepiandrosterone, DGAT1 (diacylglycerol acyltransferase 1) inhibitors, DGAT2 (diacylglycerol acyltransferase 2) inhibitors, dicarboxylate transporter inhibitors, ephedra, exendin-4 (an inhibitor of glp-1) FAS (fatty acid synthase) inhibitors (such as Cerulenin and C75), fat resorption inhibitors (such as those in WO03/053451, and the like), fatty acid transporter inhibitors, natural water soluble fibers (such as psyllium, plantago, guar, oat, pectin), galanin antagonists, galega (Goat's Rue, French Lilac), garcinia cambogia, germander (teucrium chamaedrys), ghrelin antibodies and ghrelin antagonists (such as those disclosed in WO01/87335, and WO02/08250), GLP-1 (glucagon-like peptide 1) agonists (e.g., exendin-4), glp-1 (glucagon-like peptide-1), glucocorticoid antagonists, glucose transporter inhibitors, growth hormone secretagogues (such as those disclosed and specifically described in U.S. Pat. No. 5,536,716), interleukin-6 (IL-6) and modulators thereof (as in WO03/057237, and the like), L-carnitine, Mc3r (melanocortin 3 receptor) agonists, MCH2R (melanin concentrating hormone 2R) agonist/antagonists, melanin concentrating hormone antagonists, melanocortin agonists (such as Melanotan II or those described in WO 99/64002 and WO 00/74679), nomame herba, phosphate transporter inhibitors, phytopharm compound 57 (CP 644,673), pyruvate, SCD-1 (stearoyl-CoA desaturase-1) inhibitors, T71 (Tularik, Inc., Boulder Colo.), Topiramate (Topimax®, indicated as an anti-convulsant which has been shown to increase weight loss), transcription factor modulators (such as those disclosed in WO03/026576), β-hydroxy steroid dehydrogenase-1 inhibitors (β-HSD-1), β-hydroxy-β-methylbutyrate, p57 (Pfizer), Zonisamide (Zonegran®, indicated as an anti-epileptic which has been shown to lead to weight loss), and the agents disclosed in US20030119428 paragraphs 20-26. Tests showing the efficacy of the therapy and the rationale for the combination therapy with an anti-obesity agent are presented in US20030119428.

### Agents Used to Treat Autoimmune Disorders

The compounds described herein can be used in therapeutic combination with one or more agents used to treat autoimmune disorders including, but not limited to: (a) disease modifying antirheumatic drugs, including methotrexate, gold salts, D-penicillamine, hydroxychloroquine, auranofin, sulfsalazine; (b) nonsteroidal antiinflammatory drugs, including indomethacin, naproxen, diclofenac, ibuprofen, aspirin and aspirin analogs, acetaminophen; (c) COX-2 selective inhibitors, including celecoxib, rofecoxib, etoricoxib, valdecoxib, lumiracoxib; (d) COX-1 inhibitors; (e) immunosuppressives, including calcineurin inhibitors such as cyclosporine and FK506; p70^{S6} kinase inhibitors such as sirolimus and rapamycin; inosine monophosphate dehydrogenase inhibitors such as mycophenolate (including mycophenolate mofetil); leflunomide, cyclophosphamide, azathioprine; (f) steroids, including prednisone, betamethasone, budesonide and dexamethasone; (g) biological response modifiers, including TNFα antagonists such as infliximab, adalimmab and etanercept; IL-1 receptor antagonists such as anakinra; humanized or chimeric antibodies or fusion proteins such as alefacept, efalizumab, daclizumab; anti-chemokine antibodies or interleukins; and (h) other agents useful for the treatment of autoimmune disorders, including chemokine receptor antagonists or modulators, cannabinoid receptor antagonists or modulators, inhibitors of matrix metalloproteinases including those described herein, TNFα-converting enzymes, nitric oxide synthetases or phosphodiesterase IV, such as roflumilast or cilomilast; inhibitors of p38 MAP-kinase, the NF-kappa.beta., pathway or IL-1 receptor associated kinase or inhibitors of interactions involving adhesion molecules such as LFA-1, VLA-4, ICAM-1, VCAM-1, α₄β₇, MAdCAM-1, and αᵥβ₃. Tests showing the efficacy of the therapy and the rationale for the combination therapy with agents used to treat autoimmune disorders are presented in US20040092499.

### Agents Used to Treat Demylenation and Associated Conditions

The compounds described herein can be used in therapeutic combination with one or more agents used to treat demylenation and its associated conditions including, but not limited to: beta-interferon (such as Avonex®, Biogen, Inc. and Betaseron®, Berlex Laboratories), which can decrease the frequency and occurrence of flare-ups and slow the progression to disability, glatiramer acetate (such as Copaxone®, Teva Neuroscience, Inc.), which can reduce the frequency of relapses, and/or administration of corticosteroids, such as prednisone (available from Roxane), to relieve acute symptoms. The amount of respective antidemyelination agent to be administered to the subject readily can be determined by one skilled in the art from the Physician's Desk Reference (56.sup.th Ed. 2002) at pages 1013-1016, 988995, 3306-3310 and 3064-3066, incorporated herein by reference. Tests showing the efficacy of the therapy and the rationale for the combination therapy with agents used to treat demylenation and its associated conditions are described, for example, in US20040092500.

### Agents Used to Treat Alzheimer's Disease

The compounds described herein can be used in therapeutic combination with one or more agents used to treat Alzheimer's disease including, but not limited to:

cholinesterase inhibitors (such as donepezil hydrochloride (such as Aricept® (Pfizer)), rivastigmine tartrate (such as Exelon (Novartis)), tacrine (such as Cognex® (Parke-Davis)), galanthamine and derivatives thereof (Janssen), metrifonate (Bayer Corp.), epigalanthamine, norgalanthamine, fasciculin, metrifonate, heptyl-physostigmine, norpyridostigmine, nomeostigmine, ipidacrine (Nikken Chemicals Co. Ltd.), TAK-147 & T-82 (SS Pharmaceutical Co. Ltd.), methanesulfonyl fluoride, CHF-2819, phenserine, physostigmine (Forest Laboratories, Inc.), huperzine, cymserine (Anonyx Inc.), tolserine (National Institutes of Health), ER-127528 (Eisai Co. Ltd.), and combinations thereof), muscarinic receptor agonists (such as aceclide, pilocarpine, oxotremorine, arecaidine, 5-methylfurmethiodide, cevimeline, PD-151832 (Pfizer Inc.), YM-796 (Yamanouchi Pharmaceutical Inc.), P-58 (Phytopharm plc) and combinations thereof), M2 muscarinic receptor antagonists, acetylcholine release stimulators (such as minaprine, montirelin (Grunenthal GmbH), T-588 (Toyama Chemical Co. Ltd.), XE-991 and combinations thereof, choline uptake stimulators (such as MKC-231 (Mitsubishi-Tokyo Pharmaceuticals Inc)), nicotinic cholinergic receptor agonists (such as altinicline, (SIBIA Neurosciences Inc.), SIB-1553A, ABT-089 (disclosed in U.S. Pat. No. 5,278,176, Abbot), nicotine patch, GRS-21, TC-2403 and combinations thereof), anti-Aβ vaccines (such as AN-1792), γ-secretase inhibitors or β-secretase inhibitors (such as Asn⁶⁷⁰, Sta⁶⁷¹, Val⁶⁷²-Amyloid β/A4 Protein Precursor₇₇₀ (662-675) (catalog no. H-4948; Bachem), presenilin-1, presenilin-2 and derivatives thereof comprising one or more conservative substitutions), amyloid aggregation inhibitors (such as reumacon (Conpharm AB), NC-531 (Neurochem Inc.), PPI-1019 (Praecis Pharmaceuticals Inc.) and combinations thereof), amyloid precursor protein antisense oligonucleotides, monoamine reuptake inhibitors (such as NS-2330), human stem cells, gene therapy, nootropic agents (such as oxiracetam (ISF Societa Per Azioni), pramiracetam (Warner Lambert), idebenone (Takeda Chemical Inds. Ltd.), anapsos (ASAC Pharmaceuticals Intl.), nebracetam (Boehringer Ingelheim), JTP-2942 (Japan Tobacco Inc.), fasoracetam (Nippon Shinyaku Co. Ltd.), bacosides (Central Drug Research Institute), alzene (Bar-IIan University), KA-672 (Dr. Willmar Schwabe GmbH & Co.), alaptid (VUFB), IQ-200, ALE-26015 (Allelix Pharm-Eco LP) and combinations thereof), AMPA receptor ligands (such as CX-516 & CX-691 (Cortex Pharmaceuticals Inc.) and combinations thereof), growth factors or growth factor receptor agonists (such as leteprinim), anti-inflammatory agents (such as COX2 inhibitors (such as Vioxx rofecoxib (Merck) and Celebrex celecoxib (Pfizer), cytokine inhibitors (such as thalidomide disclosed in WO95/04533 and dexanabinol), complement inhibitors, leukotriene receptor antagonists and combinations thereof, free radical scavengers (such as EGb-761 (Yuyu Industrial Co.), CPI-22, dexanabinol and combinations thereof), antioxidants, superoxide dismutase stimulators, calcium channel blockers (such as tamolarizine (Nippon Chemiphar Co., Ltd.), nimodipine (Bayer AG), PD-1 76078 (Elan Pharmaceuticals, Inc.), and combinations thereof), apoptosis inhibitors (such as acetyl-L-carnitine, CEP-1347 (Cephalon, Inc.), TCH-346 (Novartis AG) and combinations thereof), caspase inhibitors (such as pralnacasan), monoamine oxidase inhibitors (such as moclobemide (Roche Holding AG), selegiline, rasagiline (Teva Pharmaceutical Inds. Ltd.), SL-25.1188, Ro-41-1049 (Roche Holding AG), and combinations thereof), estrogens and estrogen receptor ligands, NMDA receptor antagonists (such as ketamine, phencyclidine, dizocilpine, tiletamine, dextromethorphan, amantadine, methadone, dextropropoxyphene, ketobemidone, memantine, ipenoxazone (Nippon Chemiphar Co. Ltd. and combinations thereof), Jun N-terminal kinase (JNK) inhibitors, copper/zinc chelators (such as clioquinol (PN Gerolymatos SA)), 5-HT1a receptor agonists (such as AP-159 (Asahi Kasei Corp)), NGF stimulators (such as xaliprodene (Sanofi-Synthelabo)), neuroprotective agents (such as citicholine, GS-1590 (Leo Pharmaceutical Products Ltd.) A/S, CPI- 1189 (Centaur Pharmaceuticals Inc.), SR-57667 (Sanofi-Synthelabo) and combinations thereof), H₃ histamine receptor antagonists (such as GT-2016 and GT-2331 (both available from Gliatech, Inc.) and combinations thereof), calpain inhibitors, poly ADP ribose polymerase inhibitors, prolylendopeptidase inhibitors (such as ONO-1603 (Ono Pharmaceutical Co. Ltd.), Z-321 (Zeria Pharmaceutical Co. Ltd.) and combinations thereof), calcium modulators (such as neurocalc (Apollo Biopharmaceuticals Inc)), corticortropin releasing factor receptor antagonists (such as NBI-113 (Neurocrine Biosciences, Inc)), corticortropin releasing factor binding protein inhibitors, GABA modulators (such as NGD 97-1 (Neurogen Corp)), GABA-A receptor antagonists, GABA-B receptor antagonists, neuroimmunophilin ligands, sigma receptor ligands (such as igmesine (Pfizer)), galanin receptor ligands, imidazoline/alpha adrenergic receptor antagonists (such as efaroxan (Reckitt & Colman PLC)), vasoactive intestinal peptide receptor agonists (such as stearyl-NIe-VIP), benzodiazepine receptor inverse agonists (such as S-8510 (Shionogi & Co. Ltd)), cannabinoid receptor agonists (such as dronabinol (Unimed Pharmaceuticals Inc)), thyrotropin releasing hormone receptor agonists (such as taltireline (Tanabe Seiyaku Co. Ltd) and protirelin (Takeda Chemical Inds., Inc.)), protein kinase C inhibitors, 5-HT3 receptor antagonists (such as GYKI-46903), prostaglandin receptor antagonists, topoisomerase II inhibitors (such as iododoxorubicin (Pharmacia & Upjohn AB)), steroid receptor ligand (such as GL-701 (Prestara)), nitric oxide modulators, RAGE inhibitors (such as ALT-711 (Alteon Inc)), dopamine receptor agonists (such as speramine), statine compounds disclosed in US20050090449, corticosteroid receptor antagonist (such as anticort) and combinations thereof. Tests showing the efficacy of the therapy and the rationale for the combination therapy with agents used to treat Alzheimer's disease and other memory associated therapies are described, for example, in US2003013699 and US 20040044023.

### Blood Modifiers

The compounds described herein can be used in therapeutic combination with one or more blood modifiers, i.e., agents capable of altering the number of platelets per a given volume of blood, inhibiting platelet function, including but not limited to platelet adhesion, aggregation or factor release, or reducing platelet count in patients with abnormally high levels in certain hematological malignancies to levels approximating normal levels capable of impacting negatively upon the formation of blood clots, and decreasing blood viscosity. Blood modifiers useful in the present invention include but are not limited to anti-coagulants, antithrombotic agents, fibrinogen receptor antagonists, platelet inhibitors, platelet aggregation inhibitors, lipoprotein-associated coagulation inhibitor, hemorrheologic agents, Factor VIIa inhibitors, Factor Xa inhibitors, and combinations thereof. Tests showing the efficacy of the therapy and the rationale for the combination therapy with blood modifiers are described, for example, in US20020147184.

Anti-coagulant agents are agents which inhibit the coagulation pathway by impacting negatively upon the production, deposition, cleavage and/or activation of factors essential in the formation of a blood clot. Useful anti-coagulant agents include but are not limited to argatroban (2-Piperidinecarboxylic acid, 1-[(2S)-5-[(aminoiminomethyl)amino]-1-oxo-2-[[(1,2,3,4-tetrahydro-3-methyl-8-quinolinyl)sulfonyl]amino]pentyl]-4-methyl-, CAS RN 74863-84-6), bivalirudin (CAS RN 128270-60-0), dalteparin sodium (heparin) e.g., Fragmin® Injection (Pharmacia & Upjohn), desirudin (Hirudin (Hirudo medicinalis isoform HV1), 63-desulfo CAS RN 120993-53-5), dicumarol (2H-1-Benzopyran-2-one, 3,3′-methylenebis[4-hydroxy-CAS RN 66-76-2 e.g., Mebaral® (Sanofi-Synthelabo)), lyapolate sodium (Ethenesulfonic acid, homopolymer, sodium salt CAS RN 25053-274), nafamostate mesylate (Benzoic acid, 4-[(aminoiminomethyl)amino]-, 6-(aminoiminomethyl)-2-naphthalenyl ester, dimethanesulfonate CAS RN 82956-11-4); phenprocoumon (2H-1-Benzopyran-2-one, 4-hydroxy-8-methoxy-3-(1-phenylpropyl)-CAS RN 132605-68-6), tinzaparin sodium (Heparin, sodium salt, CAS RN 9041-08-1, e.g., Innohep® Injection® (DuPont)), and warfarin sodium (3-((alpha)-acetonylbenzyl)-4-hydroxycoumarin, CAS RN 129-06-6, e.g., Coumadin for Injection (DuPont)).

Anti-thrombotic agents are agents which prevent the formation of a blood thrombus. A thrombus is an aggregation of blood factors, primarily platelets and fibrin with entrapment of cellular elements, frequently causing vascular obstruction at the point of its formation. Suitable examples of anti-thrombotic agents include, but are not limited to: melagatran; ximelagatran (Exanta®); anagrelide hydrochloride (6,7-dichloro-1,5-dihydroimidazo[2,1-b]quinazolin-2(3H)-one monohydrochloride monohydrate) e.g., Agrylin® (Shire US)); Tinzaparin sodium as described above; cilostazol (6-[4-(1-cyclohexyl-1H-tetrazol-5-yl)butoxy]-3,4-dihydro-2(1H)-quinolinone, CAS-73963-72-1, e.g., Pletal® (Pharmacia & Upjohn); Dalteparin sodium (as described above); danaparoid sodium, e.g., Orgaran® Injection (Organon); compounds disclosed in WO99/45913; Abciximab is the (Fab fragment of the chimeric human-murine monoclonal antibody 7E3. binds to the glycoprotein (GP) IIb/IIIa ((α)_{IIb} (β)₃) receptor of human platelets and inhibits platelet aggregation. Abciximab also binds to the vitronectin ((α, _{V} (β)₃) receptor found on platelets and vessel wall endothelial and smooth muscle cells, e.g., Abciximab, Reopro® (Lily)); ifetroban (Benzenepropanoic acid, 2-[[(1 S,2R,3S,4R)-3-[4-[(pentylamino)carbonyl]-2-oxazolyl]-7 oxabicyclo[2.2.1]hept-2-yl]methyl]-CAS RN 143443-90-7, disclosed in U.S. Pat. No. 5,100,889); Bivalirudin as described above; Cilostazol as described above; efegatran sulfate (L-Prolinamide, N-methyl-D-phenylalanyl-N-[(1S)-4-[(aminoiminomethyl)amino]-1-formylbutyl]-, sulfate (1:1) CAS RN 126721-07-1); dazoxiben hydrochloride (Benzoic acid, 4-[2-(1H-imidazol-1-yl)ethoxy]-, monohydrochloride CAS RN 74226-22-5); danaparoid sodium (a low molecular weight heparinoid, a mixture of the sodium salts of heparan sulfate (approximately 84%), dermatan sulfate (approximately 12%), and chondroitin sulfate (approximately 4%). It is derived from hog intestinal mucosa);

lotrafiban hydrochloride (1H-1,4-Benzodiazepine-2-acetic acid, 7-([4,4′-bipiperidin]-1-ylcarbonyl)-2,3,4,5-tetrahydro-4-methyl-3-oxo-, monohydrochloride, (2S)—) CAS RN 179599-82-7); ifetroban sodium (Benzenepropanoic acid, 2-[[(1S,2R,3S,4R)-3-[4-[(pentylamino)carbonyl]-2-oxazolyl]-7-oxabicyclo[2.2.1]hept-2-yl]methyl]-, monosodium salt, CAS RN 156715-37-6); lamifiban (Acetic acid, [[1-[(2S)-2-[[4-(aminoiminomethyl)benzoyl]amino]-3-(4-hydroxyphenyl)-1-oxopropyl]-4-piperidinyl]oxy]-, CAS RN 144412-49-7); fluretofen (1,1′-Biphenyl, 4′-ethynyl-2-fluoro-CAS RN 56917-294); enoxaparin sodium (Heparin, sodium salt, CAS RN 9041-08-1); orbofiban acetate hydrate (beta.-Alanine, N-[[[(3S)-1-[4-(aminoiminomethyl)phenyl]-2-oxo-3-pyrrolidinyl]amino]carbonyl]-, ethyl ester, acetate, hydrate (4:4:1), CAS RN 165800-05-5)); napsagatran (Glycine, N-[[(3S)-1-(aminoiminomethyl)-3-piperidinyl]methyl]-N2-(2-naphthalenylsulfonyl)-L-asparaginyl-N-cyclopropyl-, CAS RN 154397-77-0); roxifiban acetate (L-Alanine, 3-[[[(5R)-3-[4-(aminoiminomethyl)phenyl]-4,5-dihydro-5-isoxazolyl]acetyl]amino]-N-(butoxycarbonyl)-, methyl ester, monoacetate, CAS RN 176022-59-6); sibrafiban (Acetic acid, [[1-[(2S)-2-[[4-[(Z)amino(hydroxyimino)methyl]benzoyl]amino]-1-oxopropyl]-4-piperidinyl]oxy]-, ethyl ester, CAS RN 172927-65-0); zolimomab aritox, (Immunoglobulin G1, anti-(human CD5 (antigen) heavy chain) (mouse monoclonal H65-RTA.gamma.1-chain), disulfide with mouse monoclonal H65-RTA light chain, dimer, disulfide with ricin (castor bean A-chain), CAS RN 141483-72-9); trifenagrel (Ethanamine, 2-[2-(4,5-diphenyl-1H-imidazol-2-yl)phenoxy]-N,N-dimethyl-, CAS RN 84203-09-8).

Fibrinogen receptor antagonists are those agents which inhibit the common pathway of platelet aggregation. Suitable fibrinogen receptor antagonists include but are not limited toroxifiban acetate as described above; lotrafiban hydrochloride as described above, sibrafiban as described above, monoclonal antibody 7E3 (Fab fragment of the chimeric human-murine monoclonal antibody 7E3. binds to the glycoprotein (GP) IIb/IIIa ((α)_{IIb} (β)₃) receptor of human platelets and inhibits platelet aggregation); orbofiban, (beta.-Alanine, N-[[[(3S)-1-[4-(aminoiminomethyl)phenyl]-2-oxo-3-pyrrolidinyl]amino]carbonyl]-, ethyl ester, CAS RN 163250-90-6); xemilofiban (4-Pentynoic acid, 3-[[4-[[4-(aminoiminomethyl)phenyl]amino]-1,4-dioxobutyl]amino]-, ethyl ester, (3S)—, CAS RN 149820-74-6); fradafiban, (3-Pyrrolidineacetic acid, 5-[[[4′-(aminoiminomethyl)[1,1′-biphenyl]-4-yl]oxy]methyl]-2-oxo-, (3S,5S)—, CAS RN 148396-36-5); tirofiban (L-Tyrosine, N-(butylsulfonyl)-O-[4-(4-piperidinyl)butyl]-, CAS RN 144494-65-5, e.g., Aggrastat® Injection Premixed (Merck).

Platelet inhibitors are those agents that impair the ability of mature platelets to perform their normal physiological roles (i.e., their normal function). Platelets are normally involved in a number of physiological processes such as adhesion, for example, to cellular and non-cellular entities, aggregation, for example, for the purpose of forming a blood clot, and release of factors such as growth factors (e.g., platelet-derived growth factor (PDGF)) and platelet granular components. Suitable platelet inhibitors include, but are not limited to CS-747 (Eli Lilly); eptifibatide (Integrilin®); clopidogrel bisulfate, (Thieno[3,2-c]pyridine-5(4H)-acetic acid, α-(2-chlorophenyl)-6,7-dihydro-, methyl ester, (αS)-, sulfate (1:1), e.g., Plavix® (Sanofi-Synthelabo)); indomethacin, such as Indocin® I.V. (Indomethacin Sodium Trihydrate, Merck); mefenamate, (e.g., Ponstel® Kapseals (mefenamic acid) 2-{(2,3-dimethylphenyl)amino-N-2,3-xylylanthranilic acid (First Horizon)); Ticlopidine hydrochloride, (Thieno[3,2-c]pyridine, 5-[(2-chlorophenyl)methyl]-4,5,6,7-tetrahydro-, hydrochloride, e.g., Ticlid® (Roche Laboratories)); epoprostenol sodium, (Prosta-5,13-dien-1-oic acid, 6,9-epoxy-11,15-dihydroxy-, monosodium salt, (5Z,9α,11α,13E,15S) CAS RN 61849-14-7, e.g., Flolan® (Glaxo Wellcome)); aspirin, Benzoic acid, 2-(acetyloxy)-CAS RN 50-78-2); epoprostenol, (Prosta-5,13-dien-1-oic acid, 6,9-epoxy-11,15-dihydroxy-, (5Z,9α,11α,13E,15S)—, CAS RN 35121-78-9); naproxen (2-Naphthaleneacetic acid, 6-methoxy-α-methyl-, (αS) CAS RN 22204-53-1, e.g., EC-Naprosyn®Delayed-Release Tablets available from Roche Laboratories); buprofen, (Benzeneacetic acid, α-methyl-4-(2-methylpropyl)-, CAS RN 15687-27-1); droxicam, (2H,5H-1,3-Oxazino[5,6-c][1,2]benzothiazine-2,4(3H)-dione, 5-methyl-3-(2-pyridinyl)-, 6,6-dioxide, CAS RN 90101-16-9); diclofenac, (Benzeneacetic acid, 2-[(2,6-dichlorophenyl)amino]-CAS RN 15307-86-5 e.g., Arthroteo® (Searle)); sulfinpyrazone, (3,5-Pyrazolidinedione, 1,2-diphenyl-4-[2-(phenylsulfinyl)ethyl]-CAS RN 57-96-5, e.g., Sectral® (Wyeth-Ayerst)); piroxicam, (2H-1,2-Benzothiazine-3-carboxamide, 4-hydroxy-2-methyl-N-2-pyridinyl-1,1-dioxide, CAS RN 36322-90-4, e.g., Feldene® (Pfizer)); dipyridamole, (Ethanol, 2,2′,2″,2′″-[(4,8-di-1-piperidinylpyrimido[5,4-d]-pyrimidine-2,6-diyl)dinitrilo]tetrakis-CAS RN 58-32-2, e.g., Aggrenox® Capsules available from Boehringer Ingelheim); lexipafant, (L-Leucine, N-methyl-N-[[4-[(2-methyl-1H-imidazo[4,5-c]pyridin-1-yl)methyl]phenyl]sulfonyl]-, ethyl ester, CAS RN 139133-26-9); apafant Morpholine, 4-[3-[4-(2-chlorophenyl)-9-methyl-6H-thieno[3,2-f][1,2,4]triazolo[4,3-a][1,4]diazepin-2-yl]-1-oxopropyl]-, CAS RN 105219-56-5).

Platelet aggregation inhibitors as used herein refer to those compounds which reduce or halt the ability of platelets to associate physically with themselves or with other cellular and non-cellular components, thereby precluding the ability of a platelet to form a thrombus. Suitable platelet aggregation inhibitors include but are not limited to beraprost, (1H-Cyclopenta[b]benzofuran-5-butanoic acid, 2,3,3a,8b-tetrahydro-2-hydroxy-1-(3-hydroxy-4-methyl-1-octen-6-ynyl)-, CAS RN 88430-50-6); acadesine, (1H-Imidazole-4-carboxamide, 5-amino-1β-D-ribofuranosyl-, CAS RN 2627-69-2); beraprost sodium, (1-Cyclopenta[b]benzofuran-5-butanoic acid, 2,3,3a,8b-tetrahydro-2-hydroxy-1-(3-hydroxy-4-methyl-1-octen-6-ynyl)-, monosodium salt, CAS RN 88475-69-8); ciprostene calcium, (Pentanoic acid, 5-[(3aS,5R,6R,6aR)-hexahydro-5-hydroxy-6-[(1E,3S)-3-hydroxy-1-octenyl]-3a-methyl-2(1H)-pentalenylidene]-, calcium salt (2:1), (5Z)-CAS RN 81703-55-1), Itazigrel, (Thiazole, 4,5-bis(4-methoxyphenyl)-2-(trifluoromethyl)-CAS RN 70529-35-0); lifarizine (piperazine, 1-(diphenylmethyl)-4-[[5-methyl-2-(4-methylphenyl)-1H-imidazol-4-yl]methyl-1]-), CAS RN 119514-66-8); oxagrelate, (6-Phthalazinecarboxylic acid, 3,4-dihydro-1-(hydroxymethyl)-5,7-dimethyl-4-oxo-, ethyl ester, CAS RN 56611-65-5).

Hemorrheologic agent as used herein describes those compounds which improve the flow properties of blood by decreasing its viscosity. A suitable hemorrheologic agent of the present invention is pentoxifylline (TH-Purine-2,6-dione, 3,7-dihydro-3,7-dimethyl-1-(5-oxohexyl)-(9Cl) (CA INDEX NAME) Theobromine, 1-(5-oxohexyl)-, CAS RN 6493-05-6 e.g., Trentali® (Aventis)).

Pentoxifylline and its metabolites (which can be useful in the present invention) improve the flow properties of blood by decreasing its viscosity. In patients with chronic peripheral arterial disease, this increases blood flow to the affected microcirculation and enhances tissue oxygenation. The precise mode of action of pentoxifylline and the sequence of events leading to clinical improvement are still to be defined. Pentoxifylline administration has been shown to produce dose-related hemorrheologic effects, lowering blood viscosity, and improving erythrocyte flexibility. Leukocyte properties of hemorrheologic importance have been modified in animal and in vitro human studies. Pentoxifylline has been shown to increase leukocyte deformability and to inhibit neutrophil adhesion and activation. Tissue oxygen levels have been shown to be significantly increased by therapeutic doses of pentoxifylline in patients with peripheral arterial disease.

Lipoprotein-associated coagulation inhibitor (LACI) is a serum glycoprotein with a molecular weight of 38,000 Kd useful as a blood modifier of the present invention It is also known as tissue factor inhibitor because it is a natural inhibitor of thromboplastin (tissue factor) induced coagulation (U.S. Pat. No. 5,110,730 and U.S. Pat. No. 5,106,833 described tissue factor and are hereby incorporated by reference their entireties). LACI is a protease inhibitor and has 3 Kunitz domains, two of which are known to interact with factors VII and Xa respectively, while the function of the third domain is unknown. Many of the structural features of LACI can be deduced because of its homology with other well studies proteases. LACI is not an enzyme, so it probably inhibits its protease target in a stoichiometric manner; namely, one of the domains of LACI inhibits one protease molecule (see U.S. Pay. No. 606374).

Factor VIIa Inhibitors as used herein are those agents which inhibit activated Factor VIIa from acting to contribute to the formation of a fibrin clot. Suitable Factor VIIa Inhibitors include but are not limited to, 4H-31-benzoxazin-4-ones, 4H-3,1-benzoxazin-4-thiones, quinazolin-4-thiones, benzothiazin-4-ones described in U.S. Pat. No. 6,180,625, imidazolyl-boronic acid-derived peptide analogues as described in U.S. Pat. No. 5,639,739, TFPI-derived peptides described in U.S. Pat. No. 6,180,625.

Additional suitable Factor VIIa Inhibitors include but are not limited to Naphthalene-2-sulfonic acid {1-[3-(aminoiminomethyl)-benzyl]-2-oxo-pyrrolidin-3-(S)-yl}amide trifluoroacetate, dibenzofuran-2-sulfoic acid {T-[3-(aminomethyl)benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide tribluoroacetate, 3,4-dihydro-1H-isoquinoline-2-sulfonic acid {1-[3-(aminoiminomethyl)-benzyl]-2-oxo-pyrrolin-3-(S)-yl}-amide tribluoroacetate or combinations thereof.

Factor Xa inhibitors as used herein are those agents which inhibit activated Factor X from acting to contribute to the formation of a fibrin clot. Suitable agents for use in the present invention as Factor Xa inhibitors include but are not limited to disubstituted pyrazolines, disubstituted triazolines as described in U.S. Pat. No. 6,191,159, lipoprotein-associated coagulation inhibitor (LACI) (as described above), low molecular weight heparins described as below, heparinoids described as below, benzimidazolines, benzoxazolinones, bensopiperazinones, indanones, as described in U.S. Pat. No. 6,207,697, dibasic (amidinoaryl)propanoic acid derivatives as described in J. Med. Chem. 37:1200-1207 (1994), bis-arlysulfonylaminobenzamide derivatives as described in U.S. Pat. No. 5,612,378, amidinophenyl-pyrrolidines, amidinophenyl-pyrrolines, amidinophenyl-isoxazolidines as described in U.S. Pat. No. 6,057,342, amidinoindoles, amidinoazoles as described in U.S. Pat. No. 6,043,257, peptidic Factor Xa inhibitors as described below, substituted n-[(aminoiminomethyl)phenyl]propylamides, substituted n-[(aminomethyl)phenyl]propylamides as described in U.S. Pat. No. 6,080,767 or combinations thereof.

Peptidic factor Xa inhibitors such as the leech-derived, 119-amino acid protein antistasin and the soft tick derived protein TAP (tick anticoagulant peptide) accelerate clot lysis and prevented reocclusion when given as adjuncts to thrombolysis (Melloff et al., Circulation Research 70:1152-1160 (1992); Sitko et al., Circulation 85:805-815 (1992)). U.S. Pat. No. 5,385,885 issued Jan. 31, 1995 discloses smooth muscle cell proliferation inhibitory activity of both tick anticoagulant peptide and antistasin. The peptide ecotin is another selective, reversible, tight-binding inhibitor of factor Xa that exhibits protein anticoagulant activity (Seymour et al., Biochemistry 33:3949-3959 (1994); WO94/20535, Sep. 14, 1994). Ixodidae, argasin and ancylostomatin are other representative peptidic factor Xa inhibitors isolated from animals that feed on blood (Markwardt, Thrombosis and Hemostasis 72: 477-479 (1994).

These non-limiting examples of peptidic Factor Xa inhibitors which may be used in the present invention are listed below with their CAS RN (Chemical abstract services registry number). These include Proteinase inhibitor, antistasin, CAS RN 110119-38-5; tick anticoagulant peptide, (Proteinase inhibitor, TAP) CAS RN 129737-17-3; ecotin, (Proteinase inhibitor, ecotin) CAS RN 87928-05; argasin, CAS RN 53092-89-0; ancylostomatin, CAS RN 11011-09-9; Ixodidae (as described in Markwardt, 1994).

Low molecular weight heparins refer to agents derived from heparins which reduces the incidence of bleeding when compared with standard heparin. Heparins are glycosaminoglycans. MW range from 2000-10000. They may be produced from porcine intestinal mucosa and except for nadroparan, are all sodium salts. A suitable heparinoid of the present invention includes but is not limited to enoxaparin, nardroparin, dalteparin, certroparin, pamaparin, reviparin, tinzaparin and combinations thereof.

Heparinoid is a modified form of heparin which reduces the incidence of bleeding when compared with standard heparin. A suitable heparinoid of the present invention includes but is not limited to Danaparoid CAS RN 308068-55-5, (e.g., Orgaran Injection Organon).

### Hormone Replacement Agents/Compositions

The compounds described herein can be used in therapeutic combination with one or more hormone replacement agents/compositions including, but not limited to androgens, estrogens, progestins, their pharmaceutically acceptable salts and derivatives thereof. Examples of androgen and estrogen combinations include but are not limited to the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest. Examples of estrogens and estrogen combinations include but are not limited to: (a) the blend of nine (9) synthetic estrogenic substances including sodium estrone sulfate, sodium equilin sulfate, sodium 17α-dihydroequilin sulfate, sodium 17α-estradiol sulfate, sodium 17β-dihydroequilin sulfate, sodium 17α-dihydroequilenin sulfate, sodium 17β-dihydroequilenin sulfate, sodium equilenin sulfate and sodium 17β-estradiol sulfate; available from Duramed Pharmaceuticals, Inc., Cincinnati, Ohio, under the tradename Cenestin; (b) ethinyl estradiol (19-nor-17α-pregna-1,3,5(10)-trien-20-yne-3,17-diol; available by Schering Plough Corporation, Kenilworth, N.J., under the tradename Estinyl; (c) esterified estrogen combinations such as sodium estrone sulfate and sodium equilin sulfate; available from Solvay under the tradename Estratab and from Monarch Pharmaceuticals, Bristol, Term., under the tradename Menest; (d) estropipate (piperazine estra-1,3,5(10)-trien-17-one, 3-(sulfoxy)-estrone sulfate); available from Pharmacia & Upjohn, Peaack, N.J., under the tradename Ogen and from Women First Health Care, Inc., San Diego, Calif., under the tradename Ortho-Est; and (e) conjugated estrogens (17α-dihydroequilin, 17α-estradiol, and 17β-dihydroequilin); available from Wyeth-Ayerst Pharmaceuticals, Philadelphia, Pa., under the tradename Premarin. Examples of progestin and estrogen combinations include but are not limited to: (a) the combination of estradiol (estra-1,3,5 (10)-triene-3,17β-diol hemihydrate) and norethindrone (17β-acetoxy-19-nor-17α-pregn-4-en-20-yn-3-one); which is available from Pharmacia & Upjohn, Peapack, N.J., under the tradename Activella; (b) the combination of levonorgestrel (d(−)-13β-ethyl-17α-ethinyl-17β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil; (c) the combination of ethynodiol diacetate (19-nor-17α-pregn-4-en-20-yne-3β,17-diol diacetate) and ethinyl estradiol; available from G.D. Searle & Co., Chicago, Ill., under the tradename Demulen and from Watson under the tradename Zovia; (d) the combination of desogestrel (13-ethyl-11-methylene-18,19-dinor-17α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept; (e) the combination of norethindrone and ethinyl estradiol; available from Parke-Davis, Morris Plains, N.J., under the tradenames Estrostep and femhrt, from Watson under the tradenames Microgestin, Necon, and Tri-Norinyl, from Ortho-McNeil under the tradenames Modicon and Ortho-Novum, and from Warner Chilcott Laboratories, Rockaway, N.J., under the tradename Ovcon; (f) the combination of norgestrel ((±)-13-ethyl-17-hydroxy-18,19-dinor-17α-preg-4-en-20-yn-3-one) and ethinyl estradiol; available from Wyeth-Ayerst under the tradenames Ovral and Lo/Ovral, and from Watson under the tradenames Ogestrel and Low-Ogestrel; (g) the combination of norethindrone, ethinyl estradiol, and mestranol (3-methoxy-19-nor-17α-pregna-1,3,5(110)-trien-20-yn-17-ol); available from Watson under the tradenames Brevicon and Norinyl; (h) the combination of 17β-estradiol (estra-1,3,5(10)-triene-3-, 17β-diol) and micronized norgestimate (17β-17-(Acetyloxyl)-13-ethyl-18,19-dinorpregn-4-en-20-yn-3-one-3-oxime); available from Ortho-McNeil under the tradename Ortho-Prefest; (i) the combination of norgestimate (18,19-dinor-17-pregn-4-en-20-y-n-3-one, 17-(acetyloxy)-1 3-ethyl-oxime, (17(α)-(+)-) and ethinyl estradiol; available from Ortho-McNeil under the tradenames Ortho Cyclen and Ortho Tri-Cyclen; and (j) the combination of conjugated estrogens (sodium estrone sulfate and sodium equilin sulfate) and medroxyprogesterone acetate (20-dione, 17-(acetyloxy)-6-methyl-, (6(α))-pregn-4-ene-3); available from Wyeth-Ayerst under the tradenames Premphase and Prempro. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3, 20-dione); available from Solvay under the tradename Prometrium; and medroxyprogesterone acetate; available from Pharmacia & Upjohn under the tradename Provera. Tests showing the efficacy of the therapy and the rationale for the combination therapy with hormone replacement agents/compositions are presented in US20030119796.

### Chemotherapeutic Agents

The compounds described herein can be used in therapeutic combination with one or more chemotherapeutic agents including but not limited to hydrophobic, and heterocyclic cancer chemotherapeutic agents such as adriamycin (doxorubicin), phosphates, colcemid, etoposide, paclitaxel, bisantene, vincristine, and vinblastine. Tests showing the efficacy of the therapy and the rationale for the combination therapy with chemotherapeutic agents are described, for example, in WO05/030225.

Peptide which Mitigate One or More Symptoms of Atherosclerosis

The compounds described herein can be used in therapeutic combination with a the peptide which mitigates one or more symptoms of atherosclerosis as described, for example, in US20040266671, U.S. Pat. No. 6,664,230, US20030045460, US20030171277, US20030229015, US20040254120, WO/04034977, WO/02015923, and WO/05016280. This includes the peptide described as SEQ ID NO. 5 in U.S. Pat. No. 6,664,230 (CAS registry No. 631959-47-2) wherein at least one residue comprises a D-amino acid.

### Anti-Cancer Agents

The compounds described herein can be used in therapeutic combination with an anti-cancer agent, including but not limited to: steroidal or non steroidal antiandrogens (such as finasteride (Proscar®), cyproterone acetate (CPA), flutamide (4′-nitro-3′-trifluorormethyl isobutyranilide), bicalutamide (Casodex®), and nilutamide), estrogens, diethylstilbestrol (DES), conjugated estrogens (such as Premarin®), Taxanes (such as paclitaxel (Taxol®), docetaxel (Taxotere®), 7-O-methylthio-methylpaclitaxel (disclosed in U.S. Pat. No. 5,646,176), 3′-tert-butyl-3′-N-tert-butyloxycarbonyl-4-deacetyl-3′-dephenyl-3′-N-debenzoyl-4-O-methoxycarbonyl-paclitaxel (disclosed in U.S. Ser. No. 60/179,965, and example 17 therein), C-4 methyl carbonate paclitaxel (disclosed in WO 94/14787), and formulations containing taxanes, for examples those disclosed in U.S. Pat. No. 6,395,770, U.S. Pat. No. 6,380,405, and U.S. Pat. No. 6,239,167), epothilones (such as epothilone A, epothilone B, epothilone C, epothilone D, desoxyepothilone A, desoxyepothilone B, [1S-[1R*,3R*(E),7R*,10S*,11R*,12R*,16S*]]-7,11-dihydroxy-8,8,10,12,16-pentamethyl-3-[1-methyl-2-(2- -methyl-4-thiazolyl)ethenyl]-4-aza-17-oxabicyclo[14.1.0.O]hepta-decane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-dihydroxy-8-, 8,10,12,16-pentamethyl-4,17-dioxabicyclo[14.1.0]-heptadecane-5,9-dione (disclosed in U.S. Ser. No. 09/506,481 filed on Feb. 17, 2000, and examples 7 and 8 therein), and derivatives thereof), microtuble-disruptor agents, alkylating agents, anti-metabolites, epidophyllotoxin, an antineoplastic enzyme, a topoisomerase inhibitor, procarbazine, mitoxantrone, platinum coordination complexes, biological response modifiers, growth inhibitors, hormonal/antihormonal therapeutic agents, haematopoietic growth factors, the anthracycline family of drugs, vinca drugs, mitomycins, bleomycins, cytotoxic nucleosides, discodermolide, the pteridine family of drugs, diynenes, aromatase inhibitors, podophyllotoxins, doxorubicin, caminomycin, daunorubicin, idarubicin, dactinomycin, plicamycin, vinorelbine, aminopterin, methotrexate, methopterin, dichloro-methotrexate, thioguanine, hydroxyrurea, campathecins, nitrosureas, mitomycin C, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, leurosine, estramustine, cisplatin, carboplatin, cyclophosphamide, bleomycin, tamoxifen, ifosfamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, interleukins, LHRH analogs (such as goserelin acetate (Zoladex®) and leuprolide acetate (Lupron®)), and selective estrogen receptor modulator (SERM) compounds. The term selective estrogen receptor modulator includes both estrogen agonist and estrogen antagonists and refers to compounds that bind with the estrogen receptor, inhibit bone turnover and prevent bone loss. In particular, estrogen agonists are compounds capable of binding to the estrogen receptor sites in mammalian tissue, and mimicking the actions of estrogen in one or more tissue. Estrogen antagonists are compounds capable of binding to the estrogen receptor sites in mammalian tissue, and blocking the actions of estrogen in one or more tissues.

SERMs include but are not limited to tamoxifen (and associated compounds disclosed in U.S. Pat. No. 4,536,516); 4-hydroxytamoxifen (and associated compounds disclosed in U.S. Pat. No. 4,623,660); raloxifene (and associated compounds disclosed in U.S. Pat. No. 4,418,068, U.S. Pat. No. 5,393,763, U.S. Pat. No. 5,457,117, U.S. Pat. No. 5,478,847, and U.S. Pat. No. 5,641,790); droloxifene; idoxifene (and associated compounds disclosed in U.S. Pat. No. 4,839,155); lasofoxifene; TSE-424 (and other compounds disclosed in U.S. Pat. No. 5,998,402, U.S. Pat. No. 5,985,910, U.S. Pat. No. 5,780,497, U.S. Pat. No. 5,880,137, EP0802183A1); LY353381; LY117081; toremifene (and other compounds disclosed in U.S. Pat. No. 4,696,949 and U.S. Pat. No. 4,996,225); centchroman (and other compounds disclosed in U.S. Pat. No. 3,822,287); fulvestrant; 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate; 4,4′-dihydroxybenzophenone-2,4-dinitrophenylhydrazone; SH646; 6-(4-hydroxyphenyl)-5-[4-(2-piperidin-1-yl-ethoxy)-benzyl]-naphthalen-2-ol (and other compounds as disclosed in U.S. Pat. No. 5,484,795); {4-[2-(2-aza-bicyclo[2.2.1]hept-2-yl)-ethoxy]phenyl}-[6-hydroxy-2-(4-hydroxy-phenyl)-benzo[b]thiophen-3-yl]-methanone; GW 5638; GW 7604; EM-652 and EM-800 (synthesis and activity described in Gauthier et al., (1997) J. Med. Chem. 40:2117-2122); those compounds disclosed in U.S. Pat. No. 552412 (including cis-6-(4-fluoro-phenyl)-5-[4-(2-piperidin-1-yl-ethoxy)-phenyl]-5,6, -7,8-tetrahydro-naphthalene-2-ol; (−)-cis-6-phenyl-5-[4-(2-pyrrolidin-1-yl-ethoxy)-phenyl]-5,6,7,8-tetrahydro-naphthalene-2-ol; cis-6-phenyl-5-[4-(2-pyrrolidin-1-yl-ethoxy)phenyl]-5,6,7,8-tetrahydro-naphthalene-2-ol; cis-1-[6′-pyrrolidinoethoxy-3′-pyridyl]-2-phenyl-6-hydroxy-1,2,3,4-tetrahydronaphthalene; 1-(4′-pyrrolidinoethoxyphenyl)-2-(4″-fluorophenyl)-6-hydroxy-1,2,3, -4-tetrahydroisoquinoline; cis-6-(4-hydroxyphenyl)-5-[4-(2-piperidin-1-yl-ethoxy)-phenyl]-5,6-7,8-tetrahydro-naphthalene-2-ol; 1-(4′-pyrrolidinoethoxyphenyl)-2-phenyl-6-hydroxy-1,2,3,4-tetrahydroisoquinoline and the tartrate salt thereof (−)-cis-6-phenyl-5-[4-(2-pyrrolidin-1-yl-ethoxy)-phenyl-]-5,6,7,8-tetrahydro-naphthalene-2-ol), US20040259886, US20040162304, and WO95/10513; and pharmaceutically acceptable salts and esters thereof. Tests showing the efficacy of the therapy and the rationaled for the combination therapy with an anticancer agent are presented in US20040116358 and WO04/010948.

### Agents Used to Treat Bone Loss and Associated Disorders

The compounds described herein can be used in therapeutic combination with an agent used to treat bone loss and associated disorders including but not limited to: (1) SERMs (including those described above); (2) bisphosphonates including but not limited to alendronic acid and alendronate/MK-217/(Fosamax®)/alendronate sodium/alendronate monosodium trihydrate including sodium, potassium, calcium, magnesium or ammonium salts thereof (alendronic acid and alendronate are disclosed in U.S. Pat. No. 4,922,007, U.S. Pat. No. 5,019,651, U.S. Pat. No. 5,510,517, and US564849) 1; also); Yamanouchi compound YM 175/incadronate/cimadronate (cycloheptylaminomethylene-1,1-bisphosphonic acid, U.S. Pat. No. 4,970,335); 1,1-dichloromethylene-1,1-diphosphonic acid (clodronic acid), and the disodium salt (clodronate, Procter and Gamble), as described in Belgium Patent 672,205 (1966) and J. Org. Chem. 32, 4111 (1967)); EB-1053(1-hydroxy-3-(1-pyrrolidinyl)-propylidene-1,1-bisphosphonic acid); etidronic acid (1-hydroxyethane-1,1-diphosphonic acid (etidronic acid); Boehringer-Mannheim compound ibandronate/BM-210955 (1-hydroxy-3-(N-methyl-N-pentylamino)propylidene-1,1-bisphosphonic acid; disclosed in U.S. Pat. No. 4,927,814); minodronate (1-hydroxy-2-imidazo-(1,2-a)pyridin-3-yethylidene); neridronate (6-amino-1-hydroxyhexylidene-1,1-bisphosphonic acid); olpadronate (3-(dimethylamino)-1-hydroxypropylidene-1,1-bisphosphonic acid); pamidronate (3-amino-1-hydroxypropylidene-1,1-bisphosphonic acid); piridronate (2-(2-pyridinyl)ethylidene]-1,1-bisphosphonic acid; described in U.S. Pat. No. 4,761,406); risedronate (1-hydroxy-2-(3-pyridinyl)-ethylidene-1,1-bisphosphonic acid); tiludronate (4-chlorophenyl)thiomethane-1,1-disphosphonic acid; described in U.S. Pat. No. 4,876,248); zoledronate (1-hydroxy-2-(1H-imidazol-1-yl)ethylidene-1,1-bisphosphonic acid); etidronate; and pharmaceutically acceptable salts and esters thereof, and also including mixtures thereof, (3) estrogens and estrogen combinations (including those described above); (4) cathepsin K inhibitors (e.g., compounds which interfere with the activity of the cysteine protease cathepsin K) including those disclosed in WO00/55126 and WO01/49288; (5) androgen receptor modulators including but not limited to finasteride and other 5α-reductase inhibitors, nilutamide, flutamide, bicalutamide, liarozole, and abiraterone acetate; (6) inhibitors of osteoclast proton ATPase including those described in Farina et al. (1999) DDT, 4:163-172; (7) HMG-CoA reductase inhibitors (including those described above); (8) integrin receptor antagonists (including those described in US20040162304); (9) osteoblast anabolic agents (e.g., agents that build bone such as parathyroid hormone (PTH) or its amino terminal fragments (PTHrP-(1-36); Syed et al. (2001) JCEM 86:1525-1531) and analogues); (10) calcitonin; (11) vitamin D which includes, but is not limited to, vitamin D₃ (cholecalciferol), vitamin D₂ (ergocalciferol); 1α-hydroxy vitamin D; 25-hydroxy vitamin D; 1α,25-dihydroxy vitamin D; and dihydroxy vitamin D; (12) synthetic vitamin D analogues (non-naturally occurring compounds that act like vitamin D); (13) compounds disclosed in U.S. Pat. No. 5,280,040; and (14) serotonin reuptake inhibitors (including those described above).

The compounds described herein can be used in therapeutic combination with other agents including but not limited to: a thromboxane A2 (TxA2) antagonist; a CRTH2 receptor modulator (such as Ramatroban/Baynas/BAY u3405 which exhibits both TxA2 and CRTH2 antagonistic activity); ranitine; bosentan; a tyrosine kinase inhibitor such as disclosed in WO00/053605; a selective androgen receptor modulator (SARM) including LGD-2226 (Ligand) or those compounds disclosed in WO03/011824; coenzyme Q10 such as disclosed in U.S. Pat. No. 5,316,765, U.S. Pat. No. 4,933,165, and U.S. Pat. No. 4,929,437; an agent that upregulates type III endothelial cell nitric acid synthase such as disclosed in WO00/003746; a chondroprotective compound such as a polysulfated glycosaminoglycan (PSGAG), glucosamine, chondroitin sulfate (CS), hyaluronic acid (HA), pentosan polysulfate (PPS), doxycycline or minocycline, such as disclosed in EP970694; monocyte and macrophage inhibitors such as polyunsaturated fatty acids (PUFA); thyroid hormones including throxine analogues (such as CGS-26214 (a thyroxine compound with a fluorinated ring), dextrothyroxine, eitroxate, and thyropropic acid; a 5-HT reuptake inhibitor such as disclosed in WO99/44609; and anti-infective agents such as quinolones, for example, ciprofloxacin, ofloxacin, and Tequin™ (Bristol-Myers Squibb), macrolides such as erythromycin and clarithromycin (Biaxin™ (Abbott)), and azithromycin (Zithromax (Pfizer)).

It can be useful to administer a compound described herein together with 1, 2, 3, or more of an HMG-CoA reductase inhibitor (e.g., a statin such as atorvastatin, atorvastatin calcium, rosuvastatin, rosuvastatin calcium, simvastatin), a fibrate (e.g., fenofibrate (Tricor®)), niacin (including derivatives and extended release formulations (e.g., Niaspan®) thereof), a glitazone (e.g., rosiglitazone maleate (Avandia®), piogilitazone hydrochloride (Actos®)), a calcium channel blocker (e.g., amlodipine besylate (Norvasc®)), an angiotensin II receptor antagonist (e.g., valsartan (Diovan®, Diovan HCT® (valsartan and hydrochlorothiazide))), a biguanide (e.g., metformin (Glucophage®)), a sulfonylurea (e.g., glipizide (Glucotrol®, Glucotrol XL®), glyburide (Micronase®, Glynase Prestab®, Diabeta®), and Glucovance® (glyburide and metformin). It can be particularly useful to combine a compound described herein together with one or more of an HMG-CoA reductase inhibitor (e.g., a statin), a fibrate, a glitazone, niacin or a derivative thereof, a calcium channel blocker, an angiotensin II receptor antagonist, a biguanide, a sulfonylurea in a single pharmaceutical composition. The precise amount of each of the two or more active ingredients in a dosage unit will depend on the desired dosage of each component. Thus, it can be useful to create a dosage unit that will, when administered according to a particular dosage schedule (e.g., a dosage schedule specifying a certain number of units and a particular timing for administration), deliver the same dosage of each component as would be administered if the patient was being treated with only a single component. In other circumstances, it might be desirable to create a dosage unit that will deliver a dosage of one or more components that is less than that which would be administered if the patient was being treated only with a single component. Finally, it might be desirable to create a dosage unit that will deliver a dosage of one or more components that is greater than that which would be administered if the patient was being treated only with a single component. The pharmaceutical composition can include additional ingredients including but not limited to the excipients described herein. In certain embodiments, one or more therapeutic agents of the dosage unit may exist in an extended or control release formulation and additional therapeutic agents may not exist in extended release formulation. For example, a compound described herein may exist in the same dosage unit with fenofibrate (an extended release fibrate agent). For example, a compound described herein may exist in the same dosage unit with one or more additional agents including a controlled release formulation of torcetrapib.

A pharmaceutical composition can include 1% to 20% by weight of a compound described herein; from 1% to 80% by weight of an HMG-CoA reductase inhibitor such as atorvastatin, atorvastatin calcium, dihydrocompactin, bervastatin, carvastatin, cerivastatin, crilvastatin, dalvastatin, fluvastatin, glenvastatin, fluindostatin, velostatin, lovastatin, mevastatin, compactin, pitavastatin, pravastatin, rivastatin, rosuvastatin, rosuvastatin calcium, simvastatin, sirrivastatin, and CI-981; and from 0.01% to 2% by weight of a stabilizing agent such as butylated hydroxyanisole (BHA). It further can include from 1% to 80% by weight of microcrystalline cellulose; from 0.5% to 10% by weight of hydroxypropyl methylcellulose; from 0.1% to 4% by weight of magnesium stearate; and from 25% to 70% by weight of lactose. The composition may optionally include one or more of croscarmellose sodium, citric acid, ascorbic acid and propyl gallate. The composition can include or exclude one or more of citric acid, ascorbic acid and pre-gelatinized starch. As a practical matter, a single dosage unit such as a tablet or capsule should weigh from 50 mg to 1000 mg (for example, including from 100 mg to 800 mg).

A dosage unit (e.g., an oral dosage unit) can include from, for example, 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein; from 5 mg to 80 mg (e.g., 5 mg, 10 mg, 15 mg, 20 mg, 25 mg, 30 mg, 35 mg, 40 mg, 45 mg, 50 mg, 60 mg, 70 mg and 80 mg) of a statin (e.g., atorvastatin, atorvastatin calcium, rosuvastatin, rosuvastatin calcium, simvastatin, etc.); and from 0.002 mg to 0.004 mg of BHA per mg of statin. In certain embodiments, the dosage unit comprises 5 mg of a compound described herein and 5, 10, 15, 20, 25, 30, 35, 40, 45, 50, 55, 60, 65, 70, 75, or 80 mg of a statin. In certain embodiments, the dosage unit comprises 10 mg of a compound described herein and 5, 10, 15, 20, 25, 30, 35, 40, 45, 50, 55, 60, 65, 70, 75, or 80 mg of a statin. In certain embodiments, the dosage unit comprises 15 mg of a compound described herein and 5, 10, 15, 20, 25, 30, 35, 40, 45, 50, 55, 60, 65, 70, 75, or 80 mg of a statin. In certain embodiments, the dosage unit comprises 20 mg of a compound described herein and 5, 10, 15, 20, 25, 30, 35, 40, 45, 50, 55, 60, 65, 70, 75, or 80 mg of a statin. In certain embodiments, the dosage unit comprises 25 mg of a compound described herein and 5, 10, 15, 20, 25, 30, 35, 40, 45, 50, 55, 60, 65, 70, 75, or 80 mg of a statin. In certain embodiments, the dosage unit comprises 30 mg of a compound described herein and 5, 10, 15, 20, 25, 30, 35, 40, 45, 50, 55, 60, 65, 70, 75, or 80 mg of a statin. In certain embodiments, the dosage unit comprises 35 mg of a compound described herein and 5, 10, 15, 20, 25, 30, 35, 40, 45, 50, 55, 60, 65, 70, 75, or 80 mg of a statin. In certain embodiments, the dosage unit comprises 40 mg of a compound described herein and 5, 10, 15, 20, 25, 30, 35, 40, 45, 50, 55, 60, 65, 70, 75, or 80 mg of a statin. In certain embodiments, the dosage unit comprises 45 mg of a compound described herein and 5, 10, 15, 20, 25, 30, 35, 40, 45, 50, 55, 60, 65, 70, 75, or 80 mg of a statin. In certain embodiments, the dosage unit comprises 50 mg of a compound described herein and 5, 10, 15, 20, 25, 30, 35, 40, 45, 50, 55, 60, 65, 70, 75, or 80 mg of a statin. In certain embodiments, the dosage unit comprises 55 mg of a compound described herein and 5, 10, 15, 20, 25, 30, 35, 40, 45, 50, 55, 60, 65, 70, 75, or 80 mg of a statin. In certain embodiments, the dosage unit comprises 60 mg of a compound described herein and 5, 10, 15, 20, 25, 30, 35, 40, 45, 50, 55, 60, 65, 70, 75, or 80 mg of a statin. In certain embodiments, the dosage unit comprises 65 mg of a compound described herein and 5, 10, 15, 20, 25, 30, 35, 40, 45, 50, 55, 60, 65, 70, 75, or 80 mg of a statin. In certain embodiments, the dosage unit comprises 70 mg of a compound described herein and 5, 10, 15, 20, 25, 30, 35, 40, 45, 50, 55, 60, 65, 70, 75, or 80 mg of a statin. In certain embodiments, the dosage unit comprises 75 mg of a compound described herein and 5, 10, 15, 20, 25, 30, 35, 40, 45, 50, 55, 60, 65, 70, 75, or 80 mg of a statin. In certain embodiments, the dosage unit comprises 80 mg of a compound described herein and 5, 10, 15, 20, 25, 30, 35, 40, 45, 50, 55, 60, 65, 70, 75, or 80 mg of a statin. In certain embodiments, the dosage unit comprises 85 mg of a compound described herein and 5, 10, 15, 20, 25, 30, 35, 40, 45, 50, 55, 60, 65, 70, 75, or 80 mg of a statin. In certain embodiments, the dosage unit comprises 90 mg of a compound described herein and 5, 10, 15, 20, 25, 30, 35, 40, 45, 50, 55, 60, 65, 70, 75, or 80 mg of a statin. In certain embodiments, the dosage unit comprises 95 mg of a compound described herein and 5, 10, 15, 20, 25, 30, 35, 40, 45, 50, 55, 60, 65, 70, 75, or 80 mg of a statin. In certain embodiments, the dosage unit comprises 100 mg of a compound described herein and 5, 10, 15, 20, 25, 30, 35, 40, 45, 50, 55, 60, 65, 70, 75, or 80 mg of a statin. A daily dose can include 5-100 mg (e.g., 10 mg, 20 mg, 30 mg, 40 mg, 50 mg, 60 mg, 70 mg, 80 mg) of a compound described herein and 5, 10, 20, 30, 40, 50, 60, 70 or 80 mg of a statin. In certain embodiments the statin is selected from the group consisting of atorvastatin, atorvastatin calcium, rosuvastatin, rosuvastatin calcium and simvastatin. In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can include from 0.0005 mg to 0.001 mg of propyl gallate per mg of statin. For example, the dosage unit can include from 0.01 mg to 16 mg, and particularly from 0.02 mg to 0.16 mg of BHA, and additionally may be include from 0.001 mg to 0.05 mg, and particularly from 0.005 mg to 0.04 mg of propyl gallate. The dosage unit can additionally include from 1 mg to 640 mg, and particularly from 15 mg to 120 mg of microcrystalline cellulose; from 0.5 mg to 80 mg, and particularly from 2 mg to 16 mg of HPMC; from 0.1 mg to 32 mg, and particularly from 1.5 to 12 mg of magnesium stearate; and lactose. Croscarmellose sodium may optionally be included as a component in the composition. For example, an oral dosage unit may contain from 0 mg to 80 mg of croscarmellose sodium, and particularly from 3 mg to 24 mg of croscarmellose sodium. Citric acid may optionally be included as a component in the composition. For example, an oral dosage unit may contain from 0 mg to 80 mg, and particularly from 0.25 mg to 2 mg of citric acid. In addition, one or more of lactic acid, malic acid, succinic acid, tartaric acid and EDTA may optionally be included in the dosage unit. An inert component such as lactose can be added to bring the unit dosage form to a desired total weight. The dosage unit can optionally comprise other agents such as 1, 2, 3, or more of a fibrate, niacin (including derivatives thereof), a glitazone, a calcium channel blocker, an angiotensin II receptor antagonist, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 10 mg to 150 mg (e.g., 10 mg, 20 mg, 30 mg, 40 mg, 48 mg, 50 mg, 60 mg, 70 mg, 80 mg, 90 mg, 100 mg, 120 mg, 130 mg, 140 mg, 145 mg, 150 mg) of a fibrate (e.g., fenofibrate (Tricor®). In certain embodiments, the dosage unit comprises 5 mg of a compound described herein and 10, 20, 30, 40, 48, 50, 60, 70, 80, 90, 100, 120, 130, 140, 145, or 150 mg of a fibrate. In certain embodiments, the dosage unit comprises 10 mg of a compound described herein and 10, 20, 30, 40, 48, 50, 60, 70, 80, 90, 100, 120, 130, 140, 145, or 150 mg of a fibrate. In certain embodiments, the dosage unit comprises 15 mg of a compound described herein and 10, 20, 30, 40, 48, 50, 60, 70, 80, 90, 100, 120, 130, 140, 145, or 150 mg of a fibrate. In certain embodiments, the dosage unit comprises 20 mg of a compound described herein and 10, 20, 30, 40, 48, 50, 60, 70, 80, 90, 100, 120, 130, 140, 145, or 150 mg of a fibrate. In certain embodiments, the dosage unit comprises 25 mg of a compound described herein and 10, 20, 30, 40, 48, 50, 60, 70, 80, 90, 100, 120, 130, 140, 145, or 150 mg of a fibrate. In certain embodiments, the dosage unit comprises 30 mg of a compound described herein and 10, 20, 30, 40, 48, 50, 60, 70, 80, 90, 100, 120, 130, 140, 145, or 150 mg of a fibrate. In certain embodiments, the dosage unit comprises 35 mg of a compound described herein and 10, 20, 30, 40, 48, 50, 60, 70, 80, 90, 100, 120, 130, 140, 145, or 150 mg of a fibrate. In certain embodiments, the dosage unit comprises 40 mg of a compound described herein and 10, 20, 30, 40, 48, 50, 60, 70, 80, 90, 100, 120, 130, 140, 145, or 150 mg of a fibrate. In certain embodiments, the dosage unit comprises 45 mg of a compound described herein and 10, 20, 30, 40, 48, 50, 60, 70, 80, 90, 100, 120, 130, 140, 145, or 150 mg of a fibrate. In certain embodiments, the dosage unit comprises 50 mg of a compound described herein and 10, 20, 30, 40, 48, 50, 60, 70, 80, 90, 100, 120, 130, 140, 145, or 150 mg of a fibrate. In certain embodiments, the dosage unit comprises 55 mg of a compound described herein and 10, 20, 30, 40, 48, 50, 60, 70, 80, 90, 100, 120, 130, 140, 145, or 150 mg of a fibrate. In certain embodiments, the dosage unit comprises 60 mg of a compound described herein and 10, 20, 30, 40, 48, 50, 60, 70, 80, 90, 100, 120, 130, 140, 145, or 150 mg of a fibrate. In certain embodiments, the dosage unit comprises 65 mg of a compound described herein and 10, 20, 30, 40, 48, 50, 60, 70, 80, 90, 100, 120, 130, 140, 145, or 150 mg of a fibrate. In certain embodiments, the dosage unit comprises 70 mg of a compound described herein and 10, 20, 30, 40, 48, 50, 60, 70, 80, 90, 100, 120, 130, 140, 145, or 150 mg of a fibrate. In certain embodiments, the dosage unit comprises 75 mg of a compound described herein and 10, 20, 30, 40, 48, 50, 60, 70, 80, 90, 100, 120, 130, 140, 145, or 150 mg of a fibrate. In certain embodiments, the dosage unit comprises 80 mg of a compound described herein and 10, 20, 30, 40, 48, 50, 60, 70, 80, 90, 100, 120, 130, 140, 145, or 150 mg of a fibrate. In certain embodiments, the dosage unit comprises 85 mg of a compound described herein and 10, 20, 30, 40, 48, 50, 60, 70, 80, 90, 100, 120, 130, 140, 145, or 150 mg of a fibrate. In certain embodiments, the dosage unit comprises 90 mg of a compound described herein and 10, 20, 30, 40, 48, 50, 60, 70, 80, 90, 100, 120, 130, 140, 145, or 150 mg of a fibrate. In certain embodiments, the dosage unit comprises 95 mg of a compound described herein and 10, 20, 30, 40, 48, 50, 60, 70, 80, 90, 100, 120, 130, 140, 145, or 150 mg of a fibrate. In certain embodiments, the dosage unit comprises 100 mg of a compound described herein and 10, 20, 30, 40, 48, 50, 60, 70, 80, 90, 100, 120, 130, 140, 145, or 150 mg of a fibrate. A daily dose can include 5-100 mg (e.g., 10 mg, 20 mg, 30 mg, 40 mg, 50 mg, 60 mg, 70 mg, 80 mg) of a compound described herein and 10, 20, 30, 40, 48, 50, 60, 70, 80, 90, 100, 120, 130, 140, 145, or 150 mg of a fibrate. In certain embodiments, the fibrate is fenofibrate (Tricor®). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), niacin (including derivatives thereof), a glitazone, a calcium channel blocker, an angiotensin II receptor antagonist, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 1 mg to 60 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 60 mg) of a glitazone (e.g., rosiglitazone, pioglitazone). In certain embodiments, the dosage unit comprises 5 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 10 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 15 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 20 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 25 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 30 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 35 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 40 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 45 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 50 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 55 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 60 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 65 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 70 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 75 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 80 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 85 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 90 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 95 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments, the dosage unit comprises 100 mg of a compound described herein and 1, 2, 3, 4, 5, 6, 7, 8, 9, 10, 11, 12, 13, 14, 15, 20, 25, 30, 35, 40, 45, 50, or 60 mg of a glitazone. A daily dose can include 5-100 mg (e.g., 10 mg, 20 mg, 30 mg, 40 mg, 50 mg, 60 mg, 70 mg, 80 mg) of a compound described herein and 1, 2, 3, 4, 5, 6, 7, 8, 9, 10, 11, 12, 13, 14, 15, 20, 25, 30, 35, 40, 45, 50, or 60 mg of a glitazone. In certain embodiments the glitazone is rosiglitazone maleate (Avandia®). In certain embodiments the glitazone is pioglitazone (Actos®). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, niacin (including derivatives thereof), a calcium channel blocker, an angiotensin II receptor antagonist, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 100 mg to 2000 mg (e.g., 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg) of niacin or a derivative thereof. In certain embodiments, the dosage unit comprises 5 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 10 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 15 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 20 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 25 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 30 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 35 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 40 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 45 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 50 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 55 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 60 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 65 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 70 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 75 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 80 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 85 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 90 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 95 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments, the dosage unit comprises 100 mg of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof A daily dose can include 5-100 mg (e.g., 10 mg, 20 mg, 30 mg, 40 mg, 50 mg, 60 mg, 70 mg, 80 mg) of a compound described herein and 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of niacin or a derivative thereof. In certain embodiments the niacin derivative is Niaspan® (niacin extended release tablets). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, a glitazone, a calcium channel blocker, an angiotensin II receptor antagonist, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 1 mg to 15 mg (e.g., 1 mg, 2 mg, 2.5 mg, 3 mg, 4 mg, 5 mg, 6 mg, 7 mg, 7.5 mg, 8 mg, 9 mg, 10 mg, 11 mg, 12 mg, 12.5 mg, 13 mg, 14 mg, 15 mg) of a calcium channel blocker (e.g., amlodipine). In certain embodiments, the dosage unit comprises 5 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 10 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 15 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 20 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 25 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 30 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 35 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 40 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 45 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 50 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 55 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 60 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 65 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 70 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 75 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 80 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 85 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 90 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 95 mg of a compound described herein and 1, 2, 2.5, 3, 4, 5, 6, 7, 7.5, 8, 9, 10, 11, 12, 12.5, 13, 14, or 15 mg of a calcium channel blocker. In certain embodiments, the dosage unit comprises 100 mg of a compound described herein and 1, 2, 2.5, 3, 4, 5, 6, 7, 7.5, 8, 9, 10, 11, 12, 12.5, 13, 14, or 15 mg of a calcium channel blocker. A daily dose can include 5-100 mg (e.g., 10 mg, 20 mg, 30 mg, 40 mg, 50 mg, 60 mg, 70 mg, 80 mg) of a compound described herein and 1, 2, 2.5, 3, 4, 5, 6, 7, 7.5, 8, 9, 10, 11, 12, 12.5, 13, 14, or 15 mg of a calcium channel blocker. In certain embodiments the calcium channel blocker is amlodipine (Norvasc®; amlodipine beslylate). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, niacin (including derivatives thereof), a glitazone, an angiotensin II receptor antagonist, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 20 mg to 400 mg (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 220 mg, 240 mg, 260 mg, 280 mg, 320 mg, 340 mg, 360 mg, 380 mg, 400 mg) of an angiotensin II receptor antagonist (e.g., valsartan). In certain embodiments, the dosage unit comprises 5 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 10 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 15 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 20 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 25 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 30 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 35 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 40 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 45 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 50 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 55 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 60 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 65 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 70 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 75 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 80 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 85 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 90 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 95 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments, the dosage unit comprises 100 mg of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. A daily dose can include 5-100 mg (e.g., 10 mg, 20 mg, 30 mg, 40 mg, 50 mg, 60 mg, 70 mg, 80 mg) of a compound described herein and 20, 30, 40, 50, 60, 70, 80, 90, 100, 110, 120, 130, 140, 150, 160, 170, 180, 190, 200, 220, 240, 260, 280, 320, 340, 360, 380, or 400 mg of an angiotensin II receptor antagonist. In certain embodiments the angiotensin II receptor antagonist is valsartan (Diovan®). In certain embodiments the dosage unit further comprises a diuretic (e.g., hydrocholorothiazide). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, niacin (including derivatives thereof), a glitazone, a calcium channel blocker, a biguanide, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 100 mg to 3000 mg (e.g., 100 mg, 200 mg, 250 mg, 300 mg, 400 mg, 500 mg, 550 mg, 600 mg, 650 mg, 700 mg, 750 mg, 800 mg, 850 mg, 900 mg, 950 mg, 1000 mg, 1250 mg, 1500 mg, 1750 mg, 2000 mg, 2250 mg, 2500 mg, 2750 mg, 3000 mg) of a biguanide (e.g., metformin). In certain embodiments, the dosage unit comprises 5 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 10 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 15 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 20 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 25 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 30 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 35 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 40 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 45 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 50 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 55 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 60 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 65 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 70 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 75 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 80 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 85 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 90 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 95 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments, the dosage unit comprises 100 mg of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. A daily dose can include 5-100 mg (e.g., 10 mg, 20 mg, 30 mg, 40 mg, 50 mg, 60 mg, 70 mg, 80 mg) of a compound described herein and 100, 200, 250, 300, 400, 500, 550, 600, 650, 700, 750, 800, 850, 900, 950, 1000, 1250, 1500, 1750, 2000, 2250, 2500, 2750, or 3000 mg of a biguanide. In certain embodiments the biguanide is metformin (metformin hydrochloride, (Glucophage®, Glucophage® XR)). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, niacin (including derivatives thereof), a glitazone, a calcium channel blocker, an angiotensin II receptor antagonist, and a sulfonylurea.

A dosage unit (e.g., an oral dosage unit) can include, for example, from 1 to 500 mg, 2 mg to 500 mg, 1 to 300 mg, 1 to 100 mg, 5 mg to 100 mg, 1 to 30 mg, 1 to 40 mg, 5 mg to 20 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg of a compound described herein and from 1 to 40 mg (e.g., 1 mg, 1.25 mg, 1.5 mg, 1.75 mg, 2 mg, 2.5 mg, 3 mg, 4 mg, 5 mg, 6 mg, 7 mg, 7.5 mg, 8 mg, 9 mg, 10 mg, 12.5 mg, 15 mg, 20 mg, 25 mg, 30 mg, 35 mg, 40 g) of a sulfonylurea (e.g., glipizide, glyburide). In certain embodiments, the dosage unit comprises 5 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 10 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 15 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 20 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 25 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 30 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 35 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 40 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 45 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 50 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 55 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 60 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 65 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 70 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 75 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 80 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 85 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 90 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 95 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments, the dosage unit comprises 100 mg of a compound described herein and 1, 1.25, 1.5, 1.75, 2, 2.5, 3, 4, 5, 6, 7, 8, 9, 10, 15, 20, 25, 30, 35, or 40 mg of a sulfonylurea. A daily dose can include 5-100 mg (e.g., 10 mg, 20 mg, 30 mg, 40 mg, 50 mg, 60 mg, 70 mg, 80 mg) of a compound described herein and 1, 1.25, 1.5, 1.75, 2, 2.5, 3, 4, 5, 6, 7, 8, 9, 10, 15, 20, 25, 30, 35, or 40 mg of a sulfonylurea. In certain embodiments the sulfonylurea is glipizide (Glucotrol® Glucotrol XL®). In certain embodiments the sulfonylurea is glyburide (Micronase®, Glynase Prestab®, Diabeta®). In certain embodiments the dosage unit and daily dose are equivalent. In various embodiments, the dosage unit is administered with food at anytime of the day, without food at anytime of the day, with food after an overnight fast (e.g., with breakfast), at bedtime after a low fat snack. In various embodiments, the dosage unit is administered once a day, twice a day, three times a day, four times a day. The dosage unit can optionally comprise other agents such as 1, 2, 3, or more of an HMG CoA reductase inhibitor (e.g. a statin), a fibrate, niacin (including derivatives thereof), a glitazone, a calcium channel blocker, an angiotensin II receptor antagonist, and a biguanide.

It can be useful to administer a compound described herein together with a sterol or stanol composition. Sterols and stanols include but are not limited to those described herein. Plant sterols and stanols (e.g., beta-sitosterol) have been used as dietary supplements to reduce serum cholesterol levels. Plant sterol can be esterified to create stanol esters (also referred to as stanols), which are also used as food additives. Sterols are typically derived from agricultural sources, such as corn, soy-based, and pine tree mixtures. Stanols can be created through the reaction of the sterol with the suitable acid. Suitable acids include saturated, unsaturated, and polyunsaturated acids. Suitable acids include but are not limited to, stearic, butyric, lauric, palmitic, oleic, linoleic, linolenic, docohexanoic acid, and the like. Suitable methods for preparing these esters are well known in the art, see, e.g., U.S. Pat. No. 5,502,045 and U.S. Pat. No. 5,723,747. Sterols and sterol esters can be formulated a self-dispersing particles that are small enough to be effective when administered by ingestion (see, e.g., U.S. Pat. No. 6,387,411, U.S. Pat. No. 6,376,481 and US20040033202). Sterols and/or sterol esters in particle form can be combined with a compound described herein to create useful pharmaceutical compositions which can also include other agents such as 1, 2, 3, or more of an HMG-CoA reductase inhibitor (e.g., a statin such as atorvastatin, atorvastatin calcium, rosuvastatin, rosuvastatin calcium, simvastatin), a fibrate (e.g., fenofibrate (Tricor®)), niacin (including derivatives and extended release formulations (e.g., Niaspan®) thereof), a glitazone (e.g., rosiglitazone maleate (Avandia®), piogilitazone hydrochloride (Actos®)), a calcium channel blocker (e.g., amlodipine besylate (Norvasc®)), an angiotensin II receptor antagonist (e.g., valsartan (Diovan®, Diovan HCT® (valsartan and hydrochlorothiazide))), a biguanide (e.g., metformin (Glucophage®)), a sulfonylurea (e.g., glipizide (Glucotrol®, Glucotrol XL®), glyburide (Micronase®, Glynase Prestab®, Diabeta®), and Glucovance® (glyburide and metformin). The pharmaceutical composition can include additional ingredients such as stabilizers or bulking agents. The sterol particles in the composition can have any suitable size, e.g., 10-150 microns in diameter. However, to improve absorption in the body it can be desirable to use much smaller particles, e.g., less than 2000 nm in diameter as explained in US20040033202. Thus, pharmaceutical compositions that include a compound described herein can include sterol nanoparticles, such as sitosterol and/or phytosterol nanoparticles, which have an effective average particle size of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. The particles can be created by methods which include: milling, precipitation and homogenization. For example, homogenization methods are described in U.S. Pat. No. 5,510,118. The method includes dispersing sterol particles in a liquid dispersion medium in which the sterol is poorly soluble, followed by subjecting the dispersion to homogenization to reduce the particle size of the sterol to the desired effective average particle size. The sterol particles are preferably reduced in size in the presence of at least one surface stabilizer. Alternatively, the sterol particles can be contacted with one or more surface stabilizers either before or after attrition. Other compounds, such as a diluent, can be added to the sterol/surface stabilizer composition before, during, or after the size reduction process. Dispersions can be manufactured continuously or in a batch mode. Surface stabilizers can be used in the formulations. Suitable surface stabilizers include: cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, and random copolymers of vinyl acetate and vinyl pyrrolidone.

It can be useful to administer a compound described herein together with a polycosanol composition. Polycosanol compositions are complex mixtures of concentrated n-alkyl alcohols derived from, e.g., sugar cane and the wax of honey bees. Polycosanol compositions are reported to produce cholesterol lowering effects within the first 6-8 weeks of use. According to US20030232796, at a daily polycosanol dosage of 10 mg taken at night, LDL cholesterol levels typically drop by 20-25% within the first six months of use. At a dosage of 20 mg, LDL levels typically drop by 25-30%. HDL levels typically increase by 15-25% only after two months of use. The combined LDL reduction and HDL increase will produce a significant and dramatic improvement in the LDL to HDL ratio. Polycosanol can include fatty acid components including: 1-Octacosanol, 1-Triacontanol, 1-Tetracosanol, and 1-Hexacosanol. Typical usage levels range from 500-10,000 micrograms per serving/dose. Typical commercially available commercial compositions are 90% minimum fatty alcohols of (a) 1-Tetracosanol: 0-10%; (b) 1-Hexacosanol: 2-15%; (c) 1-Heptacosanol: 0-0.5%; (d) 1-Octacosanol: 55-70%; (e) 1-Nonacosanol: 0-10%; (f) 1-Triacontanol: 5-20%; (g) 1-Dotriacontanol: 0.1-10%; and (h) 1-Tetratriacontanol: 0.1-10%. Polycosanol compositions can be formulated as described above for stanols both with respect to particle size and overall formulation. In addition to the compounds described herein, the formulation can include other agents such as 1, 2, 3, or more of an HMG-CoA reductase inhibitor (e.g., a statin such as atorvastatin, atorvastatin calcium, rosuvastatin, rosuvastatin calcium, simvastatin), a fibrate (e.g., fenofibrate (Tricor®)), niacin (including derivatives and extended release formulations (e.g., Niaspan®) thereof), a glitazone (e.g., rosiglitazone maleate (Avandia®), piogilitazone hydrochloride (Actos®)), a calcium channel blocker (e.g., amlodipine besylate (Norvasc®)), an angiotensin II receptor antagonist (e.g., valsartan (Diovan®, Diovan HCT® (valsartan and hydrochlorothiazide))), a biguanide (e.g., metformin (Glucophage®)), a sulfonylurea (e.g., glipizide (Glucotrol®, Glucotrol XL®), glyburide (Micronase®, Glynase Prestab®, Diabeta®), and Glucovance® (glyburide and metformin).

Combining two or more active ingredients in single dosage form results in the possibility of chemical interactions between the active drug substances. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed multilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated wherein the active ingredients are distributed in the inner and outer phase of tablets. In an attempt to divide chemically incompatible components of proposed combination, few interacting components are converted in granules or beads using well known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include tris, arecoline, arginine, barium, betaine, bismuth, chloroprocaine, choline, clemizole, deanol, imidazole, and morpholineethanol.

The agents of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

The agents described herein can be either in their free form or as a salt can be combined with a polymer such as polylactic-glycoloic acid (PLGA), poly-(I)-lactic-glycolic-tartaric acid (P(I)LGT) (WO01/12233), polyglycolic acid (U.S. Pat. No. 3,773,919), polylactic acid (U.S. Pat. No. 4,767,628), poly(M-caprolactone) and poly(alkylene oxide) (US20030068384) to create a sustained release formulation. Such formulations can be used within implants that release a compound of the invention and/or another agent over a period of a few days, a few weeks or several months depending on the polymer, the particle size of the polymer, and the size of the implant (see, e.g., U.S. Pat. No. 6,620,422 and WO05/011769). Other sustained release formulations are described in EP0467389, WO93/241150, U.S. Pat. No. 5,612,052, WO97/40085, WO03/075887, WO01/01964, U.S. Pat. No. 5,922,356, WO94/155587, WO02/074247, WO98/25642, U.S. Pat. No. 5,968,895, U.S. Pat. No. 6,180,608, US20030171296, US20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO02/058672, WO9726015, WO97/04744, and US20020019446. In such sustained release formulations microparticles of compound are combined with microparticles of polymer. U.S. Pat. No. 6,011,011 and WO94/06452 described a sustained release formulation providing either polyethylene glycols (e.g., PEG 300 and PEG 400) or triacetin. WO03/053401 describes a formulation which may both enhance bioavailability and provide controlled release of the agent within the GI tract. Additional controlled release formulations are described in WO02/38129, EP326151, U.S. Pat. No. 5,236,704, WO02/30398, WO98/13029, US20030064105, US20030138488, US20030216307, U.S. Pat. No. 6,667,060, WO01/49249, WO01/49311, WO01/49249, WO01/49311, U.S. Pat. No. 5,877,224, WO05/030179, WO05/027878, WO05/012488 and WO05/007074.

### Controlled Release Formulations

In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

Matrix devices are a common device for controlling the release of various agents. In such devices, the agents described herein are generally present as a dispersion within the polymer matrix, and are typically formed by the compression of a polymer/drug mixture or by dissolution or melting. The dosage release properties of these devices may be dependent upon the solubility of the agent in the polymer matrix or, in the case of porous matrices, the solubility in the sink solution within the pore network, and the tortuosity of the network. In one instance, when utilizing an erodible polymeric matrix, the matrix imbibes water and forms an aqueous-swollen gel that entraps the agent. The matrix then gradually erodes, swells, disintegrates or dissolves in the GI tract, thereby controlling release of one or more of the agents described herein. In non-erodible devices, the agent is released by diffusion through an inert matrix.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. Nonlimiting examples of such devices are disclosed in U.S. patent application Ser. No. 09/495,059 filed Jan. 31, 2000.

The erodible polymeric matrix into which an agent described herein can be incorporated may generally be described as a set of excipients that are mixed with the agent following its formation that, when contacted with the aqueous environment of use imbibes water and forms a water-swollen gel or matrix that entraps the drug form. Drug release may occur by a variety of mechanisms, for example, the matrix may disintegrate or dissolve from around particles or granules of the agent or the agent may dissolve in the imbibed aqueous solution and diffuse from the tablet, beads or granules of the device. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g., chitin, chitosan, dextran and pullulan; gum agar, gum arabic, gum karaya, locust bean gum, gum tragacanth, carrageenans, gum ghatti, guar gum, xanthan gum and scleroglucan), starches (e.g., dextrin and maltodextrin), hydrophilic colloids (e.g., pectin), phosphatides (e.g., lecithin), alginates (e.g., ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

The choice of matrix material can have a large effect on the maximum drug concentration attained by the device as well as the maintenance of a high drug concentration. The matrix material can be a concentration-enhancing polymer, for example, as described in WO05/011634.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g. methyl acrylate-methyl methacrylate copolymers, polyvinyl chloride, polyethylene), hydrophilic polymers (e.g., ethyl cellulose, cellulose acetate, crosslinked polyvinylpyrrolidone (also known as crospovidone)), and fatty compounds (e.g., carnauba wax, microcrystalline wax, and triglycerides). Such devices are described further in Remington: The Science and Practice of Pharmacy, 20th edition (2000).

Matrix controlled release devices may be prepared by blending an agent described herein and other excipients together, and then forming the blend into a tablet, caplet, pill, or other device formed by compressive forces. Such compressed devices may be formed using any of a wide variety of presses used in the fabrication of pharmaceutical devices. Examples include single-punch presses, rotary tablet presses, and multilayer rotary tablet presses, all well known in the art. See for example, Remington: The Science and Practice of Pharmacy, 20th Edition, 2000. The compressed device may be of any shape, including round, oval, oblong, cylindrical, or triangular. The upper and lower surfaces of the compressed device may be flat, round, concave, or convex.

In certain embodiments, when formed by compression, the device has a strength of at least 5 Kiloponds (Kp)/cm² (for example, at least 7 Kp/cm²). Strength is the fracture force, also known as the tablet hardness required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kP/cm². Friability is a well-know measure of a device's resistance to surface abrasion that measures weight loss in percentage after subjecting the device to a standardized agitation procedure. Friability values of from 0.8 to 1.0% are regarded as constituting the upper limit of acceptability. Devices having a strength of greater than 5 kP/cm² generally are very robust, having a friability of less than 0.5%. Other methods for forming matrix controlled-release devices are well known in the pharmaceutical arts. See for example, Remington: The Science and Practice of Pharmacy, 20th Edition, 2000.

As noted above, the agents described herein may also be incorporated into an osmotic control device. Such devices generally include a core containing one or more agents as described herein and a water permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Nonlimiting examples of osmotic control devices are disclosed in U.S. patent application Ser. No. 09/495,061.

Osmotic agents create a driving force for transport of water from the environment of use into the core of the device. Osmotic agents include but are not limited to water-swellable hydrophilic polymers, and osmogens (or osmagens). Thus, the core may include water-swellable hydrophilic polymers, both ionic and nonionic, often referred to as osmopolymers and hydrogels. The amount of water-swellable hydrophilic polymers present in the core may range from about 5 to about 80 wt % (including for example, 10 to 50 wt %). Nonlimiting examples of core materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Nonlimiting examples of additives and excipients include but are not limited to those described elsewhere herein as well as microcrystalline cellulose, metallic salts of acids (e.g., aluminum stearate, calcium stearate, magnesium stearate, sodium stearate, zinc stearate), pH control agents (e.g., buffers, organic acids, organic acid salts, organic and inorganic bases), fatty acids, hydrocarbons and fatty alcohols (e.g., stearic acid, palmitic acid, liquid paraffin, stearyl alcohol, and palmitol), fatty acid esters (e.g., glyceryl(mono- and di-) stearates, triglycerides, glyceryl (palmiticstearic) ester, sorbitan esters (e.g., sorbitan monostearate, saccharose monostearate, saccharose monopalmitate, sodium stearyl fumarate), polyoxyethylene sorbitan esters), surfactants (e.g., alkyl sulfates (e.g., sodium lauryl sulfate, magnesium lauryl sulfate), polymers (e.g., polyethylene glycols, polyoxyethylene glycols, polyoxyethylene, polyoxypropylene ethers, including copolymers thereof), polytetrafluoroethylene), and inorganic materials (e.g., talc, calcium phosphate), cyclodextrins, sugars (e.g., lactose, xylitol), sodium starch glycolate). Nonlimiting examples of disintegrants are sodium starch glycolate (e.g., Explotab™ CLV, (microcrystalline cellulose (e.g., Avicel™), microcrystalline silicified cellulose (e.g., ProSolv™), croscarmellose sodium (e.g., Ac-Di-Sol™). When the agent described herein is a solid amorphous dispersion formed by a solvent process, such additives may be added directly to the spray-drying solution when forming an agent described herein/concentration-enhancing polymer dispersion such that the additive is dissolved or suspended in the solution as a slurry, Alternatively, such additives may be added following the spray-drying process to aid in forming the final controlled release device.

A nonlimiting example of an osmotic device consists of one or more drug layers containing an agent described herein, such as a solid amorphous drug/polymer dispersion, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery devices may be fabricated in various geometries including bilayer (wherein the core comprises a drug layer and a sweller layer adjacent to each other), trilayer (wherein the core comprises a sweller layer sandwiched between two drug layers) and concentric (wherein the core comprises a central sweller agent surrounded by the drug layer). The coating of such a tablet comprises a membrane permeable to water but substantially impermeable to drug and excipients contained within. The coating contains one or more exit passageways or ports in communication with the drug-containing layer(s) for delivering the drug agent. The drug-containing layer(s) of the core contains the drug agent (including optional osmagents and hydrophilic water-soluble polymers), while the sweller layer consists of an expandable hydrogel, with or without additional osmotic agents.

When placed in an aqueous medium, the tablet imbibes water through the membrane, causing the agent to form a dispensable aqueous agent, and causing the hydrogel layer to expand and push against the drug-containing agent, forcing the agent out of the exit passageway. The agent can swell, aiding in forcing the drug out of the passageway. Drug can be delivered from this type of delivery system either dissolved or dispersed in the agent that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

Other materials useful in forming the drug-containing agent, in addition to the agent described herein itself, include HPMC, PEO and PVP and other pharmaceutically acceptable carriers. In addition, osmagents such as sugars or salts, including but not limited to sucrose, lactose, xylitol, mannitol, or sodium chloride, may be added. Materials which are useful for forming the hydrogel layer include sodium CMC, PEO (e.g., polymers having an average molecular weight from about 5,000,000 to about 7,500,000 daltons), poly(acrylic acid), sodium (polyacrylate), sodium croscarmellose, sodium starch glycolate, PVP, crosslinked PVP, and other high molecular weight hydrophilic materials.

In the case of a bilayer geometry, the delivery port(s) or exit passageway(s) may be located on the side of the tablet containing the drug agent or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

The osmotic device can also be made with a homogeneous core surrounded by a semipermeable membrane coating, as in U.S. Pat. No. 3,845,770. The agent described herein can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above. In one embodiment, an osmotic device comprises: (a) a single-layer compressed core comprising: (i) an agent described herein, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the core; and (c) at least one passageway within the water-permeable layer (b) for delivering the drug to a fluid environment surrounding the tablet. In certain embodiments, the device is shaped such that the surface area to volume ratio (of a water-swollen tablet) is greater than 0.6 mm⁻¹ (including, for example, greater than 1.0 mm⁻¹). The passageway connecting the core with the fluid environment can be situated along the tablet band area. In certain embodiments, the shape is an oblong shape where the ratio of the tablet tooling axes, i.e., the major and minor axes which define the shape of the tablet, are between 1.3 and 3 (including, for example, between 1.5 and 2.5). In one embodiment, the combination of the agent described herein and the osmagent have an average ductility from about 100 to about 200 Mpa, an average tensile strength from about 0.8 to about 2.0 Mpa, and an average brittle fracture index less than about 0.2. The single-layer core may optionally include a disintegrant, a bioavailability enhancing additive, and/or a pharmaceutically acceptable excipient, carrier or diluent. Nonlimiting examples of such devices are disclosed, for example, in U.S. provisional Patent Application Ser. No. 60/353,151.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™), microcrystalline silicified cellulose (e.g., ProSolv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Water-soluble polymers are added to keep particles of the agent suspended inside the device before they can be delivered through the passageway(s) (e.g., an orifice). High viscosity polymers are useful in preventing settling. However, the polymer in combination with the agent is extruded through the passageway(s) under relatively low pressures. At a given extrusion pressure, the extrusion rate typically slows with increased viscosity. Certain polymers in combination with particles of the agent described herein form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such devices are prepared with no polymer added, which leads to poor drug delivery unless the tablet is constantly agitated to keep the particles from settling inside the core. Settling is also problematic when the particles are large and/or of high density such that the rate of settling increases.

In certain embodiments, the water-soluble polymers for such osmotic devices do not interact with the drug. In certain embodiments the water-soluble polymer is a non-ionic polymer. A nonlimiting example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol 250H™ provides effective drug delivery at concentrations as low as about 3% by weight of the core when combined with an osmagent. Natrosol 250H™ NF is a high-viscosity grade nonionic cellulose ether that is soluble in hot or cold water. The viscosity of a 1% solution of Natrosol 250H using a Brookfield LVT (30 rpm) at 25° C. is between about 1, 500 and about 2,500 cps.

In certain embodiments, hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

The osmotic controlled release devices of the present invention can also comprise a coating. In certain embodiments, the osmotic controlled release device coating exhibits one or more of the following features: is water-permeable, has at least one port for the delivery of drug, and is non-dissolving and non-eroding during release of the drug formulation, such that drug is substantially entirely delivered through the delivery port(s) or pores as opposed to delivery primarily via permeation through the coating material itself Delivery ports include any passageway, opening or pore whether made mechanically, by laser drilling, by pore formation either during the coating process or in situ during use or by rupture during use. In certain embodiments, the coating is present in an amount ranging from about 5 to 30 wt % (including, for example, 10 to 20 wt %) relative to the core weight.

One form of coating is a semipermeable polymeric membrane that has the port(s) formed therein either prior to or during use. Thickness of such a polymeric membrane may vary between about 20 and 800 μm (including, for example, between about 100 to 500 μm). The diameter of the delivery port (s) may generally range in size from 0.1 to 3000 μm or greater (including, for example, from about 50 to 3000 μm in diameter). Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. No. 5,612,059 and U.S. Pat. No. 5,698,220. The delivery port may be formed in situ by rupture of the coating, for example, when a collection of beads that may be of essentially identical or of a variable agent are used. Drug is primarily released from such beads following rupture of the coating and, following rupture, such release may be gradual or relatively sudden. When the collection of beads has a variable agent, the agent may be chosen such that the beads rupture at various times following administration, resulting in the overall release of drug being sustained for a desired duration.

Coatings may be dense, microporous or asymmetric, having a dense region supported by a thick porous region such as those disclosed in U.S. Pat. No. 5,612,059 and U.S. Pat. No. 5,698,220. When the coating is dense the coating can be composed of a water-permeable material. When the coating is porous, it may be composed of either a water-permeable or a water-impermeable material. When the coating is composed of a porous water-impermeable material, water permeates through the pores of the coating as either a liquid or a vapor. Nonlimiting examples of osmotic devices that utilize dense coatings include U.S. Pat. No. 3,995,631 and U.S. Pat. No. 3,845,770. Such dense coatings are permeable to the external fluid such as water and may be composed of any of the materials mentioned in these patents as well as other water-permeable polymers known in the art.

The membranes may also be porous as disclosed, for example, in U.S. Pat. No. 5,654,005 and U.S. Pat. No. 5,458,887 or even be formed from water-resistant polymers. U.S. Pat. No. 5,120,548 describes another suitable process for forming coatings from a mixture of a water-insoluble polymer and a leachable water-soluble additive. The porous membranes may also be formed by the addition of pore-formers as disclosed in U.S. Pat. No. 4,612,008. In addition, vapor-permeable coatings may even be formed from extremely hydrophobic materials such as polyethylene or polyvinylidene difluorid that, when dense, are essentially water-impermeable, as long as such coatings are porous. Materials useful in forming the coating include but are not limited to various grades of acrylic, vinyls, ethers, polyamides, polyesters and cellulosic derivatives that are water-permeable and water-insoluble at physiologically relevant pHs, or are susceptible to being rendered water-insoluble by chemical alteration such as by crosslinking. Nonlimiting examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxiated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes. In various embodiments, the coating agent comprises a cellulosic polymer, in particular cellulose ethers, cellulose esters and cellulose ester-ethers, i.e., cellulosic derivatives having a mixture of ester and ether substituents, the coating materials are made or derived from poly(acrylic) acids and esters, poly (methacrylic) acids and esters, and copolymers thereof, the coating agent comprises cellulose acetate, the coating comprises a cellulosic polymer and PEG, the coating comprises cellulose acetate and PEG.

Coating is conducted in conventional fashion, typically by dissolving or suspending the coating material in a solvent and then coating by dipping, spray coating or by pan-coating. In certain embodiments, the coating solution contains 5 to 15 wt % polymer. Typical solvents useful with the cellulosic polymers mentioned above include but are not limited to acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described, for example, in U.S. Pat. No. 5,612,059. Coatings may also be hydrophobic microporous layers wherein the pores are substantially filled with a gas and are not wetted by the aqueous medium but are permeable to water vapor, as disclosed, for example, in U.S. Pat. No. 5,798,119. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Hydrophobic microporous coating materials include but are not limited to polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed, for example, in U.S. Pat. No. 4,247,498, U.S. Pat. No. 4,490,431 and U.S. Pat. No. 4,744,906. Osmotic controlled-release devices may be prepared using procedures known in the pharmaceutical arts. See for example, Remington: The Science and Practice of Pharmacy, 20th Edition, 2000.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size. Other excipients, such as a binder (e.g., microcrystalline cellulose), may be blended with the agent to aid in processing and forming the multiparticulates. In the case of wet granulation, a binder such as microcrystalline cellulose may be included in the granulation fluid to aid in forming a suitable multiparticulate. See, for example, Remington: The Science and Practice of Pharmacy, 20″Edition, 2000. In any case, the resulting particles may themselves constitute the therapeutic composition or they may be coated by various film-forming materials such as enteric polymers or water-swellable or water-soluble polymers, or they may be combined with other excipients or vehicles to aid in dosing to patients.

In certain embodiments, it may be desirable to provide for the immediate release of one or more of the agents described herein, and the controlled release of one or more other agents. For example, in one embodiment, a compound described herein can be provided in an immediate release formulation together with a fibrate (e.g., Tricor) or a CETP inhibitor (e.g., torcetrapib) in a controlled release format. In another embodiment, a compound described herein can be provided together with an HMG CoA reductase inhibitor in an immediate release formulation. For example, a compound described herein can be coformulated with an HMG CoA reductase inhibitor in the immediate release formulation described in WO05/011634 (page 29, line 31 to page 33 (entire page). In other embodiments, a compound described herein is provided in a controlled release format together with another agent (e.g., an HMG CoA reductase inhibitor) in an immediate release formulation. In other embodiments, one or more agents described herein (for example, a compound described herein and an HMG CoA reductase inhibitor) can be provided in an immediate release formulation together with one or more other agents (for example, a fibrate and/or torcetrapib) in a controlled release format.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered locally, for example, at the site of injury to an injured blood vessel. The agents can be coated on a stent. The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U.S. 20030165436, and WO 96/40089 (which includes vegetable oil). Sustained release formulations suitable for inhalation are described in U.S. 20010036481 A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature

Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

### Administration and Formulation of Combitherapy Protein/Peptide Agents

Some of the agents used in combitherapy with compounds described herein are proteins (e.g., nitric oxide synthase isoforms, HDL associated proteins such as ApoA-I or Apo A-I Milano) or peptides (e.g., peptides which mitigates one or more symptoms of atherosclerosis, peptides and peptide analogues that mimic the structural and pharmacological properties of human ApoA-I, Exenatide®). In some embodiments, the recombinant or purified protein is administered together with a compound described herein. In alternative embodiments, genes encoding the protein or peptide to be delivered may be administered, rather than the protein. Gene transfer can be obtained using direct transfer of genetic material, in a plasmid or viral vector, or via transfer of genetic material in cells or carriers such as cationic liposomes. Such methods are well known in the art and readily adaptable for use in the therapies described herein. For example, studies by Wolff et al., Biotechniques 11:474-85 (1991), demonstrate injection of naked DNA into muscle allows long term and low expression levels of proteins coded for within the DNA sequence. Administration of naked DNA to smooth muscle layers can be achieved by use of an intramural device, such as an INFILTRATOR™ and allow expression of the proteins or their alpha helical domains to treat the injured vessel. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Plasmid or viral vectors are agents that transport the gene into a cell without degradation and may include a promoter yielding expression of the gene in the cell into which it is delivered. In certain embodiments vectors are derived from either a virus or a retrovirus. Viral vectors include but are not limited to those derived from Adenovirus, Adeno-associated virus, Herpes virus, Vaccinia virus, Polio virus, AIDS virus, neuronal trophic virus, Sindbis and other RNA viruses, including these viruses with the HIV backbone. Vectors from other viral families which share the properties of these viruses may make them suitable for use as vectors. Retroviral vectors include but are not limited to those derived from include Murine Maloney Leukemia virus, MMLV, and retroviruses that express the desirable properties of MMLV as a vector. In certain embodiments where non-proliferating cells are involved, retroviral vectors are not used. Retroviral vectors, in general, are described by Verma, I. M., Retroviral vectors for gene transfer. In MICROBIOLOGY—1985, American Society for Microbiology, pp. 229-232, Washington, (1985). Examples of methods for using retroviral vectors for gene therapy are described in U.S. Pat. No. 4,868,116, U.S. Pat. No. 4,980,286, WO90/02806, WO 89/07136 and Mulligan, (Science 260:926-932 (1993)).

Adenovirus vectors are relatively stable and easy to work with, have high titers, and can be delivered in aerosol formulation, and can transfect non-dividing cells. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). Adenoviral derived vectors are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Pox viral vectors can be used in the gene transfer techniques described herein. In certain embodiment the viral/retroviral vector used in the gene transfer techniques described herein have been engineered so as to suppress the immune response of the host organism, elicited by the viral antigens. In certain embodiments, these vectors carry coding regions for Interleukin 8 or 10. In certain embodiments, the viral/retroviral vectors described herein have one or more of the early genes removed and a gene or gene/promoter cassette inserted into the viral genome in place of the removed viral DNA.

The inserted genes in viral/retroviral vectors usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. Promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Promoters from the host cell (to which the viral vector is being transferred) or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell. Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell. Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Enhancers include but are not limited to the SV 40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments, the promoter and/or enhancer region act as a constitutive promoter and/or enhancer to maximize expression of the region of the transcription unit to be transcribed. In certain embodiments the promoter and/or enhancer region is active in all eukaryotic cell types. Promoters include but are not limited to the CMV promoter (650 bases), SV40 promoters, cytomegalovirus (full length promoter), and retroviral vector LTF.

Expression vectors used in eukaryotic host cells may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. In certain embodiments, the transcription unit also contains a polyadenylation region (e.g., that derived from the SV40 early polyadenylation signal consisting of about 400 bases). One benefit of this region is that it increases the likelihood that the transcribed unit will be processed and transported like mRNA. The identification and use of polyadenylation signals in expression constructs is well established. In certain embodiments, homologous polyadenylation signals are used in the transgene constructs. In certain embodiments, the transcribed units contain other standard sequences alone or in combination with the above sequences improve expression from, or stability of, the construct.

The viral/retroviral vectors can include nucleic acid sequence encoding a marker product. This marker product is used to determine if the gene has been delivered to the cell and once delivered is being expressed. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g., preventing or treating vascular diseases/disorders and conditions (including but not limited to arteriosclerosis, atherosclerosis, cardiovascular disease, cerebrovascular disease, renovascular disease, mesenteric vascular disease, pulmonary vascular disease, ocular vascular disease and peripheral vascular disease), hyperlipidemia (including but not limited to hypercholesterolemia, hypertriglyceridemia, sitosterolemia), hypertension, angina, cardiac arrhythmias, congestive heart failure, and stroke). The kit may optionally comprise a second pharmaceutical composition comprising one or more additional agents chosen from (1) a dyslipidemic agent, (2) an anti-diabetic agent, (3) an anti-hypertensive agent, (4) an anti-obesity agent, (5) an agent used to treat autoimmune disorders, (6) an agent used to treat demylenation and its associated disorders, (7) an agent used to treat Alzheimer's disease, (8) a blood modifier, (9) a hormone replacement agent/composition, (10) a chemotherapeutic agent, (11) a peptide which mitigates one or more symptoms of atherosclerosis, (12) an anti-cancer agent, and (13) an agent used to treat bone loss and associated disorders and a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### In Vivo Assay of Hypolipidemic Agents Using the Rat Cholesterol Absorption Model.

This model is based on models described by Burnett et al, (Burnett, D A. Caplen, M A, Browne, M E, Zhau, H., Altmann, S W., Davis, H R Jr., Clader, J W. Bioorg. Med. Chem. Lett. 2002, 12(3):315-8) and Sparrow et al, (Sparrow, C P., Patel, S., Baffic, J., Chao, Y-S., Hernandez, M., Lam, M-H., Montenegro, J., Wright, S D., Detmers, P A. J. Lipid Res. 1999 40: 1747-1757).

Female Sprague-Dawley rats weighing 150-250 g are separated into groups of 3 and fasted overnight. The animals (4-6/group) are dosed perorally with 300 μL test compounds in olive oil or suitable vehicle. Thirty minutes later, 3-5 microCuries ³H-cholesterol per rat are delivered perorally in 300 μL olive oil. After three hours, 200 μL serum is collected, vortexed with scintillation fluid, and measured for radioactivity in a scintillation counter. Percent inhibition is defined as 100*(1−Cₜₑₛₜ/C_{ctrl}), where Cₜₑₛₜ and C_{ctrl} refer to ³H levels in serum for the test compound and for the vehicle only control, respectively. Percent inhibition values are reported for a fixed dose. The ED₅₀ is the dose at which the half-maximal effect on serum ³H levels is observed for a given test compound.

### In Vivo Assay of Hypolipidemic Agents Using the Mouse Cholesterol Absorption Model.

Female CD-1 mice weighing 20-30 g are separated into groups of 3-8 and fasted overnight. The animals (3-8/group) are dosed perorally with 200 μL test compound in olive oil or suitable vehicle. Thirty minutes later, 3-5 microCuries ³H-cholesterol per mouse are delivered perorally in 200 μL olive oil. After three hours, 100 μL serum is collected, vortexed with scintillation fluid, and measured for radioactivity in a scintillation counter. Percent inhibition and ED₅₀ are defined as in the Rat Cholesterol Absorption Model above.

### In Vivo Assay of Hypolipidemic Agents Using the Hyperlipidemic Hamster:

Hamsters are separated into groups of six and given a controlled cholesterol diet (Purina Chow #5001 containing 0.5% cholesterol) for seven days. Diet consumption is monitored to determine dietary cholesterol exposure in the face of test compounds. The animals are dosed with the test compound once daily beginning with the initiation of diet. Dosing is by oral gavage of 0.2 mL of corn oil alone (control group) or solution (or suspension) of test compound in corn oil. All animals moribund or in poor physical condition are euthanized. After seven days, the animals are anesthetized by intramuscular (IM) injection of ketamine and sacrificed by decapitation. Blood is collected into vacutainer tubes containing EDTA for plasma lipid analysis and the liver excised for tissue lipid analysis. Lipid analysis is conducted as per published procedures (Schnitzer-Polokoff, R., et al, Comp. Biochem. Physiol., 1991 99A, 4, 665-670) and data are reported as percent reduction of lipid versus control.

### In Vivo Assay of Hypolipidemic Agents Using the Hamster Acute Cholesterol Absorption Model

Male Syrian Hamsters weighing approximately 120 g are separated into groups of 3-6 and fasted overnight. The animals (3-6/group) are dosed perorally with 200 μL test compound in olive oil or suitable vehicle. Thirty minutes later, 3-5 microCuries ³H-cholesterol per hamster are delivered perorally in 200 μL olive oil. After three hours, 100-200 μL serum is collected, vortexed with scintillation fluid, and measured for radioactivity in a scintillation counter. Percent inhibition and ED₅₀ are defined as in the Rat Cholesterol Absorption Model above.

The bioabsorption of the compounds herein described may be examined using the Caco-2 cell monolayer model of Hilgers et al. (Hilgers A R, Conradi R A, Burton P S. Pharm. Res. 1990. 7(9), 902-10)

### Pharmacokinetics

To study the pharmacokinetics of compounds, bioavailability studies are carried out in various test animals. Compounds are prepared in suitable formulations for oral and intravenous administration. Compounds are administered via intravenous injection (tail vein (rat), femoral vain (hamster), peripheral vain (monkey), cephalic vein (dog)) and orally (via a capsule (dogs) or gavage (all others)) to independent groups of test animals which are either fasted overnight or non-fasted. Serum or plasma is collected at various time points and assayed for the presence of compounds using an LC/MS/MS detection method. Experiment samples are either diluted 15-fold in 30% acetonitrile in water, injected onto an in-line sample extraction cartridge (Waters Oasis HLB Direct Connect) and loaded onto a reverse phase HPLC column fitted with a appropriate guard column or prepared using a protein crash, dried under nitrogen, resuspended in 30% acetonitrile in water and loaded onto a reverse phase HPLC column fitted with a appropriate guard column. Samples are eluted from the reverse phase HPLC column with a gradient. A Micromass Quattro Micro (Waters Corporation, Milford, Mass.) triple quadrupole mass spectrometer operated in MRM mode is used for detection. Concentrations are calculated based on a standard concentration curve of compound or standard curves generated using peak area ratio of compound to internal standard vs. concentration. MassLynx software (Waters, Corporation, Milford, Mass.) is used to calculate the absolute concentration of test compound in each serum or plasma sample. A concentration versus time plot is generated from the data in Microsoft Excel, Summit Software PK Solutions 2.0, GraphPad Prism (GraphPad Software, Inc., San Diego, Calif.) or WinNonlin Professional Version 4.1 (Pharsight Corporation, Mountain View, Calif.) to generate pharmacokinetic curves. An area under the curve (AUCₙ, n=length of experiment in minutes or hours) is calculated from the concentration vs. time data by software using the linear trapezoid method for both the orally and intravenously dosed animals. Oral Bioavailability (F) over the length of the experiment is calculated using the equation:

F=(AUCₒᵣₐₗ*Dose_{i.v.})/(AUC_{i.v.}*Doseₒᵣₐₗ).

### Determination of Acyl Coenzyme A: Cholesterol Acyltransferase (ACAT) Inhibition Activity

The ability of compounds of the invention to inhibit acyl-coenzyme A: cholesterol acyltransferase (ACAT) activity is assayed by measuring cholesterol esterification in human HepG2 and Caco2 cells. ACAT activity is measured by following the conversion of ¹⁴C-oleic acid to ¹⁴C-cholesteryl oleate in an assay based on Junquero, et al. 2001 Biochem Pharmacol 61:97-108 and Sugiyama, et al. Atherosclerosis 118: 145-53. Cells are propagated in Eagle's Minimum Essential Medium (EMEM) supplemented with fetal bovine serum (10% for HepG2 cells; 20% for Caco2 cells) and 2 mM L-glutamine. All incubations are performed at 37° C. in air with 5% CO₂. In preparation for each experiment, cells are seeded in 6-well plates and allowed to grow to 90-95% confluency. All treatments are performed in duplicate. Inhibitors are pre-incubated with cells for 4 h. The assay is initiated by adding ¹⁴C-oleate/bovine serum albumin solution to each assay well and incubating an additional 2 h at 37° C. Cell monolayers are extracted with 2 mL 3:2 hexane:isopropanol at room temperature for 30 min. Extracts are dried down under nitrogen and dissolved in 75 □L chloroform. Formation of ¹⁴C-cholesteryl oleate is determined by separation of the ACAT assay reaction products by thin-layer chromatography (TLC) and visualization by phosphorimaging. Percent inhibition is calculated for each compound dose, and IC₅₀ values are determined using GraphPad Prism by regression analysis of percent inhibition plotted as a function of the logarithmic value of the sample concentration. Values for the known ACAT inhibitor, N-[4-(2-chlorophenyl)-6,7-dimethyl-3-quinolyl]-N′-(2,4-difluorophenyl)urea (TMP-153) and the known cholesterol absorption inhibitor molecule, (3R,4S)-1-(4-fluorophenyl)-3-[(3S)-3-(4-fluorophenyl)-3-hydroxypropyl]-4-(4-hydroxyphenyl)azetidin-2-one can also be determined.

### Competitive Binding Assays

The ability of compounds of the invention to bind and compete for specific binding to a receptor in the hamster small intestine is tested. Competition binding to hamster small intestine is determined by using an in vivo assay based on Hernandez et al. 2000 (Biochim Biophys Acta 1486:232-242) in which radiolabeled compound is administered to hamsters in the presence and absence of unlabeled, test, competitor compounds. In this experiment, a compound of the invention, can be tritiated and used as a radioligand. Corresponding unlabeled compound in 1200-fold excess is used to demonstrate that the observed binding is specific. Other compounds of the invention and a known cholesterol absorption inhibitor, (3R,4S)-1-(4-fluorophenyl)-3-[(3S)-3-(4-fluorophenyl)-3-hydroxypropyl]-4-(4-hydroxyphenyl)azetidin-2-one, are evaluated for their ability to compete for binding of the tritiated radioligand when administered in 1200-fold excess. Golden Syrian hamsters are fasted overnight prior to dosing. Animals are dosed by oral gavage with 0.5 ml of either vehicle or vehicle containing 0.35 mg/kg test compound. One hour later, animals are dosed by oral gavage with 5 μCi tritiated radioligand with vehicle or vehicle containing 0.35 mg/kg test compound as above. Three hours after administration of the tritiated radioligand, animals are euthanized by CO₂ overdose, the small intestine dissected, flushed with cold saline, and placed into an empty tube on ice. The small intestine is cut into 6 cm segments. The intestinal epithelial mucosa is extruded from each segment, homogenized in PBS, and the radioactivity in the homogenate is counted by liquid scintillation counting. Results are normalized for protein content of the homogenates. Tritiated radioligand binding to the hamster small intestine in the presence and absence of test compound is determined by calculating the average bound radioactivity per mg of protein (DPM/mg) for each treatment group. Percent tritiated radioligand binding is calculated for each compound using the formulas:

Bound Radioactivity(DPM/mg)=Radioactivity(DPM)/Total Protein(mg)

Percent ³H Binding vs Vehicle Control=100%*((Bound Radioactivity)_{competitor}/(Bound Radioactivity)_{vehicle})

Statistical analysis is performed using an unpaired, two-tailed, Student's t-test (GraphPad Prism).

A desirable medicament would inhibit cholesterol absorption without affecting the acute absorption of other important molecules of dietary origin. Such a cholesterol absorption inhibitor would not interfere with the absorption of triglyceride, progesterone, ethinyl estradiol, vitamin A, vitamin D, or taurocholic acid. For example, cholestyramine, which is in clinical use to lower serum cholesterol, sequesters bile acids in the intestine, ultimately leading to a decrease in plasma cholesterol by upregulating the synthesis of bile acids from cholesterol in the liver. Two side effects of cholestyramine are gastrointestinal discomfort and the sequestration of fat-soluble vitamins. On the other hand, ezetimibe, a known cholesterol absorption inhibitor, does not appear to affect fat-soluble vitamin absorption in humans. In addition, ezetimibe does not inhibit the absorption of taurocholic acid, suggesting that certain cholesterol absorption inhibitors can lower serum cholesterol without inhibiting the ileal Na⁺/bile acid cotransporter.

### Retinol, Taurocholic Acid, Prozesterone, Sitostanol, and Cholesterol Absorption Assays

The effects of acute oral administration of several compounds of the invention on retinol, taurocholic acid, and progesterone absorption are studied in female Sprague Dawley rats. Groups of 5 rats receive 10 mg/kg of test compound or vehicle (olive oil) via oral gavage. Test compounds are administered 30 minutes prior to oral administration of ¹⁴C-cholesterol (5 μCi) in addition to either ³H-retinol (3 μCi), ³H-taurocholic acid (3 μCi), ³H-Progesterone (3 μCi), or ³H-sitostanol (3 μCi) constituted in olive oil (300 μL). Blood is sampled from all animals via the retro-orbital sinus under isoflurane anesthesia three hours after the administration of the radiolabeled cocktail and again at 24 hours. Serum radioactivity (DPM) is measured and the percent absorption (% absorption) is calculated as:

average dpm treated group/average dpm control group×100.

Assays can also be performed using the known cholesterol absorption inhibitor molecule, (3R,4S)-1-(4-fluorophenyl)-3-[(3S)-3-(4-fluorophenyl)-3-hydroxypropyl]-4-(4-hydroxyphenyl)azetidin-2-one is for comparison.

### Additivity Assay

The effects of compounds of the invention either alone and in combination with the known cholesterol absorption inhibitor, (3R,4S)-1-(4-fluorophenyl)-3-[(3S)-3-(4-fluorophenyl)-3-hydroxypropyl]-4-(4-hydroxyphenyl)azetidin-2-one can be studied in one or more of the in vivo animal models above. For example, in the case of the rat cholesterol absorption model, groups of five rats receive each of the compounds of the invention alone (1 mg/kg) or in combination with (3R,4S)-T-(4-fluorophenyl)-3-[(3S)-3-(4-fluorophenyl)-3-hydroxypropyl]-4-(4-hydroxyphenyl)azetidin-2-one (each at 1 mg/kg) or vehicle (olive oil) via oral gavage. Serum radioactivity (DPM) is measured and the average values are plotted.

### Behavioral Assay

The compounds of the invention can be tested to determine their effects on general activity (e.g., behavioural, autonomic and motor capabilities), for example, using Irwin's method (Psychopharmacologia—1968 13:222-57). Briefly, groups of rodents receive a single administration of vehicle or different doses of test compound by oral gavage. Irwin observations are performed at 30, 60, 90, 180 and 300 minutes post dosing. Animals are generally observed 7 days post dosing.

### Electrophysiological Assays

hERG channels are expressed in a human embryonic kidney (HEK293) cell line that lacks endogenous I_{Kr}. HEK293 cells are stably transfected with hERG cDNA. Stable transfectants are selected by coexpression with the G418-resistance gene incorporated into the expression plasmid. Selection pressure is maintained by including G418 in the culture medium. Cells are cultured in Dulbecco's Modified Eagle Medium/Nutrient Mixture F-12 (D-MEM/F-12) supplemented with 10% fetal bovine serum, 100 U/mL penicillin G sodium, 100 μg/mL streptomycin sulfate and 500 μg/mL G418. Cells are maintained in tissue culture incubators at 37° C. in a humidified 95% air, 5% CO₂ atmosphere, with stocks maintained in cryogenic storage. Cells used for electrophysiology are plated in plastic culture dishes.

Test solution, reference substance (E-4031, 500 nm) and positive control (terfenadine, 60 nm) are prepared fresh daily in HEPES-buffered physiological saline (HB-PS) solution (composition in mM): NaCl, 137; KCl, 4.0; CaCl₂, 1.8; MgCl₂, 1; HEPES, 10; Glucose, 10; pH adjusted to 7.4 with NaOH. All test and control solutions also contain 0.3% dimethylsulfoxide (DMSO). Thus the vehicle control solution is HB-PS+DMSO≧0.3%.

Cells are transferred to the recording chamber and superfused with vehicle control solution. Micropipette solution for whole cell patch clamp recordings is composed of (mM): potassium aspartate, 130; MgCl₂, 5; EGTA, 5; ATP, 4; HEPES, 10; pH adjusted to 7.2 with KOH. The recording is performed at a temperature of 35±2° C. Micropipettes for patch clamp recording are made from glass capillary tubing using a P-97 micropipette puller (Sutter Instruments, Novato, Calif.). A commercial patch clamp amplifier is used for whole cell recordings. Before digitization, current records are low-pass filtered at one-fifth of the sampling frequency.

Cells stably expressing hERG are held at −80 mV. Onset and steady state activation of hERG current due to test compound is measured using a pulse pattern with fixed amplitudes (conditioning prepulse: +20 mV for 1 sec; repolarizing test ramp to −80 mV (−0.5 V/s)) repeated at 5 s intervals. Each recording ends with a final application of a supramaximal concentration of the reference substance (E-4031, 500 nM), to assess the contribution of endogenous currents. The remaining unblocked current is subtracted off-line digitally from the data to determine the potency of the test substance for hERG activation.

Steady state is defined by the limiting constant rate of change with time (linear time dependence). The steady state before and after test article application is used to calculate the percentage of current inhibited at each concentration. Percent activation at each concentration in the test group is compared with the vehicle control group using one-way ANOVA followed by Dunnett's multiple comparison test (JMP Version 5.0.1, SAS Institute, Cary, N.C.).

Test compound at different concentrations is applied to cells to determine effect on hERG current amplitude. The average value of 3 cells for each group standard error of the mean (SEM) is determined and compared to the positive control terfenadine, a known hERG channel inhibitor.

Increases in QT duration and action potential duration in isolated guinea pig hearts can be used to indicate an arrhythmogenic effect. Hearts are perfused with an oxygenated Tyrode's solution, containing 0.0; 1.0; 5.0 or 10.0 μM of test compound. QT duration and action potential duration (APD) are measured from cardiac electrodes. In separate experiments, the hearts are divided into 2 subgroups receiving either the test compound or control to determine the respective effects on QT duration and APD.

To observe the effects in vivo, mongrel dogs of either sex weighing 5-20 kg are anesthetized and instrumented by standard techniques for blood pressure and EKG. A solid state transducer for dP/dT is placed in the left cardiac ventricle, and an epicardial electrode is put into place. The test compound is infused followed by terfenadine at progressively higher doses, beginning at 1 μg/kg/min for 15 minutes and increased incrementally until a cardiovascular collapse ensues. Parameters measured are: blood pressure, heart rate, dP/dT, and the QT-interval. From the QT interval and the heart rate, a QTc interval may be calculated. Measurements of hemodynamics and electrical activity are made in response to the test compound and to control.

Electrophysiological effects of test compounds as a function of extracellular potassium and cycle length can be assessed using standard microelectrode techniques in canine Purkinje fibers (Gintant et al. 2001 J. Cardiovasc. Pharmacol. 37:607-618) and in rabbit Purkinje fibers (Lu et al. 2002 Europ. J. Pharmacol. 452:183-192).

The following examples are to be considered merely as illustrative and non-limiting in nature. It will be apparent to one skilled in the art to which the present invention pertains that many modifications, permutations, and variations may be made without departing from the scope of the invention.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

The starting materials, in the case of suitably substituted azetidinones, may be obtained by the methods described in WO 02/50027, WO 97/16424, WO 95/26334, WO 95/08532 and WO 93/02048, the disclosures of which are incorporated herein by reference.

Processes for obtaining compounds of the invention are presented below.

Other compounds of the invention may be prepared in analogous fashion to those whose synthesis is exemplified herein. The procedures below illustrate such methods. Furthermore, although the syntheses found herein result in the preparation of enantiomers having a particular stereochemistry, the present invention includes compounds of formulae I and Ia in any stereoisomeric form, and preparation thereof would be obvious to one of ordinary skill in the chemical arts based on the procedures presented herein.

### EXAMPLE 1

In the following scheme (+)-(4S)-3-(bromoacetyl)-4-phenyl-2-oxazolidinone (1) prepared according to the published procedure (Pridgen, L. N.; Abdel-Magid, A. F.; Lantos, I.; Shilcrat, S.; Eggleston, D. S. J. Org. Chem. 1993, 58, 5107-5117) is treated with 1-(4-fluorophenyl)-2-mercaptoethanone (prepared from commercially available 2-bromo-1-(4-fluorophenyl)ethanone by treatment with thiolacetic acid in the presence of triethylamine to give S-[2-(4-fluorophenyl)-2-oxoethyl]ethanethioate followed by saponification of the ester according to the published procedure; Vedejs, E.; Eberlein, T. H.; Mazur, D. J.; McClure, C. K.; Perry, D. A.; Ruggeri, R.; Schwartz, E.; Stults, J. S.; Varie, D. L.; Wilde, R. G.; Wittenberger, S. J. Org. Chem. 1986, 51, 1556-1562) to give thioether 2. Treatment of 2 with borane dimethylsulfide complex in the presence of a catalytic amount of (R)-1-methyl-3,3-diphenyltetrahydro-3H-pyrrolo[1,2-c][1,3,2]oxazaborole gives (4S)-4-benzyl-3-[(5S)-5-(4-fluorophenyl)-5-hydroxypentanoyl]-1,3-oxazolidin-2-one (3a). Protection of the benzylic alcohol as the TBS ether was effected by reaction with tert-butyldimethylsilyl chloride in the presence of imidazole as a base provides 3b.

2-Hydroxy-4-bromobenzaldehyde 4 was prepared by reaction of 3-bromophenol with paraformaldehyde in the presence of magnesium chloride and excess triethylamine in acetonitrile. Condensation of 4 with aniline (5) gave the desired imine 6a that was converted to the benzyl protected derivative 6b upon treatment with benzyl bromide and potassium carbonate in DMF. In the next step, 3b is treated with titanium tetrachloride and N-ethyldiisopropylamine followed by treatment with 6b to effect enantiospecific condensation providing 7. Treatment of 7 with excess N,O-bistrimethylsilyl-acetamide followed by a catalytic amount of tetrabutylammonium fluoride hydrate results in ring closure to the desired beta-lactam (8) while maintaining the TBS protecting group on the benzylic alcohol. Commercially available 4-bromophenylboronic acid (9) is converted to the corresponding pinacol ester 10 by stirring with pinacol in toluene. Treatment of 10 with a mixture of trimethylphosphite, AIBN, and tris(trimethylsilyl)silane in toluene produces the dimethylphosphonate derivative 11 (an adaptation of the published procedure; Jiao, X. Y.; Bentrude, W. G. J. Org. Chem. 2003, 68, 3303-3306). Suzuki coupling of 8 with 11 gives the expected biphenyl derivative 13 that is deprotected by hydrogenolysis over palladium on carbon, treatment with bromotrimethylsilane, and treatment with aqueous HF to give the product 12.

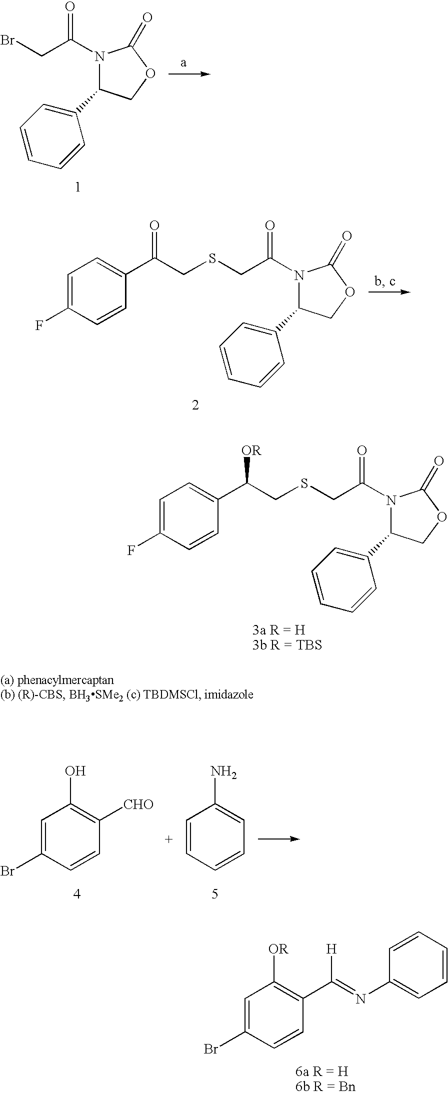

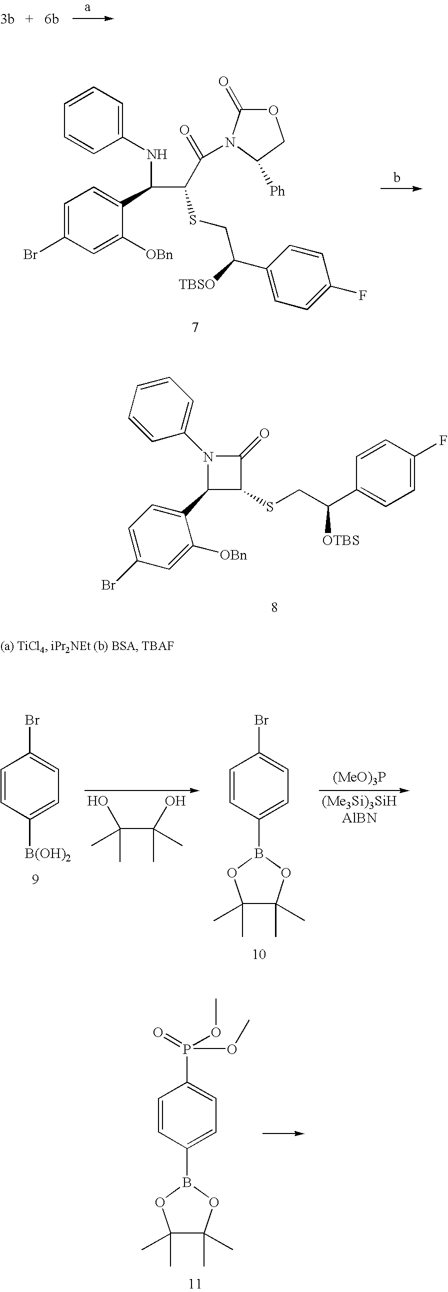

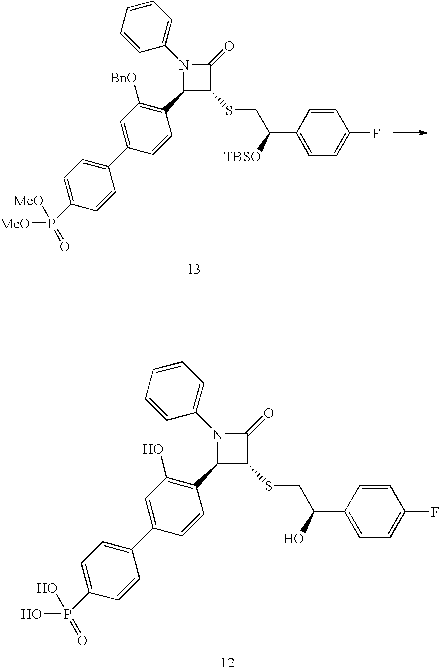

### EXAMPLE 2

In the following sequence the key protected intermediate 13 from Example 1 is treated with one equivalent of oxone to produce sulfoxide 14. Deprotection of 14 by hydrogenolysis over palladium on carbon, treatment with bromotrimethylsilane, and treatment with aqueous HF gives the product 15.

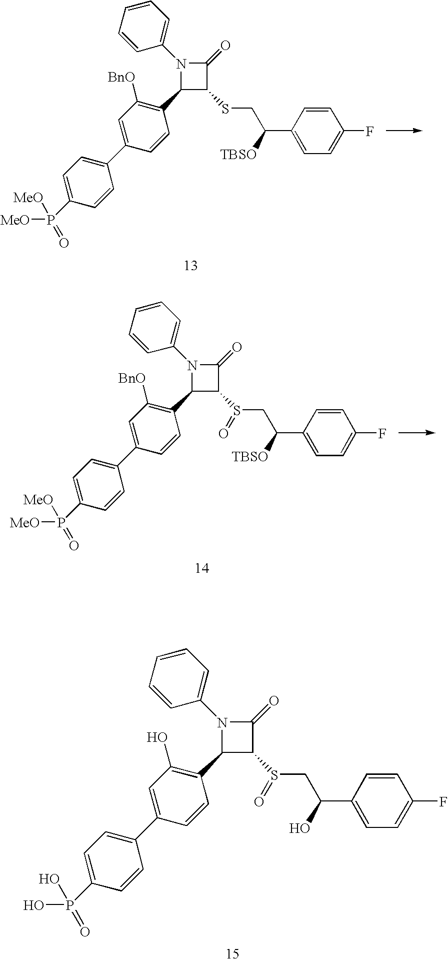

### EXAMPLE 3

In the following sequence the key protected intermediate 13 from Example 1 is treated with two equivalents of oxone to produce sulfone 15. Deprotection of 15 by hydrogenolysis over palladium on carbon, treatment with bromotrimethylsilane, and treatment with aqueous HF gives the product 16.

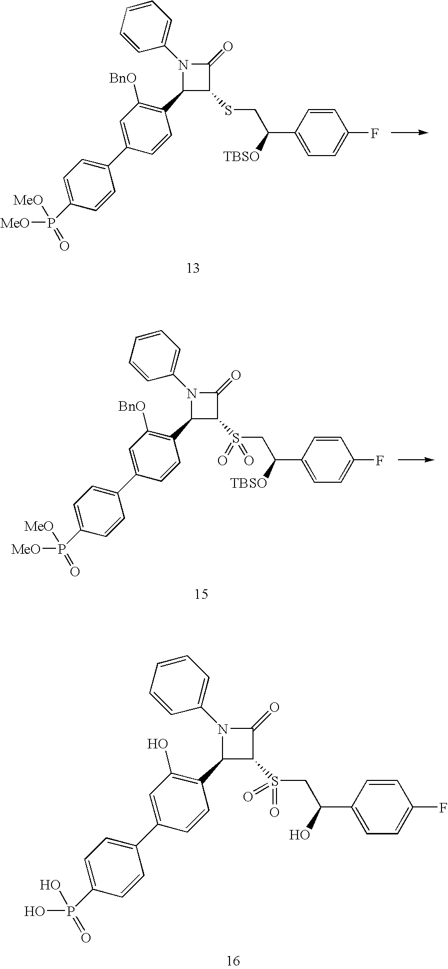

### EXAMPLES 4-6

Treatment of 12, 15, and 16 with Dess-Martin periodinane gives the corresponding ketones 17, 18, and 19, respectively.

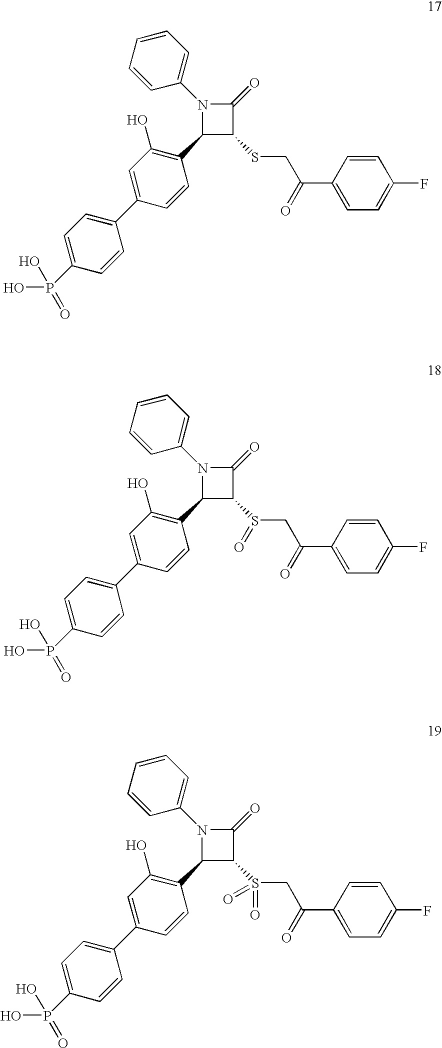

### EXAMPLE 7

Treatment of diglycolic anhydride (20) with 4-fluorophenylmagnesium bromide (21) according to the published procedure (Soulier, R. Bulletin de la Societe Chimique de France 1968, 4, 1530-1534) gives acid 22. Compound 22 is converted to the corresponding mixed anhydride by treatment with pivaloyl chloride and DMAP in DMF and coupled to (+)-(4S)-4-phenyl-2-oxazolidinone (S)-4-phenyl-2-oxazolidinone to afford the imide (23). Treatment of 23 with borane dimethylsulfide complex in the presence of a catalytic amount of (R)-1-methyl-3,3-diphenyltetrahydro-3H-pyrrolo[1,2-c][1,3,2]oxazaborole produced the desired (4S)-4-benzyl-3-[(5S)-5-(4-fluorophenyl)-5-hydroxypentanoyl]-1,3-oxazolidin-2-one (24a) in good yield. Protection of the benzylic alcohol as the TBS ether is accomplished by reaction with tert-butyldimethylsilyl chloride in the presence of imidazole providing 24b. In the next step, 24b is treated with titanium tetrachloride and N-ethyldiisopropylamine followed by treatment with 6b to effect enantiospecific condensation giving 25. Treatment of 25 with excess N,O-bistrimethylsilyl-acetamide followed by a catalytic amount of tetrabutylammonium fluoride hydrate results in ring closure to the desired beta-lactam (26) while maintaining the TBS protecting group on the benzylic alcohol. Suzuki coupling of 26 with 11 gives the expected biphenyl derivative that is deprotected by hydrogenolysis over palladium on carbon, treatment with bromotrimethylsilane, and treatment with aqueous HF to give the product 27. Treatment of 27 Dess-Martin periodinane gives the corresponding ketone 28.

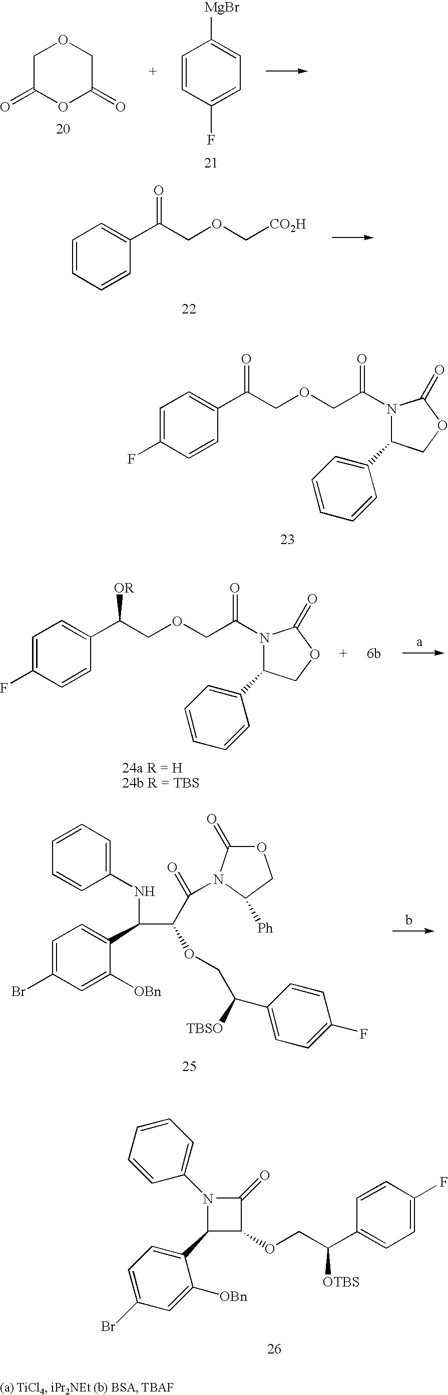

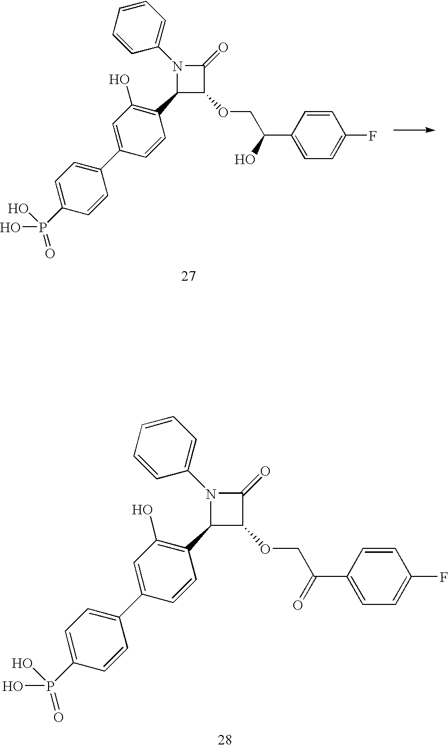

### EXAMPLE 8

(S)-3-Hydroxy-gamma-lactone (29) is converted into beta-lactam 30 according to the published procedure (Wu, G.; Wong, Y. S.; Chen, X.; Ding, Z. J. Org. Chem. 1999, 64, 3714-3718). Selective mesylation of the primary alcohol in 30 followed by treatment with base promotes oxirane formation. The epoxide is then treated with sodium 4-fluorophenoxide to give 31a that is protected as the TBS derivative by treatment with tert-butyldimethylsilyl chloride and imidazole to give 31b. Suzuki coupling of 31b with 11 gives the expected biphenyl derivative that is deprotected by hydrogenolysis over palladium on carbon, treatment with bromotrimethylsilane, and treatment with aqueous HF to give the product 32. Treatment of 32 Dess-Martin periodinane gives the corresponding ketone 33.

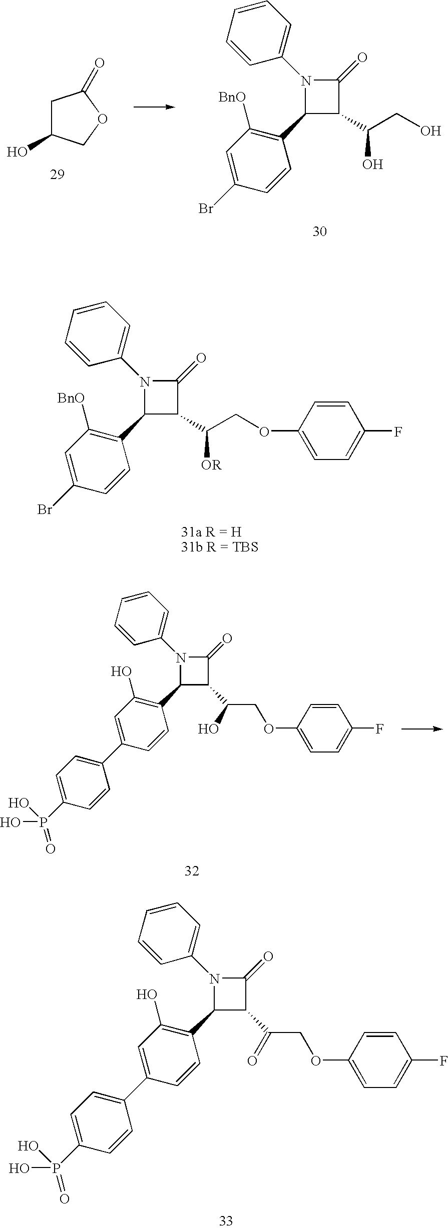

### EXAMPLES 9 AND 10

Substituting sodium 4-fluorothiophenoxide for sodium 4-fluorophenoxide in Example 8 permits the preparation of thioether derivatives 34 and 35.

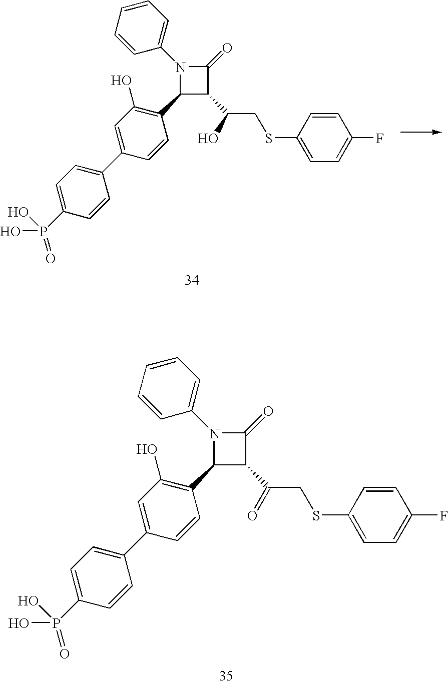

### EXAMPLE 11

Suzuki coupling of 8 from Example 1 with meta-hydroxyphenylboronic acid gives the expected biphenyl derivative 36 that is deprotected by hydrogenolysis over palladium on carbon and treatment with aqueous HF to give the product 37.

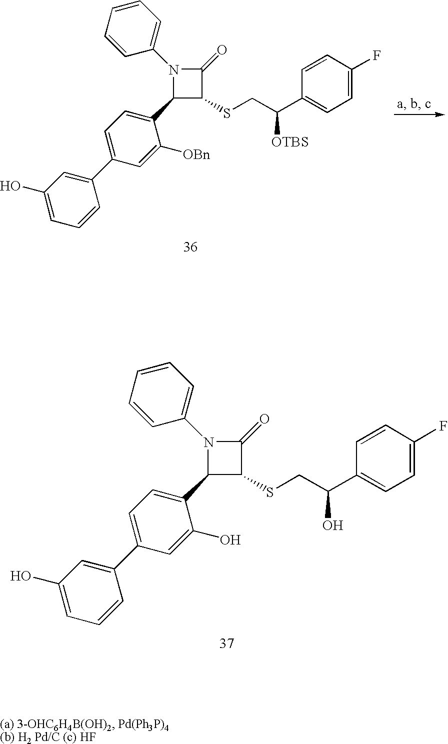

### EXAMPLE 12

In the following sequence the key protected intermediate 36 from Example 11 is treated with one equivalent of oxone to produce sulfoxide 38. Deprotection of 38 by hydrogenolysis over palladium on carbon, treatment with bromotrimethylsilane, and treatment with aqueous HF gives the product 39.

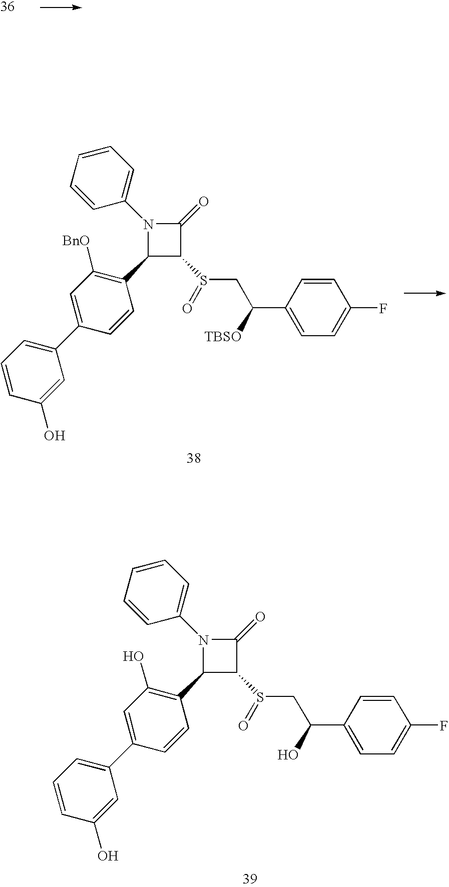

### EXAMPLE 13

In the next sequence the key protected intermediate 36 from Example 11 is treated with two equivalents of oxone to produce sulfone 40. Deprotection of 40 by hydrogenolysis over palladium on carbon and treatment with aqueous HF gives the product 41.

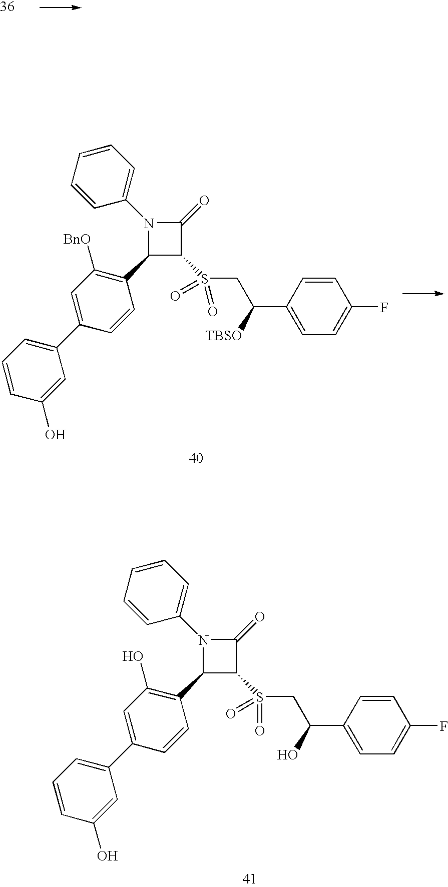

### EXAMPLES 14-16

Treatment of 37, 39, and 41 with Dess-Martin periodinane gives the corresponding ketones 42, 43, and 44 respectively.

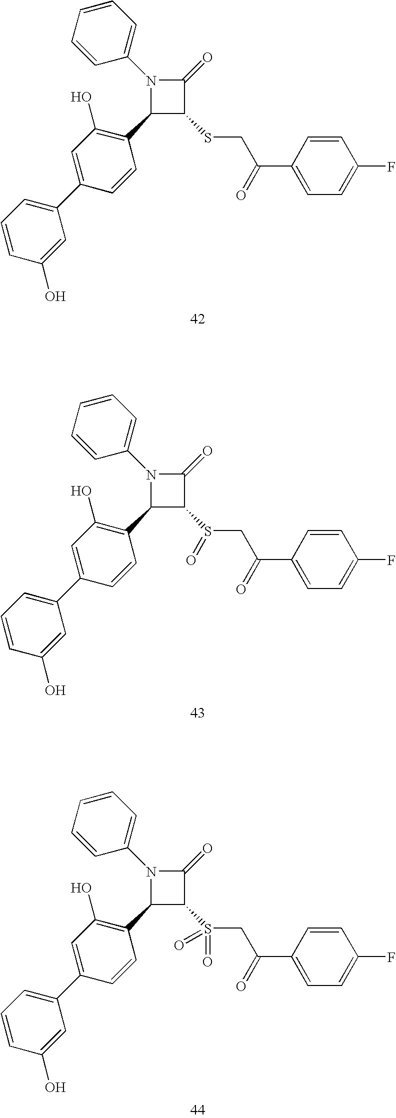

### EXAMPLE 17

In the scheme shown below, the synthesis of a C-glycosyl containing molecule is described. The sequence commences with the reaction of peracetyl D-glucose with 33% HBr in acetic acid to produce the anomeric bromide 45. Treatment of 45 with excess Grignard reagent 46, generated from 1,4-dibromobenzene and magnesium, followed by treatment of the crude product with acetic anhydride in pyridine provides the desired bromophenyl derivative 47. Conversion of 47 to the corresponding pinacol boronate ester 49 was accomplished by reaction with bis(pinicolato)diboron (48) under the influence of palladium catalysis. Suzuki coupling of 48 with 8 gave the expected biphenyl derivative 50 that was deprotected by first hydrolysis in aqueous methanol and triethylamine, hydrogenolysis over palladium on carbon and finally treatment with aqueous HF to give the desired product 51.

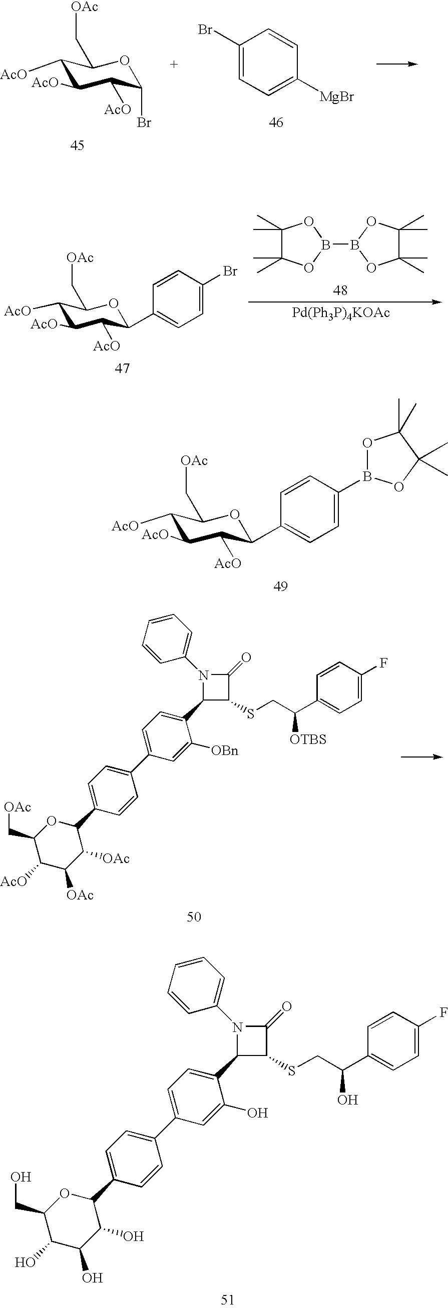

### EXAMPLE 18

In the next sequence the key protected intermediate 50 from Example 17 is treated with one equivalent of oxone to produce sulfoxide 52. Deprotection of 52 by first hydrolysis in aqueous methanol and triethylamine, hydrogenolysis over palladium on carbon and finally treatment with aqueous HF to give the desired product of 53.

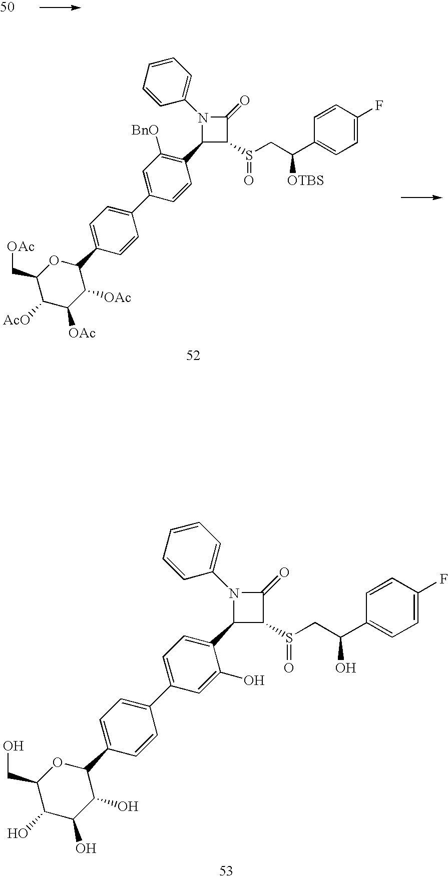

### EXAMPLE 19

In the next sequence the key protected intermediate 50 from Example 17 is treated with two equivalents of oxone to produce sulfone 54. Deprotection of 54 is accomplished in three steps: 1) hydrolysis in aqueous methanol and triethylamine to remove the acetyl groups; 2) hydrogenolysis over palladium on carbon to remove the O-benzyl group: and 3) treatment with aqueous HF to remove the TBS group giving compound 55.

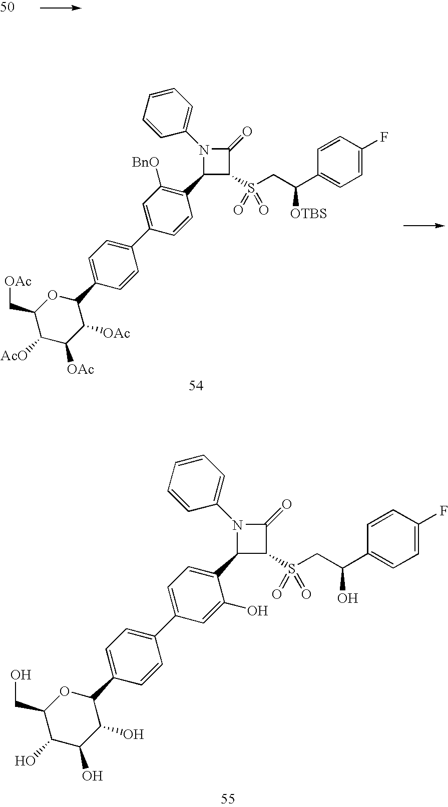

### EXAMPLES 20-25

Treatment of 50, 52, and 54 with aqueous HF followed by hydrogenolysis over palladium on carbon gives compounds 56, 57 and 58. Treatment of 56, 57 and 58 with Dess-Martin periodinane and hydrolysis with aqueous methanol and triethylamine gives the corresponding ketones 59, 60, and 61 respectively.

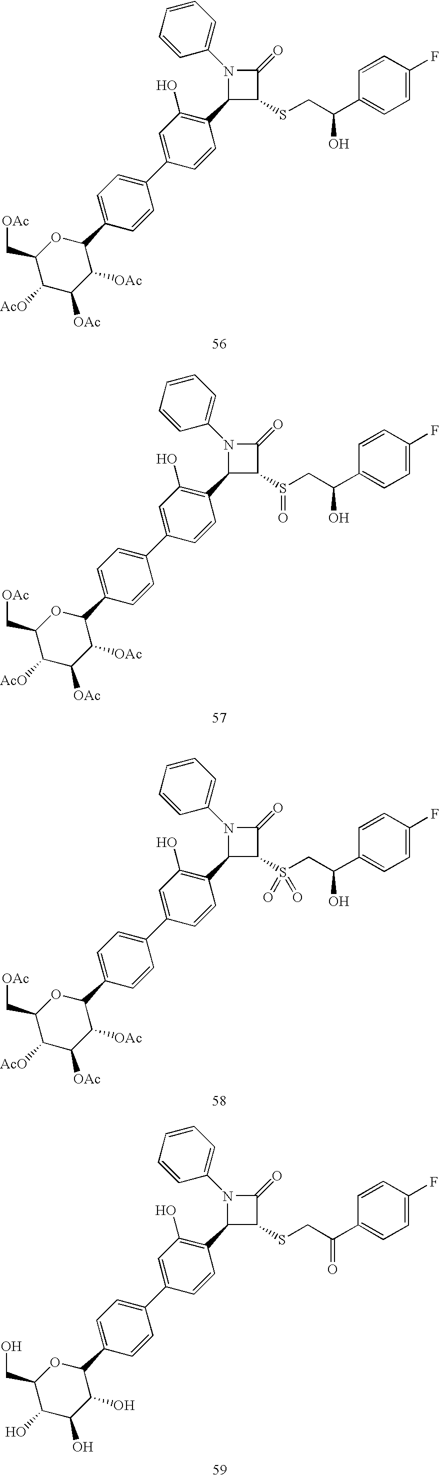

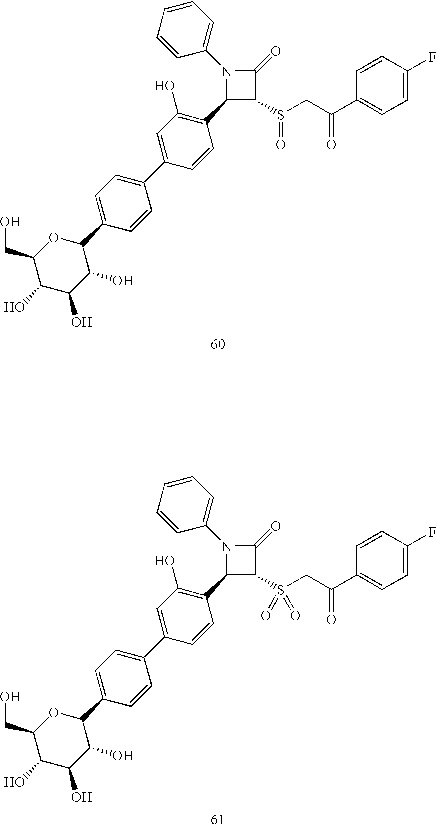

### EXAMPLE 26

The scheme below shows the synthesis of a sulfonic acid containing compound. The sequence begins with the Suzuki coupling of commercially available 4-methylthiophenylboronic acid (62) with 26 to provide the expected biphenyl derivative 63. The methylthio group was elaborated to the corresponding sulfonic acid by the following sequence: 1) conversion to the sulfoxide with metachloroperbenzoic acid: 2) Pummerer rearrangement with trifluoroacetic anhydride: 3) hydrolysis of the trifluoroacetoxymethylthio ether with aqueous methanol in the presence of triethylamine: and 4) oxidation of the thiol to the corresponding sulfonic acid with meta-chloroperbenzoic acid. After conversion of the sulfonic acid to the corresponding sodium salt by ion exchange, the compound was deprotected by first hydrogenolysis over palladium on carbon. Treatment with aqueous HF and finally ion exchange gives the desired product 64.

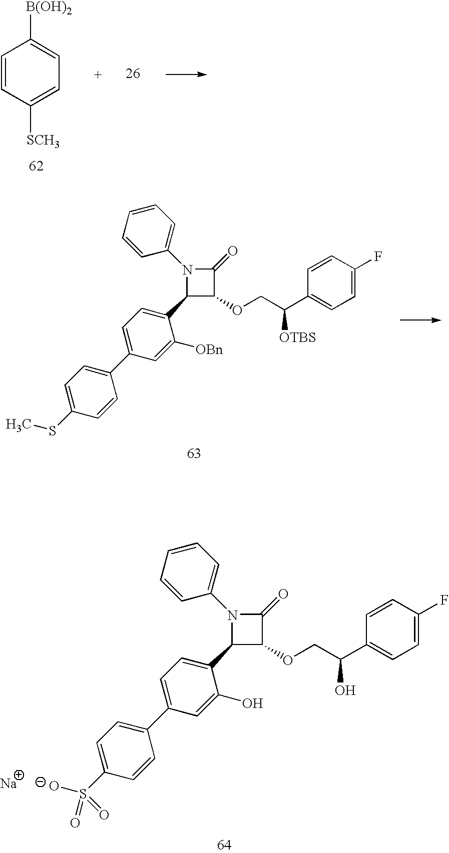

### EXAMPLE 27

In any of the above Examples 1-26 substituting a substituted aniline, for example 4-fluoroaniline (65), for aniline may be employed for the preparation of derivatives in which the N-phenyl moiety is substituted. Thus, condensation of 4 with 65 leads to the formation of imine 66, which is condensed with 3b to give 67, as described in Example 1. Ring closure of 67, as described in Example 1, provides key intermediate 68, which can serve as a precursor for 4-fluoro substituted compounds.

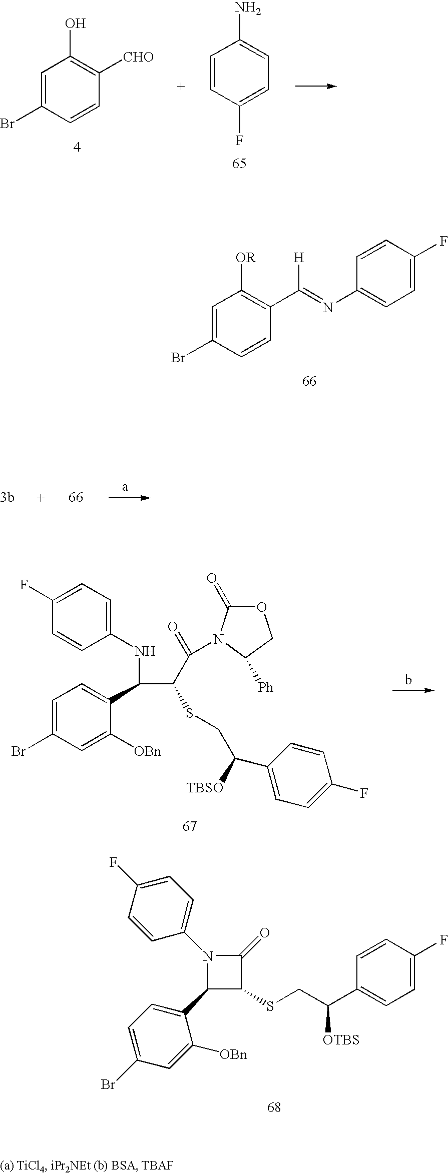

### EXAMPLES 28-31

Using the key intermediate 68 from Example 27 allows the preparation of a variety of compounds such as the following:

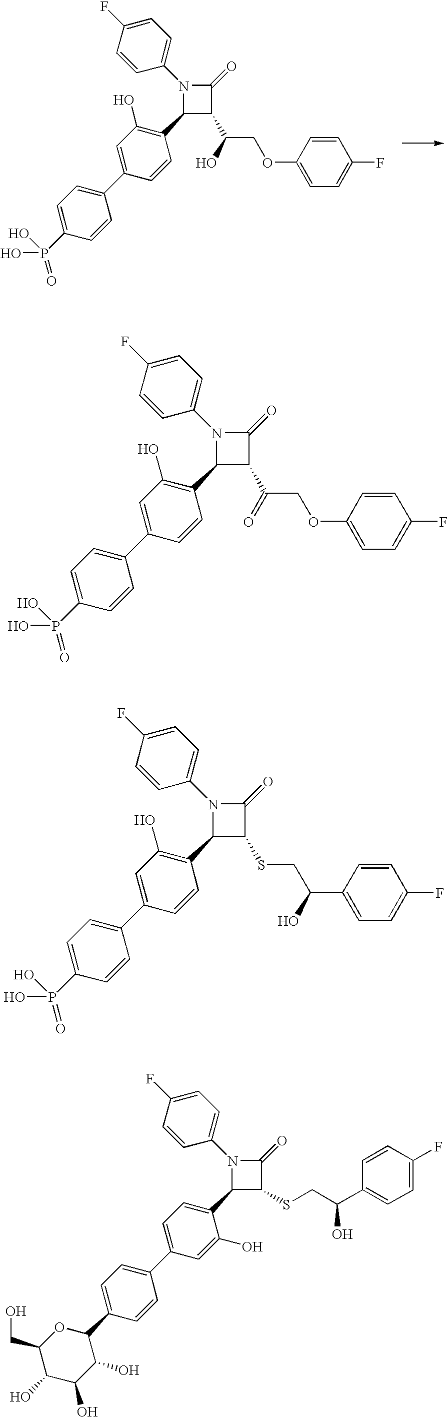

### EXAMPLE 32

Compound 36 from Example 8 is reacted with trichloroacetimidate donor methyl 2,3,4-tri-O-acetyl-1-O-(2,2,2-trichloroethanimidoyl)-□-D-glucopyranuronate (prepared according to Urban, F.; Moore, B.; Breitenbach, R. Tetrahedron Lett. 1990, 31, 4221) in methylene chloride at −25° C. with boron trifluoride etherate as an activator for the coupling giving 69. After glycosylation, hydrolysis of the ester groups was accomplished by addition of TEA:MeOH:H₂O (1:1:3.5, 0.01M) to afford the unprotected glucuronic acid. Removal of the TBS group by treatment with aqueous HF and catalytic (Pd/C) hydrogenolysis of the benzyl group gives the glucuronide derivative 70.

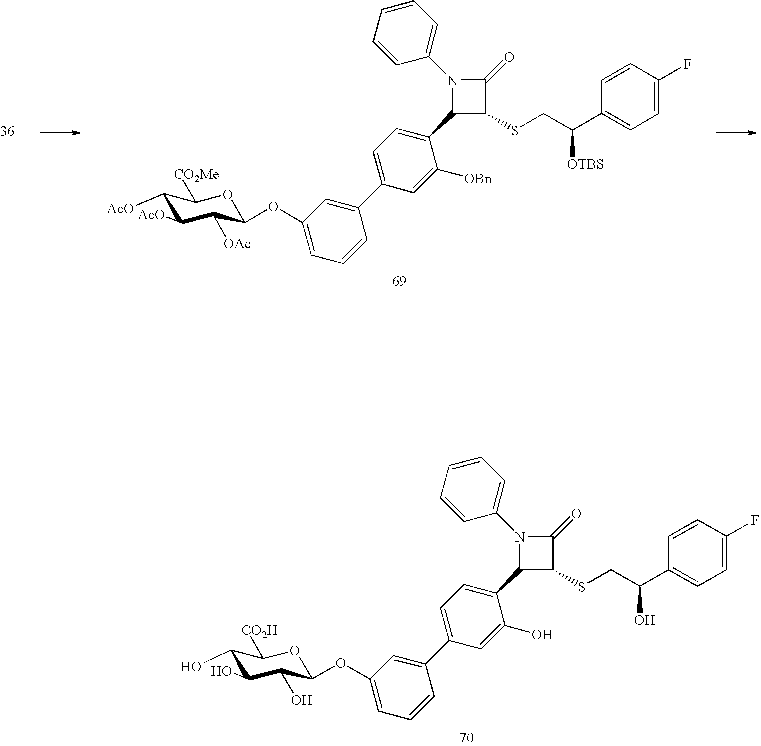

### EXAMPLE 33

The scheme shown below illustrates the synthesis of a sugar carbamate-containing compound. Compound 36 from Example 11 is treated with aqueous HF to provide the corresponding deprotected benzylic alcohol 71. Treatment of 71 by standard means (acetic anhydride, TEA, DMAP) followed by selective phenolic acetate cleavage with guanidine and MeOH (in accord with the method of Kunesch, N.; Miet, C.; Poisson, J. Tetrahedron Lett. 1987, 28, 3569 with the slight modification of the addition of catalytic DMAP) affords desired compound 72. In the next step, compound 72 is reacted with heptaacetyl-beta-D-cellobioslyl bromide (73) in acetonitrile in the presence of anhydrous zinc fluoride to give 74. The ester groups of 74 are removed by addition to as solution of TEA:MeOH:H₂O (1:1:3.5, 0.01M) to afford 75. The sugar carbamate is prepared by first protecting the 4″,6″-hydroxyls of 75 as a p-methoxybenzylidene followed by exhaustive acylation with chloroacetic anhydride to give the fully protected derivative 76. The benzylidene is then cleaved with acid to give diol 77. The diol 77 is then reacted with 2-fluorophenyl isocyanate with copper (I) catalysis to give 78. The chloroacetates are removed by treatment with sodium methoxide in methanol to give 79 which is hydrogenated over palladium on carbon to provide 80.

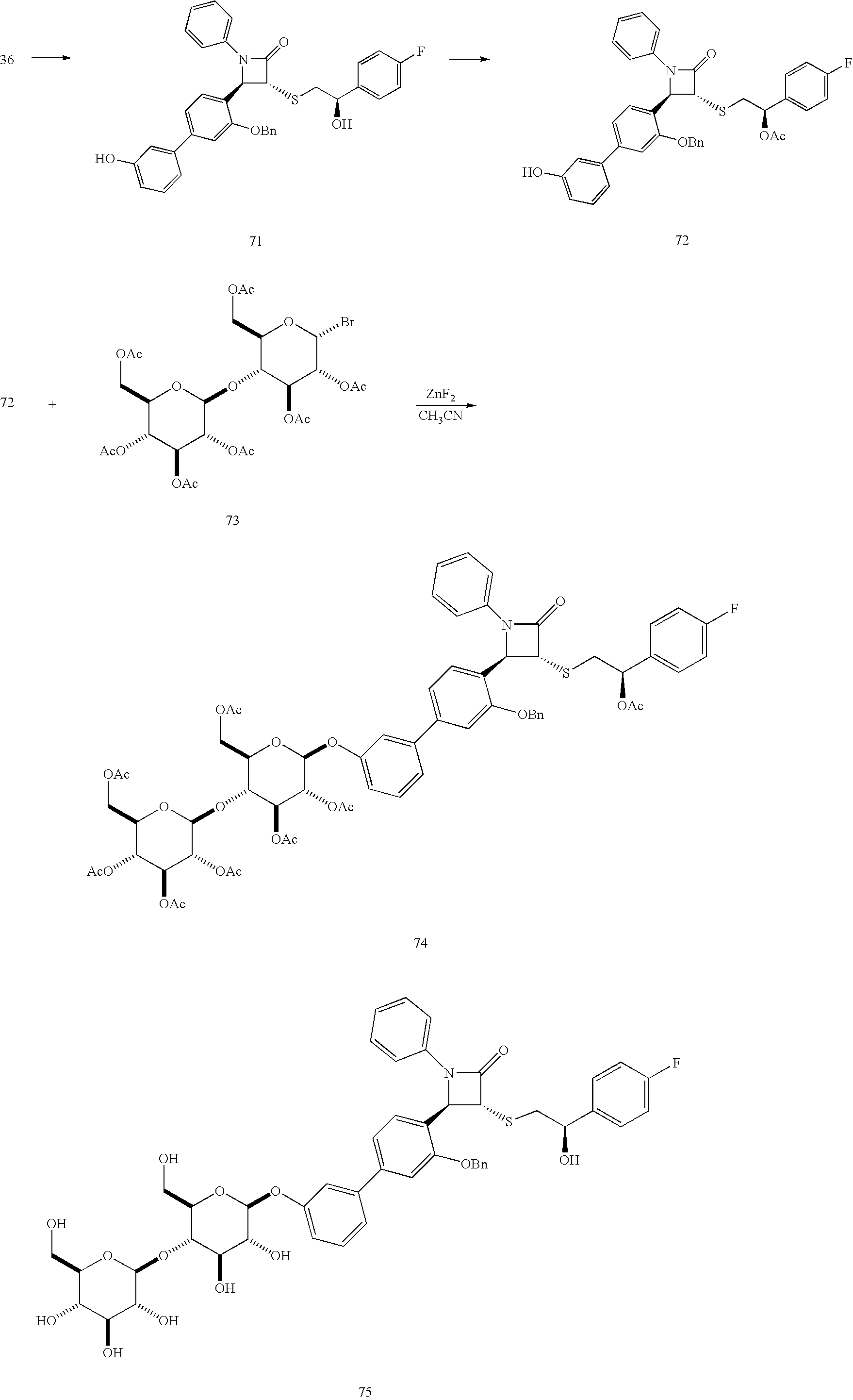

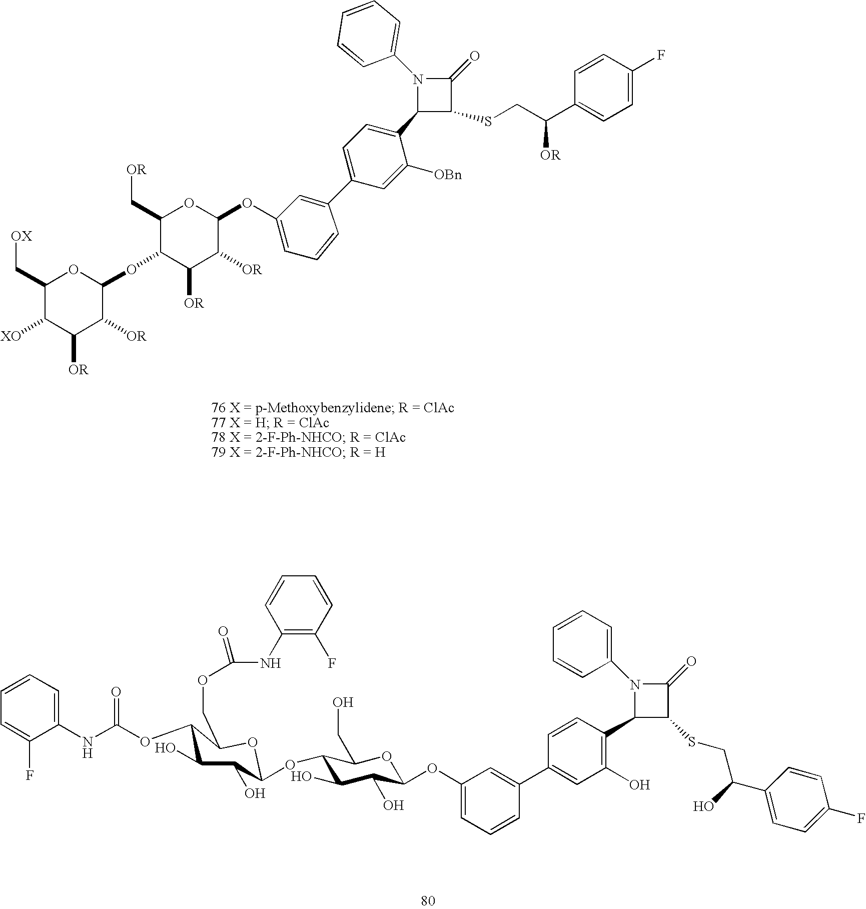

### EXAMPLE 34

The scheme shown below illustrates the synthesis of a sugar carbamate-containing compound. Chemoselective reaction of the primary hydroxyl groups of 75 with 2-fluorophenyl isocyanate gives bis-carbamate 81a. Hydrogenolysis of 81a over palladium on carbon gives compound 81b.

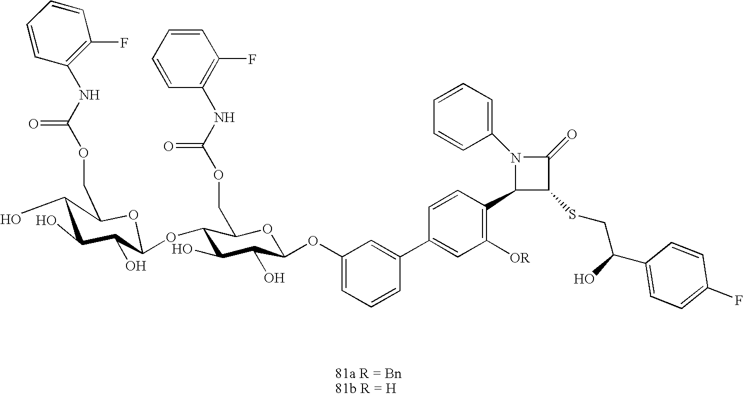

### EXAMPLES 35-37

Treatment of commercially available 3-chloro-4′-fluoropropiophenone (82) with diisopinocamphenylchloroborane, (Srebnik, M.; Ramachandran, P. V.; Brown, H. C. J. Org. Chem. 1988, 53, 2916-2920), gives [S]-(−)-3-chloro-1-(4-fluorophenyl)propanol (83a). Treatment of 83a with tert-butyldimethylsilyl chloride and imidazole gives 83b. Reaction of 83b with sodium mercaptoacetate in the presence of DBU gives 84. Compound 84 is converted to the corresponding mixed anhydride by treatment with pivaloyl chloride and DMAP in DMF and then coupled to (S)-4-phenyl-2-oxazolidinone to afford the imide (85). In the next step, 85 is treated with titanium tetrachloride and N-ethyldiisopropylamine followed by treatment with 6b to effect enantiospecific condensation providing 86. Treatment of 86 with excess N, O-bis(trimethylsilyl)acetamide followed by a catalytic amount of tetrabutylammonium fluoride hydrate results in ring closure to the desired beta-lactam (87) while maintaining the TBS protecting group on the benzylic alcohol. Suzuki coupling of 87 with 11 gives the expected biphenyl derivative that is deprotected by hydrogenolysis over palladium on carbon, treatment with bromotrimethylsilane, and treatment with aqueous HF to give the product 88. Using the methods described above compounds 89 and 90 can also be prepared from key intermediate 87.

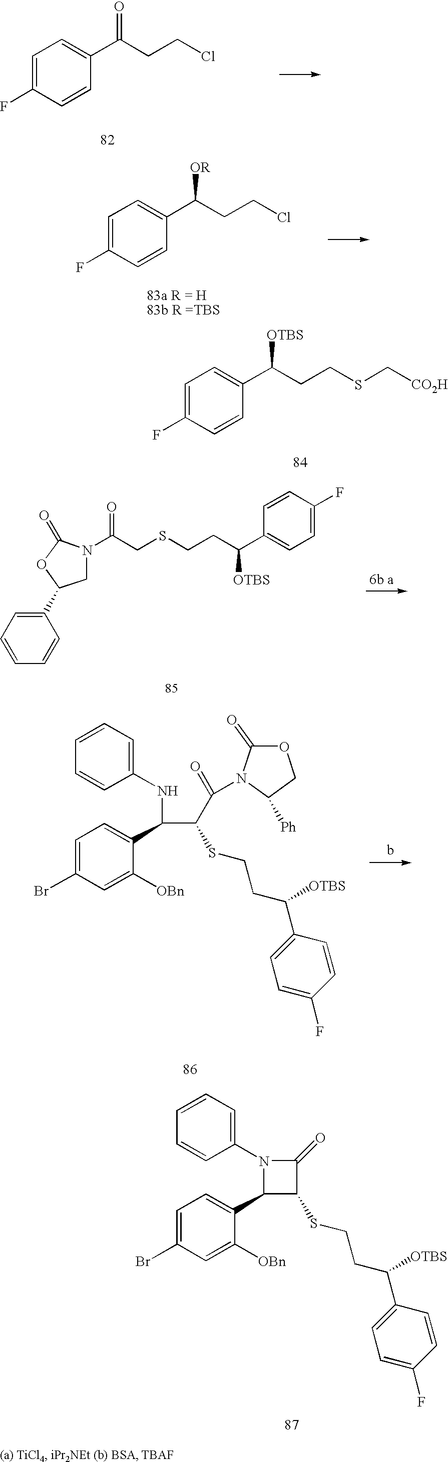

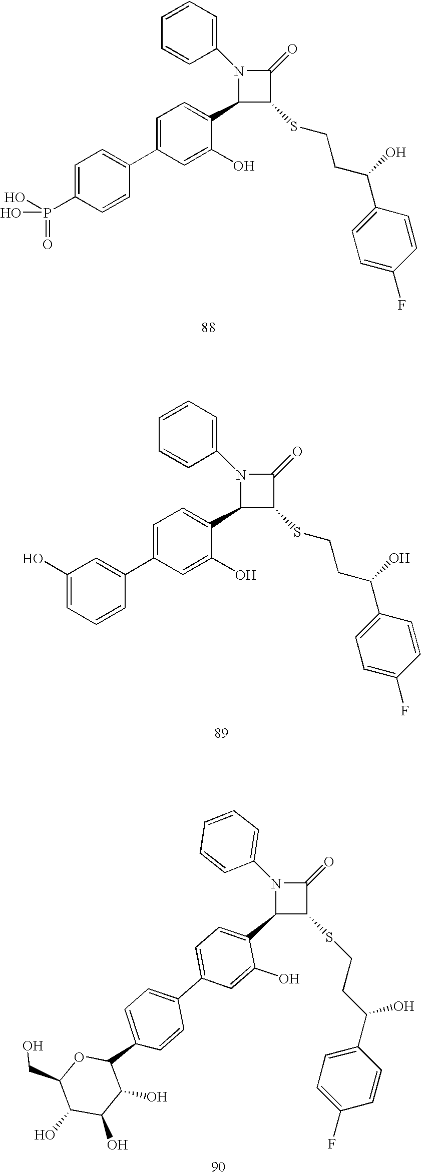

### EXAMPLES 38-39

The Suzuki coupling product that serves as a precursor to 88, namely compound 91, is a useful intermediate for the preparation of the sulfoxide 94 and sulfone 95 analogs. Treatment of 91 with one or two equivalents of oxone produces the protected compounds 92 or 93 respectively. Deprotection of 92 and 93 as described above provides compounds 94 and 95.

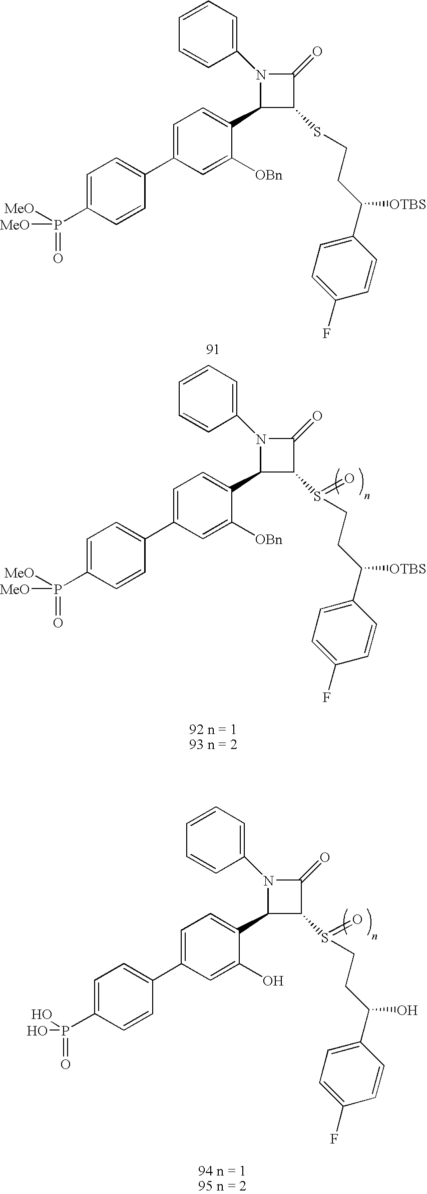

### EXAMPLES 40-43

Using the same experimental procedures described above, compounds 96-99 can be prepared from the appropriate starting materials and the proper number of equivalents of oxone, as would be known by one of ordinary skill in the chemical arts.

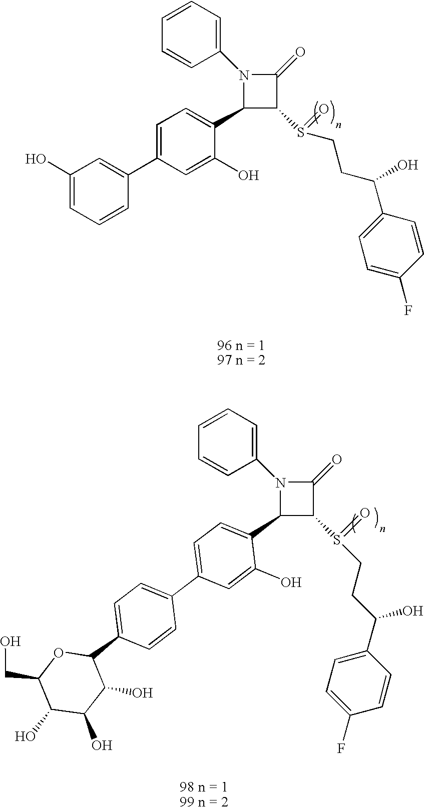

### EXAMPLES 44-52

The ketone containing inhibitors 100-108 can be prepared from the protected intermediates described above by selective removal of the TBS moiety with HF and oxidation of the resulting alcohol with the Dess-Martin periodinane reagent. If desired, the oxidation state of the sulfur atom can be altered by treatment with one or two equivalents of oxone to provide the corresponding sulfoxides and sulfones.

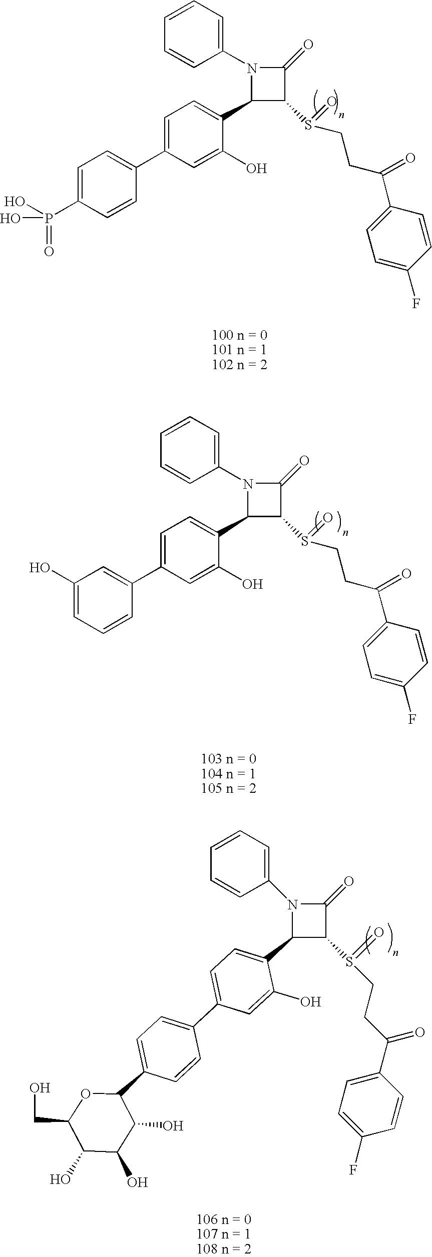

### EXAMPLES 53-55

Treatment of commercially available 2-bromo-4′-fluoroacetophenone (109) with diisopinocamphenylchloroborane, (Srebnik, M.; Ramachandran, P. V.; Brown, H. C. J. Org. Chem. 1988, 53, 2916-2920), gives (1R)-2-bromo-1-(4-fluorophenyl)ethanol (110a). Treatment of 110a with tert-butyldimethylsilyl chloride and imidazole gives 110b. Reaction of 110b with sodium mercaptoacetate in the presence of DBU gives 111. Compound 111 is converted to the corresponding mixed anhydride by treatment with pivaloyl chloride and DMAP in DMF and then coupled to (S)-4-phenyl-2-oxazolidinone to afford the imide (112). In the next step, 112 is treated with titanium tetrachloride and N-ethyldiisopropylamine followed by treatment with 6b to effect enantiospecific condensation providing 113. Treatment of 113 with excess N,O-bis(trimethylsilyl)acetamide followed by a catalytic amount of tetrabutylammonium fluoride hydrate results in ring closure to the desired beta-lactam (114) while maintaining the TBS protecting group on the benzylic alcohol. Suzuki coupling of 114 with 11 gives the expected biphenyl derivative that is deprotected by hydrogenolysis over palladium on carbon, treatment with bromotrimethylsilane, and treatment with aqueous HF to give the product 115. Using the methods described above compounds 116 and 117 can also be prepared from key intermediate 114.

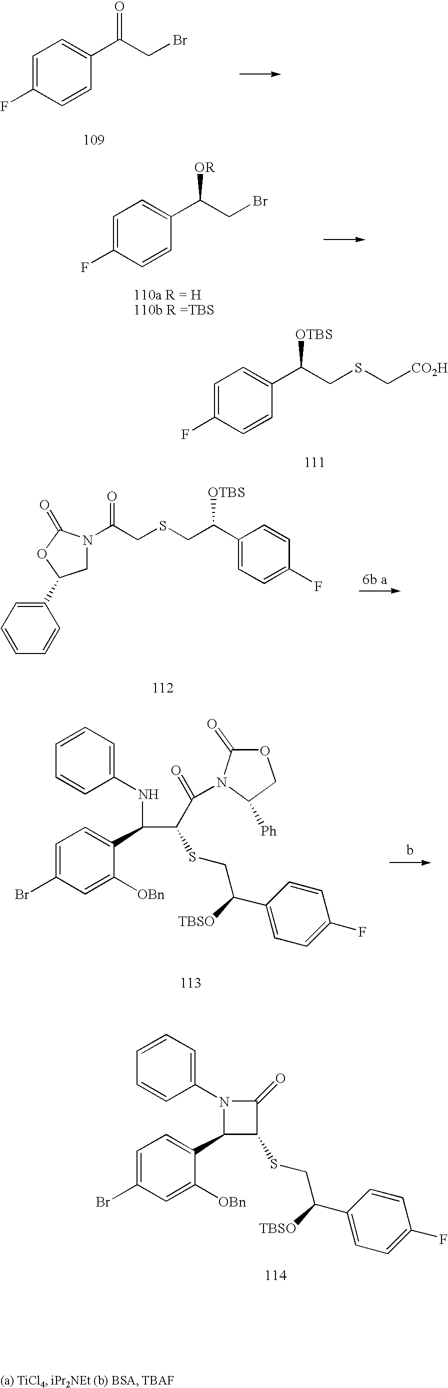

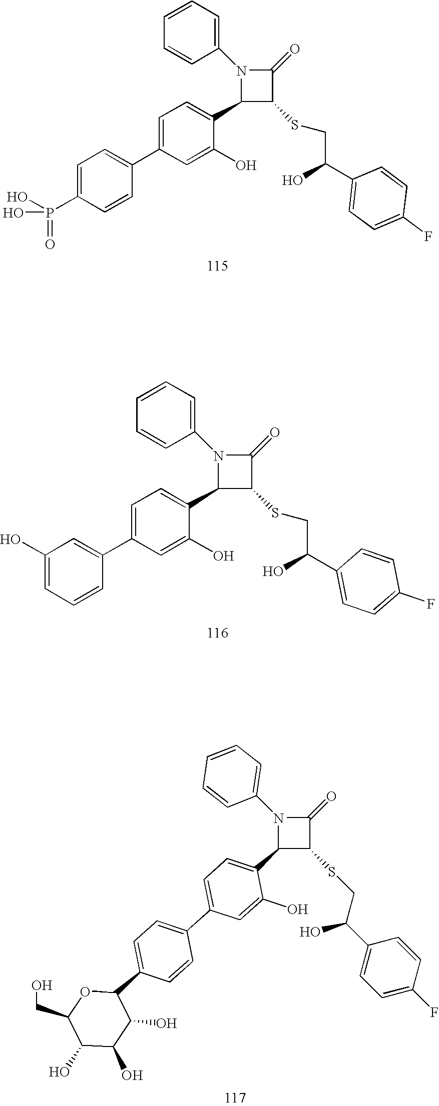

### EXAMPLES 56-58

Commercially available 3-(4-fluorobenzoyl)propionic acid (118) is converted to the corresponding mixed anhydride by treatment with pivaloyl chloride and DMAP in DMF and then coupled to (S)-4-phenyl-2-oxazolidinone to afford the imide (119). Treatment of 119 with borane dimethylsulfide complex in the presence of a catalytic amount of (R)-1-methyl-3,3-diphenyltetrahydro-3H-pyrrolo[1,2-c][1,3,2]oxazaborole gives compound (120a). Protection of the benzylic alcohol as the TBS ether is accomplished by reaction with tert-butyldimethylsilyl chloride in the presence of imidazole as a base to provide 120b. In the next step, 120b is treated with titanium tetrachloride and N-ethyldiisopropylamine followed by treatment with 6b to effect enantiospecific condensation providing 121. Treatment of 121 with excess N, O-bis(trimethylsilyl)acetamide followed by a catalytic amount of tetrabutylammonium fluoride hydrate results in ring closure to the desired beta-lactam (122) while maintaining the TBS protecting group on the benzylic alcohol. Suzuki coupling of 122 with 11 gives the expected biphenyl derivative that is deprotected by hydrogenolysis over palladium on carbon, treatment with bromotrimethylsilane, and treatment with aqueous HF to give the product 123. Using the methods described above compounds 124 and 125 can also be prepared from key intermediate 122.

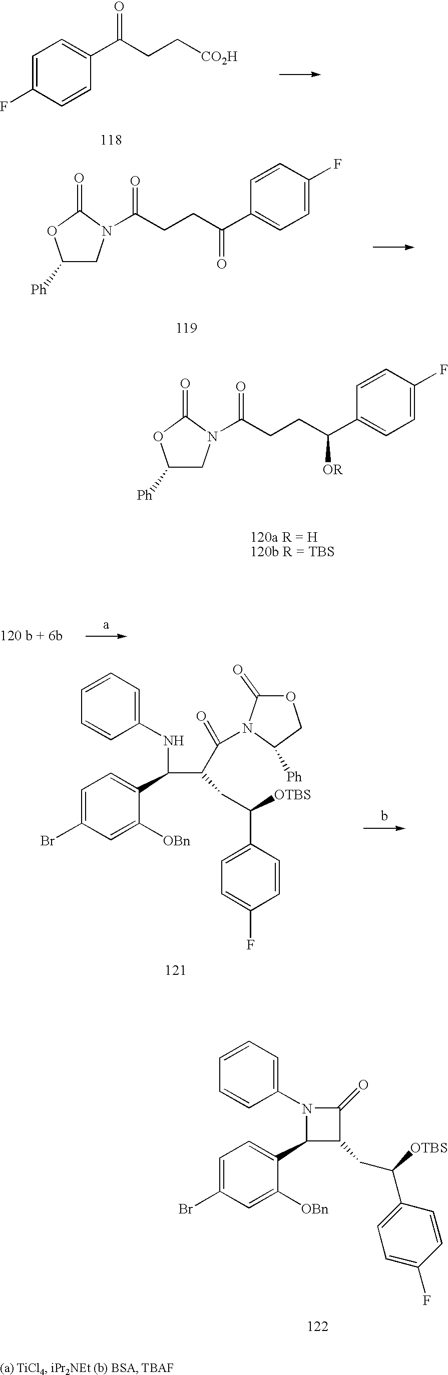

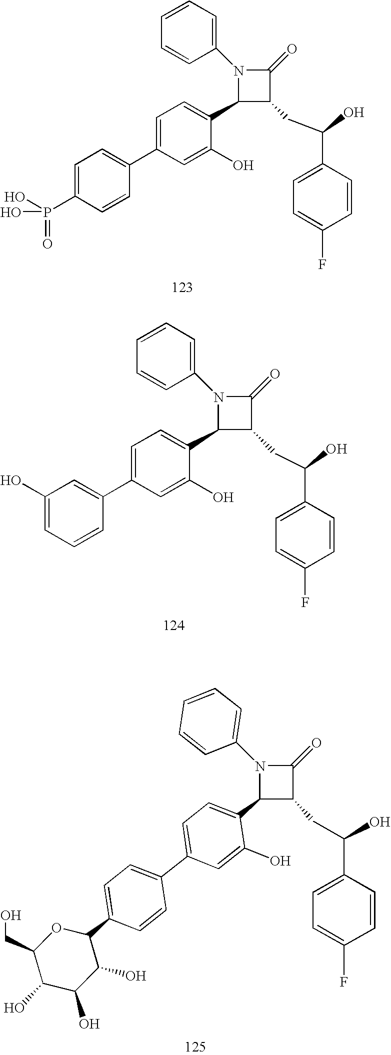

### EXAMPLES 59-61

Methyl adipoyl chloride (126) is treated with 4-fluorophenylzinc bromide (127) in the presence of cuprous iodide to give methyl 5-(4-fluorophenyl)-5-oxopentanoic 128 (R=Me). Hydrolysis of the ester moiety with aqueous lithium hydroxide in methanol gives the acid (128 R═OH). The mixed anhydride of 128 is formed by treatment with pivaloyl chloride and DMAP in DMF and then coupled to (S)-4-phenyl-2-oxazolidinone to afford the imide (129). Treatment of 129 with borane dimethylsulfide complex in the presence of a catalytic amount of (R)-1-methyl-3,3-diphenyltetrahydro-3H-pyrrolo[1,2-c][1,3,2]oxazaborole gives compound (130a). Protection of the benzylic alcohol as the TBS ether is accomplished by reaction with tert-butyldimethylsilyl chloride in the presence of imidazole as a base to provide 130b. In the next step, 130b is treated with titanium tetrachloride and N-ethyldiisopropylamine followed by treatment with 6b to effect enantiospecific condensation providing 131. Treatment of 131 with excess N, O-bis(trimethylsilyl)acetamide followed by a catalytic amount of tetrabutylammonium fluoride hydrate results in ring closure to the desired beta-lactam (132) while maintaining the TBS protecting group on the benzylic alcohol. Suzuki coupling of 132 with 11 gives the expected biphenyl derivative that is deprotected by hydrogenolysis over palladium on carbon, treatment with bromotrimethylsilane, and treatment with aqueous HF to give the product 133. Using the methods described above compounds 134 and 135 can also be prepared from key intermediate 132.

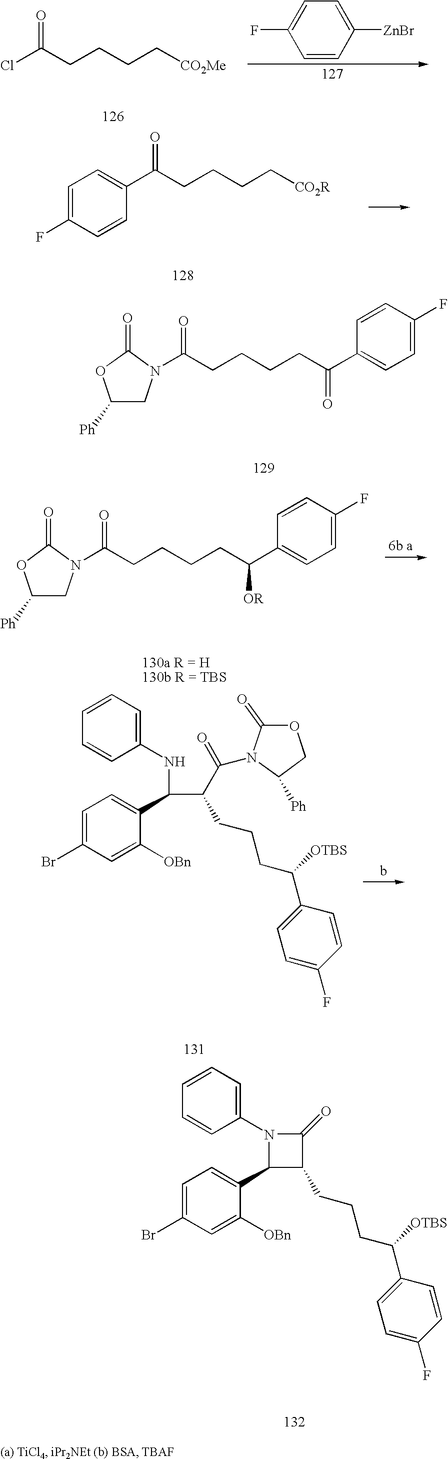

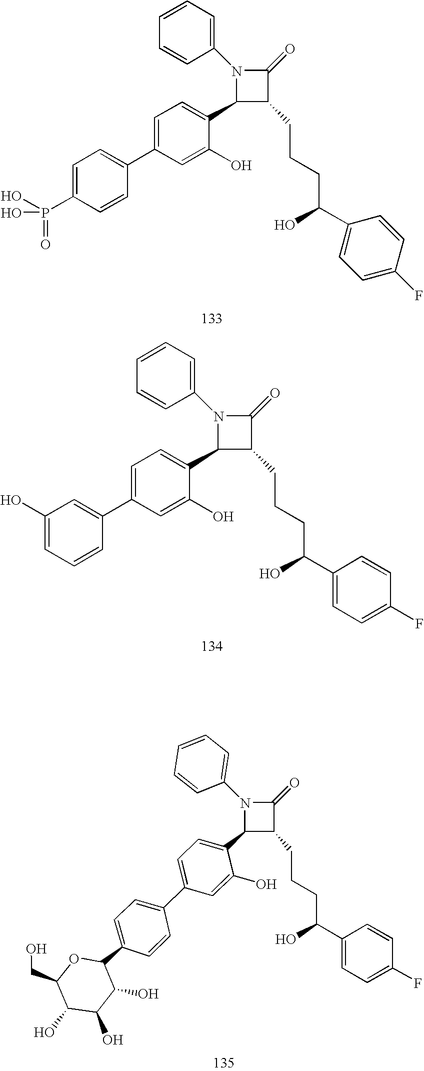

### EXAMPLES 62-64

In a sequence modeled after a published procedure, (Soulier, R.; Vieles, P. Bull. Soc. Chim. France 1968, 394-401), oxybis(acetic acid) (136) is converted to the corresponding anhydride 137 with acetic anhydride. Treatment of 137 with 4-fluorophenylmagnesium bromide gives keto acid 138. Compound 138 is converted to the corresponding mixed anhydride by treatment with pivaloyl chloride and DMAP in DMF and then coupled to (S)-4-phenyl-2-oxazolidinone to afford the imide (139). Treatment of 139 with borane dimethylsulfide complex in the presence of a catalytic amount of (R)-1-methyl-3,3-diphenyltetrahydro-3H-pyrrolo[1,2-c][1,3,2]oxazaborole gives compound (140a). Protection of the benzylic alcohol as the TBS ether is accomplished by reaction with tert-butyldimethylsilyl chloride in the presence of imidazole as a base to provide 140b. In the next step, 140b is treated with titanium tetrachloride and N-ethyldiisopropylamine followed by treatment with 6b to effect enantiospecific condensation providing 141. Treatment of 141 with excess N,O-bis(trimethylsilyl)acetamide followed by a catalytic amount of tetrabutylammonium fluoride hydrate results in ring closure to the desired beta-lactam (142) while maintaining the TBS protecting group on the benzylic alcohol. Suzuki coupling of 142 with 11 gives the expected biphenyl derivative that is deprotected by hydrogenolysis over palladium on carbon, treatment with bromotrimethylsilane, and treatment with aqueous HF to give the product 143. Using the methods described above compounds 144 and 145 can also be prepared from key intermediate 142.

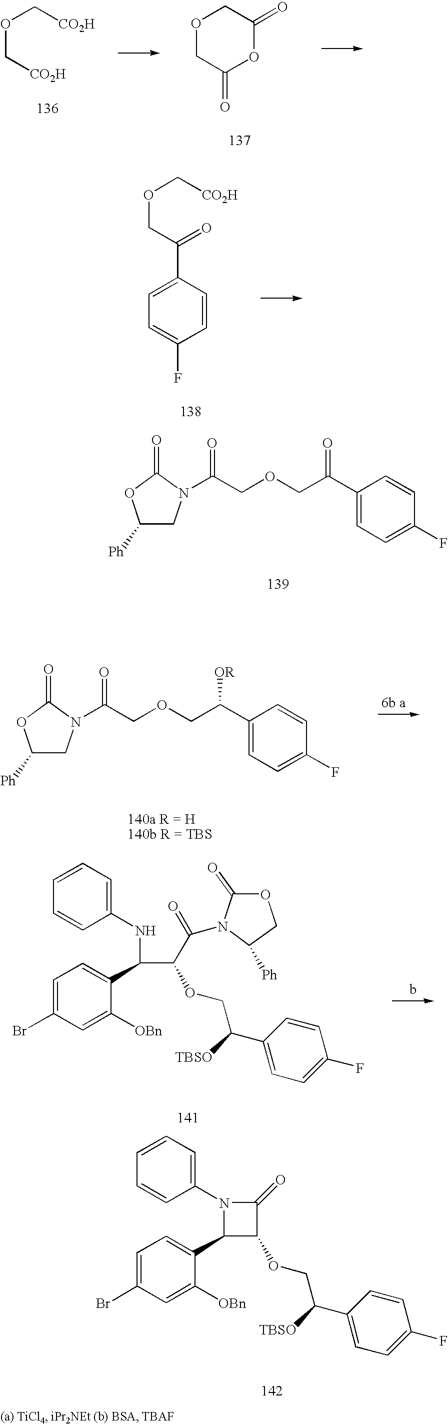

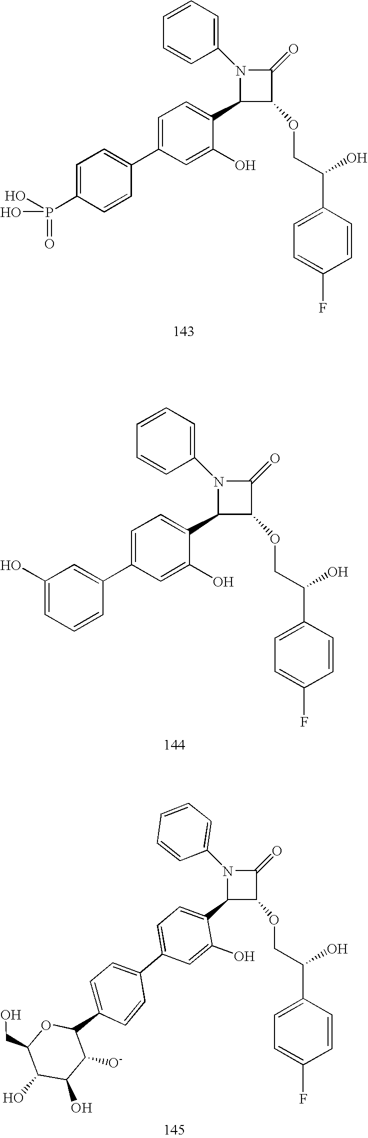

### EXAMPLES 65-67

Reaction of 83b with disodium hydroxyacetate in DMF gives 146. Compound 146 is converted to the corresponding mixed anhydride by treatment with pivaloyl chloride and DMAP in DMF and then coupled to (S)-4-phenyl-2-oxazolidinone to afford the imide (147). In the next step, 147 is treated with titanium tetrachloride and N-ethyldiisopropylamine followed by treatment with 6b to effect enantiospecific condensation providing 148. Treatment of 148 with excess N,O-bis(trimethylsilyl)acetamide followed by a catalytic amount of tetrabutylammonium fluoride hydrate results in ring closure to the desired beta-lactam (149) while maintaining the TBS protecting group on the benzylic alcohol. Suzuki coupling of 149 with 11 gives the expected biphenyl derivative that is deprotected by hydrogenolysis over palladium on carbon, treatment with bromotrimethylsilane, and treatment with aqueous HF to give the product 150. Using the methods described above compounds 151 and 152 can also be prepared from key intermediate 149.

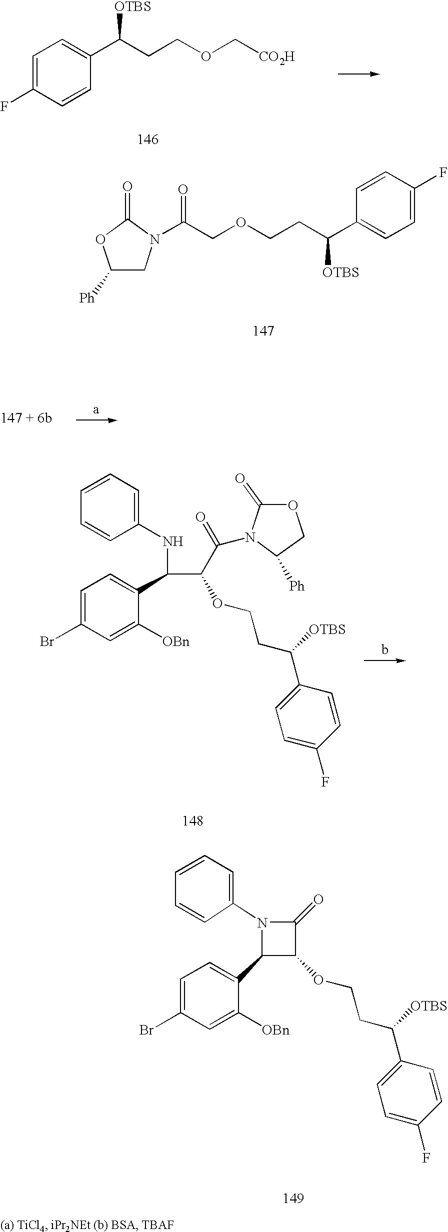

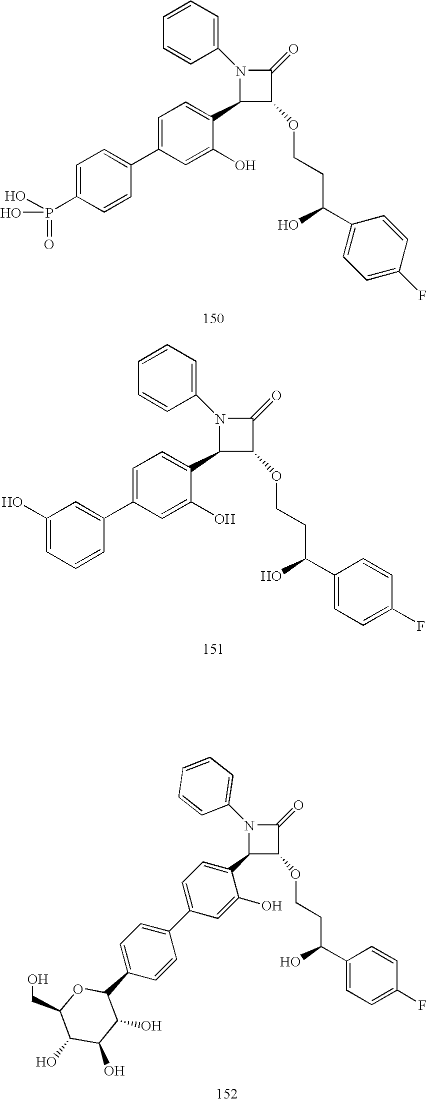

### EXAMPLE 68

Treatment of 152 with Dess-Martin periodinane gives the corresponding ketone 152a.

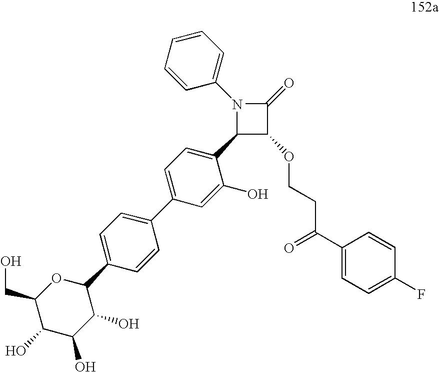

### EXAMPLES 69-71

Compound 153 is available by the literature methods; (Bonini, C.; Bianco, A.; Di Fabio, R.; Mecozzi, S.; Proposito, A.; Righi, G. Gazz. Chim. Ital. 1991, 121, 75-80 and Kunath, A.; Henkel, B.; Wagner, J. J. Chrom. 1993, 634, 119-24). Protection of the alcohols as the benzaldehyde acetal and saponification of the ester with aqueous lithium hydroxide in methanol gives the corresponding acid. The acid is converted to the corresponding mixed anhydride by treatment with pivaloyl chloride and DMAP in DMF and then coupled to (S)-4-phenyl-2-oxazolidinone to afford the imide (154). In the next step, 154 is treated with titanium tetrachloride and N-ethyldiisopropylamine followed by treatment with 6b to effect enantiospecific condensation providing 155. Treatment of 155 with excess N,O-bis(trimethylsilyl)acetamide followed by a catalytic amount of tetrabutylammonium fluoride hydrate results in ring closure to the desired beta-lactam (156). Suzuki coupling of 156 with 11 gives the expected biphenyl derivative that is deprotected by hydrogenolysis over palladium on carbon and treatment with bromotrimethylsilane to give compound 157. Using the methods described above compounds 158 and 159 can also be prepared from key intermediate 156.

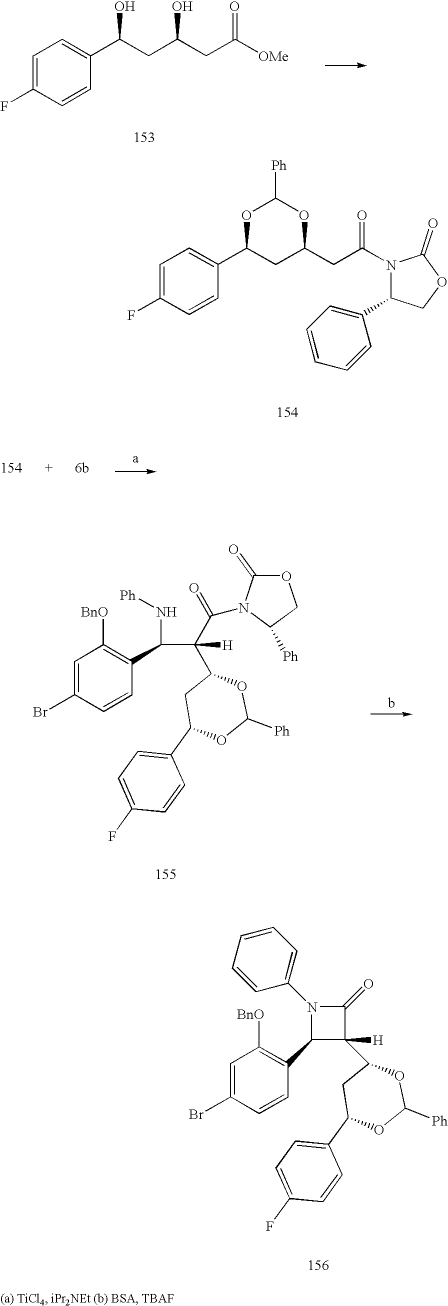

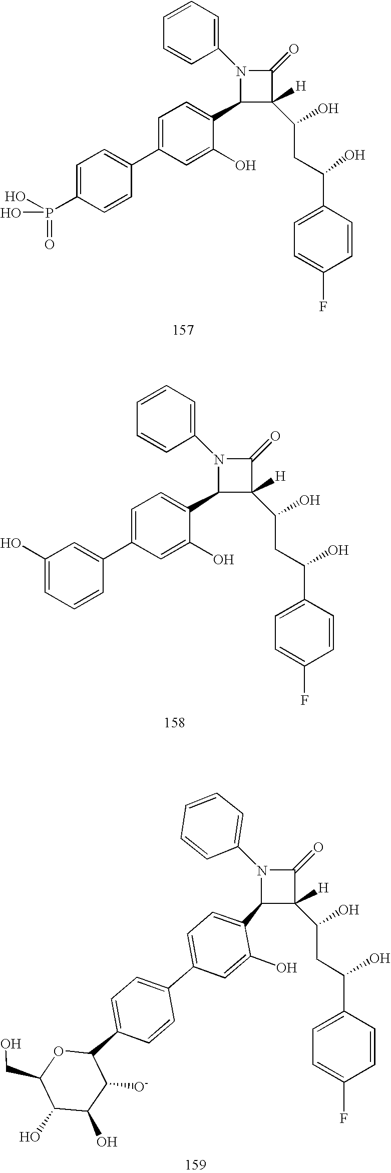

### EXAMPLE 72

Suzuki coupling of bromide 87 and 3-benzyloxyphenylboronic acid using 2.0 M aqueous potassium carbonate as a base and palladium (0) tetrakis(triphenylphosphine) as the cross-coupling catalyst gives biphenyl 160. Treatment of 160 with aqueous HF removes the silyl protecting group giving alcohol 161 that is reacted with trichloroacetimidate donor methyl 2,3,4-tri-O-acetyl-1-O-(2,2,2-trichloroethanimidoyl)-□-D-glucopyranuronate (prepared according to Urban, F.; Moore, B.; Breitenbach, R. Tetrahedron Lett. 1990, 31, 4221) in methylene chloride at −25° C. with boron trifluoride etherate as an activator for the coupling. After glycosylation, hydrolysis of the ester groups is accomplished by addition of TEA:MeOH:H₂O (1:1:3.5, 0.01M) to afford desired glucuronic acid 162. Finally, hydrogenolysis of the benzyl groups over Pearlman's catalyst gives 163.

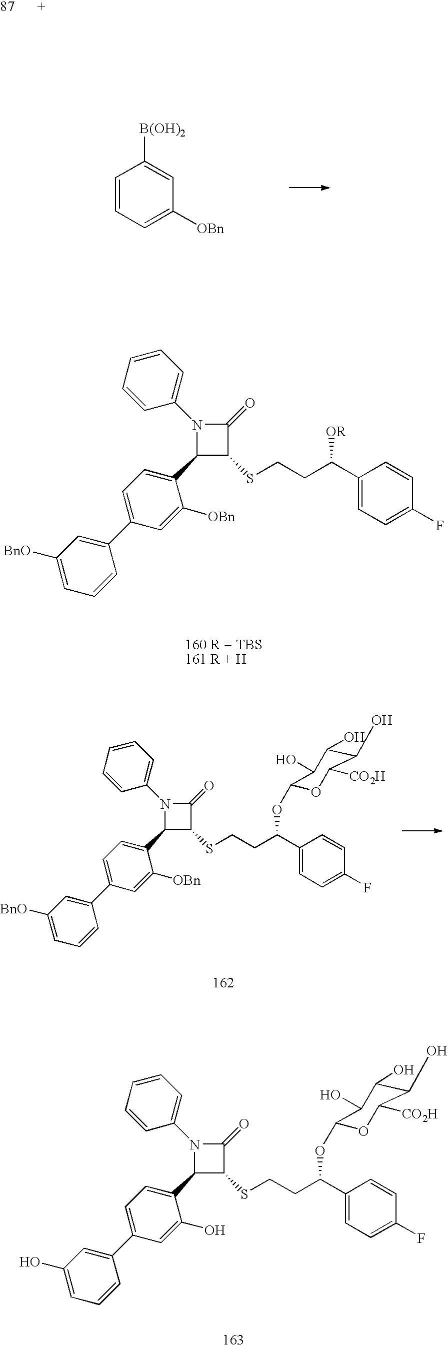

### EXAMPLE 73

Treatment of 54 with aqueous HF removes the silyl protecting group giving alcohol 164 that is reacted with trichloroacetimidate donor methyl 2,3,4-tri-O-acetyl-1-O-(2,2,2-trichloroethanimidoyl)-□-D-glucopyranuronate (prepared according to Urban, F.; Moore, B.; Breitenbach, R. Tetrahedron Lett. 1990, 31, 4221) in methylene chloride at −25° C. with boron trifluoride etherate as an activator for the coupling giving 165. Hydrolysis of the ester groups 165 by treatment with TEA:MeOH:H₂O (1:1:3.5, 0.01M) gives glucuronic acid 166. Finally, hydrogenolysis of the benzyl group over Pearlman's catalyst gives 167.

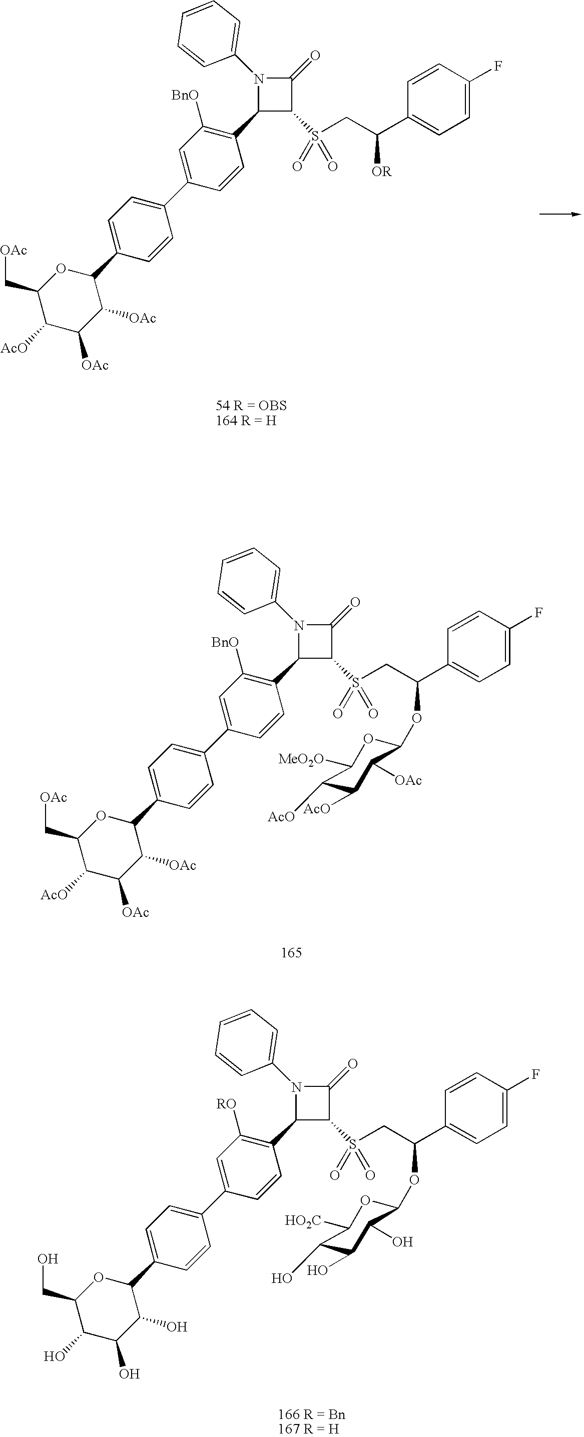

### EXAMPLE 74

Reaction of 153 with di-tert-butylsilyl ditriflate in the presence of triethylamine followed by saponification of the ester with aqueous lithium hydroxide in methanol gives the corresponding acid. The acid is converted to the corresponding mixed anhydride by treatment with pivaloyl chloride and DMAP in DMF and then coupled to (S)-4-phenyl-2-oxazolidinone to afford the imide (168). In the next step, 168 is treated with titanium tetrachloride and N-ethyldiisopropylamine followed by treatment with 6b to effect enantiospecific condensation providing 169. Treatment of 169 with excess N,O-bis(trimethylsilyl)acetamide followed by a catalytic amount of tetrabutylammonium fluoride hydrate results in ring closure to the desired beta-lactam (170). Suzuki coupling of 170 with 11 gives biphenyl derivative 171. Treatment of 171 with aqueous HF removes the silyl protecting group to give diol 172. Reaction of 172 with two equivalents of trichloroacetimidate donor methyl 2,3,4-tri-O-acetyl-T-O-(2,2,2-trichloroethanimidoyl)-□-D-glucopyranuronate (prepared according to Urban, F.; Moore, B.; Breitenbach, R. Tetrahedron Lett. 1990, 31, 4221) in methylene chloride at −25° C. with boron trifluoride etherate as an activator for the coupling gives 173. Treatment of 173 with bromotrimethylsilane removes the ester moieties from the phosphorus. Hydrolysis of the remaining ester group by treatment with TEA:MeOH:H₂O (1:1:3.5, 0.01M) gives glucuronic acids. Finally, hydrogenolysis of the benzyl group over Pearlman's catalyst gives 174.

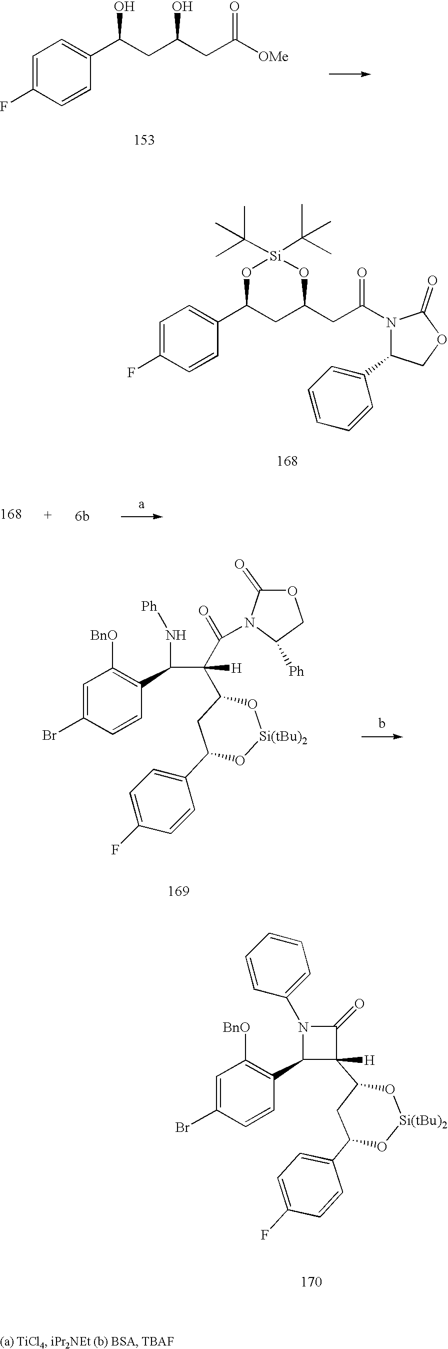

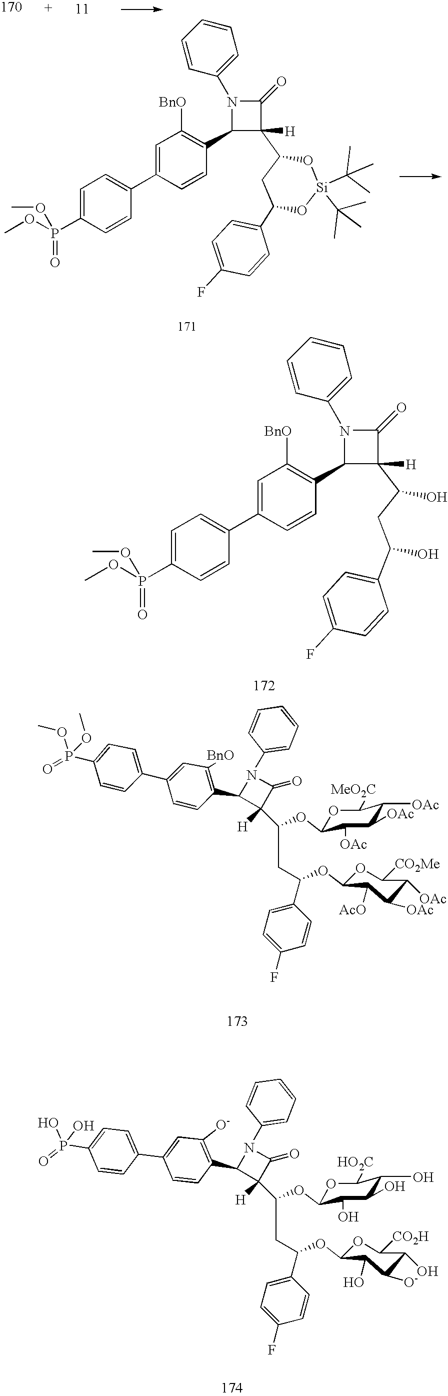

Using the procedures set forth in the above examples, the following additional glucuronide derivatives may also be prepared. However, the invention is not limited to these glucuronide derivatives nor to the compounds provided in the above examples.

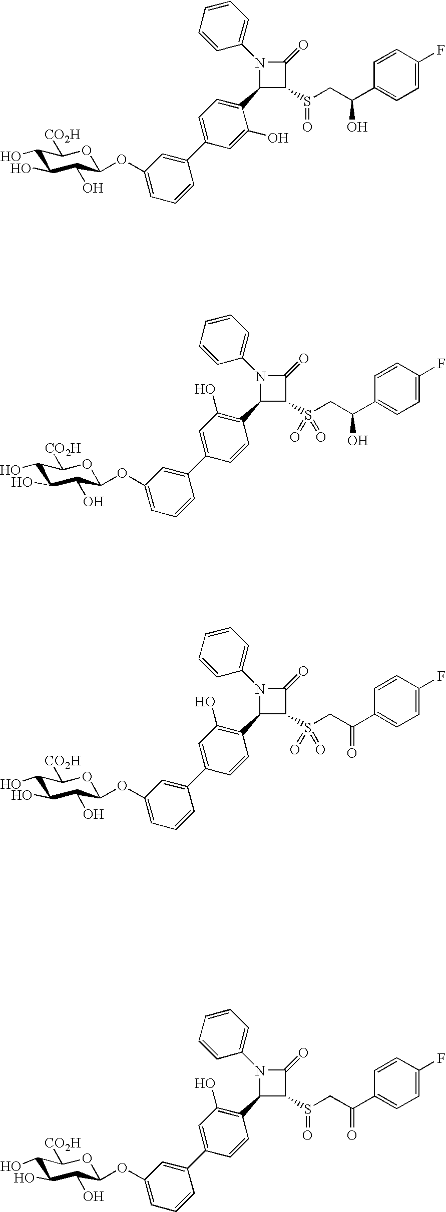

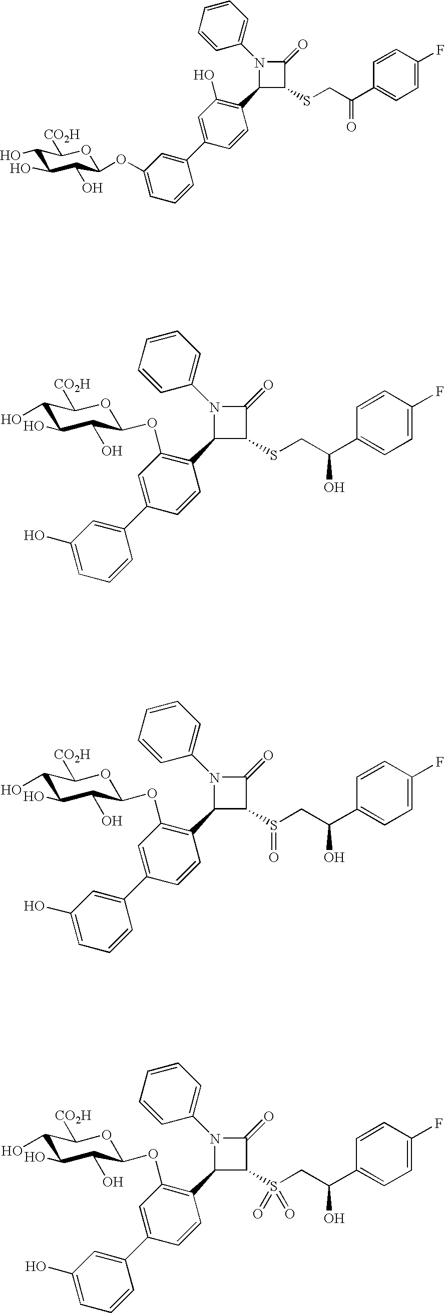

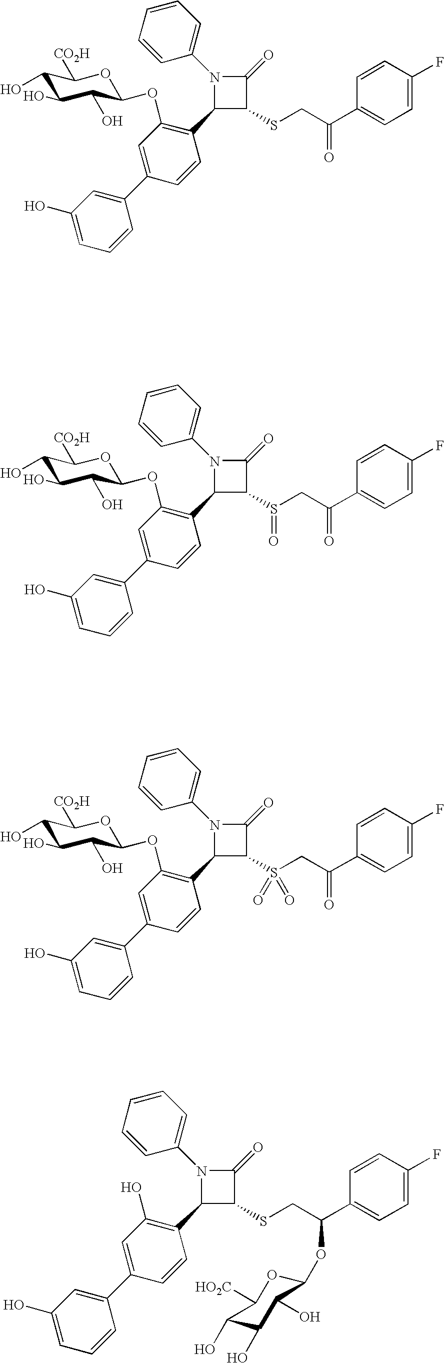

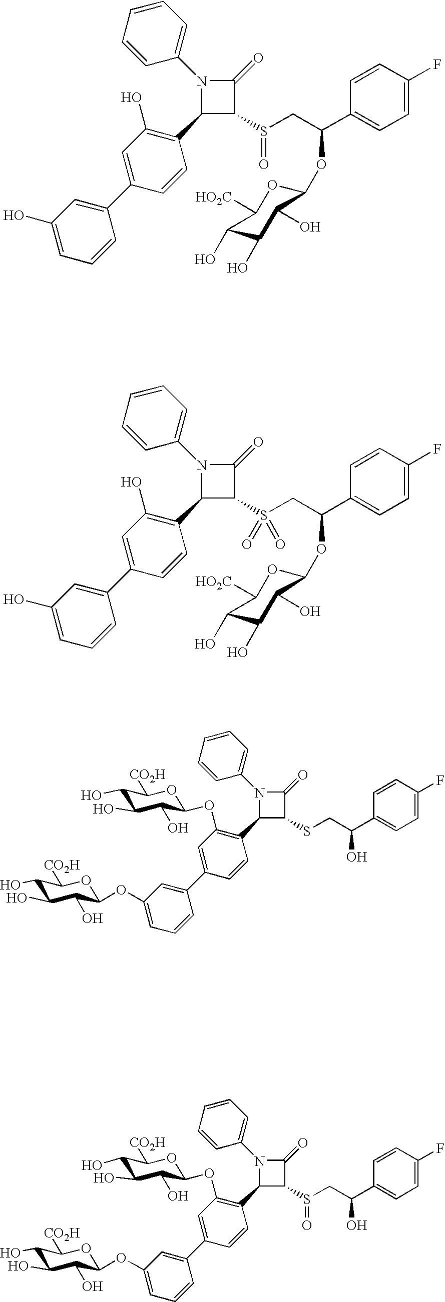

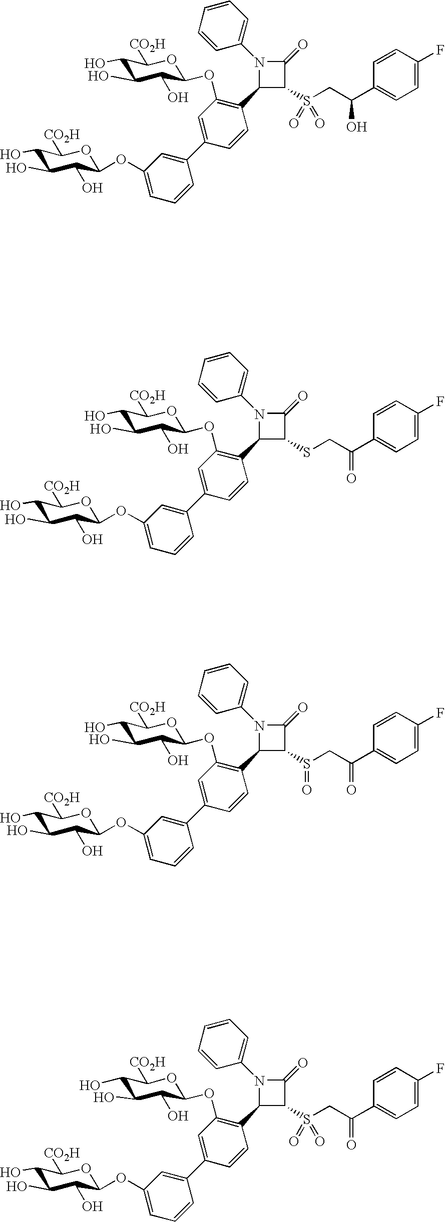

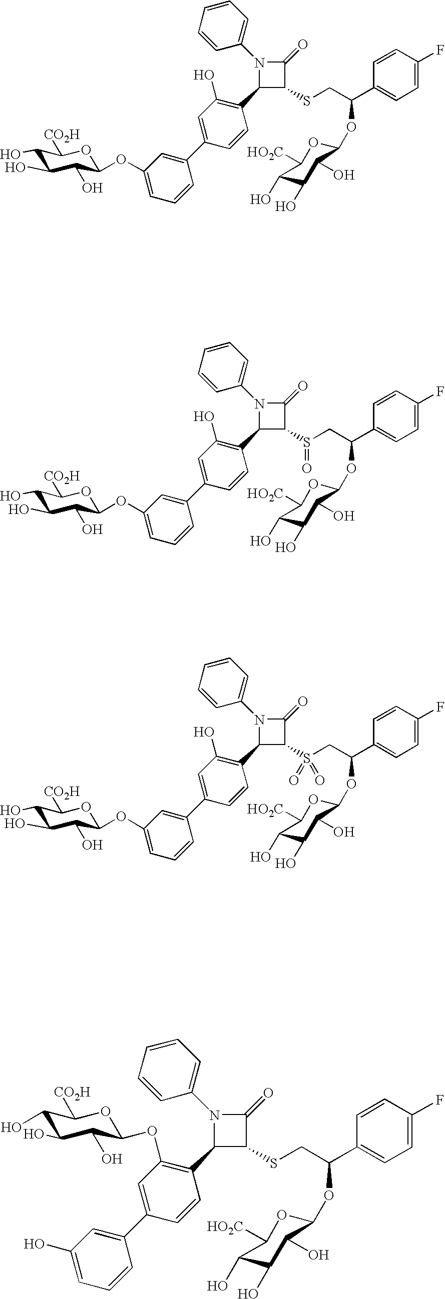

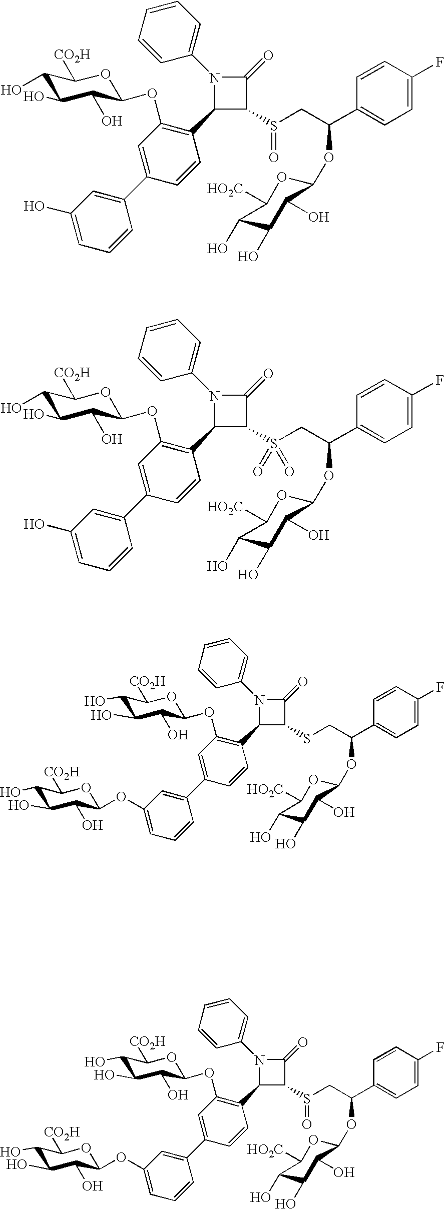

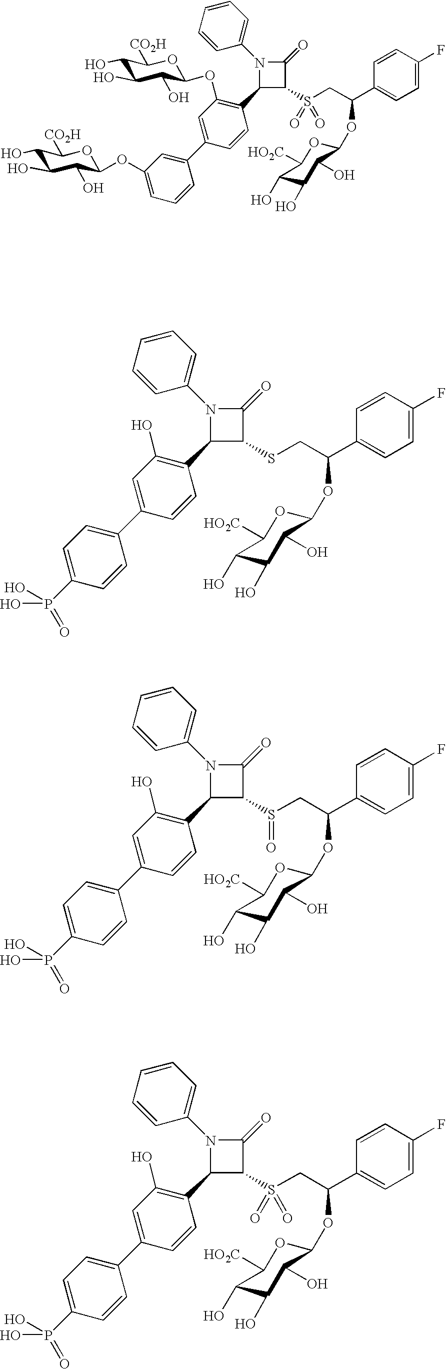

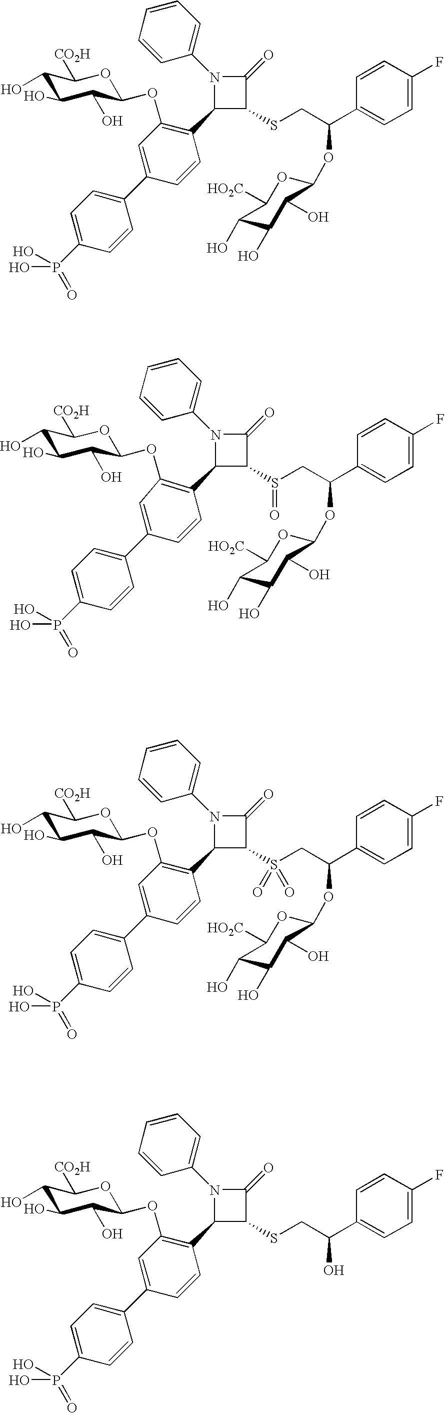

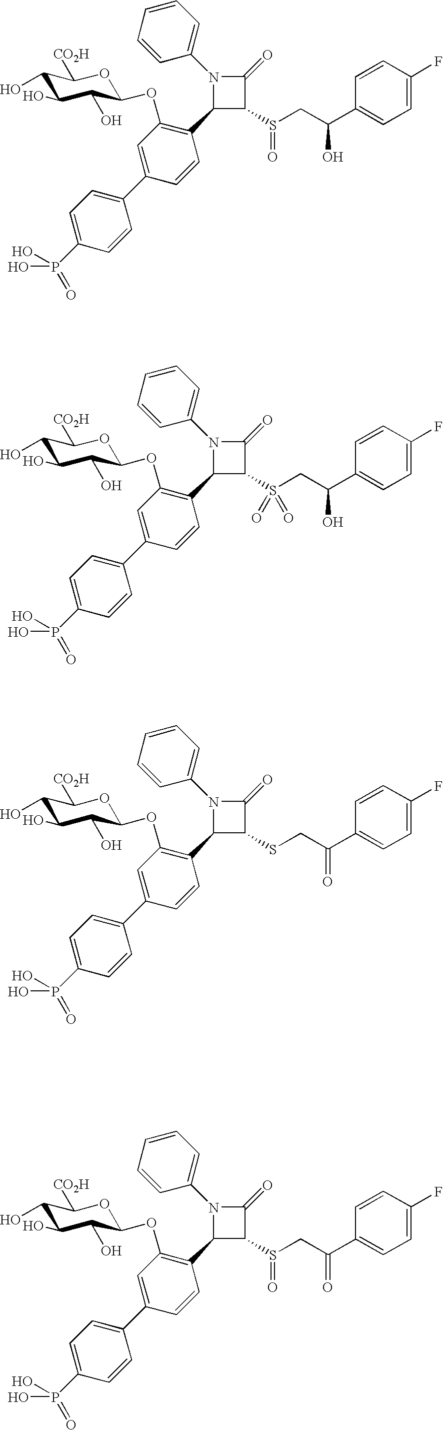

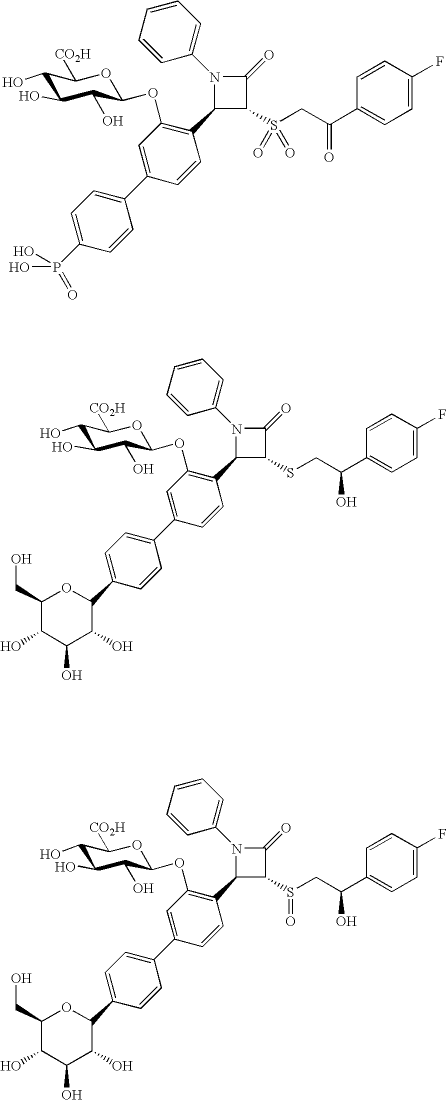

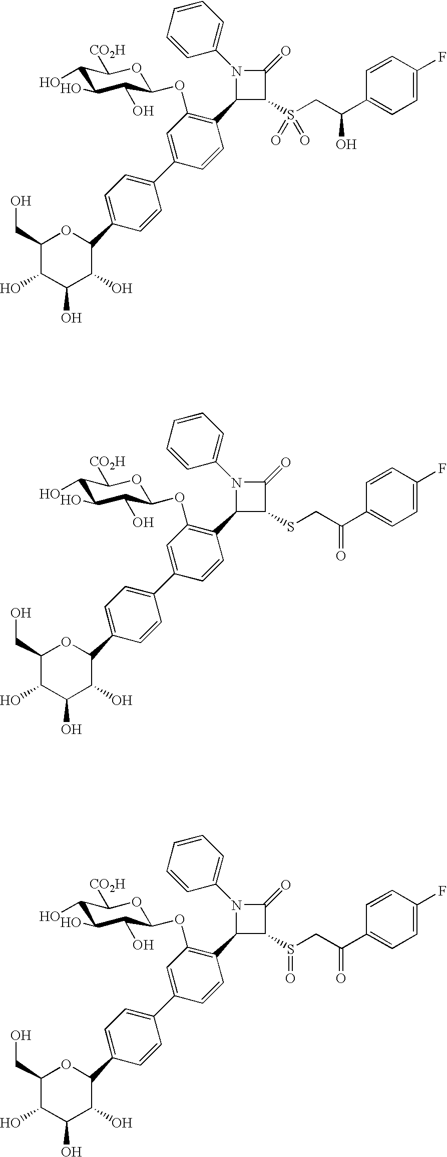

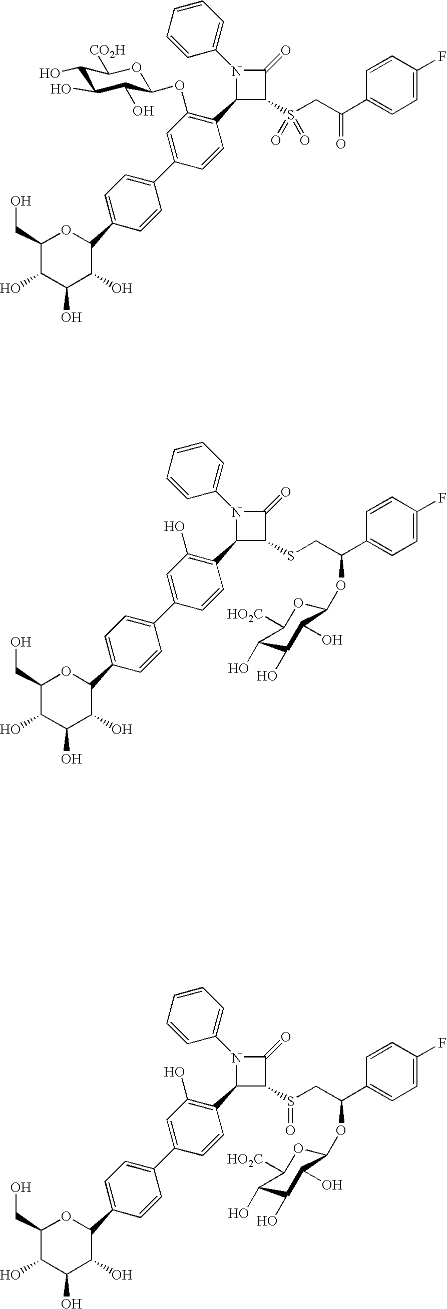

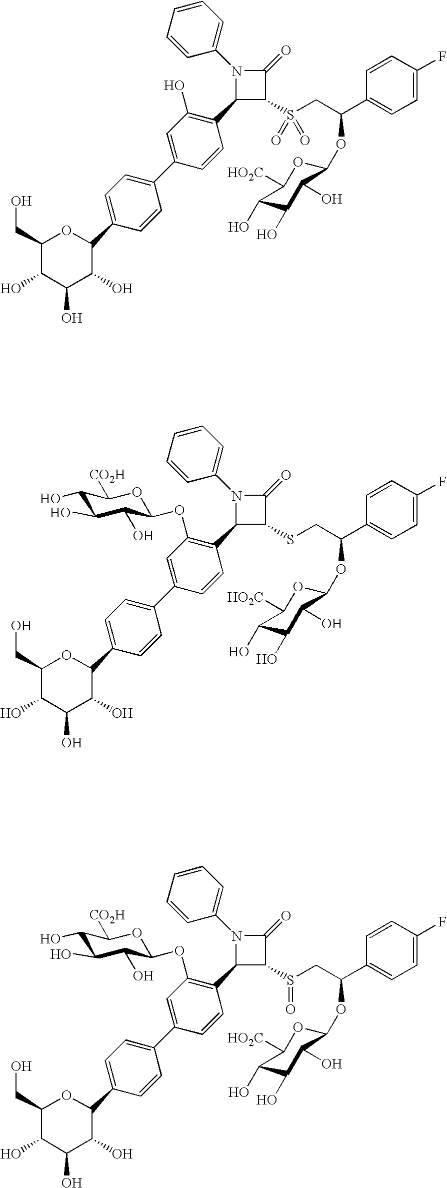

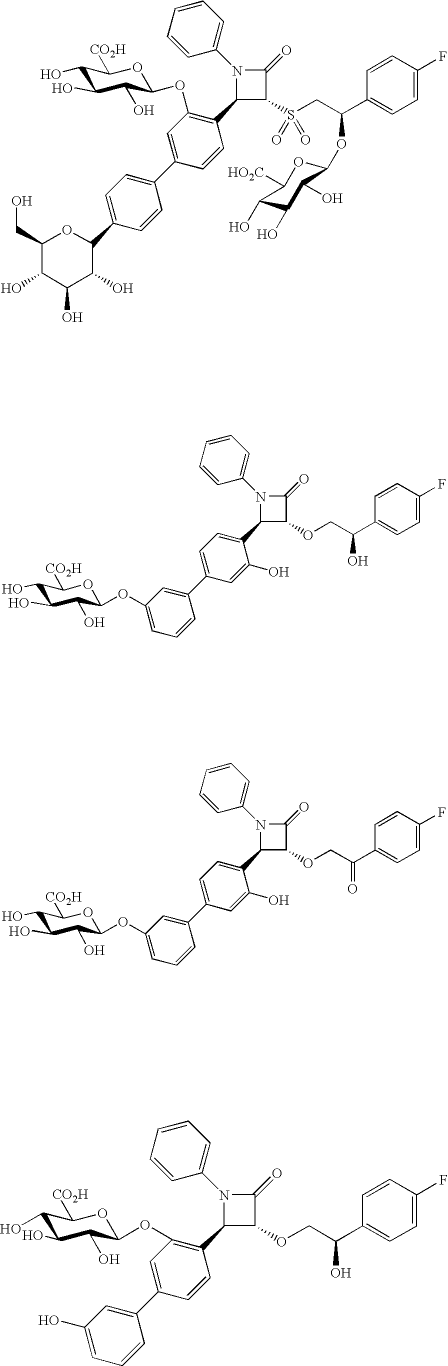

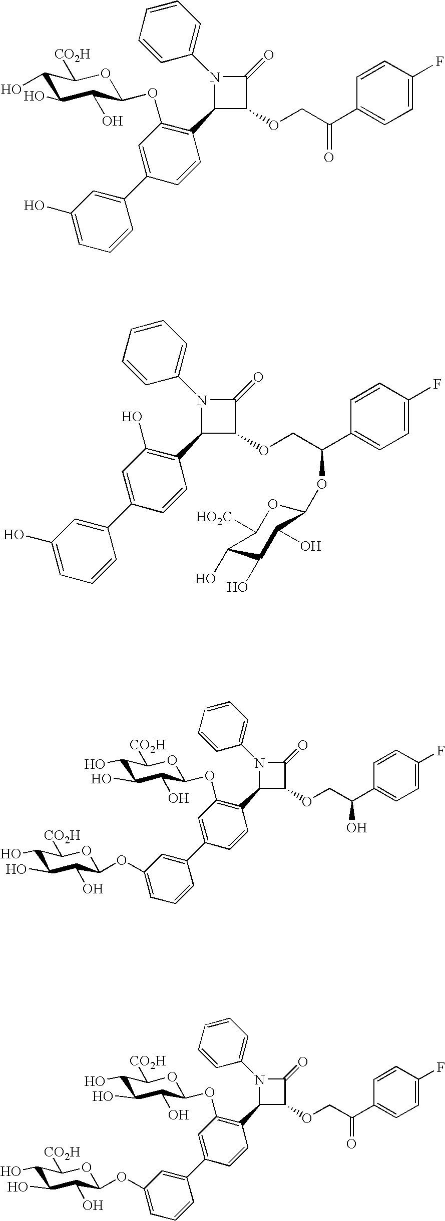

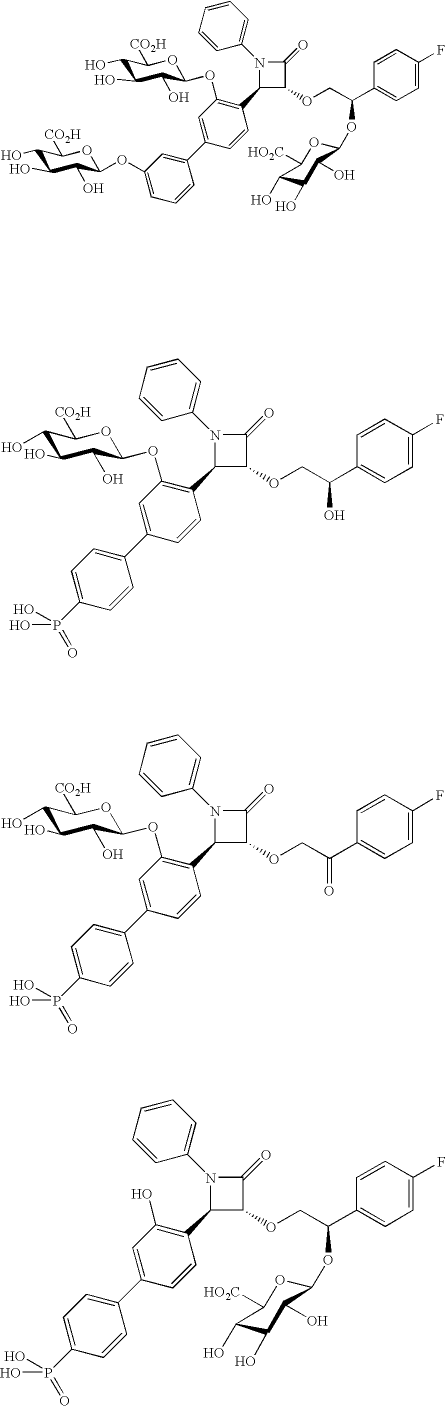

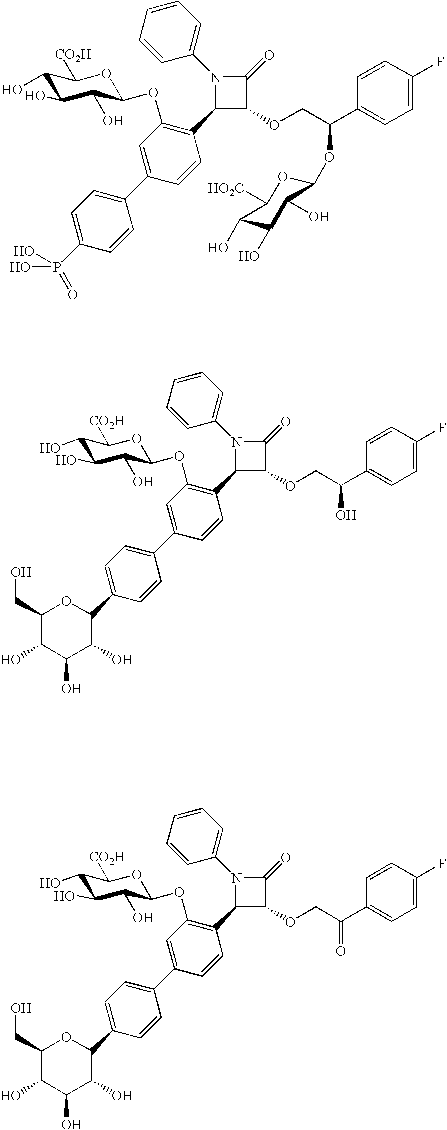

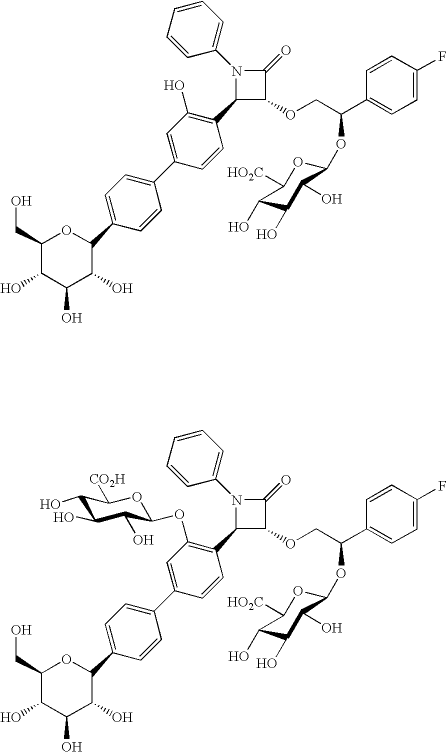

In addition, the following compounds of formula Ia may be prepared using the methods set forth in the above examples. Because no stereochemistry is provided, each structural drawing is intended to encompass the compound in any stereoisomeric form. Furthermore, the invention is not limited to these compounds nor to the compounds provided in the above examples.

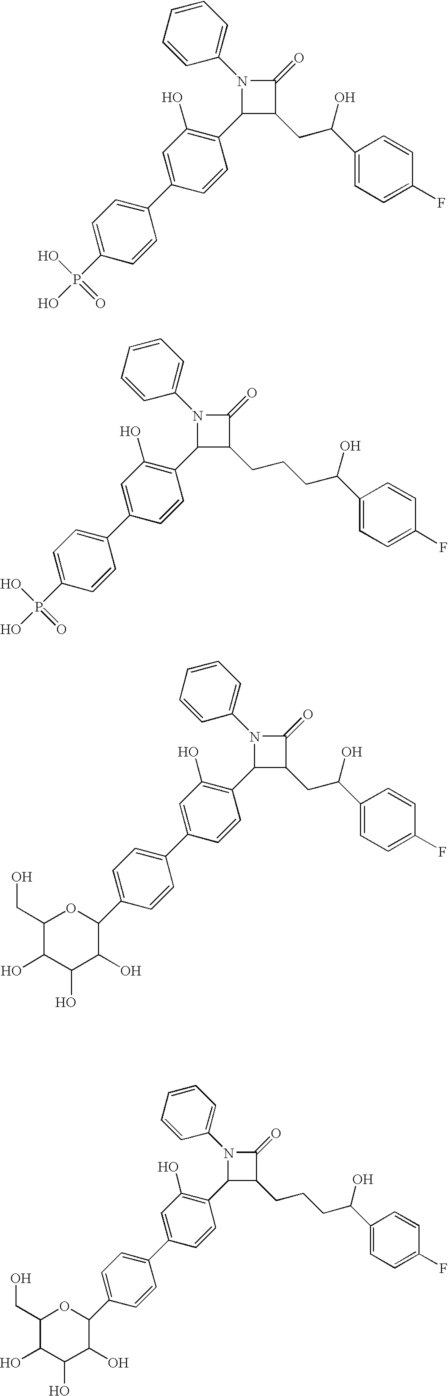

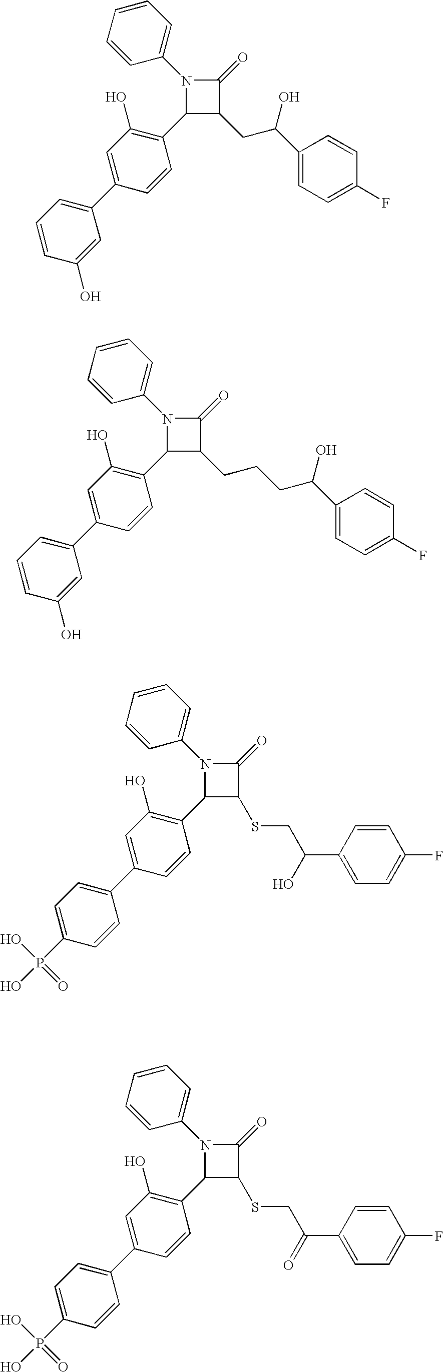

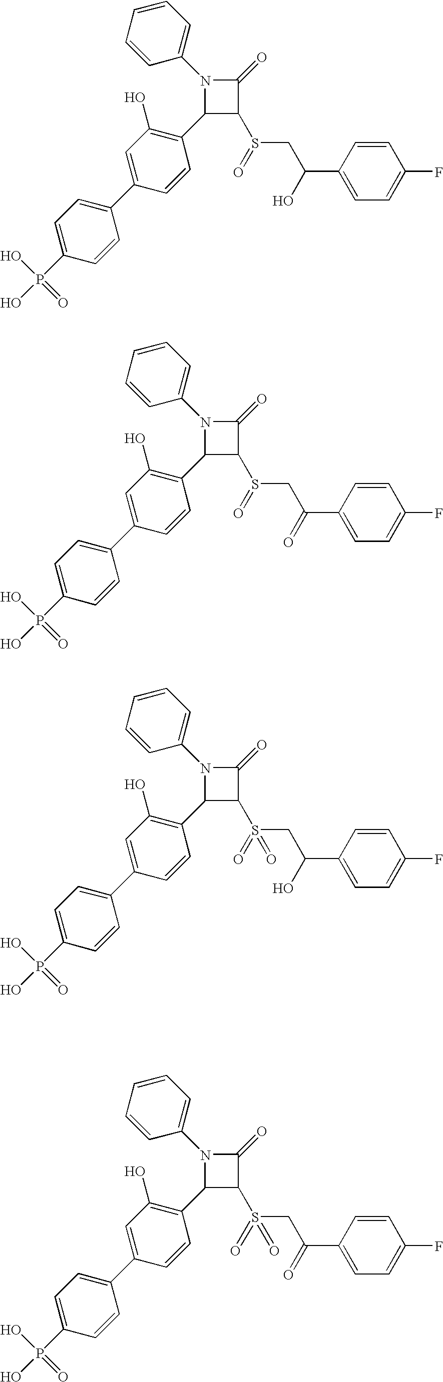

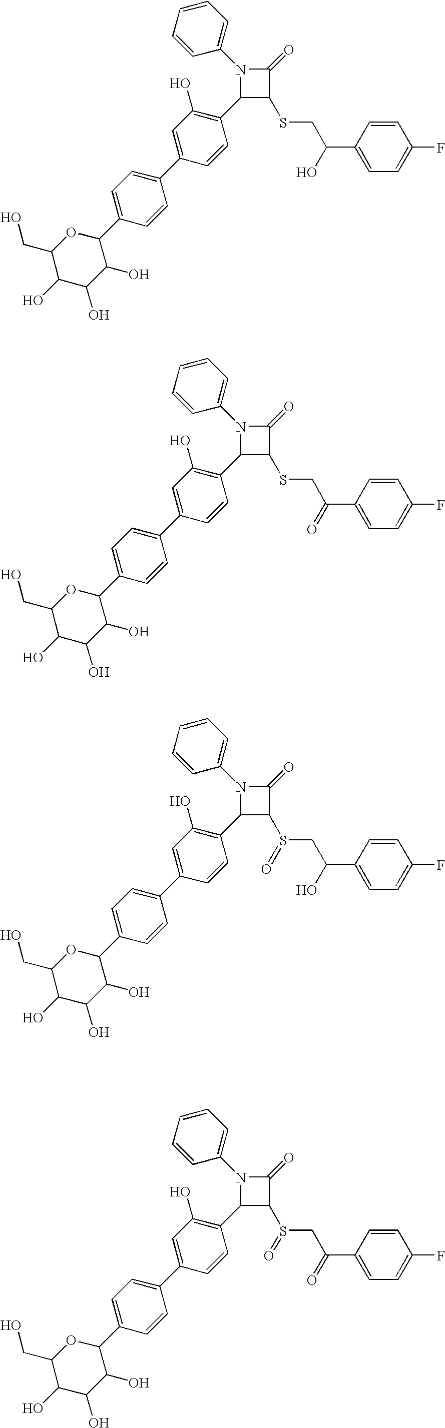

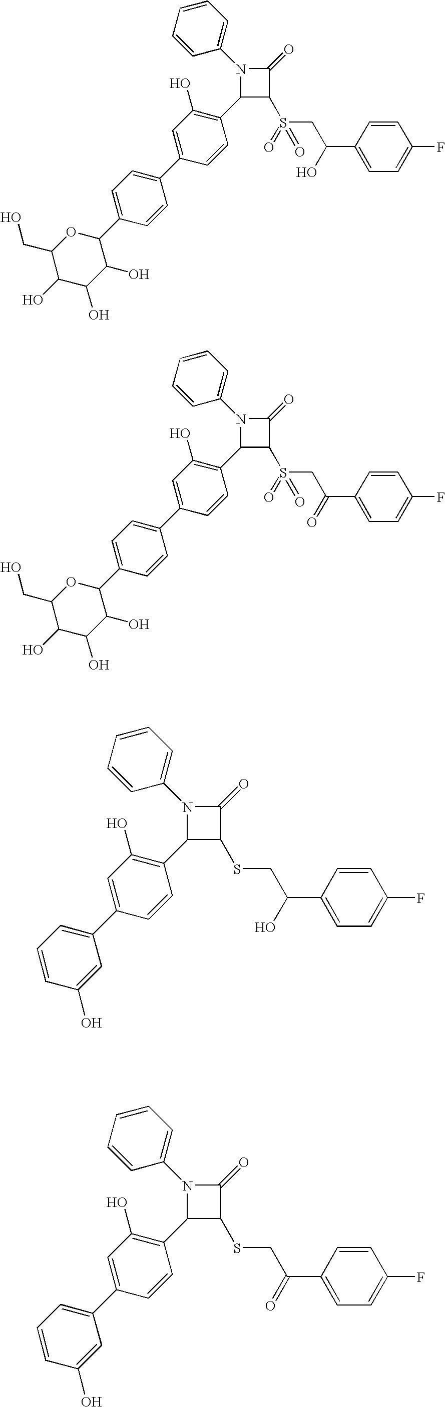

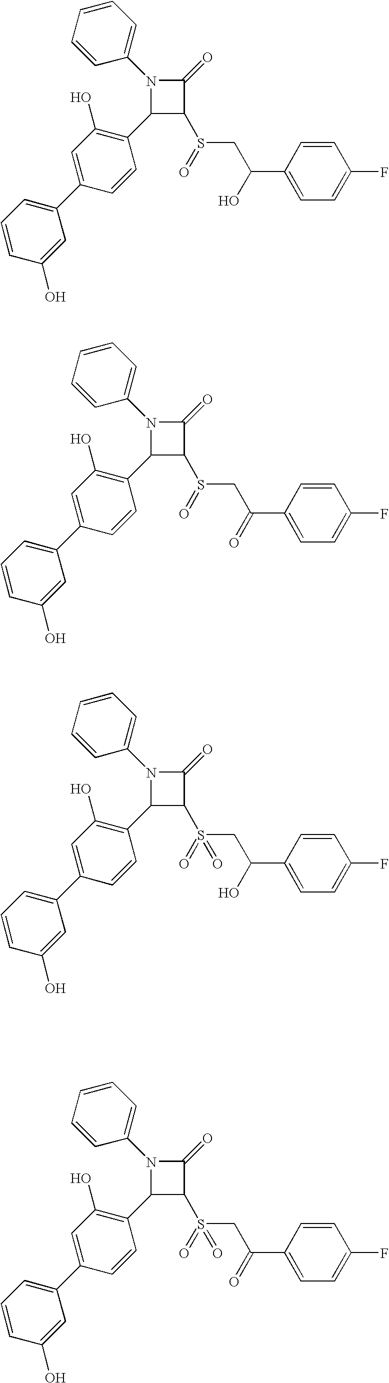

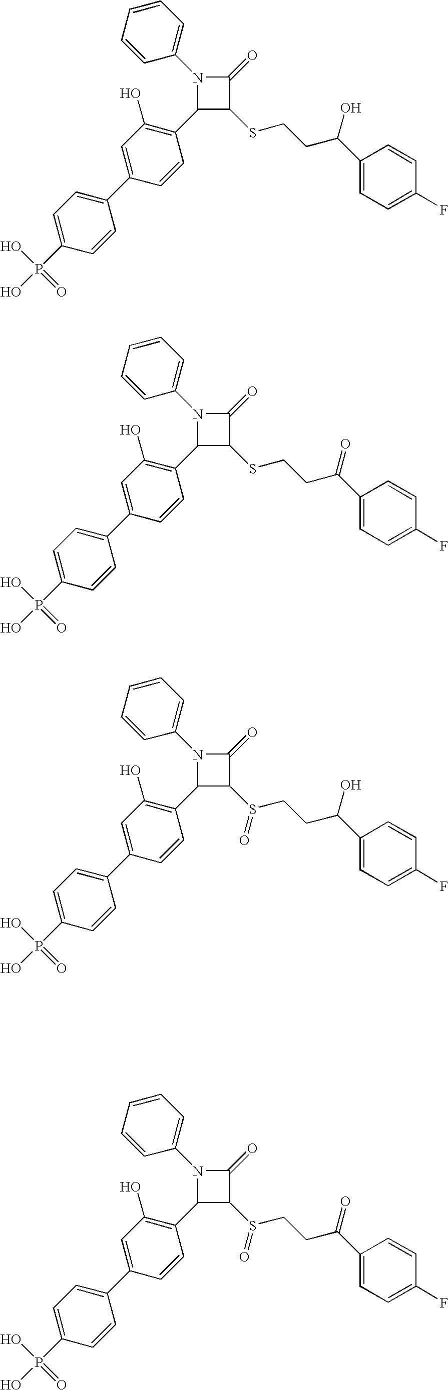

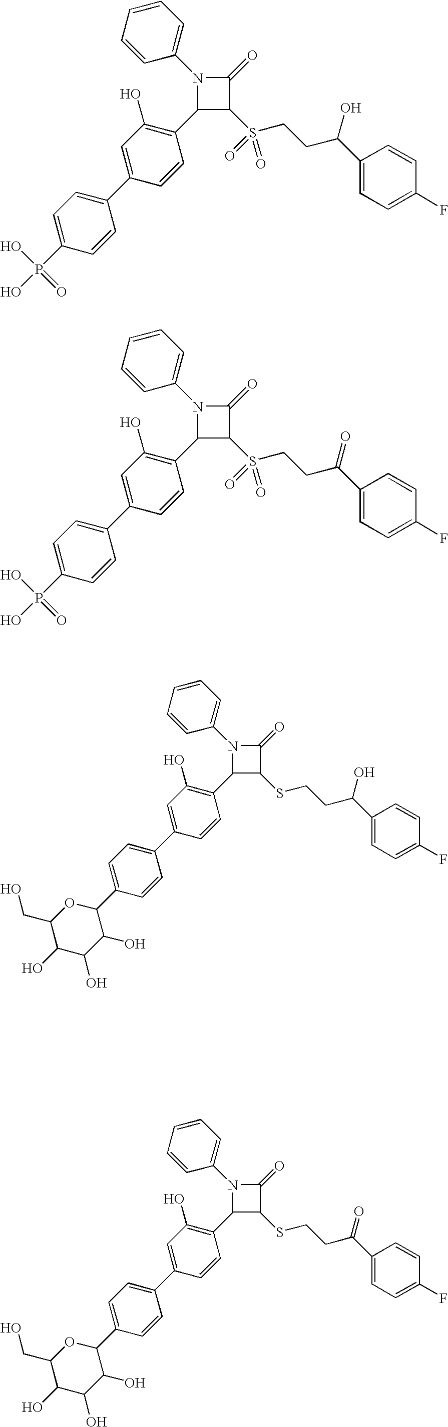

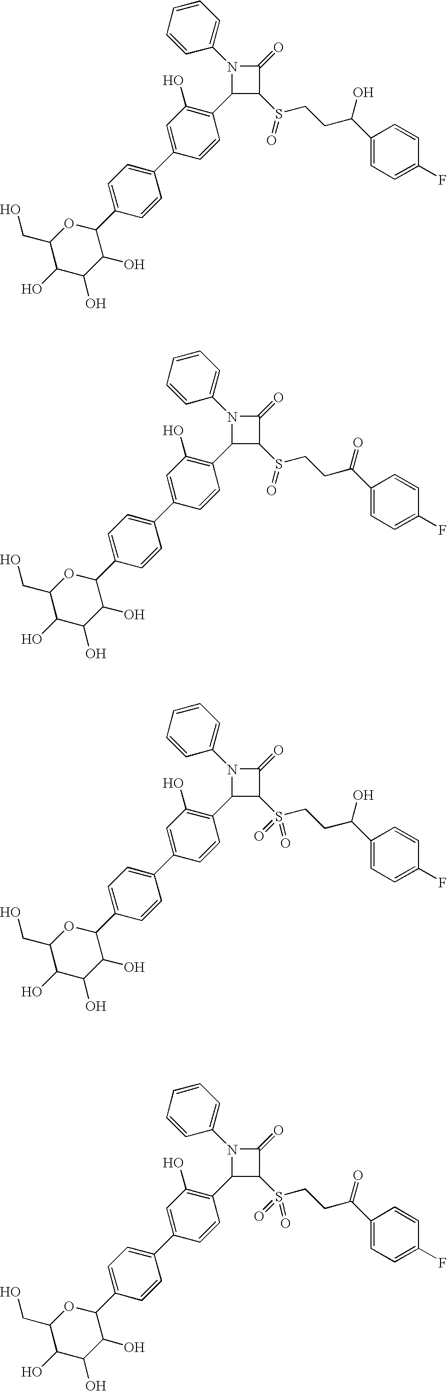

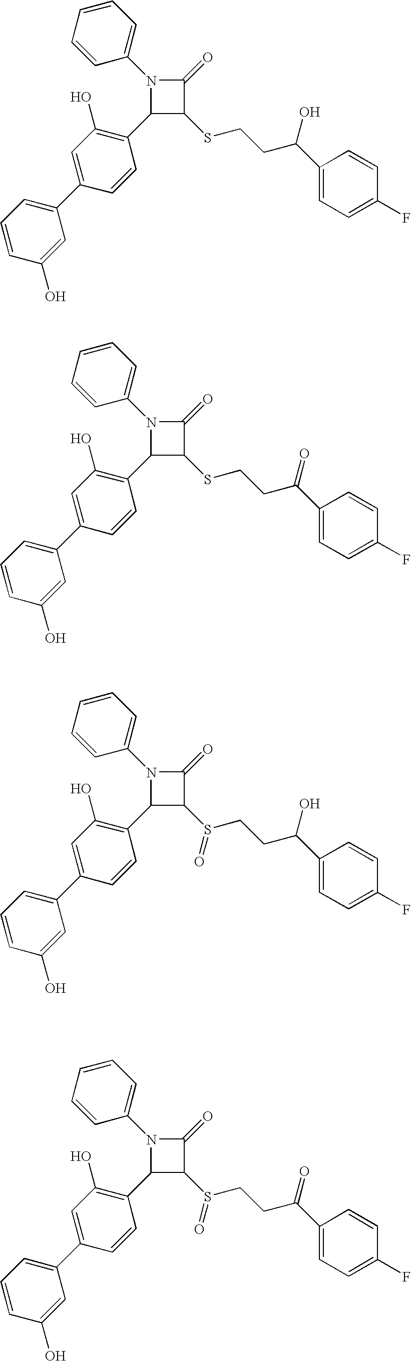

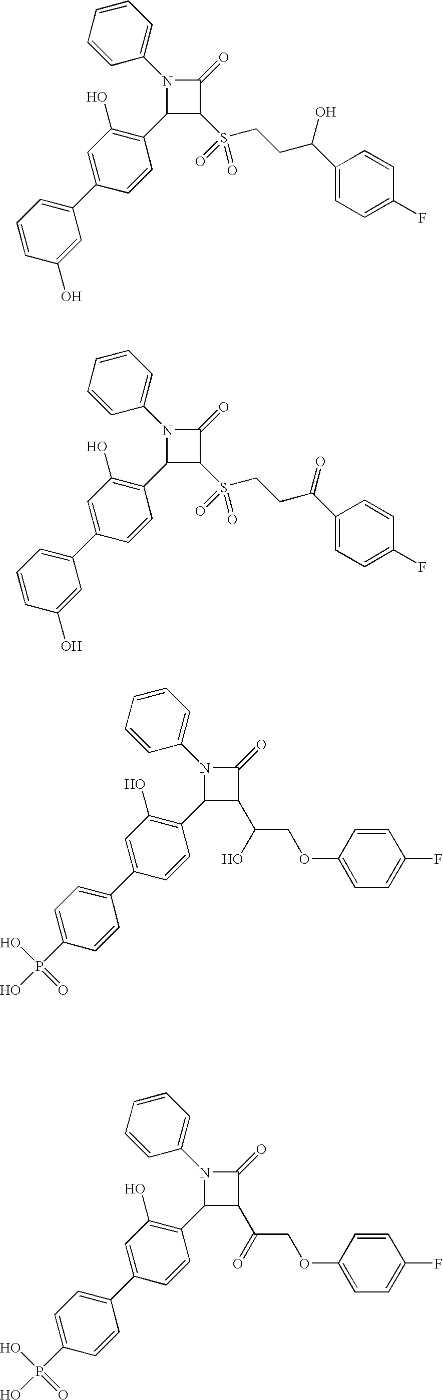

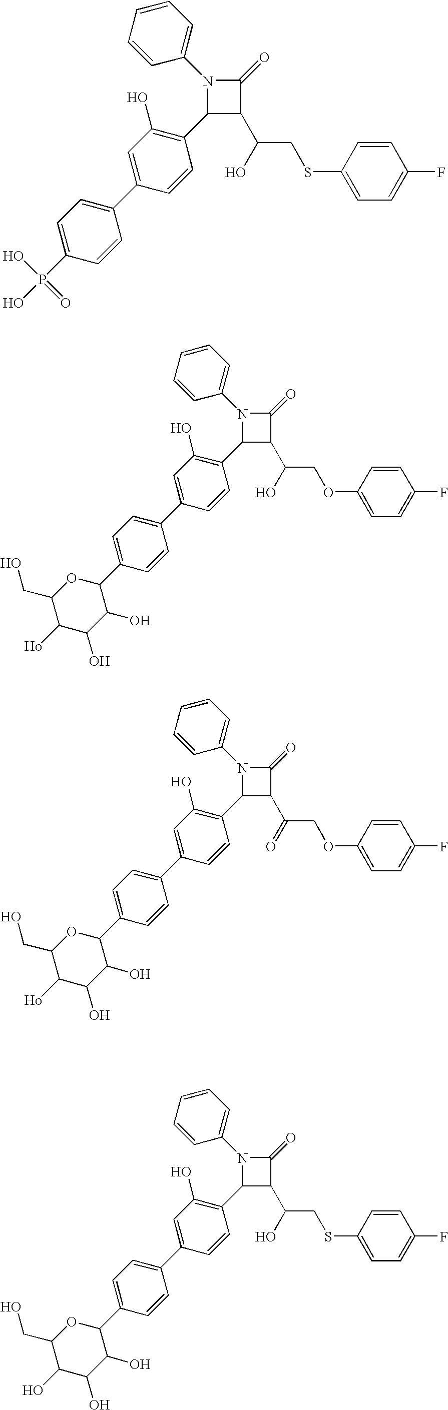

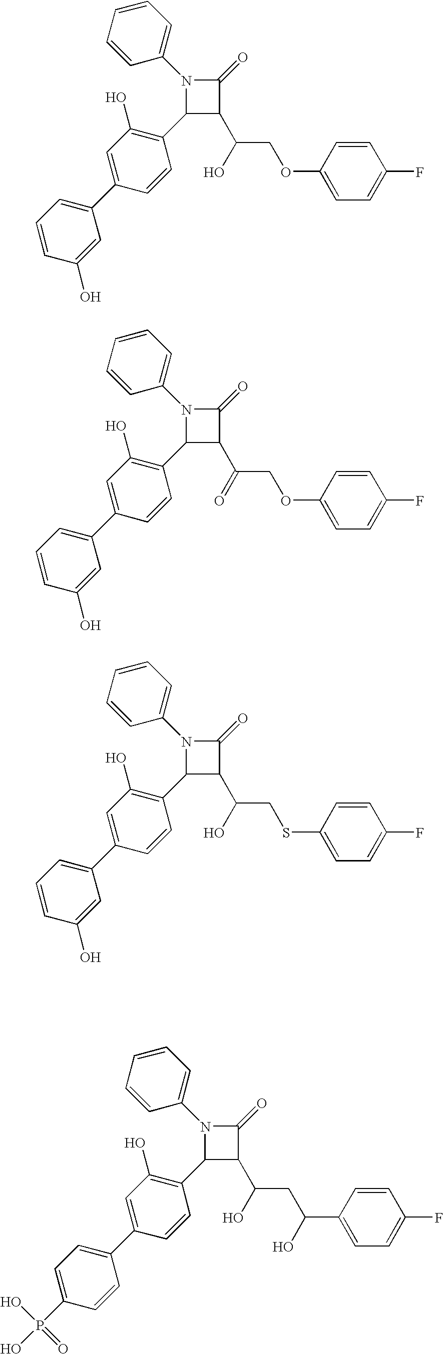

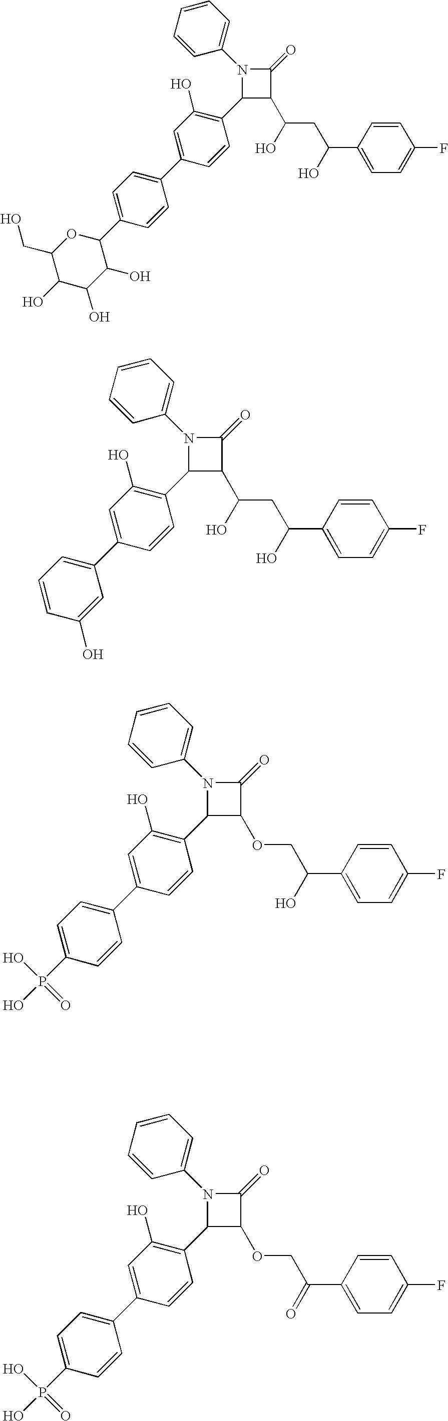

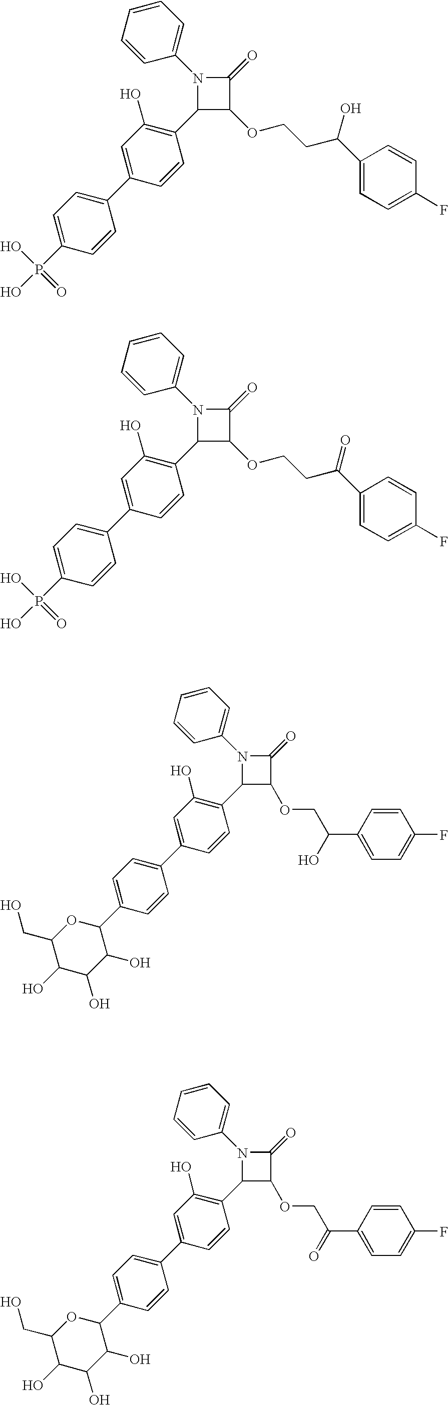

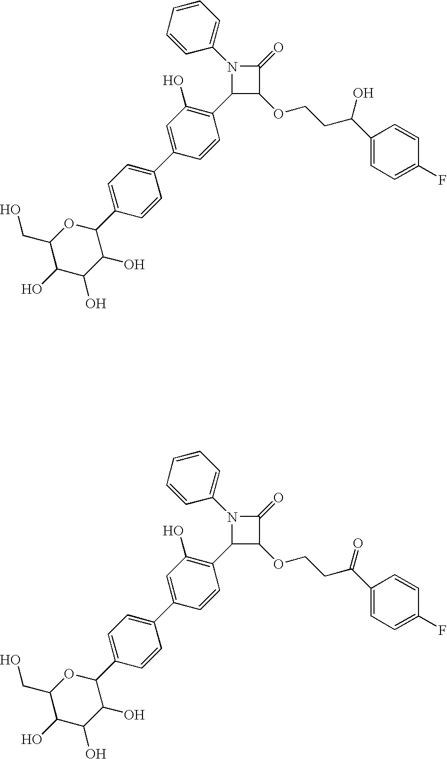

Each of the patents, patent applications, patent publications, and references mentioned herein is hereby incorporated by reference in its entirety.

The invention has been described in detail with particular reference to preferred embodiments thereof, but it will be understood by those skilled in the art that variations and modifications can be effected within the spirit and scope of the invention.

We claim:

1. A compound of formula I 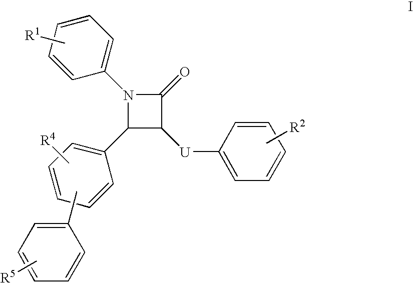
wherein
R¹ and R² are selected independently from the group consisting of hydrogen, halogen, —OH, and lower alkoxy;
R⁴ is selected from the group consisting of hydrogen, —OH, and a glucuronide;
R⁵ is selected from the group consisting of —OH, —PO₃H₂, —SO₃H, a sugar, a polyol, a glucuronide and a sugar carbamate; and
U is (C₂-C₆)-alkylene in which one or more individual —CH₂— is replaced by a radical chosen from —S—, —S(═O)—, —SO₂—, —O—, —C(═O)—, —CH(glucuronide)-, and —CH(OH)—, or a pharmaceutically acceptable salt thereof, in any stereoisomeric form, or a mixture of any such compounds in any ratio.

2. A compound according to claim 1 of formula Ia

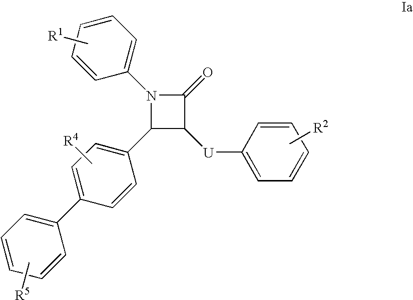

3. A compound according to claim 1, wherein U is (C₂-C₄)-alkylene in which one or more individual —CH₂— is replaced.

4. A compound according to claim 3, wherein one or more individual —CH₂— is replaced by —CH(OH)—.

5. A compound according to claim 3, wherein one or more individual —CH₂— is replaced by —CH(glucuronide)-, —SCH₂CH(OH)—, —S(═O)CH₂CH(OH)—, —SO₂CH₂CH(OH)—, —SCH₂C(═O)—, —S(═O)CH₂C(═O)—, —SO₂CH₂C(═O)— —OCH₂CH(OH)—, —OCH₂C(═O)—, —CH(OH)CH₂O—, —C(═O)CH₂O—, —CH(OH)CH₂S—, —C(═O)CH₂S—, —CH₂CH(OH)—, —CH₂CH₂CH₂CH(OH)—, —OCH₂CH₂C(═O)—, SCH₂CH₂CH(OH)—, —SCH₂CH₂C(═O)—, —S(═O)CH₂CH₂CH(OH)—, —S(═O)CH₂CH₂C(═O)—, —SO₂CH₂CH₂CH(OH)—, —SO₂CH₂CH₂C(═O)—, —CH(OH)CH₂CH(OH)—, or —OCH₂CH₂CH(OH)—.

6-28. (canceled)

29. A compound according to claim 4, wherein one or more —CH(OH)— is replaced with —CH(glucuronide)-.

30. A compound according to claim 1, wherein the 3-position of the azetidinone ring is of the (S)-configuration, and the 4-position of the azetidinone ring is of the (R)-configuration.

31. A compound according to claim 1, wherein the 3-position of the azetidinone ring is of the (R)-configuration, and the 4-position of the azetidinone ring is of the (S)-configuration.

32. A compound according to claim 1, wherein U is (C₂-C₄)-alkylene wherein at least one —CH₂— is replaced by —CH(OH)— or by —CH(glucuronide)-, and wherein each carbon bearing an (OH) or (glucuronide) substituent is of the (S)-configuration.

33. A compound according to claim 1, wherein U is (C₂-C₄)-alkylene wherein at least one —CH₂— is replaced by —CH(OH)— or by —CH(glucuronide)-, and wherein each carbon bearing an (OH) or (glucuronide) substituent is of the (R)-configuration.

34. A compound according to claim 1, wherein R⁴ is —OH.

35. A compound according to claim 1, wherein R⁴ is a glucuronide.

36. A compound according to claim 1, wherein R⁴ is in the ortho position relative to the azetidinone ring.

37. A compound according to claim 1, wherein R² is fluorine.

38. A compound according to claim 37, wherein R² is in the para position.

39. A compound according to claim 1, wherein R¹ is hydrogen.

40. A compound according to claim 1, wherein R⁵ is in the para position.

41. A compound according to claim 1, wherein R⁵ is in the meta position.

42. A pharmaceutical formulation comprising a compound according to claim 1 and a pharmaceutically acceptable carrier.

43. A method for treating hypercholesterolemia comprising administering to a mammal a therapeutically effective amount of a compound according to claim 1.
